(19) United States
(12) Patent Application Publication
(10) Pub. No.: US 2026/0048099 A1
(43) Pub. Date: Feb. 19, 2026

(54) SYNERGISTIC INTERACTIONS FOR IMPROVED CANCER TREATMENT

(71) Applicant: THE UNITED STATES OF AMERICA, AS REPRESENTED BY THE SECRETARY, DEPARMENT OF HEALTH AND HUMAN SERVICE, Bethesda, MD (US)

(72) Inventors: Barbara K. Felber, Rockville, MD (US); George N. Pavlakis, Rockville, MD (US); Sevasti Karaliota, New Market, MD (US); Dimitrios Stellas, New Market, MD (US)

(21) Appl. No.: 19/101,938

(22) PCT Filed: Aug. 16, 2023
(86) PCT No.: PCT/US2023/072333

§ 371 Date: Feb. 7, 2025

Related U.S. Application Data
(60) Provisional application No. 63/398,450, filed on Aug. 16, 2022.

(51) Int. Cl. A61K 38/20; A61K 31/17; A61K 31/197; A61K 31/255; A61K 31/337; A61K 31/375; A61K 31/4184; A61K 31/506; A61K 31/661; A61K 31/675; A61K 31/69; A61K 31/704; A61K 31/7068; A61K 31/7076; A61K 33/36; A61K 38/07; A61K 38/13; A61K 38/17; A61K 39/395; A61P 35/00

(52) U.S. Cl. CPC A61K 38/208; A61K 31/17; A61K 31/197; A61K 31/255; A61K 31/337; A61K 31/375; A61K 31/4184; A61K 31/506; A61K 31/661; A61K 31/675; A61K 31/69; A61K 31/704; A61K 31/7068; A61K 31/7076; A61K 33/36; A61K 38/07; A61K 38/13; A61K 38/1793; A61K 38/2086; A61K 39/3955; A61P 35/00

(57) ABSTRACT

This disclosure provides compositions and methods comprising combinations of IL-15 or an IL-15/IL-15Ra complex with one or more other active agents for the treatment of cancer. This disclosure also provides compositions and methods comprising combinations of IL-15 or an IL-15/IL-15Rct complex fused to IL-12 or a derivative thereof, and with one or more other active agents for the treatment of cancer. In some embodiments, the one or more active agents comprises an activator of PPAR. In some embodiments, the one or more active agents comprises inhibitor of FLT3. In some embodiments, the one or more active agents comprises a chemotherapeutic agent.

### CROSS-REFERENCE TO RELATED APPLICATIONS

This application claims the benefit of U.S. Provisional Application No. 63/398,450, filed Aug. 16, 2022, which is incorporated by reference herein in its entirety.

### STATEMENT REGARDING FEDERALLY SPONSORED RESEARCH

This work was made with government support under the National Institutes of Health, National Institutes of Health Intramural program of CCR/NCI. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Aug. 16, 2023, is named “22-1190-WO_Sequence-Listing.xml” and is 67,011 bytes in size.

### BACKGROUND

### Field of the Disclosure

This disclosure generally relates to compositions and methods for treating cancer. The compositions and methods comprise the combination of IL-15 with one or more other active agents.

### Description of Related Art

Interleukin-15 (IL-15) is a member of the four alpha-helix bundle family of lymphokines and plays a pivotal role in modulating the activity of both the innate and adaptive immune system (e.g., expansion and maintenance of the memory T-cell response to invading pathogens, and induction of Natural Killer (NK) cell proliferation and cytotoxic activity). IL-15 is expressed as a heterodimer (hetIL-15) of the IL-15 polypeptide chain and the IL-15 receptor alpha (IL-15Rα). IL-15 specifically binds to the IL-15Rα with high affinity via the “sushi domain” in exon 2 of the extracellular domain of the receptor. Endogenous heterodimeric IL-15 is found in two forms, as a membrane-bound form that is expressed by antigen presenting and stroma cells in various tissues; and as a soluble extracellular complex of IL-15 to the soluble IL-15Rα, which is produced by cleavage of the membrane-anchored IL-15Rα by cellular proteases. IL-15 has been widely studied for its antitumor effects as a heterodimer (hetIL-15 and variants) or as a single chain IL-15.

Based on its critical and complex roles in the immune system, studies have continued to explore the therapeutic use of IL-15. In particular, as shown herein it has been discovered that novel combinations with IL-15 enhance the anti-tumor effects of hetIL-15 in certain cancer models.

### SUMMARY

It is against the above background that the present disclosure provides certain advantages over the prior art.

Although this disclosure as provided herein is not limited to specific advantages or functionalities, the disclosure provides compositions and methods comprising the combination of IL-15 with one or more other active agents.

In one aspect, this disclosure provides for methods for treating cancer in a subject, comprising administering to the subject a composition comprising:
(a) an IL-15 or a derivative thereof or an IL-15/IL-15 receptor alpha (IL-15Rα) complex or a derivative thereof; and
(b) one or more active agents.

In certain embodiments of the methods disclosed herein, the one or more active agents comprises an activator of PPAR and/or an inhibitor of FLT3. In certain embodiments, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments of the methods disclosed herein, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments of the methods disclosed herein, the one or more active agents comprises a chemotherapeutic agent. In some embodiments, the chemotherapeutic agent comprises a chemotherapeutic agent selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof. In certain embodiments, the one or more active agents is gemcitabine.

In certain embodiments of the methods disclosed herein, the IL-15 or derivative thereof or the IL-15/IL-15 receptor alpha (IL-15Rα) complex or derivative thereof comprises any of the sequences as disclosed in Table 1 or Table 4. In certain embodiments of the methods disclosed herein, the IL-15 or derivative thereof or the IL-15/IL15Rα complex or derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the methods disclosed herein, the one or more active agents is a PPAR activator and the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the one or more active agents is a FLT3 inhibitor and the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the IL-15 or derivative thereof or the IL-15/IL15Rα complex or derivative thereof is administered via locoregional administration to the cancer. In certain embodiments, the IL-15 or derivative thereof or the IL15-/IL15Rα complex or derivative thereof is administered subcutaneously, intramuscularly, intravenously, by peritumoral injection, or by intratumoral injection.

In certain embodiments of the methods disclosed herein, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, the disclosure provides for methods for treating cancer in a subject, comprising administering to the subject a composition comprising:
(a) an IL-15 or a derivative thereof or an IL-15/IL-15 receptor alpha (IL-15Rα) complex or a derivative thereof; and
(b) an activator of PPAR.

In certain embodiments of the methods disclosed herein, the activator of PPAR is Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, or WY-14643 (Pirinixic Acid). In certain embodiments, the activator of PPAR is fenofibrate.

In certain embodiments, the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the IL-15 or derivative thereof or the IL-15/IL-15 receptor alpha (IL-15Rα) complex or derivative thereof comprises any of the sequences as disclosed in Table 1 or Table 4.

In certain embodiments of the methods disclosed herein, the IL-15 or derivative thereof or the IL-15/IL15Rα complex or derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the methods disclosed herein, the IL-15 or derivative thereof or the IL-15/IL15Rα complex or derivative thereof is administered via locoregional administration to the cancer. In certain embodiments, the IL-15 or derivative thereof or the IL-15/IL15Rα complex or derivative thereof is administered subcutaneously, intramuscularly, intravenously, by peritumoral injection, or by intratumoral injection.

In certain embodiments of the methods disclosed herein, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, the disclosure provides for methods for treating cancer in a subject, comprising administering to the subject a composition comprising:
(a) an IL-15 or a derivative thereof or an IL-15/IL-15 receptor alpha (IL-15Rα) complex or a derivative thereof; and
(b) an inhibitor of FLT3.

In certain embodiments of the methods disclosed herein, the inhibitor of FLT3 is AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), or XL 999. In certain embodiments, the inhibitor of FLT3 is quizartinib (AC220).

In certain embodiments of the methods disclosed herein, the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the IL-15 or derivative thereof or the IL-15/IL-15 receptor alpha (IL-15Rα) complex or derivative thereof comprises any of the sequences as disclosed in Table 1 or Table 4.

In certain embodiments of the methods disclosed herein, the IL-15 or derivative thereof or the IL-15/IL15Rα complex or derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the methods disclosed herein, the IL-15 or derivative thereof or the IL-15/IL15Rα complex or derivative thereof is administered via locoregional administration to the cancer. In certain embodiments, the IL-15 or derivative thereof or the IL-15/IL15Rα complex or derivative thereof is administered intravenously, by peritumoral injection, or by intratumoral injection.

In certain embodiments of the methods disclosed herein, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, the disclosure provides compositions comprising:
(a) an IL-15 or a derivative thereof or an IL-15/IL-15 receptor alpha (IL-15Rα) complex or a derivative thereof; and
(b) one or more active agents.

In certain embodiments of the compositions disclosed herein, the one or more active agents comprises an activator of PPAR and/or an inhibitor of FLT3.

In certain embodiments of the compositions disclosed herein, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments of the compositions disclosed herein, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments of the methods disclosed herein, the one or more active agents comprises a chemotherapeutic agent. In some embodiments, the chemotherapeutic agent comprises a chemotherapeutic agent selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof. In certain embodiments, the one or more active agents is gemcitabine.

In certain embodiments of the compositions disclosed herein, the IL-15 or derivative thereof or the IL-15/IL-15 receptor alpha (IL-15Rα) complex or derivative thereof comprises any of the sequences as disclosed in Table 1 or Table 4.

In certain embodiments of the compositions disclosed herein, the IL-15 or derivative thereof or the IL-15/IL15Rα complex or derivative thereof dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the compositions disclosed herein, the one or more active agents is a PPAR activator and the PPAR activator dose in the composition is about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the compositions disclosed herein, the one or more active agents is a FLT3 inhibitor and the FLT3 inhibitor dose in the composition is about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In another aspect, the disclosure provides compositions, comprising:
(a) an IL-15 or a derivative thereof or an IL-15/IL-15 receptor alpha (IL-15Rα) complex or a derivative thereof; and
(b) an activator of PPAR.

In certain embodiments of the compositions disclosed herein, the activator of PPAR is Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, or WY-14643 (Pirinixic Acid). In certain embodiments, the activator of PPAR is fenofibrate.

In certain embodiments of the compositions disclosed herein, the PPAR activator dose in the compositions is about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the compositions disclosed herein, the IL-15 or derivative thereof or the IL-15/IL-15 receptor alpha (IL-15Rα) complex or derivative thereof comprises any of the sequences as disclosed in Table 1 or Table 4.

In certain embodiments of the compositions disclosed herein, the IL-15 or derivative thereof or the IL-15/IL15Rα complex or derivative thereof dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In another aspect, the disclosure provides compositions for treating cancer in a subject, comprising:
(a) an IL-15 or a derivative thereof or an IL-15/IL-15 receptor alpha (IL-15Rα) complex or a derivative thereof; and
(b) an inhibitor of FLT3.

In certain embodiments of the compositions disclosed herein, the inhibitor of FLT3 is AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), or XL 999. In certain embodiments, the inhibitor of FLT3 is quizartinib (AC220).

In certain embodiments of the compositions disclosed herein, the FLT3 inhibitor dose in the composition is about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the compositions disclosed herein, the IL-15 or derivative thereof or the IL-15/IL-15 receptor alpha (IL-15Rα) complex or derivative thereof comprises any of the sequences as disclosed in Table 1 or Table 4.

In certain embodiments of the compositions disclosed herein, the IL-15 or derivative thereof or the IL-15/IL15Rα complex or derivative dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, or about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the compositions disclosed herein, the IL-15 or derivative thereof or the IL-15/IL15Rα complex or derivative thereof is formulated to be administered intravenously, by peritumoral injection, or by intratumoral injection.

In an aspect, the disclosure provides for pharmaceutical compositions comprising any of the compositions as disclosed herein.

In another aspect, the disclosure provides for methods for treating cancer in a subject, comprising administering to the subject a composition, comprising:
(a) an agonistic compound engaging heterodimeric IL-2/IL-15 Receptor beta-gamma; and
(b) one or more active agents.

In certain embodiments of the methods disclosed herein, the one or more active agents comprises an activator of PPAR and/or an inhibitor of FLT3.

In certain embodiments of the methods disclosed herein, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments of the methods disclosed herein, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments of the methods disclosed herein, the one or more active agents comprises a chemotherapeutic agent. In some embodiments, the chemotherapeutic agent comprises a chemotherapeutic agent selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof. In certain embodiments, the one or more active agents is gemcitabine.

In certain embodiments of the methods disclosed herein, the agonistic compound engaging heterodimeric IL-2/IL-15 Receptor beta-gamma comprises:
(a) molecules binding to IL-2R-beta and/or IL-2Receptor-Gamma, and not preferentially binding to the trimeric IL-2 Receptor, which contains in addition IL-2Receptor alpha;
(b) Alt-803 (N-803);
(c) fusion molecules of IL-15 to the Sushi domain of IL-15Ra;
(d) modified cytokines IL-2/IL-15 Receptor beta-gamma and preventing binding to IL-2Ra; or
(e) fusion molecules that have dual function as IL-15 and something else, and are used to either enhance the function of IL-15 or to target IL-15 to specific locations.

In certain embodiments of the methods disclosed herein, the agonistic compound engaging heterodimeric IL-2/IL-15 Receptor beta-gamma is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, or about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the methods disclosed herein, the one or more active agents is a PPAR activator and the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the one or more active agents is a FLT3 inhibitor and the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments of the methods disclosed herein, the agonistic compound engaging heterodimeric IL-2/IL-15 Receptor beta-gamma is administered via locoregional administration to the cancer. In certain embodiments, the agonistic compound engaging heterodimeric IL-2/IL-15 Receptor beta-gamma is administered subcutaneously, intramuscularly, intravenously, by peritumoral injection, or by intratumoral injection.

In certain embodiments of the methods disclosed herein, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, the disclosure provides compositions comprising:
(a) an agonistic compound engaging heterodimeric IL-2/IL-15 Receptor beta-gamma; and
(b) one or more active agents.

In certain embodiments of the compositions disclosed herein, the one or more active agents comprises an activator of PPAR and/or an inhibitor of FLT3.

In certain embodiments of the compositions disclosed herein, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments of the compositions disclosed herein, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments of the methods disclosed herein, the one or more active agents comprises a chemotherapeutic agent. In some embodiments, the chemotherapeutic agent comprises a chemotherapeutic agent selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof. In certain embodiments, the one or more active agents is gemcitabine.

In certain embodiments of the compositions disclosed herein, the agonistic compound engaging heterodimeric IL-2/IL-15 Receptor beta-gamma comprises:
(a) molecules binding to IL-2R-beta and/or IL-2Receptor-Gamma, and not preferentially binding to the trimeric IL-2 Receptor, which contains in addition IL-2Receptor alpha;
(b) Alt-803 (N-803);
(c) fusion molecules of IL-15 to the Sushi domain of IL-15Ra;
(d) modified cytokines to prevent binding to IL-2Ra; or
(e) fusion molecules that have dual function as IL-15 and something else, and are used to either enhance the function of IL-15 or to target IL-15 to specific locations.

In certain embodiments of the compositions disclosed herein, the agonistic compound engaging heterodimeric IL-2/IL-15 Receptor beta-gamma dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, or about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the compositions disclosed herein, the one or more active agents is a PPAR activator and the PPAR activator dose in the composition is about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the compositions disclosed herein, the one or more active agents is a FLT3 inhibitor and the FLT3 inhibitor dose in the composition is about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195.

In yet another aspect, this disclosure provides a composition comprising a fusion protein comprising: (a) IL-15 or a derivative thereof or IL-15 receptor alpha (IL-15Rα) complex or a derivative thereof; and (b) IL-12 or a derivative thereof.

In certain embodiments of the compositions disclosed herein, the compositions further comprise one or more active agents. In certain embodiments, the one or more active agents comprises an activator of PPAR, an inhibitor of FLT3, and/or a chemotherapeutic agent.

In certain embodiments, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments, the one or more active agents comprises a chemotherapeutic agent, and the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In certain embodiments, the IL-15 or derivative thereof or the IL-15/IL-15 receptor alpha (IL-15Rα) complex or derivative thereof comprises any of the sequences as disclosed in Table 1 or Table 4.

In certain embodiments, the IL-15 or derivative thereof or the IL-15/IL15Rα complex or derivative thereof dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the one or more active agents is a PPAR activator and the PPAR activator dose in the composition is about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments, the one or more active agents is a FLT3 inhibitor and the FLT3 inhibitor dose in the composition is about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In an aspect, this disclosure provides a method for treating cancer in a subject, comprising administering to the subject a composition comprising: (a) IL-15 or a derivative thereof or IL-15/IL-15 receptor alpha (IL-15Rα) complex or a derivative thereof; and (b) IL-12 or a derivative thereof.

In certain embodiments of the methods disclosed herein, the method further comprises administering one or more active agents. In certain embodiments, the one or more active agents comprises an activator of PPAR, an inhibitor of FLT3, and/or a chemotherapeutic agent.

In certain embodiments, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments, the one or more active agents comprises a chemotherapeutic agent, and the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In certain embodiments, the IL-15 or derivative thereof or the IL-15/IL-15 receptor alpha (IL-15Rα) complex or derivative thereof comprises any of the sequences as disclosed in Table 1 or Table 4.

In certain embodiments, the IL-15 or derivative thereof or the IL-15/IL15Rα complex or derivative thereof dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the one or more active agents is a PPAR activator and the PPAR activator dose in the composition is about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments, the one or more active agents is a FLT3 inhibitor and the FLT3 inhibitor dose in the composition is about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments, the IL-15 or derivative thereof or the IL-15/IL15Rα complex or derivative thereof is administered via locoregional administration to the cancer.

In certain embodiments, the IL-15 or derivative thereof or the IL15-/IL15Rα complex or derivative thereof is administered subcutaneously, intramuscularly, intravenously, by peritumoral injection, or by intratumoral injection.

In certain embodiments, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, this disclosure provides a fusion protein for the treatment of cancer in a subject, wherein fusion protein comprises: (a) IL-15 or a derivative thereof or IL-15/IL-15 receptor alpha (IL-15Rα) complex or a derivative thereof; and (b) IL-12 or a derivative thereof.

In certain embodiments, the fusion protein further comprising one or more active agents.

In certain embodiments, the one or more active agents comprises an activator of PPAR, an inhibitor of FLT3, and/or a chemotherapeutic agent.

In certain embodiments, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, wherein the one or more active agents is fenofibrate.

In certain embodiments, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments, the one or more active agents comprises a chemotherapeutic agent, and the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In certain embodiments, the IL-15 or derivative thereof or the IL-15/IL-15 receptor alpha (IL-15Rα) complex or derivative thereof comprises any of the sequences as disclosed in Table 1 or Table 4.

In certain embodiments, the IL-15 or derivative thereof or the IL-15/IL15Rα complex or derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the one or more active agents is a PPAR activator and the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments, the one or more active agents is a FLT3 inhibitor and the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments, IL-15 or derivative thereof or the IL-15/IL15Rα complex or derivative thereof is administered via locoregional administration to the cancer.

In certain embodiments, the IL-15 or derivative thereof or the IL15-/IL15Rα complex or derivative thereof is administered subcutaneously, intramuscularly, intravenously, by peritumoral injection, or by intratumoral injection.

In certain embodiments, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In an aspect, this disclosure provides, combination therapy for treating cancer in a subject, wherein the combination therapy comprises: (a) an IL-15 or a derivative thereof or an IL-15/IL-15 receptor alpha (IL-15Rα) complex or a derivative thereof; and (b) one or more active agents.

In certain embodiments of the combination therapy, the one or more active agents comprises an activator of PPAR, an inhibitor of FLT3, and/or a chemotherapeutic agent.

In certain embodiments, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the one or more active agents is fenofibrate.

In certain embodiments, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, the one or more active agents is quizartinib (AC220).

In certain embodiments, the one or more active agents comprises a chemotherapeutic agent, and the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In certain embodiments, the IL-15 or derivative thereof or the IL-15/IL-15 receptor alpha (IL-15Rα) complex or derivative thereof comprises any of the sequences as disclosed in Table 1 or Table 4.

In certain embodiments, the IL-15 or derivative thereof or the IL-15/IL15Rα complex or derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the one or more active agents is a PPAR activator and the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments, the one or more active agents is a FLT3 inhibitor and the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments, the IL-15 or derivative thereof or the IL-15/IL15Rα complex or derivative thereof is administered via locoregional administration to the cancer.

In certain embodiments, the IL-15 or derivative thereof or the IL15-/IL15Rα complex or derivative thereof is administered subcutaneously, intramuscularly, intravenously, by peritumoral injection, or by intratumoral injection.

In certain embodiments, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

In another aspect, this disclosure provides a combination therapy for treating cancer in a subject, wherein the combination therapy comprises: (a) an agonistic compound engaging heterodimeric IL-2/IL-15 Receptor beta-gamma; and (b) one or more active agents.

In certain embodiments of the combination therapy, the one or more active agents comprises an activator of PPAR, an inhibitor of FLT3, and/or a chemotherapeutic agent.

In certain embodiments of the combination therapy, the one or more active agents is an activator of PPAR and the activator of PPAR is selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments of the combination therapy, the one or more active agents is fenofibrate.

In certain embodiments of the combination therapy, the one or more active agents is an inhibitor of FLT3 and the inhibitor of FLT3 is selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments of the combination therapy, the one or more active agents is quizartinib (AC220).

In certain embodiments of the combination therapy, the one or more active agents comprises a chemotherapeutic agent, and the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

In certain embodiments of the combination therapy, the agonistic compound engaging heterodimeric IL-2/IL-15 Receptor beta-gamma comprises: (a) molecules binding to IL-2R-beta and/or IL-2Receptor-Gamma, and not preferentially binding to the trimeric IL-2 Receptor, which contains in addition IL-2Receptor alpha; (b) Alt-803 (N-803); (c) fusion molecules of IL-15 to the Sushi domain of IL-15Ra; (d) modified cytokines IL-2/IL-15 Receptor beta-gamma and preventing binding to IL-2Ra; or (e) fusion molecules that have dual function as IL-15 and something else, and are used to either enhance the function of IL-15 or to target IL-15 to specific locations.

In certain embodiments of the combination therapy, the agonistic compound engaging heterodimeric IL-2/IL-15 Receptor beta-gamma is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, or about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments of the combination therapy, the one or more active agents is a PPAR activator and the PPAR activator is administered at a dose of about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more.

In certain embodiments of the combination therapy, the one or more active agents is a FLT3 inhibitor and the FLT3 inhibitor is administered at a dose of about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg about 250 mg, about 300 mg, about 400 mg, about 450 mg, about 500 mg, about 600 mg, about 700 mg, about 800 mg, about 900 mg, about 1000 mg, or more.

In certain embodiments of the combination therapy, the agonistic compound engaging heterodimeric IL-2/IL-15 Receptor beta-gamma is administered via locoregional administration to the cancer.

In certain embodiments of the combination therapy, the agonistic compound engaging heterodimeric IL-2/IL-15 Receptor beta-gamma is administered subcutaneously, intramuscularly, intravenously, by peritumoral injection, or by intratumoral injection.

In certain embodiments of the combination therapy, the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present disclosure can be best understood when read in conjunction with the following drawings, where like structure is indicated with like reference numerals and in which:

FIG. 1A-1H show hetIL-15 administration resulted in significant EO771 tumor growth delay and increased survival. FIG. 1A, Timeline of the tumor treatment. On day −1, C57BL/6 mice were inoculated with 3×10⁵ EO771 cells (orthotopically in the 4th mammary pad). Mice with palpable tumors were distributed in different groups 7 days later and treated with five locoregional [in the vicinity of the tumor within the breast fat pad] hetIL-15 injections (5 μg/mouse/dose) every 4 days. FIG. 1B, FIG. 1C, Tumor growth (bold lines represent average values) (FIG. 1B) of C57BL/6 wt mice through day 28, when all the mice were alive and Kaplan-Meier survival curve (FIG. 1C) of EO771 tumor allografts in C57BL/6 mice treated with hetIL-15 or vehicle (control). Data shown are from one experiment with 8-10 mice per group and shown as mean±SEM. FIG. 1D-FIG. 1G, Tumor immune infiltrates were analyzed by flow cytometry to determine absolute numbers of cells per gram of tissue: CD8⁺T (FIG. 1D), NK (FIG. 1E), Granzyme B⁺ or ki67⁺ CD8⁺T (FIG. 1F) and Granzyme B⁺ or ki67⁺ NK (FIG. 1G) cells. Data of three independent experiments with 4-6 mice per group were combined; bars represent mean±SEM. FIG. 1H, Gene expression analysis of EO771 tumors recovered from mice treated with either PBS (control, n=3) or hetIL-15 (5 μg/dose/mouse every 4 days) (n=3) was performed by the Nanostring technology using a panel of 780 immune-oncology related gene probes (PanCancer Immune Profiling Panel). The analysis was conducted 48 hours post 3^{rd} hetIL-15 injection. Volcano plot depicts differentially expressed genes between the two treatment groups, highlighting upregulated genes on hetIL-15 treatment. Dashed line represents adjusted p-value=0.05 and dotted lines represent log 2(FC)=1 and log 2(FC)=−1.

FIG. 2A-2G show treatment resulted in increased oxygen consumption rate (OCR), mitochondrial function and fatty acid uptake, revealing a more metabolically active phenotype. FIG. 2A-FIG. 2C, Oxygen consumption rate (OCR) (FIG. 2A), spare respiratory capacity (SRC) (FIG. 28) and OCR/ECAR (Extracellular acidification rate) ratio (FIG. 2C) of tumor-infiltrating CD8⁺T cells from EO771-bearing mice. SRC is calculated as the difference between initial, basal OCR values, and the maximal OCR values achieved after FCCP uncoupling. Data are representative of three independent experiments. All error bars represent SEM. FIG. 2D-FIG. 2G, Timeline of the tumor hetIL-15 and Fenofibrate (FF) combined treatment (FIG. 2D). On day −1, C57BL/6 mice were inoculated with 3×10⁵ EO771 cells (in the 4th mammary pad). Mice with palpable tumors were distributed in different groups 7 days later and treated with three locoregional hetIL-15 injections (5 μg/mouse/dose) every 4 days and FF (50 mg/kg), daily by gavage. FIG. 2E, OCR of tumor-infiltrating CD8⁺T cells was measured using an extracellular flux analyzer. FIG. 2F, FIG. 2G, Mitotracker (FIG. 2F) and Bodipy FL C₁₆ (FIG. 2G) incorporation in tumor-infiltrating CD8⁺T cells from EO771-bearing mice. Data are from one experiment and error bars represent SEM.

FIG. 3A-FIG. 3B show combined treatment of IL-15 immunotherapy and FF resulted in statistically significant EO771 tumor growth delay and complete eradication of the tumors in 85% of mice. FIG. 3A, Timeline of the tumor treatment. On day −1, C57BL/6 mice were inoculated with 3×10⁵ EO771 cells (orthotopically in the 4th mammary pad). Mice with palpable tumors were distributed in different groups 7 days later and treated with four locoregional hetIL-15 injections (5 μg/mouse/dose) every 4 days and FF (50 mg/kg) daily by gavage. FIG. 36, Tumor growth of C57BL/6 mice through day 19 after the beginning of the treatment.

FIG. 4A-FIG. 4G show a novel dendritic cell population is detected in the hetIL-15 treated tumors. FIG. 4A-FIG. 4C, Flow cytometry analysis of intratumoral CD103⁺cDC1 (FIG. 4A), CD11b⁺cDC2 (FIG. 4B) and CD103^{int}CD11b⁺DC (FIG. 4C) populations in controls and hetIL-15 treated mice. Data in graph are given as absolute numbers of cells per gram of tissue and represented as mean±SEM. FIG. 4D. Pearson correlation analysis between tumor volume (mm³) and number of tumor-infiltrating DCs per gram of tissue. Data shown in FIG. 4A-FIG. 4D are pooled from three different experiments with n=4-6 mice. FIG. 4E-4G, Histogram plots show the expression levels of CD24, CD64, CD169, CXC3R1, Ly6C (FIG. 4E), F4/80 (FIG. 4F) and XCR1, IRF8 (FIG. 4G) on CD103⁺cDC1 (red), CD11b⁺cDC2 (blue), CD103^{int}CD11b⁺DC (green) populations and macrophages (gray). Data shown in FIG. 4E-FIG. 4G are representative of three independent replicates.

FIG. 5A-5B show transcriptional analysis highlights distinct profile of tumor CD103^{int}CD11b⁺DC. Sorted tumor-infiltrating DC subpopulations (CD103⁺cDC1, CD11b⁺cDC2 and CD103^{int}CD11b⁺DC) and macrophages. RNA isolation and bulk RNA sequencing (RNA-seq) analysis was performed to the sorted populations. FIG. 5A, Principal component analysis (PCA) of CD103⁺cDC1, CD11b⁺cDC2 and CD103^{int}CD11b⁺DC populations and macrophages based on RNA-seq global transcriptional profiles. FIG. 5B, Heat map of log 2-transformed expression from RNA-seq across populations for DC canonical markers [85] as well as from macrophage/monocyte markers. Red and green gene names indicate genes that are upregulated and downregulated, respectively.

FIG. 6A-6C show single cell-RNA sequencing (scRNA-seq) analysis revealed that hetIL-15 induced CD103^{int}CD11b⁺DCs share transcriptomic similarities with the monocyte-derived DCs (moDCs) and cDCs. Isolated tumor-infiltrating CD11c⁺ populations from control and hetIL15-treated EO771-tumor bearing mice were processed into single-cell suspension. FIG. 6A, UMAP plot of scRNA-seq analysis of CD11c⁺ tumor-infiltrating cells serially annotated with SingleR86. FIG. 6B, Scaled density UMAP plot showing sample origin of clustered cells in each cluster. 0 to 1 indicating 100% of cells originating from control sample or hetIL15-treated sample, respectively. FIG. 6C, Heatmap reporting scaled, imputed expression of the top 10 differentially expressed genes for each cluster across all cells, identified in FIG. 6A. Genes of interest are shown in red.

FIG. 7A-7C show combined treatment of hetIL-15 immunotherapy and AC220 resulted in statistically significant EO771 tumor growth delay and complete eradication of the tumors in 50% of mice. FIG. 7A, Timeline of the tumor treatment. On day −1, C57BL/6 mice were inoculated with 3×10⁵ EO771 cells (orthotopically in the 4th mammary pad). Mice with palpable tumors were distributed in different groups 7 days later and treated with 3 locoregional hetIL-15 injections (5 μg/mouse/dose) every 4 days and AC220 (i.p, 5 mg/kg) every 3 days. FIG. 76, Tumor growth of C57BL/6 mice through days 16. FIG. 7C, Flow cytometry analysis of intratumoral CD103⁺cDC1, CD11b⁺cDC2 and CD103^{int}CD11b⁺ DC populations in control, hetIL-15 and/or AC220 treated mice. Data in graph are from one experiment (n=6), are given as absolute numbers of cells per gram of tissue and represented as mean±SEM.

FIG. 8 shows GEMM KPC mouse model of pancreatic cancer was used to test hetIL-15 anti-tumor activity as single agent and in combination with the chemotherapeutic agent gemcitabine. GEMM KPC mice develop pancreatic tumors at the age of around 15-weeks-old. Tumor growth was measured via ultrasound imaging and when the tumor reached the size of around 40 mm³, the mice were randomized in four groups: (i) control (PBS), (ii) gemcitabine (100 mg/kg), (iii) hetIL-15 (3 μg) and (iv) gemcitabine plus hetIL-15. Gemcitabine monotherapy was given sequentially as this treatment scheme is followed in the clinic.

FIG. 9 shows that hetIL-15 increases blood lymphocytes in GEMM KPC mouse model without and with gemcitabine. Kinetic analysis of blood lymphocyte counts during the treatment with hetIL-15 or/and gemcitabine in GEMM KPC model. Graphs represent mean±SEM. hetIL-15 corrects gemcitabine-induced lymphopenia, and it can be administered in combination with gemcitabine.

FIG. 10 shows a decrease in GEMM KPC tumor volume by hetIL-15+Gemcitabine combination at day 26 (end of treatment). Mean (±SEM) of the pancreatic tumor size on day 26. Pancreatic tumor-bearing GEMM KPC mice in the four groups.

FIG. 11 shows increased necrotic areas in treated GEMM KPC tumors. Ultrasound in situ imaging and H&E staining of excised tumors from mice in control, hetIL-15 or/and gemcitabine-treated groups using the GEMM KPC model. Scale bar, 3000 μm.

FIG. 12 shows increased necrotic areas in treated GEMM KPC tumors. Tumors from GEMM KPC models were H&E-stained (as in FIG. 11) and the areas of necrosis were calculated for each group. The evaluation of necrosis with H&E staining is possible as the necrotic areas are depicted with a paler pink derived from the eosin-stained proteins that are released by the necrotic cells. Comparison of similar size tumors of mice with endpoints from day 26-50 showed extensive intratumoral necrosis upon hetIL-15 monotherapy, which was significantly higher compared to Gemcitabine. Results are expressed as mean±SEM.

FIG. 13A-13B show increased CD8/CD4 ratio in spleens and tumors of hetIL-15 treated GEMM KPC mice. CD8+T/CD4+T cell ratio in spleen (FIG. 13A) and tumor (FIG. 13B) in control, hetIL-15 or/and gemcitabine-treated groups of the GEMM KPC model at the endpoint (from day 26-50). Results are expressed as percentages of CD4+ or CD8+, gated on the viable CD3+/CD45+. Data are presented as mean±SEM.

FIG. 14 shows flow cytometry analysis revealed increased infiltration of CD8+T cells in hetIL-15 treated groups of GEMM KPC tumors, including combination. Flow cytometric analysis of tumor-infiltrating CD8+T cells in control, hetIL-15 or/and gemcitabine-treated groups of the GEMM KPC model at the endpoint (days 26-50). Data in graph are given as percentage of live CD45+ cells and represented as mean±SEM.

FIG. 15 shows hetIL-15 increased infiltration of CD8+ cells in GEMM KPC tumors. Representative IHC anti-CD8-staining of tumors in control, hetIL-15 or/and gemcitabine-treated groups of the GEMM KPC model at the endpoint (from day 26-50).

FIG. 16A-16B show hetIL-15 treatment reduced metastatic disease in the lungs of GEMM KPC bearing mice. Representative images of H&E-stained lungs sections (FIG. 16A) and the number of metastatic foci in lungs (FIG. 16B) of control and hetIL-15 or/and gemcitabine-treated animals in the transgenic GEMM KPC model. Results are expressed as mean±SEM.

FIG. 17A-17B show hetIL-15 treatment controls the metastatic disease in the lungs in an I.V. model. The anti-metastatic effect of hetIL-15 treatment was evaluated by H&E-staining of the lungs sections of mice using the induced metastatic IV KPC pancreatic cancer model. The IV model is used to evaluate the effects of the treatment directly on the metastatic disease. KPC cells inoculated through the tail vain preferably colonize the lungs. The H&E-staining revealed that hetIL-15 monotherapy decreased the total number of the metastatic foci also in the IV KPC model. Analysis was done by HALO software.

FIG. 18A-18B show hetIL-15 treatment increased CD8+ cells accumulation in the lungs. IHC analysis (FIG. 18A) in the mouse lungs of the induced metastatic IV KPC pancreatic cancer model, using the anti-CD8 antibody. IHC staining revealed that hetIL-15 therapy increased CD8 positive cells in the lungs of the mice in IV KPC model (FIG. 18B). Data in graph are given as percentage of live CD45+ cells and represented as mean±SEM. Analysis was done by HALO software.

FIG. 19A-19B show hetIL-15 Increased CD103^{int}CD11b⁺ DCs also in KPC tumors. FIG. 19A shows flow cytometry identified a novel Dendritic cell population infiltrating pancreatic tumors, increased by hetIL-15. FIG. 19B shows RNA scope imaging, indicating the topology of those DCs into the pancreatic tumors.

FIG. 20A-20D show a comparison of peritumoral administration of hetIL-15 vs systemic administration in 4T1 breast and KPC Pancreatic cancer models. In FIG. 20A and FIG. 20B the tumor growth curves of the 4T1 breast CA orthotopic model are depicted, (FIG. 20A) treated with systemic administration of hetIL-15 or (FIG. 20B) with locoregional administration. In FIG. 20C, the tumor growth curve of the GEMM KPC pancreatic CA model is depicted, treated with systemic administration of hetIL-15. In (FIG. 20D), the tumor growth curve of the KPC heterotopic model into the mammary fat tissue is depicted, treated with locoregional administration of hetIL-15.

FIG. 21 shows a schematic of an IL-15:IL-12 chimera that can be generated with either mouse or human sequences (GAGA linker is SEQ ID NO:42).

FIG. 22 shows hetIL-15 determination by Eliza method, which detects the formation of heterodimeric IL-15 in cell extracts, supernatants or the sum of the two as total production. Addition of sIL-15Ra expression vector leads to the formation of heterodimeric IL-15.

FIG. 23 shows expression of IL-15:IL-12p40(L) chimera in supernatant of embryonic fiberblasts transfected with 100 ng of DNA expressing the chimera.

FIG. 24 shows that the IL-15:IL-12p40 fusion protein maintains the ability to interact with IL-12p35 and IL-15sRα. HEK293 were transiently transfected with plasmids encoding for IL-12p35 together with the fusion protein (cloned with FLAG-tagged IL-12p40) or with IL-15sRa and harvested at 48 hours. Band shift suggests an interaction between IL-15:IL-12, IL-12p35, and IL-15sRa. Primary antibody: Anti-FLAG.

FIG. 25 shows that IFN-γ production by NK-92 cells stimulated by IL-15:IL-12p40 fusion protein or by IL-12p70.

FIG. 26A-26B show that co-delivery of IL-12 and IL-15 into BALB/c mice has synergistic effects on IFN-γ production and CD8+T cell proliferation. FIG. 26A shows plasma IFN-gamma levels at Day 1. FIG. 26B shows flow-cytometry analysis demonstrating CD8+ T-cell proliferation in the spleen at Day 4.

FIG. 27 shows that co-delivery of a IL-15:IL-12 fusion protein and IL-12p35 promotes CD8+T cell proliferation similar to a combination of heterodimeric IL-15 and IL-12.

FIG. 28 shows the anti-tumor effects of IL-15:IL-12p40+IL12p35+IL15sRa chimera (CS70). B16 cells were injected IV into mice on day −1, then mice were hydrodynamically injected on day 0 and 7: 10 ng each DNA, and lung nodules were evaluated on day 21. The results demonstrate a greater than 2-fold decrease in metastases to the lung.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION OF THE DISCLOSURE

All publications, patents and patent applications cited herein are hereby expressly incorporated by reference for all purposes. In particular, the disclosures of WO2007084342, WO2009002562, WO2011020047, WO2014066527 and WO2016018920 are hereby expressly incorporated by reference in their entirety.

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “about” and “approximately,” when used to modify numeric value or numeric range, indicate that reasonable deviations from the value or range, typically 5% or 10% above and 5% or 10% below the value or range, remain within the intended meaning of the recited value or range.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

As used herein, the terms “prevent,” “preventing” and “prevention” in the context of the administration of a therapy to a subject refer to the inhibition of the onset or recurrence of a disease or disorder in a subject.

As used herein, the terms “manage,” “managing,” and “management,” in the context of the administration of a therapy to a subject, refer to the beneficial effects that a subject derives from a therapy, which does not result in a cure of a disease or disorder. In certain embodiments, a subject is administered one or more therapies to “manage” a disease or disorder so as to prevent the progression or worsening of symptoms associated with a disease or disorder.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this disclosure. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker. In the present disclosure, a “nucleic acid” molecule can include, DNA, cDNA and genomic DNA sequences, RNA, messenger RNA, and synthetic nucleic acid sequences. In some embodiments, the nucleic acid molecules are codon-optimized for expression. Thus, “nucleic acid” also encompasses embodiments in which analogs of DNA and RNA are employed. In some embodiments, the nucleic acid component may comprises one or more RNA molecules, such as viral RNA molecules or mRNA molecules that encode the protein of interest.

As used herein, the terms “subject” and “patient” are used interchangeably and refer to a mammal such as a non-primate (e.g., cows, pigs, horses, cats, dogs, rats etc.) and a primate (e.g., monkey and human). In certain embodiments, a subject or patient is a human.

As used herein, the terms “purified” and “isolated” when used in the context of a compound or agent (including proteinaceous agents such as antibodies and polypeptides) that can be obtained from a natural source, e.g., cells, refers to a compound or agent which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

As used herein, the terms “therapies” and “therapy” can refer to any protocol(s), method(s), compositions, formulations, and/or agent(s) that can be used in the prevention, treatment, management, or amelioration of a disease, e.g., cancer, infectious disease, autoimmune disease, graft versus host disease, and transplantation rejection, or a symptom associated therewith. In certain embodiments, the terms “therapies” and “therapy” refer to biological therapy, supportive therapy, and/or other therapies useful in treatment, management, prevention, or amelioration of a disease or a symptom associated therewith known to one of skill in the art.

As used herein, the term “modulating” or “modulate” refers to an effect of altering a biological activity, especially a biological activity associated with a particular biomolecule such as a protein kinase. For example, an agonist or antagonist of a particular biomolecule modulates the activity of that biomolecule, e.g., an enzyme, by either increasing (e.g., agonist, activator), or decreasing (e.g., antagonist, inhibitor) the activity of the biomolecule, such as an enzyme. Such activity is typically indicated in terms of an inhibitory concentration (IC₅₀) or excitation concentration (EC₅₀) of the compound for an inhibitor or activator, respectively, with respect to, for example, an enzyme.

As used herein, the terms “protein(s)” and “polypeptide(s)” interchangeably to refer to a chain of amino acids linked together by peptide bonds. In some embodiments, the terms “protein(s)” and “polypeptide(s)” refer to a macromolecule which comprises amino acids that are linked together by peptide bonds.

As used herein, the term “fragment” is the context of a fragment of a protein or polypeptide refers to a fragment that is composed of 8 or more contiguous amino acids, 10 or more contiguous amino acids, 15 or more contiguous amino acids, 20 or more contiguous amino acids, 25 or more contiguous amino acids, 50 or more contiguous amino acids, 75 or more contiguous amino acids, 100 or more contiguous amino acids, 150 or more contiguous amino acids, 200 or more contiguous amino acids, 10 to 150 contiguous amino acids, 10 to 200 contiguous amino acids, 10 to 250 contiguous amino acids, 10 to 300 contiguous amino acids, 50 to 100 contiguous amino acids, 50 to 150 contiguous amino acids, 50 to 200 contiguous amino acids, 50 to 250 contiguous amino acids or 50 to 300 contiguous amino acids of a protein or polypeptide, e.g., IL-15 and IL-15Rα polypeptides.

As used herein, the term “in combination” refers to the use of more than one therapies (e.g., one or more active agents). The use of the term “in combination” does not restrict the order in which therapies are administered to a subject with a disease or disorder. For example, a first therapy (e.g., IL-15 or IL-15/IL-15Rα complex) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., one or more active agents) to a subject with a disease or disorder or a symptom thereof (e.g., cancer).

This disclosure provides compositions and methods comprising combinations of IL-15 with one or more other active agents. In some embodiments, the one or more active agents comprises an activator of PPAR (Peroxisome proliferator-activated receptor). In some embodiments, the one or more active agents comprises inhibitor of FLT3.

### IL-15

Interleukin-15 (IL-15) is a homeostatic cytokine of the gamma-chain family of cytokines. IL-15 has been shown to induce and regulate a wide range of immune functions. Specifically, IL-15 is critical for lymphoid development and peripheral maintenance of innate immune cells and memory of T cells, such as natural killer (NK) and CD8⁺T cells. IL-15 does not promote the function of CD4⁺CD25⁺FOXP3⁺ regulatory T cells (Tregs), suggesting its use as a therapeutic agent in cancer immunotherapy. The native form of IL-15 circulating in the plasma exists as a complex of the IL-15 chain with the IL-15 receptor alpha chain (IL-15Rα) that are together termed heterodimeric IL-15 (hetIL-15), and the functional cytokine in vivo is the heterodimer. The two chains IL-15 and IL-15Rα are produced from the same cell and associate in the endoplasmic reticulum due to their high binding affinity (kd=10⁻¹¹). The IL-15 heterodimer is then transported to the cell surface and released as bioactive soluble heterodimeric molecule, upon proteolytic cleavage of IL-15Rα. IL-15 has shown anticancer activity in many model systems and is presently in multiple clinical trials for cancer immunotherapy (NCT02452268; NCT04261439). hetIL-15 delivery also increased the intratumoral CD103⁺cDC1s of flank MC-38 and TC-1 tumors.

As used herein, the terms “IL-15” and “interleukin-15 in the context of proteins or polypeptides refer to any mammalian interleukin-15 amino acid sequences, including immature or precursor and mature forms. Non-limiting examples of GeneBank Accession Nos. for the amino acid sequence of various species of native mammalian interleukin-15 include NP_000576 (human, immature form), CAA62616 (human, immature form), NP_001009207 (Felis catus, immature form), AAB94536 (Rattus, immature form), AAB41697 (Rattus, immature form), NP_032383 (Mus musculus, immature form), AAR19080 (canine), AAB60398 (Macaca mulatta, immature form), AA100964 (human, immature form), AAH23698 (Mus musculus, immature form), and AAH18149 (human). The amino acid sequence of the immature/precursor form of native human IL-15, which comprises the long signal peptide (underlined) and the mature human native IL-15 (italicized), is provided below.

(SEQ ID NO: 01)
MRISKPHLRSISIQCYLCLLLNSHFLTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS

In some embodiments, IL-15 is the immature or precursor form of a mammalian IL-15. In other embodiments, IL-15 is the mature form of a mammalian IL-15. In a specific embodiment, IL-15 is the precursor form of human IL-15. In another embodiment, IL-15 is the mature form of human IL-15. In one embodiment, the IL-15 protein/polypeptide is isolated or purified.

As used herein, the terms “IL-15 derivative” and “interleukin-15 derivative” in the context of proteins or polypeptides refer to: (a) a polypeptide that is at least 40%, 45%, 50%, 55%, 60%, 65%, 70%, 75%, 80%, 85%, 90%, 95%, 98% or 99% identical to an IL-15 polypeptide; (b) a polypeptide encoded by a nucleic acid sequence that is at least 40%, 45%, 50%, 55%, 60%, 65%, 70%, 75%, 80%, 85%, 90%, 95%, 98% or 99% identical a nucleic acid sequence encoding an IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-15 polypeptide; (d) a polypeptide encoded by nucleic acids can hybridize under high, moderate or typical stringency hybridization conditions to nucleic acids encoding an IL-15 polypeptide; (e) a polypeptide encoded by a nucleic acid sequence that can hybridize under high, moderate or typical stringency hybridization conditions to a nucleic acid sequence encoding a fragment of an IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a mammalian IL-15 polypeptide. IL-15 derivatives also include a polypeptide that comprises the amino acid sequence of a mature form of a mammalian IL-15 polypeptide and a heterologous signal peptide amino acid sequence. In a specific embodiment, an IL-15 derivative is a derivative of a native human IL-15 polypeptide. In another embodiment, an IL-15 derivative is a derivative of an immature or precursor form of human IL-15 polypeptide. In another embodiment, an IL-15 derivative is a derivative of a mature form of human IL-15 polypeptide. In one embodiment, an IL-15 derivative is isolated or purified.

In certain embodiments, IL-15 derivatives retain at least 50%, 55%, 60%, 65%, 70%, 75%, 80%, 85%, 90%, 95%, 98% or 99% of the function of native mammalian IL-15 polypeptide to bind IL-15Rα polypeptide, as measured by assays well known in the art, e.g., ELISA, Biacore, co-immunoprecipitation. In some embodiments, IL-15 derivatives retain at least 50%, 55%, 60%, 65%, 70%, 75%, 80%, 85%, 90%, 95%, 98% or 99% of the function of native mammalian IL-15 polypeptide to induce IL-15-mediated signal transduction, as measured by assays well-known in the art, e.g., electromobility assays, electromobility shift assays, ELISAs and other immunoassays or live cell bioassays.

As used herein, the terms “IL-15Rα” and “interleukin-15 receptor alpha” in the context of proteins or polypeptides refer to any mammalian interleukin-15 receptor alpha (“IL-15Rα”) amino acid sequence, including immature or precursor and mature forms and naturally occurring isoforms. Non-limiting examples of GeneBank Accession Nos. for the amino acid sequence of various native mammalian IL-15Rα include NP_002180 (human), ABK41438 (Macaca mulatta), NP_032384 (Mus musculus), Q60819 (Mus musculus), CA141082 (human). The amino acid sequence of the immature form of the native full length human IL-15Rα is provided below.

(SEQ ID NO: 03)
MAPRRARGCRTLGLPALLLLLLLRPPATRGITCPPPMSVEHADIW
VKSYSLYSRERYICNSGFKRKAGTSSLTECVLNKATNVAHWTTPS
LKCIRDPALVHQRPAPPSTVTTAGVTPQPESLSPSGKEPAASSPS
SNNTAATTAAIVPGSQLMPSKSPSTGTTEISSHESSHGTPSQTTA
KNWELTASASHQPPGVYPQGHSDTTVAISTSTVLLCGLSAVSLLA
CYLKSRQTPPLASVEMEAMEALPVTWGTSSRDEDLENCSHHL

The amino acid sequence of the immature form of the soluble human IL-15Rα is provided below.

(SEQ ID NO: 04)
MAPRRARGCRTLGLPALLLLLLLRPPATRGITCPPPMSVEHADIW
VKSYSLYSRERYICNSGFKRKAGTSSLTECVLNKATNVAHWTTPS
LKCIRDPALVHQRPAPPSTVTTAGVTPQPESLSPSGKEPAASSPS
SNNTAATTAAIVPGSQLMPSKSPSTGTTEISSHESSHGTPSQTTA
KNWELTASASHQPPGVYPQGHSDTT

In some embodiments, IL-15Rα is the immature form of a mammalian IL-15Rα polypeptide. In other embodiments, native IL-15Rα is the mature form of a mammalian IL-15Rα polypeptide. In certain embodiments, IL-15Rα is a soluble form of a mammalian IL-15Rα polypeptide. In other embodiments, IL-15Rα is the full-length form of a mammalian IL-15Rα polypeptide. In a specific embodiment, IL-15Rα is the immature form of a human IL-15Rα polypeptide. In another embodiment, IL-15Rα is the mature form of a human IL-15Rα polypeptide. In certain embodiments, IL-15Rα is the soluble form of a human IL-15Rα polypeptide. In other embodiments, IL-15Rα is the full-length form of a human IL-15Rα polypeptide. In one embodiment, the IL-15Rα protein or polypeptide is isolated or purified.

As used herein, the terms “IL-15Rα derivative” and “interleukin-15 receptor alpha derivative” in the context of a protein or polypeptide refer to: (a) a polypeptide that is at least 40%, 45%, 50%, 55%, 60%, 65%, 70%, 75%, 80%, 85%, 90%, 95%, 98% or 99% identical to a mammalian IL-15 polypeptide; (b) a polypeptide encoded by a nucleic acid sequence that is at least 40%, 45%, 50%, 55%, 60%, 65%, 70%, 75%, 80%, 85%, 90%, 95%, 98% or 99% identical a nucleic acid sequence encoding a mammalian IL-15Rα polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a mammalian IL-15Rα polypeptide; (d) a polypeptide encoded by a nucleic acid sequence that can hybridize under high, moderate or typical stringency hybridization conditions to a nucleic acid sequence encoding a mammalian IL-15Rα polypeptide; (e) a polypeptide encoded by a nucleic acid sequence that can hybridize under high, moderate or typical stringency hybridization conditions to nucleic acid sequences encoding a fragment of a mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a mammalian IL-15Rα polypeptide. IL-15Rα derivatives also include a polypeptide that comprises the amino acid sequence of a naturally occurring mature form of mammalian IL-15Rα polypeptide and a heterologous signal peptide amino acid sequence. In a specific embodiment, an IL-15Rα derivative is a derivative of a human IL-15Rα polypeptide. In another embodiment, an IL-15Rα derivative is a derivative of an immature form of human IL-15 polypeptide. In another embodiment, an IL-15Rα derivative is a derivative of a mature form of human IL-15 polypeptide. In one embodiment, an IL-15Rα derivative is the soluble form of a mammalian IL-15Rα polypeptide. In a specific embodiment, an IL-15Rα derivative is purified or isolated.

The proteolytic cleavage of membrane-bound human IL-15Rα takes place between Gly170 and His171 in human IL-15Rα (Chertova et al., 2013, Journal of Biological Chemistry 288(25):18093-103). Thus, the proteolytic cleavage of human IL-15Rα takes place between the residues (i.e., Gly170 and His171) in the provided amino acid sequence of the immature form of the native full length human IL-15Rα:

(SEQ ID NO: 03)
MAPRRARGCRTLGLPALLLLLLLRPPATRGITCPPPMSVEHADIW
VKSYSLYSRERYICNSGFKRKAGTSSLTECVLNKATNVAHWTTPS
LKCIRDPALVHQRPAPPSTVTTAGVTPQPESLSPSGKEPAASSPS
SNNTAATTAAIVPGSQLMPSKSPSTGTTEISSHESSHGTPSQTTA
KNWELTASASHQPPGVYPQGHSDTTVAISTSTVLLCGLSAVSLLA
CYLKSRQTPPLASVEMEAMEALPVTWGTSSRDEDLENCSHHL

In certain embodiments, provided herein is a soluble form of human IL-15Rα (e.g., a purified soluble form of human IL-15Rα), wherein the amino acid sequence of the soluble form of human IL-15Rα terminates at the site of the proteolytic cleavage of the native membrane-bound human IL-15Rα. In certain embodiments, provided herein is a soluble form of human IL-15Rα (e.g., a purified soluble form of human IL-15Rα), wherein the amino acid sequence of the soluble form of human IL-15Rα terminates with PQG, wherein G is Gly170. In certain embodiments, provided herein is a soluble form of human IL-15Rα (e.g., a purified soluble form of human IL-15Rα) which has the following amino acid sequence:

(SEQ ID NO: 10)
ITCPPPMSVEHADIWVKSYSLYSRERYICNSGFKRKAGTSSLTEC
VLNKATNVAHWTTPSLKCIRDPALVHQRPAPPSTVTTAGVTPQPE
SLSPSGKEPAASSPSSNNTAATTAAIVPGSQLMPSKSPSTGTTEI
SSHESSHGTPSQTTAKNWELTASASHQPPGVYPQGHSDTT.

In some embodiments, provided herein is an IL-15Rα derivative (e.g., a purified and/or soluble form of IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:10; and (ii) terminates with the amino acid sequence PQG.

In certain embodiments, provided herein is a soluble form of human IL-15Rα (e.g., a purified soluble form of human IL-15Rα) which has the following amino acid sequence:

(SEQ ID NO: 11)
ITCPPPMSVEHADIWVKSYSLYSRERYICNSGFKRKAGTSSLTECVLNK
ATNVAHWTTPSLKCIRDPALVHQRPAPPSTVTTAGVTPQPESLSPSGKE
PAASSPSSNNTAATTAAIVPGSQLMPSKSPSTGTTEISSHESSHGTPSQ
TTAKNWELTASASHQPPGVYPQG.

In some embodiments, provided herein is an IL-15Rα derivative (e.g., a purified and/or soluble form of an IL-15Rα derivative), which is a polypeptide that is at least 40%, 45%, 50%, 55%, 60%, 65%, 70%, 75%, 80%, 85%, 90%, 95%, 98% or 99% identical to SEQ ID NO:11, and, optionally, wherein the amino acid sequence of the soluble form of the IL-15Rα derivative terminates with PQG.

In some embodiments, provided herein are IL-15Rα derivatives that are truncated, soluble forms of naturally occurring human IL-15Rα. In certain embodiments, provided herein is a soluble form of human IL-15Rα (e.g., a purified soluble form of human IL-15Rα), wherein the amino acid sequence of the soluble form of human IL-15Rα terminates with PQGH (SEQ ID No:25). In particular embodiments, provided herein is a soluble form of human IL-15Rα (e.g., a purified soluble form of human IL-15Rα) which has the following amino acid sequence:

(SEQ ID NO: 12)
MAPRRARGCRTLGLPALLLLLLLRPPATRGITCPPPMSVEHADIWVKSY
SLYSRERYICNSGFKRKAGTSSLTECVLNKATNVAHWTTPSLKCIRDPA
LVHQRPAPPSTVTTAGVTPQPESLSPSGKEPAASSPSSNNTAATTAAIV
PGSQLMPSKSPSTGTTEISSHESSHGTPSQTTAKNWELTASASHQPPGV
YPQGH.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:12; and (ii) terminates with the amino acid sequence PQGH (SEQ ID NO:25).

In other particular embodiments, provided herein is a soluble form of human IL-15Rα (e.g., a purified soluble form of human IL-15Rα) which has the following amino acid sequence:

(SEQ ID NO: 13)
ITCPPPMSVE HADIWVKSYS LYSRERYICN SGFKRKAGTS SLTECVLNKA TNVAHWTTPS
LKCIRDPALV HQRPAPPSTV TTAGVTPQPE SLSPSGKEPA ASSPSSNNTA ATTAAIVPGS
QLMPSKSPST GTTEISSHES SHGTPSQTTA KNWELTASAS HQPPGVYPQGH.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:13; and (ii) has the amino acid sequence of the soluble form of the IL-15Rα derivative terminates with PQGH (SEQ ID NO:25).

In certain embodiments, provided herein is a soluble form of human IL-15Rα (e.g., a purified soluble form of human IL-15Rα), wherein the amino acid sequence of the soluble form of human IL-15Rα terminates with PQGHS (SEQ ID NO:26). In particular embodiments, provided herein is a soluble form of human IL-15Rα (e.g., a purified soluble form of human IL-15Rα) which has the following amino acid sequence:

(SEQ ID NO: 14)
MAPRRARGCR TLGLPALLLL LLLRPPATRG ITCPPPMSVE HADIWVKSYS LYSRERYICN
SGFKRKAGTS SLTECVLNKA TNVAHWTTPS LKCIRDPALV HQRPAPPSTV TTAGVTPQPE
SLSPSGKEPA ASSPSSNNTA ATTAAIVPGS QLMPSKSPST GTTEISSHES SHGTPSQTTA
KNWELTASAS HQPPGVYPQGHS

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:14; and (ii) terminates with the amino acid sequence PQGHS (SEQ ID NO:26).

In other particular embodiments, provided herein is a soluble form of human IL-15Rα (e.g., a purified soluble form of human IL-15Rα) which has the following amino acid sequence:

(SEQ ID NO: 15)
ITCPPPMSVE HADIWVKSYS LYSRERYICN SGFKRKAGTS SLTECVLNKA TNVAHWTTPS
LKCIRDPALV HQRPAPPSTV TTAGVTPQPE SLSPSGKEPA ASSPSSNNTA ATTAAIVPGS
QLMPSKSPST GTTEISSHES SHGTPSQTTA KNWELTASAS HQPPGVYPQGHS.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:15; and (ii) terminates with the amino acid sequence PQGHS (SEQ ID NO:26).

In certain embodiments, provided herein is a soluble form of human IL-15Rα (e.g., a purified soluble form of human IL-15Rα), wherein the amino acid sequence of the soluble form of human IL-15Rα terminates with PQGHSD (SEQ ID NO:27). In particular embodiments, provided herein is a soluble form of human IL-15Rα (e.g., a purified soluble form of human IL-15Rα) which has the following amino acid sequence:

(SEQ ID NO: 16)
MAPRRARGCR TLGLPALLLL LLLRPPATRG ITCPPPMSVE HADIWVKSYS LYSRERYICN
SGFKRKAGTS SLTECVLNKA TNVAHWTTPS LKCIRDPALV HQRPAPPSTV TTAGVTPQPE
SLSPSGKEPA ASSPSSNNTA ATTAAIVPGS QLMPSKSPST GTTEISSHES SHGTPSQTTA
KNWELTASAS HQPPGVYPQGHSD.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:16; and (ii) terminates with the amino acid sequence PQGHSD (SEQ ID NO:27).

In other particular embodiments, provided herein is a soluble form of human IL-15Rα (e.g., a purified soluble form of human IL-15Rα) which has the following amino acid sequence:

(SEQ ID NO: 17)
ITCPPPMSVE HADIWVKSYS LYSRERYICN SGFKRKAGTS SLTECVLNKA TNVAHWTTPS
LKCIRDPALV HQRPAPPSTV TTAGVTPQPE SLSPSGKEPA ASSPSSNNTA ATTAAIVPGS
QLMPSKSPST GTTEISSHES SHGTPSQTTA KNWELTASAS HQPPGVYPQGHSD.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:17; and (ii) terminates with the amino acid sequence PQGHSD (SEQ ID NO:27).

In certain embodiments, provided herein is a soluble form of human IL-15Rα (e.g., a purified soluble form of human IL-15Rα), wherein the amino acid sequence of the soluble form of human IL-15Rα terminates with PQGHSDT (SEQ ID NO:28). In particular embodiments, provided herein is a soluble form of human IL-15Rα (e.g., a purified soluble form of human IL-15Rα) which has the following amino acid sequence:

(SEQ ID NO: 18)
MAPRRARGCR TLGLPALLLL LLLRPPATRG ITCPPPMSVE HADIWVKSYS LYSRERYICN
SGFKRKAGTS SLTECVLNKA TNVAHWTTPS LKCIRDPALV HQRPAPPSTV TTAGVTPQPE
SLSPSGKEPA ASSPSSNNTA ATTAAIVPGS QLMPSKSPST GTTEISSHES SHGTPSQTTA
KNWELTASAS HQPPGVYPQGHSDT.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:18; and (ii) terminates with the amino acid sequence PQGHSDT (SEQ ID NO:28).

In other particular embodiments, provided herein is a soluble form of human IL-15Rα (e.g., a purified soluble form of human IL-15Rα) which has the following amino acid sequence:

(SEQ ID NO: 19)
ITCPPPMSVE HADIWVKSYS LYSRERYICN SGFKRKAGTS SLTECVLNKA TNVAHWTTPS
LKCIRDPALV HQRPAPPSTV TTAGVTPQPE SLSPSGKEPA ASSPSSNNTA ATTAAIVPGS
QLMPSKSPST GTTEISSHES SHGTPSQTTA KNWELTASAS HQPPGVYPQGHSDT.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:19; and (ii) terminates with the amino acid sequence PQGHSDT (SEQ ID NO:28).

In certain embodiments, provided herein is a soluble form of human IL-15Rα (e.g., a purified soluble form of human IL-15Rα), wherein the amino acid sequence of the soluble form of human IL-15Rα terminates with PQGHSDTT (SEQ ID NO:29). In particular embodiments, provided herein is a soluble form of human IL-15Rα (e.g., a purified soluble form of human IL-15Rα) which has the following amino acid sequence:

(SEQ ID NO: 20)
MAPRRARGCR TLGLPALLLL LLLRPPATRG ITCPPPMSVE HADIWVKSYS LYSRERYICN
SGFKRKAGTS SLTECVLNKA TNVAHWTTPS LKCIRDPALV HQRPAPPSTV TTAGVTPQPE
SLSPSGKEPA ASSPSSNNTA ATTAAIVPGS QLMPSKSPST GTTEISSHES SHGTPSQTTA
KNWELTASAS HQPPGVYPQGHSDTT.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:20; and (ii) terminates with the amino acid sequence PQGHSDTT (SEQ ID NO:29).

In other particular embodiments, provided herein is a soluble form of human IL-15Rα (e.g., a purified soluble form of human IL-15Rα) which has the following amino acid sequence:

(SEQ ID NO: 21)
ITCPPPMSVE HADIWVKSYS LYSRERYICN SGFKRKAGTS SLTECVLNKA TNVAHWTTPS
LKCIRDPALV HQRPAPPSTV TTAGVTPQPE SLSPSGKEPA ASSPSSNNTA ATTAAIVPGS
QLMPSKSPST GTTEISSHES SHGTPSQTTA KNWELTASAS HQPPGVYPQGISDTT.

In some embodiments, provided herein is an IL-15Rα derivative (e.g., a purified and/or soluble form of an IL-15Rα derivative), which is a polypeptide that: (i) is at least 40%, 45%, 50%, 55%, 60%, 65%, 70%, 75%, 80%, 85%, 90%, 95%, 98% or 99% identical to SEQ ID NO:21; and (ii) terminates with the amino acid sequence PQGHSDTT (SEQ ID NO:29).

In some embodiments, provided herein is an IL-15Rα derivative of naturally occurring human IL-15Rα, wherein the IL-15Rα derivative is soluble and: (a) the last amino acids at the C-terminal end of the IL-15Rα derivative consist of amino acid residues PQGHSDTT (SEQ ID NO:29), wherein T is at the C-terminal end of the amino acid sequence; (b) the last amino acids at the C-terminal end of the IL-15Rα derivative consist of amino acid residues PQGHSDT (SEQ ID NO:28), wherein T is at the C-terminal end of the amino acid sequence; (c) the last amino acids at the C-terminal end of the IL-15Rα derivative consist of amino acid residues PQGHSD (SEQ ID NO:27), wherein D is at the C-terminal end of the amino acid sequence; (d) the last amino acids at the C-terminal end of the IL-15Rα derivative consist of amino acid residues PQGHS (SEQ ID NO:26), wherein S is at the C-terminal end of the amino acid sequence; or (e) the last amino acids at the C-terminal end of the IL-15Rα derivative consist of amino acid residues PQGH (SEQ ID NO:25), wherein H is at the C-terminal end of the amino acid sequence. In certain embodiments, the amino acid sequences of these IL-15Rα derivatives are at least 75%, at least 85%, at least 90%, at least 95%, at least 96%, at least 97%, at least 98%, or at least 99% identical to the amino acid sequence of SEQ ID NO:21. In some embodiments, provided herein is an IL-15Rα derivative of a naturally occurring human IL-15Rα, wherein the IL-15Rα derivative: (i) is soluble; (ii) comprises an amino acid sequence that is at least 75%, at least 80%, at least 90%, at least 95%, at least 96%, at least 97%, or at least 98% identical to the amino acid sequence of SEQ ID NO:21; and (iii) terminates with the amino acid sequence PQG, wherein G is at the C-terminal end of the amino acid sequence of the IL-15Rα derivative. In some embodiments, these IL-15Rα derivatives are purified.

In another aspect, provided herein are IL-15Rα derivatives in which the cleavage site for an endogenous protease that cleaves native IL-15Rα has been mutated. In one embodiment, provided herein are IL-15Rα derivatives comprising one, two, three, four, five, six, seven or eight mutations (e.g., additions, deletions or substitutions; such as deletions or substitutions of one, two, three, four, five, six, seven or eight amino acid residues) in the extracellular domain cleavage site of IL-15Rα such that cleavage of the IL-15Rα by an endogenous protease that cleaves native IL-15Rα is inhibited. As discussed above, the proteolytic cleavage of membrane-bound human IL-15Rα takes place between Gly170 and His171 in human IL-15Rα. In one embodiment, these amino acid residues or surrounding amino acid residues are mutated such that cleavage of IL-15Rα by an endogenous protease that cleaves native IL-15Rα is inhibited. In certain embodiments, the amino acid sequence PQGHSDTT (SEQ ID NO:29) is mutated such that cleavage by endogenous proteases that cleave native human IL-15Rα is inhibited. In specific embodiments, one, two, three, four, five, six, seven, or eight amino acid substitutions and/or deletions (such as substitutions and/or deletions of one, two, three, four, five, six, seven or eight amino acid residues) are introduced into the amino acid sequence PQGHSDTT (SEQ ID NO:29) of human IL-15Rα such that cleavage by endogenous proteases that cleave native human IL-15Rα is inhibited. In certain embodiments, the amino acid sequence PQGHSDTT (SEQ ID NO:29) is replaced with a cleavage site that is recognized and cleaved by a heterologous protease. Non-limiting examples of such heterologous protease cleavage sites include Arg-X-X-Arg (SEQ ID NO:45), which is recognized and cleaved by furin protease; and A-B-Pro-Arg-X-Y (A and B are hydrophobic amino acids and X and Y are nonacidic amino acids; SEQ ID NO:46) and Gly-Arg-Gly, which are recognized and cleaved by the thrombin protease.

In another aspect, provided herein are IL-15Rα derivatives, wherein the IL-15Rα derivatives: (i) comprises a mutated extracellular cleavage site that inhibits cleavage by an endogenous protease that cleaves native IL-15Rα, and (ii) lack all or a fragment of the transmembrane domain of native IL-15Rα. In certain embodiments, provided herein are IL-15Rα derivatives, wherein the IL-15Rα derivatives comprise: (i) one, two, three, four, five, six, seven or eight mutations (e.g., substitutions and/or deletions) in the extracellular cleavage site of IL-15Rα such that cleavage of IL-15Rα by an endogenous protease that cleaves native IL-15Rα is inhibited, and (ii) all or a fragment of a transmembrane domain of a heterologous molecule in place of all or a fragment of the transmembrane domain of native IL-15Rα. In some embodiments, provided herein are IL-15Rα derivatives, wherein the IL-15Rα derivatives comprise: (i) one, two, three, four, five, six, seven or eight mutations (e.g., substitutions and/or deletions) in the amino acid sequence PQGHSDTT (SEQ ID NO:29) such that cleavage of IL-15Rα by an endogenous protease that cleaves native IL-15Rα is inhibited, and (ii) all or a fragment of a transmembrane domain of a heterologous molecule in place of all or a fragment of the transmembrane domain of native IL-15Rα. In accordance with these embodiments, the IL-15Rα derivatives may or may not comprise all or a fragment of the cytoplasmic tail of native IL-15Rα. In certain embodiments, the heterologous molecule is CD4, CD8, or major histocompatibility complex (MHC).

In another aspect, provided herein are glycosylated forms of IL-15Rα (e.g., purified glycosylated forms of IL-15Rα), wherein the glycosylation of the IL-15Rα accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Rα as assessed by techniques known to one of skill in the art. The percentage of the mass (molecular weight) of IL-15Rα (e.g., purified IL-15Rα) that glycosylation of IL-15Rα accounts for can be determined using, for example and without limitation, gel electrophoresis and quantitative densitometry of the gels, and comparison of the average mass (molecular weight) of a glycosylated form of IL-15Rα (e.g., a purified glycosylated form of IL-15Rα) to the non-glycosylated form of IL-15Rα (e.g., a purified non-glycosylated form of IL-15Rα). In one embodiment, the average mass (molecular weight) of IL-15Rα (e.g., purified IL-15Rα) can be determined using MALDI-TOF MS spectrum on Voyager De-Pro equipped with CovalX HM-1 high mass detector using sinapic acid as matrix, and the mass of a glycosylated form of IL-15Rα (e.g., purified glycosylated form of IL-15Rα) can be compared to the mass of the non-glycosylated form of IL-15Rα (e.g., purified non-glycosylated form of IL-15Rα) to determine the percentage of the mass that glycosylation accounts for.

In certain embodiments, provided herein is a glycosylated IL-15Rα (e.g., human IL-15Rα), wherein the glycosylation accounts for at least 20%, 25%, 30%, 35%, 40%, 45%, 50%, 55%, 60%, 65%, 70%, 75%, 80%, 85%, 90%, 95% or more of the mass (molecular weight) of the IL-15Rα. In some embodiments, provided herein is a glycosylated IL-15Rα (e.g., human IL-15Rα), wherein the glycosylation accounts for 20% to 25%, 20% to 30%, 25% to 30%, 25% to 35%, 30% to 35%, 30% to 40%, 35% to 40%, 35% to 45%, 40% to 50%, 45% to 50%, 20% to 40%, 25% to 50%, 50% to 75%, 75% to 95%, or 75% to 100% of the mass (molecular weight) of the IL-15Rα. In certain embodiments, provided herein is a glycosylated IL-15Rα (e.g., human IL-15Rα), wherein the glycosylation accounts for about 20%, about 25%, about 30%, about 35%, about 40%, about 45%, about 50%, about 55%, about 60%, about 65%, about 70%, about 75%, about 80%, about 85%, about 90%, or about 95% of the mass (molecular weight) of the IL-15Rα. In specific embodiments, the glycosylated IL-15Rα is a native IL-15Rα (e.g., a native human IL-15Rα). In other specific embodiments, the glycosylated IL-15Rα is an IL-15Rα derivative (e.g., an IL-15Rα derivative of naturally occurring human IL-15Rα). In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In some embodiments, the glycosylated IL-15Rα is glycosylated atone, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα. In certain embodiments, the glycosylated IL-15Rα is purified or isolated.

In certain embodiments, provided herein is a composition comprising IL-15 and glycosylated IL-15Rα (e.g., human IL-15Rα), wherein the glycosylation of the IL-15Rα accounts for at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the mass (molecular weight) of the IL-15Rα as assessed by techniques known to one of skill in the art. In some embodiments, provided herein is provided herein is a composition comprising IL-15 and glycosylated IL-15Rα (e.g., human IL-15Rα), wherein the glycosylation of the IL-15Rα accounts for 20% to 25%, 20% to 30%, 25% to 30%, 25% to 35%, 30% to 35%, 30% to 40%, 35% to 40%, 35% to 45%, 40% to 50%, 45% to 50%, 20% to 40%, 25% to 50%, 50% to 75%, or 75% to 95% of the mass (molecular weight) of the IL-15Rα as assessed by techniques known to one of skill in the art. In other embodiments, provided herein is a composition comprising IL-15 and glycosylated IL-15Rα (e.g., human IL-15Rα), wherein the glycosylation of the IL-15Rα accounts for about 20%, about 25%, about 30%, about 35%, about 40%, about 45%, about 50%, about 55%, about 60%, about 65%, about 70%, about 75%, about 80%, about 85%, about 90%, or about 95% of the mass (molecular weight) of the IL-15Rα as assessed by techniques known to one of skill in the art. In certain embodiments, the IL-15 is glycosylated. In specific embodiments, the glycosylated IL-15Rα is a native IL-15Rα (e.g., a native human IL-15Rα). In other specific embodiments, the glycosylated IL-15Rα is an IL-15Rα derivative (e.g., an IL-15Rα derivative of naturally occurring human IL-15Rα). In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In some embodiments, the glycosylated IL-15Rα is glycosylated at one, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα.

In certain embodiments, provided herein is an IL-15/IL-15Rα complex comprising glycosylated IL-15Rα (e.g., human IL-15Rα), wherein the glycosylation of the IL-15Rα accounts for at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the mass (molecular weight) of the IL-15Rα as assessed by techniques known to one of skill in the art. In some embodiments, provided herein is an IL-15/IL-15Rα complex comprising glycosylated IL-15Rα (e.g., human IL-15Rα), wherein the glycosylation of the IL-15Rα accounts for 20% to 25%, 20% to 30%, 25% to 30%, 25% to 35%, 30% to 35%, 30% to 40%, 35% to 40%, 35% to 45%, 40% to 50%, 45% to 50%, 20% to 40%, 25% to 50%, 50% to 75%, or 75% to 95% of the mass (molecular weight) of the IL-15Rα as assessed by techniques known to one of skill in the art. In other embodiments, provided herein is an IL-15/IL-15Rα complex comprising glycosylated IL-15Rα (e.g., human IL-15Rα), wherein the glycosylation of the IL-15Rα accounts for about 20%, about 25%, about 30%, about 35%, about 40%, about 45%, about 50%, about 55%, about 60%, about 65%, about 70%, about 75%, about 80%, about 85%, about 90%, or about 95% of the mass (molecular weight) of the IL-15Rα as assessed by techniques known to one of skill in the art. In specific embodiments, the glycosylated IL-15Rα is a native IL-15Rα (e.g., a native human IL-15Rα). In other specific embodiments, the glycosylated IL-15Rα is an IL-15Rα derivative (e.g., an IL-15Rα derivative of naturally occurring human IL-15Rα). In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In some embodiments, the glycosylated IL-15Rα is glycosylated at one, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα. In certain embodiments, the IL-15/IL-15Rα complex is purified or isolated.

In another aspect, provided herein are glycosylated forms of IL-15Rα, wherein the IL-15Rα is glycosylated (N- or O-glycosylated) at certain amino acid residues. In certain embodiments, provided herein is a human IL-15Rα which is glycosylated at one, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα. In specific embodiments, the glycosylated IL-15Rα is a native human IL-15Rα. In other specific embodiments, the glycosylated IL-15Rα is an IL-15Rα derivative of naturally occurring human IL-15Rα. In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In certain embodiments, the glycosylated IL-15Rα is purified or isolated.

In certain embodiments, provided herein is a composition comprising IL-15 and human IL-15Rα, wherein the human IL-15Rα is glycosylated at one, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα. In specific embodiments, the glycosylated IL-15Rα is a native human IL-15Rα. In other specific embodiments, the glycosylated IL-15Rα is an IL-15Rα derivative of naturally occurring human IL-15Rα. In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In certain embodiments, the glycosylated IL-15Rα is purified or isolated.

In certain embodiments, provided herein is an IL-15/IL-15Rα complex comprising human IL-15Rα which is glycosylated at one, two, three, four, five, six, seven, or all, of the following glycosylation sites: (i) O-glycosylation on Thr5 of amino acid sequence NWELTASASHQPPGVYPQG (SEQ ID NO:30) in the IL-15Rα; (ii) O-glycosylation on Ser7 of amino acid sequence NWELTASASHQPPGVYPQG (SEQ ID NO:30) in the IL-15Rα; (iii) N-glycosylation on Ser 8 of amino acid sequence ITCPPPMSVEHADIWVK (SEQ ID NO:31) in the IL-15Rα, or Ser 8 of amino acid sequence ITCPPPMSVEHADIWVKSYSLYSRERYICNS (SEQ ID NO:32) in the IL-15Rα; (iv) N-glycosylation on Ser 18 of amino acid sequence ITCPPPMSVEHADIWVKSYSLYSRERYICNS (SEQ ID NO:32) in the IL-15Rα; (v) N-glycosylation on Ser 20 of amino acid sequence ITCPPPMSVEHADIWVKSYSLYSRERYICNS (SEQ ID NO:32) in the IL-15Rα; (vi) N-glycosylation on Ser 23 of amino acid sequence ITCPPPMSVEHADIWVKSYSLYSRERYICNS (SEQ ID NO:32) in the IL-15Rα; and/or (vii) N-glycosylated on Ser 31 of amino acid sequence ITCPPPMSVEHADIWVKSYSLYSRERYICNS (SEQ ID NO:32) in the IL-15Rα. In specific embodiments, the glycosylated IL-15Rα is a native human IL-15Rα. In other specific embodiments, the glycosylated IL-15Rα is an IL-15Rα derivative of naturally occurring human IL-15Rα. In some embodiments, the glycosylated IL-15Rα is a native soluble human IL-15Rα. In other embodiments, the glycosylated IL-15Rα is an IL-15Rα derivative that is a soluble form of human IL-15Rα. In specific embodiments, the glycosylated IL-15Rα has the amino acid sequence of SEQ ID NOs:10-21. In particular embodiments, the glycosylated IL-15Rα has an amino acid sequence that is at least 40%, 45%, 50%, 55%, 60%, 65%, 70%, 75%, 80%, 85%, 90%, 95%, 98% or 99% identical to SEQ ID NOs:10-21. In certain embodiments, the IL-15/IL-15Rα complex is purified or isolated.

In certain embodiments, provided herein is a glycosylated form of IL-15Rα (e.g., human IL-15Rα), wherein the glycosylation accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Rα, and which is glycosylated on at least one, at least two, at least three, at least four, at least five, at least six, or at least seven of the following sites: (i) Thr5 of amino acid sequence NWELTASASHQPPGVYPQG (SEQ ID NO:30) in the IL-15Rα (e.g., O-glycosylated); (ii) Ser7 of amino acid sequence NWELTASASHQPPGVYPQG (SEQ ID NO:30) in the IL-15Rα (e.g., O-glycosylated); (iii) Ser 8 of amino acid sequence ITCPPPMSVEHADIWVK (SEQ ID NO:31) or amino acid sequence ITCPPPMSVEHADIWVKSYSLYSRERYICNS (SEQ ID NO:32) in the IL-15Rα (e.g., N-glycosylated); (iv) Ser 18 of amino acid sequence ITCPPPMSVEHADIWVKSYSLYSRERYICNS (SEQ ID NO:32) in the IL-15Rα (e.g., N-glycosylated); (v) Ser 20 of amino acid sequence ITCPPPMSVEHADIWVKSYSLYSRERYICNS (SEQ ID NO:32) in the IL-15Rα (e.g., N-glycosylated); (vi) Ser 23 of amino acid sequence ITCPPPMSVEHADIWVKSYSLYSRERYICNS (SEQ ID NO:32) in the IL-15Rα (e.g., N-glycosylated); (vii) Ser 31 of amino acid sequence ITCPPPMSVEHADIWVKSYSLYSRERYICNS (SEQ ID NO:32) in the IL-15Rα (e.g., N-glycosylated). In a particular embodiment, the glycosylated human IL-15Rα comprises amino acid sequence of SEQ ID NOs:10-21. In another embodiment, the glycosylated human IL-15Rα is: (i) soluble; and (ii) (a) the last amino acids at the C-terminal end of the soluble form of human IL-15Rα consist of amino acid residues PQGHSDTT (SEQ ID NO:29), wherein T is at the C-terminal end of the amino acid sequence; (b) the last amino acids at the C-terminal end of the soluble form of human IL-15Rα consist of amino acid residues PQGHSDT (SEQ ID NO:28), wherein T is at the C-terminal end of the amino acid sequence; (c) the last amino acids at the C-terminal end of the soluble form of human IL-15Rα consist of amino acid residues PQGHSD (SEQ ID NO:27), wherein D is at the C-terminal end of the amino acid sequence; (d) the last amino acids at the C-terminal end of the soluble form of IL-15Rα consist of amino acid residues PQGHS (SEQ ID NO:26), wherein S is at the C-terminal end of the amino acid sequence; (e) the last amino acids at the C-terminal end of the soluble form of human IL-15Rα consist of amino acid residues PQGH (SEQ ID NO:25), wherein H is at the C-terminal end of the amino acid sequence; or (f) the last amino acids at the C-terminal end of the soluble form of human IL-15Rα consist of amino acid residues PQG, wherein G is at the C-terminal end of the amino acid sequence. In specific embodiments, the glycosylated IL-15Rα is part of a composition comprising IL-15. In certain embodiments, the glycosylated IL-15Rα is part of an IL-15/IL-15Rα complex.

In certain embodiments, an IL-15/IL-15Rα complex is associated with a cell. In a specific embodiment, the extracellular domain cleavage site of IL-15Rα that is cleaved by an endogenous processing enzyme is replaced with a heterologous domain (e.g., heterologous transmembrane domain) or a synthetic amino acid sequence that does not allow cleavage and generation of soluble IL-15Rα. In certain embodiments, the extracellular domain cleavage site of IL-15Rα that is cleaved by an endogenous processing enzyme is mutated to inhibit cleavage and generation of soluble IL-15Rα.

In addition to IL-15 and IL-15Rα, the IL-15/IL-15Rα complexes may comprise a heterologous molecule. The heterologous molecule may be conjugated to IL-15 and/or IL-15Rα. The heterologous molecule is conjugated to IL-15 or IL-15Rα in a manner that does not interfere or prevent IL-15 and IL-15Rα from binding to one another and does not interfere or prevent the interaction between the IL-15/IL-15Rα complex and the beta-gamma subunits of the IL-15 receptor. In some embodiments, the heterologous molecule is an antigen associated with a disease that one intends to prevent, treat and/or manage. Non-limiting examples of such antigens include viral antigens, bacterial antigens, parasitic antigens, and tumor antigens. In other embodiments, the heterologous molecule is an antibody that specifically binds to an antigen associated with a disease that one intends to prevent, treat and/or manage. In some embodiments, the antibody specifically binds to a cellular antigen (e.g., a receptor) expressed by a cell that one desires to target. In some embodiments, the heterologous molecule increases protein stability. In certain embodiments, the heterologous molecule is an Fc domain of an immunoglobulin or a fragment thereof. In certain embodiments, IL-15Rα is conjugated/fused to the Fc domain of an immunoglobulin (e.g., an IgG1). In other embodiments, the heterologous molecule is not an Fc domain of an immunoglobulin molecule or a fragment thereof.

Also provided herein are nucleic acids that encode IL-15 and IL-15Rα. The nucleic acids encode IL-15 and IL-15Rα that are capable of covalently or noncovalently binding to each other to form the IL-15/IL-15Rα complexes described herein. Such IL-15/IL-15Rα complexes can bind to the beta-gamma receptor complex and induce IL-15-mediated signal transduction.

Nucleic acid sequences encoding native IL-15 are well known in the art and have been described, for a review, see, Fehniger and Caligiuri, Blood, 2001, 97:14-32, which is incorporated by reference herein in its entirety. For example, the nucleic acid sequences encoding native IL-15 can be readily found in publicly available publications and databases, e.g., National Center for Biotechnology Information website at ncbi.nlm.nih.gov. Nucleic acid sequences encoding native IL-15Rα have been described, e.g., see International Publication No. WO 95/30695, and can also be readily found in publicly available publications and databases, e.g., National Center for Biotechnology Information website at ncbi.nlm.nih.gov. Cloning techniques well known in the art can be used to generate nucleic acids encoding IL-15 and IL-15Rα. See, e.g., Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999).

In a specific embodiment, provided herein are nucleic acids that encode the IL-15 and IL-15Rα polypeptides described herein. In a particular embodiment, provided herein are nucleic acids that encode an IL-15Rα polypeptide described herein. In another embodiment, provided herein are nucleic acids that encode an IL-15Rα polypeptide comprising the amino acid sequence of SEQ ID NOs:10-21. In another embodiment, provided herein is a nucleic acid sequence that encodes an IL-15Rα polypeptide, wherein the nucleic acid sequence comprises:

| (SEQ ID NO: 22) | |
|---|---|
| atggccccgc ggcgggcgcg cggctgccgg accctcggtc tcccggcgct gctactgctg | 60 |
| ctgctgctcc ggccgccggc gacgcggggc atcacgtgcc ctccccccat gtccgtggaa | 120 |
| cacgcagaca tctgggtcaa gagctacagc ttgtactcca gggagcggta catttgtaac | 180 |
| tctggtttca agcgtaaagc cggcacgtcc agcctgacgg agtgcgtgtt gaacaaggcc | 240 |
| acgaatgtcg cccactggac aacccccagt ctcaaatgca ttagagaccc tgccctggtt | 300 |
| caccaaaggc cagcgccacc ctccacagta acgacggcag gggtgacccc acagccagag | 360 |
| agcctctccc cttctggaaa agagcccgca gcttcatctc ccagctcaaa caacacagcg | 420 |
| gccacaacag cagctattgt cccgggctcc cagctgatgc cttcaaaatc accttccaca | 480 |
| ggaaccacag agataagcag tcatgagtcc tcccacggca ccccctctca gacaacagcc | 540 |
| aagaactggg aactcacagc atccgcctcc caccagccgc caggtgtgta tccacagggc | 600 |
| cacagcgaca ccactgtggc tatctccacg tccactgtcc tgctgtgtgg gctgagcgct | 660 |
| gtgtctctcc tggcatgcta cctcaagtca aggcaaactc ccccgctggc cagcgttgaa | 720 |
| atggaagcca tggaggctct gccggtgact tgggggacca gcagcagaga tgaagacttg | 780 |
| gaaaactgct ctcaccacct atga | 804 |
| or | |
| (SEQ ID NO: 23) | |
| atggccccgc ggcgggcgcg cggctgccgg accctcggtc tcccggcgct gctactgctg | 60 |
| ctgctgctcc ggccgccggc gacgcggggc atcacgtgcc ctccccccat gtccgtggaa | 120 |
| cacgcagaca tctgggtcaa gagctacagc ttgtactcca gggagcggta catttgtaac | 180 |
| tctggtttca agcgtaaagc cggcacgtcc agcctgacgg agtgcgtgtt gaacaaggcc | 240 |
| acgaatgtcg cccactggac aacccccagt ctcaaatgca ttagagaccc tgccctggtt | 300 |
| caccaaaggc cagcgccacc ctccacagta acgacggcag gggtgacccc acagccagag | 360 |
| agcctctccc cttctggaaa agagcccgca gcttcatctc ccagctcaaa caacacagcg | 420 |
| gccacaacag cagctattgt cccgggctcc cagctgatgc cttcaaaatc accttccaca | 480 |
| ggaaccacag agataagcag tcatgagtcc tcccacggca ccccctctca gacaacagcc | 540 |
| aagaactggg aactcacagc atccgcctcc caccagccgc caggtgtgta tccacagggc | 600 |
| cacagcgaca ccact | 615 |

In another embodiment, provided herein is a nucleic acid sequence that encodes an IL-15 polypeptide comprising the amino acid sequence of SEQ ID NO:1 from Table 1 or amino acid residues 49 to 162 of SEQ ID NO:1 from Table 1. In another embodiment, provided herein is a nucleic acid sequence that encodes an IL-15 polypeptide, wherein the nucleic acid sequence comprises:

| (SEQ ID NO: 24) | |
|---|---|
| atgagaattt cgaaaccaca tttgagaagt atttccatcc agtgctactt gtgtttactt | 60 |
| ctaaacagtc attttctaac tgaagctggc attcatgtct tcattttggg ctgtttcagt | 120 |
| gcagggcttc ctaaaacaga agccaactgg gtgaatgtaa taagtgattt gaaaaaaatt | 180 |
| gaagatctta ttcaatctat gcatattgat gctactttat atacggaaag tgatgttcac | 240 |
| cccagttgca aagtaacagc aatgaagtgc tttctcttgg agttacaagt tatttcactt | 300 |
| gagtccggag atgcaagtat tcatgataca gtagaaaatc tgatcatcct agcaaacaac | 360 |
| agtttgtctt ctaatgggaa tgtaacagaa tctggatgca aagaatgtga ggaactggag | 420 |
| gaaaaaaata ttaaagaatt tttgcagagt tttgtacata ttgtccaaat gttcatcaac | 480 |
| acttcttga | 489 |

In another specific embodiment, the nucleic acids that encode IL-15 and/or IL-15Rα that are optimized, e.g., by codon/RNA optimization, replacement with heterologous signal sequences, and elimination of mRNA instability elements. Methods to generate optimized nucleic acids encoding IL-15 and IL-15Rα for expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, for IL-15 and IL-15Rα. The contents of each of these references are incorporated by reference herein in its entirety. See also, U.S. Provisional Application No. 60/812,566, filed on Jun. 9, 2006, and 60/758,819, filed on Jan. 13, 2007, and International Patent Application Publication Nos. WO 2007/084342 and WO 2010/020047, which are also incorporated by reference herein in their entireties. For example, potential splice sites and instability elements (e.g., A/T or A/U rich elements) within the RNA of IL-15 and IL-15Rα can be mutated without altering the amino acids encoded by the nucleic acid sequences to increase stability of the RNA for expression. The alterations utilize the degeneracy of the genetic code, e.g., using an alternative codon for an identical amino acid. In some embodiments, it may be desirable to alter one or more codons to encode a conservative mutation, e.g., a similar amino acid with similar chemical structure and properties and/or function as the original amino acid. Such methods can increase expression of IL-15 and/or IL-15Rα proteins by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of IL-15 and/or IL-15Rα proteins encoded by native nucleic acid sequences.

Further, the native signal peptide sequence of IL-15 and/or IL-15Rα can be replaced with a heterologous signal peptide, e.g., a signal peptide of human GM-CSF, tissue plasminogen activator (tPA), preprolactin, growth hormone or an immunoglobulin protein (e.g., IgE). In a specific embodiment, the signal peptide of IL-15 is replaced with the signal sequence of tPA. In other specific embodiments, the signal peptide of IL-15 is replaced with the signal peptide of human GM-CSF. Such alternations can increase expression of IL-15 and/or IL-15Rα proteins/polypeptides by at least 1-fold, 2-fold, 3-fold, 4-fold, 5-fold, 10-fold, 20-fold, 30-fold, 40-fold, 50-fold, 60-fold, 70-fold, 80-fold, 90-fold, or 100-fold or more relative to the expression of IL-15 and/or IL-15Rα proteins with the respective native signal peptide, as measured/detected by a technique known to one of skill in the art, e.g., ELISA.

In some embodiments, an optimized nucleotide sequence encoding IL-15 or IL-15Rα hybridizes to the nucleotide sequence encoding native IL-15 or IL-15Rα, respectively. In specific embodiments, an optimized nucleotide sequence encoding IL-15 or IL-15Rα hybridizes under high stringency conditions to a nucleotide sequence encoding native IL-15 or IL-15Rα, respectively, or a fragment thereof. In a specific embodiment, an optimized nucleotide sequence encoding IL-15 or IL-15Rα hybridizes under high stringency, intermediate or lower stringency hybridization conditions to a nucleotide sequence encoding native IL-15 or IL-15Rα, respectively, or a fragment thereof. Information regarding hybridization conditions have been described, see, e.g., U.S. Patent Application Publication No. US 2005/0048549 (e.g., paragraphs 72-73).

Also provided herein are nucleic acids encoding IL-15, IL-15Rα, and a heterologous molecule in a form that allows IL-15 to covalently or noncovalently bind to the IL-15Rα to form IL-15/IL-15Rα complexes. In some embodiments, the heterologous molecule is an antigen associated with a disease that one intends to prevent, treat and/or manage. In other embodiments, the heterologous molecule is an antibody that specifically binds to an antigen associated with a disease that one intends to prevent, treat and/or manage. In some embodiments, the antibody specifically binds to a cellular surface antigen (e.g., a receptor) expressed by a cell that one desires to target. In some embodiments, the heterologous molecule increases protein stability. Non-limiting examples of such molecules include polyethylene glycol (PEG), Fc domain of an IgG immunoglobulin or a fragment thereof, or albumin that increase the half-life of IL-15 or IL-15Rα in vivo. In certain embodiments, the heterologous molecules is not an Fc domain of an immunoglobulin molecule or a fragment thereof.

In those IL-15/IL-15Rα complexes comprising a heterologous molecule, the heterologous molecule may be conjugated to IL-15 and/or IL-15Rα. In one embodiment, the heterologous molecule is conjugated to IL-15Rα. In another embodiment, the heterologous molecule is conjugated to IL-15.

In specific embodiments, IL-15 and IL-15Rα are encoded by one nucleic acid construct (e.g., bicistronic construct). In some embodiments, IL-15 and IL-15Rα are encoded by one nucleic acid construct comprising a single open reading frame (ORF) of IL-15 and IL-15Rα. In some embodiments, IL-15 or IL-15Rα encoded by a nucleic acid construct may be linked to a nucleic acid encoding a heterologous molecule (to produce a fusion molecule), such heterologous molecules being an antigen, a targeting protein or an antibody of interest. In other embodiments, IL-15 and IL-15Rα are encoded by two nucleic acid constructs, wherein a first nucleic acid construct encodes IL-15 and a second nucleic acid construct encodes IL-15Rα. The IL-15 encoded by the first nucleic acid construct may be linked to a nucleic acid encoding a heterologous molecule, such as an antigen or an antibody of interest, to produce a fusion protein. Alternatively, or in addition, the IL-15Rα encoded by the second nucleic acid construct may be linked to a nucleic acid encoding a heterologous molecule, such as an antigen or an antibody of interest.

TABLE 1
IL-15 sequences

Immature/precursor form of native human IL-15
Sequence was identified using sequence identifier number 1 in
PCT/US2008/008084 (WO 2009/002562)
MRISKPHLRSISIQCYLCLLLNSHFLTEAGIHVFILGCFSAGLPKTEANWVNVISDLKKIEDLIQSMHI
DATLYTESDVHPSCKVTAMKCFLLELQVISLESGDASIHDTVENLIILANNSLSSNGNVTESGCKECEE
LEEKNIKEFLQSFVHIVQMFINTS (SEQ ID NO: 01)
>residues 49-162 of SEQ ID NO: 01 above
NWVNVISDLKKIEDLIQSMHIDATLYTESDVHPSCKVTAMKCFLLELQVISLESGDASIHDTVENLIIL
ANNSLSSNGNVTESGCKECEELEEKNIKEFLQSFVHIVQMFINTS (SEQ ID NO: 02)
Immature form of the native full length human IL-15 receptor alpha
Sequence was identified using sequence identifier number 3 in
PCT/US2008/008084 (WO 2009/002562)
MAPRRARGCRTLGLPALLLLLLLRPPATRGITCPPPMSVEHADIWVKSYSLYSRERYICNSGFKRKAGT
SSLTECVLNKATNVAHWTTPSLKCIRDPALVHQRPAPPSTVTTAGVTPQPESLSPSGKEPAASSPSSNN
TAATTAAIVPGSQLMPSKSPSTGTTEISSHESSHGTPSQTTAKNWELTASASHQPPGVYPQGHSDTTVA
ISTSTVLLCGLSAVSLLACYLKSRQTPPLASVEMEAMEALPVTWGTSSRDEDLENCSHHL (SEQ ID
NO: 03)
Immature form of the native soluble human IL-15 receptor alpha
Sequence was identified using sequence identifier number in
PCT/US2008/008084 (WO 2009/002562)
MAPRRARGCRTLGLPALLLLLLLRPPATRGITCPPPMSVEHADIWVKSYSLYSRERYICNSGFKRKAGT
SSLTECVLNKATNVAHWTTPSLKCIRDPALVHQRPAPPSTVTTAGVTPQPESLSPSGKEPAASSPSSNN
TAATTAAIVPGSQLMPSKSPSTGTTEISSHESSHGTPSQTTAKNWELTASASHQPPGVYPQGHSDTT
(SEQ ID NO: 04)
>residues 31-205 of SEQ ID NO: 04 above
ITCPPPMSVEHADIWVKSYSLYSRERYICNSGFKRKAGTSSLTECVLNKATNVAHWTTPSLKCIRDPAL
VHQRPAPPSTVTTAGVTPQPESLSPSGKEPAASSPSSNNTAATTAAIVPGSQLMPSKSPSTGTTEISSH
ESSHGTPSQTTAKNWELTASASHQPPGVYPQGHSDTT (SEQ ID NO: 05)
AG32 huIL15opt-amino acid sequence of optimized human IL-15
Sequence was identified using sequence identifier number 10 in
PCT/US2008/008084 (WO 2009/002562)
MRISKPHLRSISIQCYLCLLLNSHFLTEAGIHVFILGCFSAGLPKTEANWVNVISDLKKIEDLIQSMHI
DATLYTESDVHPSCKVTAMKCFLLELQVISLESGDASIHDTVENLIILANNSLSSNGNVTESGCKECEE
LEEKNIKEFLQSFVHIVQMFINTS (SEQ ID NO: 01)
AG59 CMV huIL15tPA6-amino acid sequence of optimized human IL-15
Sequence was identified using sequence identifier number 12 in
PCT/US2008/008084 (WO 2009/002562)
MDAMKRGLCCVLLLCGAVFVSPSQEIHARFRRGARNWVNVISDLKKIEDLIQSMHIDATLYTESDVHPS
CKVTAMKCFLLELQVISLESGDASIHDTVENLIILANNSLSSNGNVTESGCKECEELEEKNIKEFLQSF
VHIVQMFINTS (SEQ ID NO: 06)
AG79 huIL15Rα-amino acid sequence of optimized human IL-15Rα
Sequence was identified using sequence identifier number 14 in
PCT/US2008/008084 (WO 2009/002562)
MAPRRARGCRTLGLPALLLLLLLRPPATRGITCPPPMSVEHADIWVKSYSLYSRERYICNSGFKRKAGT
SSLTECVLNKATNVAHWTTPSLKCIRDPALVHQRPAPPSTVTTAGVTPQPESLSPSGKEPAASSPSSNN
TAATTAAIVPGSQLMPSKSPSTGTTEISSHESSHGTPSQTTAKNWELTASASHQPPGVYPQGHSDTTVA
ISTSTVLLCGLSAVSLLACYLKSRQTPPLASVEMEAMEALPVTWGTSSRDEDLENCSHHL (SEQ ID
NO: 07)
AG98 CMV hu sIL15Rα-amino acid sequence of optimized human IL-15Rα
Sequence was identified using sequence identifier number 16 in
PCT/US2008/008084 (WO 2009/002562)
MAPRRARGCRTLGLPALLLLLLLRPPATRGITCPPPMSVEHADIWVKSYSLYSRERYICNSGFKRKAGT
SSLTECVLNKATNVAHWTTPSLKCIRDPALVHQRPAPPSTVTTAGVTPQPESLSPSGKEPAASSPSSNN
TAATTAAIVPGSQLMPSKSPSTGTTEISSHESSHGTPSQTTAKNWELTASASHQPPGVYPQGHSDTT
(SEQ ID NO: 08)
AG151 huIL-15 huGM-CSF-amino acid sequence of optimized human IL-15
with a signal peptide of human GM-CSF
MWLQSLLLLGTVACSISNWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTS (SEQ
ID NO: 09)

In some embodiments, the IL-15 or IL-15/IL-15Rα complexes disclosed herein comprise any of the IL-15 sequences disclosed in WO2007084342, WO2009002562, WO2011020047, WO2014066527 or WO2016018920, which are hereby expressly incorporated by reference in their entirety.

In some embodiments, the IL-15 or IL-15/IL-15Rα complexes disclosed herein comprise any of the IL-15 sequence disclosed in Table 1 or Table 4.

In some embodiments, IL-15 is at least 95% identical to the amino acid sequence comprising amino acid residues 49 to 162 of SEQ ID NO:1 in Table 1.

In some embodiments, IL-15 comprises amino acid residues 49 to 162 of SEQ ID NO:01 in Table 1.

In some embodiments, IL-15 comprises amino acid residues 30 to 162 of SEQ ID NO:01 in Table 1.

In some embodiments, IL-15 comprises amino acid residues 36 to 149 of SEQ ID NO:06 in Table 1.

In some embodiments, IL-15 comprises amino acid residues 18 to 131 of SEQ ID NO:08 in Table 1.

In some embodiments, IL-15Rα is at least 95% identical to the amino acid sequence comprising amino acid residues 31 to 267 of SEQ ID NO:3 in Table 1.

In some embodiments, IL-15Rα comprises amino acid residues 31 to 267 of SEQ ID NO:3 in Table 1.

In some embodiments, IL-15Rα is at least 95% identical to the amino acid sequence comprising amino acid residues 31 to 205 of SEQ ID NO: 04 in Table 1.

In some embodiments, IL-15Rα comprises amino acid residues 31 to 205 of SEQ ID NO: 04 in Table 1.

In some embodiments, IL15Rα comprises amino acid residues 31 to 267 of SEQ ID NO:07 in Table 1.

In some embodiments, IL-15Rα comprises amino acid residues 31 to 205 of SEQ ID NO:08 in Table 1.

In certain embodiments, IL-15Rα derivatives retain at least 50%, 55%, 60%, 65%, 70%, 75%, 80%, 85%, 90%, 95%, 98% or 99% of the function of a mammalian IL-15Rα polypeptide to bind an IL-15 polypeptide, as measured by assays well known in the art, e.g., electromobility assays, electromobility shift assays, live cell bioassays, ELISA, Biacore, co-immunoprecipitation. In another preferred embodiment, IL-15Rα derivatives retain at least 50%, 55%, 60%, 65%, 70%, 75%, 80%, 85%, 90%, 95%, 98% or 99% of the function of a mammalian IL-15Rα polypeptide to induce IL-15-mediated signal transduction, as measured by assays well-known in the art, e.g., electromobility assays, electromobility shift assays, ELISA's, live cell bioassays, and other immunoassays.

As used herein, the term “IL-15/IL-15Rα complex” refers to a complex comprising IL-15 and IL-15Rα covalently or noncovalently bound to each other. In some embodiments, the IL-15Rα has a relatively high affinity for IL-15, e.g., K_{d} of 10 to 50 pM as measured by a technique known in the art, e.g., KinEx A assay, plasma surface resonance (e.g., BIAcore assay). In another preferred embodiment, the IL-15/IL-15Rα complex induces IL-15-mediated signal transduction, as measured by assays well-known in the art, e.g., electromobility assays, electromobility shift assays, ELISA's and other immunoassays or live cell bioassays.

Compositions comprising IL-15 or an IL-15/IL-15Rα complex can be administered once or multiple times. In some embodiments, treating a subject may comprise administration performed more than once, for example, 2, 3, 4, 5, 6, 7, 8, 10, 15, 20 or more times as needed to induce the desired response. Multiple administrations can be administered, for example, daily, every other day, bi-weekly, weekly, bi-monthly, monthly, or more or less often, as needed, for a time period sufficient to achieve the desired response.

The compositions comprising IL-15 or an IL-15/IL-15Rα complex may be directly fused, using either non-covalent bonds or covalent bonds (e.g., by combining amino acid sequences via peptide bonds), and/or may be combined using one or more linkers. In a specific embodiment, IL-15 and IL-15Rα are directly fused to each other using either non-covalent bonds or covalent bonds (e.g., by combining amino acid sequences via peptide bonds), and/or may be combined using one or more linkers. In specific embodiments, a polypeptide comprising IL-15 and IL-15Rα directly fused to each other using either non-covalent bonds or covalent bonds is functional (e.g., capable of specifically binding to the IL-15R beta-gamma complex and inducing IL-15-mediated signal transduction and/or IL-15-mediated immune function). Linkers suitable for preparing the IL-15/IL-15Rα complexes comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (“PEG”). In some embodiments, the linker is a peptide that is 1, 2, 3, 4, 5, 6, 7, 8, 9, 10, 11, 12, 13, 14, 15, 16, 17, 18, 19, 20 or more amino acids long. In a specific embodiment, the linker is long enough to preserve the ability of IL-15 to bind to the IL-15Rα. In other embodiments, the linker is long enough to preserve the ability of the IL-15/IL-15Rα complex to bind to the beta-gamma receptor complex and to act as an agonist to mediate IL-15 signal transduction.

In some embodiments, the IL-15 or an IL-15/IL-15Rα complex of this disclosure are administered to a mammalian subject. The mammalian subject usually is a human.

In certain embodiments, the IL-15 or IL-15/IL-15Rα complex is administered subcutaneously to a subject in accordance with the methods described herein. In some embodiments, the IL-15 and/or IL-15/IL-15Rα complex is administered intravenously or intramuscularly to a subject in accordance with the methods described herein. In certain embodiments, the IL-15 and/or IL-15/IL-15Rα is administered intratumorally to a subject in accordance with the methods described herein. In some embodiments, the IL-15 or IL-15/IL-15Rα complex is administered locally to a site (e.g., a tumor site, a site of infection) in a subject in accordance with the methods described herein.

Suitable quantities of the IL-15 or an IL-15/IL-15Rα complex can be about 0.5 μg to about 200 mg. For example, in some embodiments a suitable quantity of IL-15 is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, or about 100 μg/kg, or higher. For example, in some embodiments a suitable quantity of an IL-15/IL-15Rα complex is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher. For example, in some embodiments a suitable quantity of an IL-15/IL-15Rα complex fused to an Fc molecule is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the IL-15 or an IL-15/IL-15Rα complex can be administered by methods well known in the art. One skilled in the art would know that the choice of a pharmaceutically acceptable carrier, including a physiologically acceptable compound, depends, for example, on the route of administration.

### IL-12

Interleukin-12 (IL-12) is IL12 is a heterodimeric cytokine encoded by two separate genes, IL-12A (p35) and IL-12B (p40). The active heterodimer (referred to as ‘p70’), and a homodimer of p40 are formed following protein synthesis. IL-12A is composed of a bundle of four alpha helices. IL-12B has three beta sheet domains. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. T cells that produce IL-12 have a coreceptor, CD30, which is associated with IL-12 activity. IL-12 plays an important role in the activities of natural killer cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-31 and IL-12R-32, maintaining the expression of a critical protein involved in IL-12 signaling in NK cells. Enhanced functional response is demonstrated by IFN-γ production and killing of target cells.

As used herein, the terms “IL-12” and “interleukin-12 in the context of proteins or polypeptides refer to any mammalian interleukin-12 amino acid sequences, including immature or precursor and mature forms. Non-limiting examples of IL-12 sequences can include NCBI Reference Sequence: NM_000882.4; UniProt accession P29459; NCBI Reference Sequence: NM_002187.3; UniProt accession accession P29460. Amino acid sequences of the IL-12 are provided in the table below.

Table-IL-12 and fusion protein sequences

MOUSE IL-15_IL-12p40 fusion Protein
Underlined: mouse GM-CSF signal peptide
Bold: mouse IL-15
Italics: IL-12p40 with signal peptide underlined
Alternative fusion molecules contain a GAGA (SEQ ID NO: 42) or other
appropriate amino acid linker at the junction between IL-15 and IL-12p40.
The presence of the Linker indicated with: (L) in examples.
MWLQNLLFLGIVVYSLSNWIDVRYDLEKIESLIQSIHIDTTLYTDSDFHPSCKVTAMNCFLLELQVILHEYSNMT
LNETVRNVLYLANSTLSSNKNVAESGCKECEELEEKTFTEFLQSFIRIVQMFINTSMWELEKDVYVVEVDWTPDA
PGETVNLTCDTPEEDDITWTSDQRHGVIGSGKTLTITVKEFLDAGQYTCHKGGETLSHSHLLLHKKENGIWSTEI
LKNFKNKTFLKCEAPNYSGRFTCSWLVQRNMDLKFNIKSSSSSPDSRAVTCGMASLSAEKVTLDQRDYEKYSVSC
QEDVTCPTAEETLPIELALEARQQNKYENYSTSFFIRDIIKPDPPKNLQMKPLKNSQVEVSWEYPDSWSTPHSYF
SLKFFVRIQRKKEKMKETEEGCNQKGAFLVEKTSTEVQCKGGNVCVQAQDRYYNSSCSKWACVPCRVRS
(SEQ ID NO: 47)
DNA coding sequence of mouse IL-12_IL-15 fusion open reading frame
ATGTGGCTCCAGAACCTGCTATTCCTGGGGATCGTGGTCTACAGCCTCTCGAACTGGATCGATGTGCGCTACGAC
CTGGAGAAGATCGAGTCCCTCATCCAGTCGATCCACATCGACACCACGCTGTACACGGACTCGGACTTCCACCCG
TCGTGCAAGGTCACGGCGATGAACTGCTTCCTCCTGGAGCTGCAAGTCATCCTGCACGAGTACTCGAACATGACC
CTCAACGAGACGGTGCGCAACGTGCTGTACCTGGCGAACTCGACCCTGTCGTCGAACAAGAACGTCGCCGAGTCG
GGCTGCAAGGAGTGCGAGGAGCTGGAGGAGAAGACCTTCACGGAGTTCCTGCAGTCGTTCATCCGCATCGTCCAG
ATGTTCATCAACACGTCGATGTGGGAGCTGGAGAAGGACGTCTACGTCGTGGAGGTGGACTGGACGCCCGACGCC
CCCGGGGAGACGGTGAACCTCACCTGCGACACGCCGGAGGAGGACGACATCACCTGGACCAGCGACCAGCGGCAC
GGCGTCATCGGCAGCGGCAAGACCCTGACGATCACCGTCAAGGAGTTCCTCGACGCCGGCCAGTACACCTGCCAC
AAGGGCGGCGAGACGCTGAGCCACAGCCACCTGCTGCTCCACAAGAAGGAGAACGGCATCTGGTCCACCGAGATC
CTCAAGAACTTCAAGAACAAGACCTTCCTGAAGTGCGAGGCGCCCAACTACTCCGGCCGGTTCACGTGCTCCTGG
CTGGTGCAGCGCAACATGGACCTCAAGTTCAACATCAAGTCGTCCAGCAGTTCCCCCGACTCCCGGGCGGTGACC
TGCGGCATGGCGTCCCTGTCGGCGGAGAAGGTCACGCTGGACCAGAGGGACTACGAGAAGTACTCGGTGTCCTGC
CAGGAGGACGTCACCTGCCCCACGGCCGAGGAGACCCTGCCCATCGAGCTGGCGCTGGAGGCCCGGCAGCAGAAC
AAGTACGAGAACTACAGCACCAGCTTCTTCATCCGGGACATCATCAAGCCCGACCCGCCCAAGAACCTGCAGATG
AAGCCCCTGAAGAACAGCCAGGTGGAGGTCAGCTGGGAGTACCCGGACTCCTGGAGCACGCCCCACTCCTACTTC
AGCCTGAAGTTCTTCGTCAGAATCCAGCGCAAGAAAGAGAAGATGAAGGAGACGGAGGAGGGGTGCAACCAGAAG
GGCGCGTTCCTCGTCGAGAAGACCTCCACCGAGGTCCAGTGCAAGGGCGGGAACGTCTGCGTGCAGGCTCAGGAC
CGCTACTACAACTCCAGCTGCAGCAAGTGGGCCTGCGTCCCCTGCAGGGTCCGGTCCTAGTAA (SEQ ID
NO: 48)
Mouse IL-15_IL-12p40 fusion Protein without p40 signal peptide
Underlined: mouse GM-CSF signal peptide
Bold: mouse IL-15
Italics: IL-12p40 without signal peptide
Alternative fusion molecules contain a GAGA (SEQ ID NO: 42) or other
appropriate amino acid linker at the junction between IL-15 and IL-12p40
MWLQNLLFLGIVVYSLSNWIDVRYDLEKIESLIQSIHIDTTLYTDSDFHPSCKVTAMNCFLLELQVILHEYSNMT
LNETVRNVLYLANSTLSSNKNVAESGCKECEELEEKTFTEFLQSFIRIVQMFINTSTVNLTCDTPEEDDITWTSD
QRHGVIGSGKTLTITVKEFLDAGQYTCHKGGETLSHSHLLLHKKENGIWSTEILKNFKNKTFLKCEAPNYSGRFT
CSWLVQRNMDLKFNIKSSSSSPDSRAVTCGMASLSAEKVTLDQRDYEKYSVSCQEDVTCPTAEETLPIELALEAR
QQNKYENYSTSFFIRDIIKPDPPKNLQMKPLKNSQVEVSWEYPDSWSTPHSYFSLKFFVRIQRKKEKMKETEEGC
NQKGAFLVEKTSTEVQCKGGNVCVQAQDRYYNSSCSKWACVPCRVRS (SEQ ID NO: 49)
HUMAN IL-15_IL-12p40 fusion Protein
Underlined: human GM-CSF signal peptide
Bold: human IL-15
Italics: human IL-12p40 with signal peptide underlined
Alternative fusion molecules contain a GAGA (SEQ ID NO: 42) or other
appropriate amino acid linker at the junction between IL-15 and IL-12p40
MWLQSLLLLGTVACSISNWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTSMCHQQLVISWFSLVFLASP
LVAIWELKKDVYVVELDWYPDAPGEMVVLTCDTPEEDGITWTLDQSSEVLGSGKTLTIQVKEFGDAGQYTCHKGG
EVLSHSLLLLHKKEDGIWSTDILKDQKEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKLKYENYTSSFFIRDIIKPDPPKNLQLK
PLKNSRQVEVSWEYPDTWSTPHSYFSLTFCVQVQGKSKREKKDRVFTDKTSATVICRKNASISVRAQDRYYSSSW
SEWASVPCS (SEQ ID NO: 50)
DNA sequence of human IL-12_IL-15 fusion open reading frame
ATGTGGCTCCAGAGCCTGCTACTCCTGGGGACGGTGGCCTGCAGCATCTCGAACTGGGTGAACGTGATCTCGGAC
CTGAAGAAGATCGAGGACCTCATCCAGTCGATGCACATCGACGCGACGCTGTACACGGAGTCGGACGTCCACCCG
TCGTGCAAGGTCACGGCGATGAAGTGCTTCCTCCTGGAGCTCCAAGTCATCTCGCTCGAGTCGGGGGACGCGTCG
ATCCACGACACGGTGGAGAACCTGATCATCCTGGCGAACAACTCGCTGTCGTCGAACGGGAACGTCACGGAGTCG
GGCTGCAAGGAGTGCGAGGAGCTGGAGGAGAAGAACATCAAGGAGTTCCTGCAGTCGTTCGTGCACATCGTCCAG
ATGTTCATCAACACGTCGATGTGCCACCAGCAGCTCGTGATCAGCTGGTTCAGCCTCGTCTTCCTCGCGTCGCCG
CTGGTCGCGATCTGGGAGCTGAAGAAGGACGTCTACGTGGTGGAGCTGGACTGGTATCCCGACGCGCCCGGGGAG
ATGGTCGTGCTGACGTGCGACACGCCGGAGGAGGACGGGATCACGTGGACGCTCGACCAGTCCTCGGAGGTGCTC
GGGTCCGGGAAGACGCTCACGATCCAGGTCAAGGAGTTCGGCGACGCCGGGCAGTACACGTGCCACAAGGGCGGC
GAGGTCCTCTCGCACTCCCTGCTCCTCCTGCACAAGAAGGAGGACGGCATCTGGAGCACGGACATCCTGAAGGAC
CAGAAGGAGCCGAAGAACAAGACGTTCCTGCGCTGCGAGGCGAAGAACTACTCCGGGCGCTTCACGTGCTGGTGG
CTCACCACGATCTCGACGGACCTGACGTTCTCGGTGAAGTCGTCGCGGGGCTCCTCGGACCCGCAAGGCGTCACC
TGCGGCGCAGCGACGCTGTCGGCGGAGCGCGTGCGGGGCGACAACAAGGAGTACGAGTACTCGGTCGAGTGCCAG
GAGGACTCGGCGTGCCCGGCAGCGGAGGAGTCGCTGCCGATCGAGGTGATGGTCGACGCGGTCCACAAGCTGAAG
TACGAGAACTACACGTCGTCCTTCTTCATCCGCGACATCATCAAGCCCGACCCGCCGAAGAACCTCCAGCTCAAG
CCGCTCAAGAACTCGCGCCAGGTCGAGGTGTCGTGGGAGTACCCCGACACGTGGTCGACGCCCCACTCCTACTTC
TCGCTCACCTTCTGCGTCCAAGTGCAGGGCAAGTCGAAGCGGGAGAAGAAGGACCGCGTGTTCACCGACAAGACC
AGCGCGACCGTGATCTGCCGGAAGAACGCGTCGATCTCGGTGCGGGCGCAGGACCGGTACTACTCGTCGTCGTGG
TCGGAGTGGGCCTCGGTGCCGTGCAGCTAGTAA (SEQ ID NO: 51)
Human IL-15_IL-12p40 fusion Protein without p40 signal peptide
Underlined: human GM-CSF signal peptide
Bold: human IL-15
Italics: human IL-12p40 without signal peptide
Alternative fusion molecules contain a GAGA (SEQ ID NO: 42) or other
appropriate amino acid linker at the junction between IL-15 and IL-12p40
MWLQSLLLLGTVACSISNWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTSIWELKKDVYVVELDWYPDA
PGEMVVLTCDTPEEDGITWTLDQSSEVLGSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSSDPQGVTCGAATLSAERVRGDNKEYEYSV
ECQEDSACPAAEESLPIEVMVDAVHKLKYENYTSSFFIRDIIKPDPPKNLQLKPLKNSRQVEVSWEYPDTWSTPH
SYFSLTFCVQVQGKSKREKKDRVFTDKTSATVICRKNASISVRAQDRYYSSSWSEWASVPCS
(SEQ ID NO: 52)
AG282
Expressed protein: Mouse GM-CSF-IL-15_IL-12p40
5362 base pairs
Underlined: Optimized coding sequence for expressed fusion protein
Bold: terminators
CCTGGCCATTGCATACGTTGTATCCATATCATAATATGTACATTTATATTGGCTCATGTCCAACATTACCGCCAT
GTTGACATTGATTATTGACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGTCAATGATGGTAAATGGCCCG
CCTGGCATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTCGTAACAACT
CCGCCCCATTGACGCAAATGGGCGGTAGGCGTGTACGGTGGGAGGTCTATATAAGCAGAGCTCGTTTAGTGAACC
GTCAGATCGCCTGGAGACGCCATCCACGCTGTTTTGACCTCCATAGAAGACACCGGGACCGATCCAGCCTCCGCG
GGCGCGCGTCGAGGAATTCTCTAGAAAGAAATGTGGCTCCAGAACCTGCTATTCCTGGGGATCGTGGTCTACAGC
CTCTCGAACTGGATCGATGTGCGCTACGACCTGGAGAAGATCGAGTCCCTCATCCAGTCGATCCACATCGACACC
ACGCTGTACACGGACTCGGACTTCCACCCGTCGTGCAAGGTCACGGCGATGAACTGCTTCCTCCTGGAGCTGCAA
GTCATCCTGCACGAGTACTCGAACATGACCCTCAACGAGACGGTGCGCAACGTGCTGTACCTGGCGAACTCGACC
CTGTCGTCGAACAAGAACGTCGCCGAGTCGGGCTGCAAGGAGTGCGAGGAGCTGGAGGAGAAGACCTTCACGGAG
TTCCTGCAGTCGTTCATCCGCATCGTCCAGATGTTCATCAACACGTCGATGTGGGAGCTGGAGAAGGACGTCTAC
GTCGTGGAGGTGGACTGGACGCCCGACGCCCCCGGGGAGACGGTGAACCTCACCTGCGACACGCCGGAGGAGGAC
GACATCACCTGGACCAGCGACCAGCGGCACGGCGTCATCGGCAGCGGCAAGACCCTGACGATCACCGTCAAGGAG
TTCCTCGACGCCGGCCAGTACACCTGCCACAAGGGCGGCGAGACGCTGAGCCACAGCCACCTGCTGCTCCACAAG
AAGGAGAACGGCATCTGGTCCACCGAGATCCTCAAGAACTTCAAGAACAAGACCTTCCTGAAGTGCGAGGCGCCC
AACTACTCCGGCCGGTTCACGTGCTCCTGGCTGGTGCAGCGCAACATGGACCTCAAGTTCAACATCAAGTCGTCC
AGCAGTTCCCCCGACTCCCGGGCGGTGACCTGCGGCATGGCGTCCCTGTCGGCGGAGAAGGTCACGCTGGACCAG
AGGGACTACGAGAAGTACTCGGTGTCCTGCCAGGAGGACGTCACCTGCCCCACGGCCGAGGAGACCCTGCCCATC
GAGCTGGCGCTGGAGGCCCGGCAGCAGAACAAGTACGAGAACTACAGCACCAGCTTCTTCATCCGGGACATCATC
AAGCCCGACCCGCCCAAGAACCTGCAGATGAAGCCCCTGAAGAACAGCCAGGTGGAGGTCAGCTGGGAGTACCCG
GACTCCTGGAGCACGCCCCACTCCTACTTCAGCCTGAAGTTCTTCGTCAGAATCCAGCGCAAGAAAGAGAAGATG
AAGGAGACGGAGGAGGGGTGCAACCAGAAGGGCGCGTTCCTCGTCGAGAAGACCTCCACCGAGGTCCAGTGCAAG
GGCGGGAACGTCTGCGTGCAGGCTCAGGACCGCTACTACAACTCCAGCTGCAGCAAGTGGGCCTGCGTCCCCTGC
AGGGTCCGGTCCTAGTAAAGATCTGATATCGGATCTGCTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGAGGATTGGGAAGAC
AATAGCAGGCATGCTGGGGATGCGGTGGGCTCTATGGGTACCCAGGTGCTGAAGAATTGACCCGGTTCCTCCTGG
GCCAGAAAGAAGCAGGCACATCCCCTTCTCTGTGACACACCCTGTCCACGCCCCTGGTTCTTAGTTCCAGCCCCA
CTCATAGGACACTCATAGCTCAGGAGGGCTCCGCCTTCAATCCCACCCGCTAAAGTACTTGGAGCGGTCTCTCCC
TCCCTCATCAGCCCACCAAACCAAACCTAGCCTCCAAGAGTGGGAAGAAATTAAAGCAAGATAGGCTATTAAGTG
CAGAGGGAGAGAAAATGCCTCCAACATGTGAGGAAGTAATGAGAGAAATCATAGAATTTCTTCCGCTTCCTCGCT
CACTGACTCGCTGCGCTCGGTCGTTCGGCTGCGGCGAGCGGTATCAGCTCACTCAAAGGCGGTAATACGGTTATC
CACAGAATCAGGGGATAACGCAGGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTACGGCTACAC
TAGAAGAACAGTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGAAAAAAAGGATC
TCAAGAAGATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAAAGTAT
ATATGAGTAAACTTGGTCTGACAGTTACCAATGCTTAATCAGTGAGGCACCTATCTCAGCGATCTGTCTATTTCG
TTCATCCATAGTTGCCTGACTCGGGGGGGGGGGGCGCTGAGGTCTGCCTCGTGAAGAAGGTGTTGCTGACTCATA
CCAGGCCTGAATCGCCCCATCATCCAGCCAGAAAGTGAGGGAGCCACGGTTGATGAGAGCTTTGTTGTAGGTGGA
CCAGTTGGTGATTTTGAACTTTTGCTTTGCCACGGAACGGTCTGCGTTGTCGGGAAGATGCGTGATCTGATCCTT
CAACTCAGCAAAAGTTCGATTTATTCAACAAAGCCGCCGTCCCGTCAAGTCAGCGTAATGCTCTGCCAGTGTTAC
AACCAATTAACCAATTCTGATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGGCAGTTCCATAGGATGGCA
AGATCCTGGTATCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGCTTATGCATTTCTTTC
CAGACTTGTTCAACAGGCCAGCCATTACGCTCGTCATCAAAATCACTCGCATCAACCAAACCGTTATTCATTCGT
GATTGCGCCTGAGCGAGACGAAATACGCGATCGCTGTTAAAAGGACAATTACAAACAGGAATCGAATGCAACCGG
CGCAGGAACACTGCCAGCGCATCAACAATATTTTCACCTGAATCAGGATATTCTTCTAATACCTGGAATGCTGTT
TTCCCGGGGATCGCAGTGGTGAGTAACCATGCATCATCAGGAGTACGGATAAAATGCTTGATGGTCGGAAGAGGC
ATAAATTCCGTCAGCCAGTTTAGTCTGACCATCTCATCTGTAACATCATTGGCAACGCTACCTTTGCCATGTTTC
AGAAACAACTCTGGCGCATCGGGCTTCCCATACAATCGATAGATTGTCGCACCTGATTGCCCGACATTATCGCGA
GCCCATTTATACCCATATAAATCAGCATCCATGTTGGAATTTAATCGCGGCCTCGAGCAAGACGTTTCCCGTTGA
ATATGGCTCATAACACCCCTTGTATTACTGTTTATGTAAGCAGACAGTTTTATTGTTCATGATGATATATTTTTA
TCTTGTGCAATGTAACATCAGAGATTTTGAGACACAACGTGGCTTTCCCCCCCCCCCCATTATTGAAGCATTTAT
CAGGGTTATTGTCTCATGAGCGGATACATATTTGAATGTATTTAGAAAAATAAACAAATAGGGGTTCCGCGCACA
TTTCCCCGAAAAGTGCCACCTGACGTCTAAGAAACCATTATTATCATGACATTAACCTATAAAAATAGGCGTATC
ACGAGGCCCTTTCGTCTCGCGCGTTTCGGTGATGACGGTGAAAACCTCTGACACATGCAGCTCCCGGAGACGGTC
ACAGCTTGTCTGTAAGCGGATGCCGGGAGCAGACAAGCCCGTCAGGGCGCGTCAGCGGGTGTTGGCGGGTGTCGG
GGCTGGCTTAACTATGCGGCATCAGAGCAGATTGTACTGAGAGTGCACCATATGCGGTGTGAAATACCGCACAGA
TGCGTAAGGAGAAAATACCGCATCAGATTGGCTATTGG (SEQ ID NO: 53)
AG283
Expressed protein: Mouse GM-CSF-IL-15_Linker IL-12p40
5375 base pairs
Underlined: Optimized coding sequence for expressed fusion protein
Bold: terminators
CCTGGCCATTGCATACGTTGTATCCATATCATAATATGTACATTTATATTGGCTCATGTCCAACATTACCGCCAT
GTTGACATTGATTATTGACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGTCAATGATGGTAAATGGCCCG
CCTGGCATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTCGTAACAACT
CCGCCCCATTGACGCAAATGGGCGGTAGGCGTGTACGGTGGGAGGTCTATATAAGCAGAGCTCGTTTAGTGAACC
GTCAGATCGCCTGGAGACGCCATCCACGCTGTTTTGACCTCCATAGAAGACACCGGGACCGATCCAGCCTCCGCG
GGCGCGCGTCGAGGAATTCTCTAGAAAGAAATGTGGCTCCAGAACCTGCTATTCCTGGGGATCGTGGTCTACAGC
CTCTCGAACTGGATCGATGTGCGCTACGACCTGGAGAAGATCGAGTCCCTCATCCAGTCGATCCACATCGACACC
ACGCTGTACACGGACTCGGACTTCCACCCGTCGTGCAAGGTCACGGCGATGAACTGCTTCCTCCTGGAGCTGCAA
GTCATCCTGCACGAGTACTCGAACATGACCCTCAACGAGACGGTGCGCAACGTGCTGTACCTGGCGAACTCGACC
CTGTCGTCGAACAAGAACGTCGCCGAGTCGGGCTGCAAGGAGTGCGAGGAGCTGGAGGAGAAGACCTTCACGGAG
TTCCTGCAGTCGTTCATCCGCATCGTCCAGATGTTCATCAACACGTCGGGAGCAGGTGCTATGTGGGAGCTGGAG
AAGGACGTCTACGTCGTGGAGGTGGACTGGACGCCCGACGCCCCCGGGGAGACGGTGAACCTCACCTGCGACACG
CCGGAGGAGGACGACATCACCTGGACCAGCGACCAGCGGCACGGCGTCATCGGCAGCGGCAAGACCCTGACGATC
ACCGTCAAGGAGTTCCTCGACGCCGGCCAGTACACCTGCCACAAGGGCGGCGAGACGCTGAGCCACAGCCACCTG
CTGCTCCACAAGAAGGAGAACGGCATCTGGTCCACCGAGATCCTCAAGAACTTCAAGAACAAGACCTTCCTGAAG
TGCGAGGCGCCCAACTACTCCGGCCGGTTCACGTGCTCCTGGCTGGTGCAGCGCAACATGGACCTCAAGTTCAAC
ATCAAGTCGTCCAGCAGTTCCCCCGACTCCCGGGCGGTGACCTGCGGCATGGCGTCCCTGTCGGCGGAGAAGGTC
ACGCTGGACCAGAGGGACTACGAGAAGTACTCGGTGTCCTGCCAGGAGGACGTCACCTGCCCCACGGCCGAGGAG
ACCCTGCCCATCGAGCTGGCGCTGGAGGCCCGGCAGCAGAACAAGTACGAGAACTACAGCACCAGCTTCTTCATC
CGGGACATCATCAAGCCCGACCCGCCCAAGAACCTGCAGATGAAGCCCCTGAAGAACAGCCAGGTGGAGGTCAGC
TGGGAGTACCCGGACTCCTGGAGCACGCCCCACTCCTACTTCAGCCTGAAGTTCTTCGTCAGAATCCAGCGCAAG
AAAGAGAAGATGAAGGAGACGGAGGAGGGGTGCAACCAGAAGGGCGCGTTCCTCGTCGAGAAGACCTCCACCGAG
GTCCAGTGCAAGGGCGGGAACGTCTGCGTGCAGGCTCAGGACCGCTACTACAACTCCAGCTGCAGCAAGTGGGCC
TGCGTCCCCTGCAGGGTCCGGTCCTAGTAAAGATCTGATATCGGATCTGCTGTGCCTTCTAGTTGCCAGCCATCT
GTTGTTTGCCCCTCCCCCGTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGGTACCCAGGTGCTGAAGAATTGACCC
GGTTCCTCCTGGGCCAGAAAGAAGCAGGCACATCCCCTTCTCTGTGACACACCCTGTCCACGCCCCTGGTTCTTA
GTTCCAGCCCCACTCATAGGACACTCATAGCTCAGGAGGGCTCCGCCTTCAATCCCACCCGCTAAAGTACTTGGA
GCGGTCTCTCCCTCCCTCATCAGCCCACCAAACCAAACCTAGCCTCCAAGAGTGGGAAGAAATTAAAGCAAGATA
GGCTATTAAGTGCAGAGGGAGAGAAAATGCCTCCAACATGTGAGGAAGTAATGAGAGAAATCATAGAATTTCTTC
CGCTTCCTCGCTCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCAAAAGGCCAGGAA
CCGTAAAAAGGCCGCGTTGCTGGCGTTTTTCCATAGGCTCCGCCCCCCTGACGAGCATCACAAAAATCGACGCTC
AAGTCAGAGGTGGCGAAACCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTAAAAATGAAGTTTTAAATC
AATCTAAAGTATATATGAGTAAACTTGGTCTGACAGTTACCAATGCTTAATCAGTGAGGCACCTATCTCAGCGAT
CTGTCTATTTCGTTCATCCATAGTTGCCTGACTCGGGGGGGGGGGGCGCTGAGGTCTGCCTCGTGAAGAAGGTGT
TGCTGACTCATACCAGGCCTGAATCGCCCCATCATCCAGCCAGAAAGTGAGGGAGCCACGGTTGATGAGAGCTTT
GTTGTAGGTGGACCAGTTGGTGATTTTGAACTTTTGCTTTGCCACGGAACGGTCTGCGTTGTCGGGAAGATGCGT
GATCTGATCCTTCAACTCAGCAAAAGTTCGATTTATTCAACAAAGCCGCCGTCCCGTCAAGTCAGCGTAATGCTC
TGCCAGTGTTACAACCAATTAACCAATTCTGATTAGAAAAACTCATCGAGCATCAAATGAAACTGCAATTTATTC
ATATCAGGATTATCAATACCATATTTTTGAAAAAGCCGTTTCTGTAATGAAGGAGAAAACTCACCGAGGCAGTTC
CATAGGATGGCAAGATCCTGGTATCGGTCTGCGATTCCGACTCGTCCAACATCAATACAACCTATTAATTTCCCC
TCGTCAAAAATAAGGTTATCAAGTGAGAAATCACCATGAGTGACGACTGAATCCGGTGAGAATGGCAAAAGCTTA
TGCATTTCTTTCCAGACTTGTTCAACAGGCCAGCCATTACGCTCGTCATCAAAATCACTCGCATCAACCAAACCG
TTATTCATTCGTGATTGCGCCTGAGCGAGACGAAATACGCGATCGCTGTTAAAAGGACAATTACAAACAGGAATC
GAATGCAACCGGCGCAGGAACACTGCCAGCGCATCAACAATATTTTCACCTGAATCAGGATATTCTTCTAATACC
TGGAATGCTGTTTTCCCGGGGATCGCAGTGGTGAGTAACCATGCATCATCAGGAGTACGGATAAAATGCTTGATG
GTCGGAAGAGGCATAAATTCCGTCAGCCAGTTTAGTCTGACCATCTCATCTGTAACATCATTGGCAACGCTACCT
TTGCCATGTTTCAGAAACAACTCTGGCGCATCGGGCTTCCCATACAATCGATAGATTGTCGCACCTGATTGCCCG
ACATTATCGCGAGCCCATTTATACCCATATAAATCAGCATCCATGTTGGAATTTAATCGCGGCCTCGAGCAAGAC
GTTTCCCGTTGAATATGGCTCATAACACCCCTTGTATTACTGTTTATGTAAGCAGACAGTTTTATTGTTCATGAT
GATATATTTTTATCTTGTGCAATGTAACATCAGAGATTTTGAGACACAACGTGGCTTTCCCCCCCCCCCCATTAT
TGAAGCATTTATCAGGGTTATTGTCTCATGAGCGGATACATATTTGAATGTATTTAGAAAAATAAACAAATAGGG
GTTCCGCGCACATTTCCCCGAAAAGTGCCACCTGACGTCTAAGAAACCATTATTATCATGACATTAACCTATAAA
AATAGGCGTATCACGAGGCCCTTTCGTCTCGCGCGTTTCGGTGATGACGGTGAAAACCTCTGACACATGCAGCTC
CCGGAGACGGTCACAGCTTGTCTGTAAGCGGATGCCGGGAGCAGACAAGCCCGTCAGGGCGCGTCAGCGGGTGTT
GGCGGGTGTCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGATTGGCTATTGG (SEQ ID NO: 54)
Additional information and sequences for IL-12 can be found for example,
in U.S. Pat. No. 7, 833, 754, U.S. Pat. No. 8, 715, 964, U.S. Pat. No.
9,546, 202, each of which is incorporated by reference herein in their
entirety.
Additional IL-15 and IL-15R- information and sequences can be found in
PCT/US2007/000774; US Patent Application Pub. No. 2016/0102128 A1; U.S.
Pat. No. 9,303,080, and U.S. Pat. No. 9,725,492, each of which is
incorporated by reference herein in their entirety.

In some embodiments, IL-12 is the immature or precursor form of a mammalian IL-12. In other embodiments, IL-12 is the mature form of a mammalian IL-12. In a specific embodiment, IL-12 is the precursor form of human IL-12. In another embodiment, IL-12 is the mature form of human IL-12. In one embodiment, the IL-12 protein/polypeptide is isolated or purified.

As used herein, the terms “IL-12 derivative” and “interleukin-12 derivative” in the context of proteins or polypeptides refer to: (a) a polypeptide that is at least 40%, 45%, 50%, 55%, 60%, 65%, 70%, 75%, 80%, 85%, 90%, 95%, 98% or 99% identical to an IL-12 polypeptide; (b) a polypeptide encoded by a nucleic acid sequence that is at least 40%, 45%, 50%, 55%, 60%, 65%, 70%, 75%, 80%, 85%, 90%, 95%, 98% or 99% identical a nucleic acid sequence encoding an IL-12 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a native mammalian IL-12 polypeptide; (d) a polypeptide encoded by nucleic acids can hybridize under high, moderate or typical stringency hybridization conditions to nucleic acids encoding an IL-12 polypeptide; (e) a polypeptide encoded by a nucleic acid sequence that can hybridize under high, moderate or typical stringency hybridization conditions to a nucleic acid sequence encoding a fragment of an IL-12 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a mammalian IL-12 polypeptide. IL-12 derivatives also include a polypeptide that comprises the amino acid sequence of a mature form of a mammalian IL-12 polypeptide and a heterologous signal peptide amino acid sequence. In a specific embodiment, an IL-12 derivative is a derivative of a native human IL-15 polypeptide. In another embodiment, an IL-12 derivative is a derivative of an immature or precursor form of human IL-12 polypeptide. In another embodiment, an IL-12 derivative is a derivative of a mature form of human IL-12 polypeptide. In one embodiment, an IL-12 derivative is isolated or purified.

In certain embodiments, IL-12 derivatives retain at least 50%, 55%, 60%, 65%, 70%, 75%, 80%, 85%, 90%, 95%, 98% or 99% of the function of native mammalian IL-12 polypeptide to bind IL-12 receptors, as measured by assays well known in the art, e.g., ELISA, Biacore, co-immunoprecipitation. In some embodiments, IL-12 derivatives retain at least 50%, 55%, 60%, 65%, 70%, 75%, 80%, 85%, 90%, 95%, 98% or 99% of the function of native mammalian IL-12 polypeptide to induce IL-12-mediated signal transduction, as measured by assays well-known in the art, e.g., electromobility assays, electromobility shift assays, ELISAs and other immunoassays or live cell bioassays.

### Fusion Proteins

In some embodiments, IL-15 or IL-15 derivates or fragments thereof are linked with IL-12 or IL-12 derivates or fragments thereof to form a IL-15:IL-12 fusion protein. In some embodiments, the IL-15:IL-12 fusion protein can be linked by an intra-peptide linker. For example, a polypeptide linker located between the C-terminal of the first peptide and the N terminal of the second peptide. With or without the intra-peptide linker, the IL-15 or IL-15 derivates or fragments thereof and the IL-12 or IL-12 derivates or fragments thereof may be positioned in any order. For example, the IL-15 or IL-15 derivates or fragments thereof may be positioned at the N-terminal portion of the fusion protein and the IL-12 or IL-12 derivates or fragments thereof may be positioned at the C-terminal portion of the fusion protein. Or, the IL-12 or IL-12 derivates or fragments thereof may be positioned at the N-terminal portion of the fusion protein and the IL-15 or IL-15 derivates or fragments thereof may be positioned at the C-terminal portion of the fusion protein.

In some embodiments, the fusion protein comprises a linker domain between the IL-15 and the IL-12 components. Linkers can comprise flexible amino acid residues (e.g., glycine or serine) to permit adjacent domains to move freely related to one another. In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g., Gly) or polar (e.g., Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser)n, (Gly-Gly-Gly-Ser)n (SEQ ID NO:33) or (Gly-Gly-Gly-Gly-Ser)n (SEQ ID NO:34), where n=1-5. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. In certain embodiments, when present, the linker can be an amino acid sequence selected from the group consisting of GGGS (SEQ ID NO:33), GGGSGGGS (SEQ ID NO:35), GGGSGGGSGGGS (SEQ ID NO:36), GGGSGGGSGGGSGGGS (SEQ ID NO:37), GGGGS (SEQ ID NO:34), GGGGSGGGGS (SEQ ID NO:38), GGGGSGGGGSGGGGS (SEQ ID NO:39), and GGGGSGGGGSGGGGSGGGGS (SEQ ID NO:40).

When the IL-15 or IL-15 derivates or fragments thereof are linked with IL-12 or IL-12 derivates or fragments thereof to form a fusion protein, the linker may be a cleavable linker. As used herein, the term “cleavable linker” refers to any linker between the IL-15 or IL-15 derivates or fragments thereof are linked with IL-1 or IL-12 derivates or fragments thereof that promotes or otherwise renders the IL-15 or IL-15 derivates or fragments thereof are linked with IL-1 or IL-12 derivates or fragments thereof more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some compositions, the cleavable linker is a protease-sensitive dipeptide or oligopeptide cleavable linker. In certain embodiments, the cleavable linker is sensitive to cleavage by a protease of the trypsin family of proteases. In some compositions, the cleavable linker comprises an amino acid sequence selected from the group consisting of arginine-arginine, arginine-valine-arginine-arginine (RVRR; SEQ ID NO:41), valine-citrulline, valine-arginine, valine-lysine, valine-alanine, phenylalanine-lysine, glycine-alanine-glycine-alanine (GAGA; SEQ ID NO:42), alanine-glycine-alanine-glycine (AGAG; SEQ ID NO:43), and lysine-glycine-lysine-glycine (KGKG: SEQ ID NO:44). In some compositions, the cleavable linker is arginine-arginine. In some embodiments, the linker comprises an amino acid sequence any one of AA, AAA, KK, KKK, SS, SSS, AGAG (SEQ ID NO:43), GG, GGG, GAGA (SEQ ID NO:42), and KGKG (SEQ ID NO:44).

### Nucleic Acids

The disclosure further provides nucleic acids encoding any of IL-15 or IL-15 derivates or fragments thereof or the IL-12 or IL-12 derivates or fragments thereof as disclosed herein. The nucleic acid compositions as disclosed herein, can comprise, consist of, or consist essentially of, a first nucleic acid sequence encoding IL-15 or IL-15 derivates or fragments thereof and a second nucleic acid sequence the IL-12 or IL-12 derivates or fragments thereof as disclosed herein. In certain embodiments, the IL-15 or IL-15 derivates or fragments thereof and the IL-12 or IL-12 derivates or fragments thereof may be encoded by the same nucleic acid sequence. In some embodiments, the nucleic acid sequences may also encode a linker to a carrier and/or a C-terminal cysteine. In addition, when a single nucleic acid sequence encodes both peptides, the sequence may also encode an intra-peptide linker. The nucleic acid compositions described herein (pharmaceutical compositions) can be used in methods for treating or effecting prophylaxis and/or prevention of cancer. In another embodiment, the nucleic acid compositions as disclosed herein can be administered with one or more active agents as disclosed herein.

### PPARα Activators

Peroxisome proliferator-activated receptors (PPARs) are members of three ligand-inducible transcription factors, which belong to the nuclear receptor super-family. PPARs play an important role in regulating the expression of a variety of genes regarding the metabolic homeostasis of glucose and lipid, adipogenesis, and inflammation. In mammals, there are three subtypes of PPARs: PPAR-α, PPAR-γ, and PPAR-β/δ, possessing varying expression levels in different tissues, biological effects, and ligand affinities. PPAR-α is mainly expressed in brown adipose, skeletal muscle, heart, liver, and intestinal mucosa tissues, adjusting glucose and lipid metabolism and homeostasis, inflammation, immune response, and angiogenesis. PPAR-α maintains lipid metabolism and homeostasis via the modulation of genes of lipoprotein lipase, apolipoprotein (e.g., APOA1, APOA2, APOA5, and APOC3), as well as those involved in fatty acid transport and oxidation (e.g., FABP1, FABP3, ACS, ACO, CPT1, and CPT2), high-density lipoprotein (HDL) metabolism (e.g., PLTP), and ketone synthesis (e.g., HMGCS2), which take place in mitochondria, peroxisomes, and microsomes.

The term “PPAR activator” refers to compounds that activate and/or modulate peroxisome proliferator activator receptor (PPAR) activity in mammals (for example, in humans). Any PPAR activator may be used in the combination aspect of this disclosure. Activation is readily determined by those skilled in the art according to standard assays known in the literature (see, for example, Rosenson, “Fenofibrate: treatment of hyperlipidemia and beyond” Expert Review of Cardiovascular Therapy; Volume 6, 2008—Issue 10: Pages 1319-1330|Published online: 10 Jan. 2014; or Huang et al., “The PPARα agonist fenofibrate suppresses B-cell lymphoma in mice by modulating lipid metabolism” Biochim Biophys Acta. 2013 October; 1831(10):1555-1565). In certain embodiments, a PPAR activator can be selected from Bavachinin, BMS-687453, CDDO-Im (CDDO-Imidazolide, RTA-403, TP-235), Clofibric Acid, Elafibranor, Fenofibric acid, Fenofibrate (NSC-281319, Tricor), Gemfibrozil, GW6471, GW9662, Lanifibranor (IVA-337), Palmitoylethanolamide, Phytol, Saroglitazar, and WY-14643 (Pirinixic Acid). In certain embodiments, the PPAR activator is fenofibrate.

Fenofibrate (FF) is an agent of the fibrate class that has been used since 1975 to reduce cholesterol (LDL and VLDL) and triglyceride levels and increase HDL in patients at risk of cardiovascular disease and for treatment of atherosclerosis. FF is one of the most commonly prescribed fibrates and has an excellent efficacy and tolerability profile. FF activates the PPAR-α, that leads to transcription of multiple metabolic genes, such as those related to fatty acid oxidation or inhibition of glycolysis. More specifically, activation of PPAR-α stimulates lipoprotein lipase, lowers apoprotein CIII, and improves blood triglycerides and HDL-cholesterol levels. In addition to its hypolipidemic action, it has also become apparent that FF exerts pleiotropic properties affecting other organs and tissues. For instance, fenofibrate was found to protect against diabetic retinopathy and other microvascular complications in patients with type I or II diabetes as well as against intrahepatic cholostasis. FF was established to afford myocardial protection through its direct effects on the cardiovascular system and promotes angiogenesis in rodent models of ischemia. Most recently, PPAR-α-specific agonists were reported to have anticancer effects in a large number of human cancer types, such as acute myeloid leukemia, chronic lymphocytic leukemia, and solid tumors, including those of the liver breast, skin, and lungs, liver and ovary. Furthermore, FF exerts robust ‘anti-cancer’ activity and elicits inhibitory effects in several types of cancers, including lymphoma, glioblastoma, prostate cancer, angiosarcoma and breast cancer.

Suitable quantities of a PPAR activator can be about 1 mg to about 1500 mg, or about 25 mg to 200 mg, but lower levels such as 1-25 mg can be employed. For example, about 1 mg, about 10 mg, about 20 mg, about 25 mg, about 30 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more. In certain embodiments, a PPAR activator can be administered in the form of a pharmaceutical composition individually or together in any conventional oral, parenteral, rectal or transdermal dosage form. For oral administration a pharmaceutical composition may take the form of solutions, suspensions, tablets, pills, capsules, powders, and the like. In certain embodiments, multiple doses of a PPAR activator can be administered, for example, daily, every other day, bi-weekly, weekly, bi-monthly, monthly, or more or less often, as needed, for a time period sufficient to achieve the desired response. In certain embodiments, a PPAR activator can be administered twice daily, daily, every other day, once every three days, weekly, bi-weekly, monthly, bi-monthly, or longer. In certain embodiments, a PPAR activator can be administered daily. In certain embodiments, a PPAR activator can be administered for about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months or longer. In certain embodiments, a PPAR activator can be administered indefinitely, or as needed to effectively treat a patient.

### FLT3 Inhibitor

The FLT3 receptor (Fms-like tyrosine kinase 3) belongs to class Ill family of the receptor tyrosine kinases (RTKs) and comprises four regions. (1) an N-terminal extracellular region consisting of five immunoglobulin-like subdomains, (2) a transmembrane domain, (3) a juxtamembrane (JM) domain, and (4) an intracellular C-terminal kinase domain consisting of two substructures (N-lobe and C-lobe) that are connected by an activation-loop (A-loop). The extracellular region of FLT3 is glycosylated and contains a ligand binding domain as well as a dimerization domain. The nonglycosylated form of the receptor is not anchored to the plasma membrane. The JM domain plays an important regulatory role through direct contact with the catalytic kinase domain. Finally, the kinase domain transmits activation signal to downstream targets and is regulated by the conformation of the A-loop and the JM domain as well as ATP binding. There has been a sustained effort to develop FLT3 inhibitors since the discovery of FLT3 mutations. FLT3 inhibitors are small molecules that compete with ATP to bind the active pocket of the kinase domain, inhibiting auto-phosphorylation and phosphorylation of downstream targets. FLT3 inhibitors can broadly be categorized into first- and second-generation inhibitors. The first-generation FLT3 inhibitors are multi-kinase inhibitors and thus not selective to FLT3; some examples include midostaurin, sorafenib, sunitinib, and ponatinib. The second-generation FLT3 inhibitors are developed to selectively inhibit FLT3 and include quizartinib, gilteritinib, and crenolanib.

In certain embodiments, a FLT3 inhibitor can be selected from AG1295, AG1296, amuvatinib (MP-470, HPK 56), CEP-5214, CEP-7055, CHIR-258, crenolanib (CP-868596, ARO 002), dovitinib (TK1258, CHIR258), EB10, gilteritinib (ASP2215), GTP 14564, lestaurtinib (CEP 701, formerly KT-555), HM43239, linifanib (ABT-869, AL39324, RG3635), IMC-EB10, midostaurin (PKC412), pacritinib (SB1518), ponatinib, tandutinib (MLN-518, formerly CT53518), SKLB4771 (FLT3-IN-1), sorafenib (BAY 43-9006), sunitinib (SU11248), SU5614 (Chloro-SU5416, Chloro-Semaxanib), tandutinib (MLN518, CT53518, NSC726292), tozasertib (VX-680, MK-0457), quizartinib (AC220), and XL 999. In certain embodiments, a FLT3 inhibitor can be quizartinib, gilteritinib, or crenolanib. In some embodiments, a FLT3 inhibitor is quizartinib.

Suitable quantities of a FLT3 inhibitor can be about 1 mg to about 1500 mg, about 100 mg to about 350 mg, or about 15 mg to about 200 mg, but lower levels such as 1-15 mg can be employed. For example, about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more. In certain embodiments, about 100 mg to about 350 mg can be administered. In certain embodiments, about 100 mg to about 350 mg can be administered daily. In certain embodiments, a FLT3 inhibitor can be administered in the form of a pharmaceutical composition individually or together in any conventional oral, parenteral, rectal or transdermal dosage form. For oral administration a pharmaceutical composition may take the form of solutions, suspensions, tablets, pills, capsules, powders, and the like. In certain embodiments, multiple doses of a FLT3 inhibitor can be administered, for example, daily, every other day, bi-weekly, weekly, bi-monthly, monthly, or more or less often, as needed, for a time period sufficient to achieve the desired response. In certain embodiments, a FLT3 inhibitor can be administered twice daily, daily, every other day, once every three days, weekly, bi-weekly, monthly, bi-monthly, or longer. In certain embodiments, a FLT3 inhibitor can be administered daily. In certain embodiments, a FLT3 inhibitor can be administered for about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months or longer. In certain embodiments, a FLT3 inhibitor can be administered indefinitely, or as needed to effectively treat a patient.

In some embodiments, a FLT3 inhibitor is quizartinib. Quizartinib (AC220): Quizartinib (AC220, Daiichi Sankyo) is an extremely potent Fms-like tyrosine kinase 3 (Flt3) inhibitor, was originally developed by Ambit Biosciences. Quizartinib was found to have good efficacy and tolerability in xenograft models and with activity in the low nanomolar range in cell culture assays, and animal models at doses as low as 1 mg/kg. From this promising pre-clinical data, Quizartinib was taken into clinical trials (Tables 2 and 3).

TABLE 2
Summary of Quizartinib clinical trials

| | | Disease | | |
|---|---|---|---|---|
| Trial name | Clinical Trial # | Status | Phase | Reference |
| Open-Label, Dose Escalation Study of | NCT02675478 | R/R AML | Phase I | [66] |
| Quizartinib, an Oral FLT3 Inhibitor, in | | | | |
| Japanese Patients with Relapsed or | | | | |
| Refractory Acute Myeloid Leukemia- | | | | |
| A Phase I Study of AC220 in Patients | NCT00462761 | R/R AML | Phase I | [67] |
| with Relapsed/Refractory Acute Myeloid | | | | |
| Leukemia Regardless of FLT3 Status | | | | |
| A Study of AC220 Given After | NCT01468467 | CRI AML | Phase I | [68] |
| Transplant in Subjects with Acute | | | | |
| Myeloid Leukemia (AML) | | | | |
| A Study to Assess AC220 Given in | NCT01390337 | ND AML | Phase I | [69] |
| Combination with Induction and | | | | |
| Consolidation Therapy in Newly | | | | |
| Diagnosed Acute Myeloid Leukemia | | | | |
| (AML) | | | | |
| Open-Label Study to Evaluate Safety | NCT01565668 | R/R AML | Phase 2 | [70] |
| and Efficacy of 2 Doses of Quizartinib in | | | | |
| Patients with Relapsed or Refractory | | | | |
| Acute Myeloid Leukemia | | | | |
| Phase 2 Study of Quizartinib in | NCT02984995 | R/R AML | Phase 2 | [71] |
| Participants with Phase 2 Study of | | | | |
| Quizartinib in Participants With | | | | |
| (QUANTUM-R): An Open-label Study of | NCT02039726 | R/R AML | Phase 3 | [72] |
| Quizartinib Monotherapy vs Salvage | | | | |
| Chemotherapy in Acute Myeloid | | | | |
| Leukemia (AML) Subjects Who Are | | | | |
| FLT3-ITD Positive | | | | |
| Milademetan Plus Quizartinib | NCT03552029 | R/R AML | Phase 1/Terminated | |
| Combination Study in FLT3-ITD Mutant | | | | |

Acute Myeloid Leukemia (AML)

TABLE 3
Ongoing and future Quizartinib clinical trials

| | | Disease | | |
|---|---|---|---|---|
| Trial name | Clinical Trial # | Status | Phase | Status |
| A Study to Evaluate the Safety and | NCT03723681 | ND AML | Phase 1 | Recruiting |
| Pharmacokinetics of Quizartinib in | | | | |
| Combination with Standard Induction | | | | |
| Therapy and ConsolidationTherapy in | | | | |
| Chinese Patients With Newly Diagnosed | | | | |
| Acute Myeloid Leukemia | | | | |
| Venetoclax and Quizartinib in Treating | NCT03735875 | R/R AML | Phase | Recruiting |
| Patients with FLT3-mutated Recurrent or | | | 1b/2 | |
| Refractory Acute Myeloid Leukemia | | | | |
| Quizartinib and Decitabine in Treating | NCT03661307 | ND or R/R | Phase | Recruiting |
| Participants with Untreated or Relapsed | | AML | 1/2 | |
| FLT3-ITD Mutated Acute Myeloid | | | | |
| Leukemia or Myelodysplastic Syndrome | | | | |
| Safety and Effectiveness of Quizartinib in | NCT03793478 | R/R AML | Phase | Recruiting |
| Children and Young Adults with Acute | | | 1/2 | |
| Myeloid Leukemia (AML), a Cancer of the | | | | |
| Blood | | | | |
| Quizartinib With Azacitidine or Cytarabine | NCT01892371 | R/R AML | Phase 2 | Recruiting |
| in Treating Participants with Relapsed or | | | | |
| Refractory Acute Myeloid Leukemia | | | | |
| orMyelodysplastic Syndrome | | | | |
| Combination of Quizartinib and | NCT03135054 | ND AML | Phase 2 | Active, not |
| Omacetaxine Mepesuccinate for AML | | | | Recruiting |
| Carrying FLT3-ITD | | | | |
| Cladribine, Idarubicin, Cytarabine, and | NCT04047641 | ND AML or | Phase 2 | Recruiting |
| Quizartinib in Treating Patients with | | R/R AML | | |
| Newly Diagnosed, Relapsed, or | | | | |
| Refractory Acute Myeloid Leukemia or | | | | |
| High-Risk Myelodysplastic Syndrome | | | | |
| Quizartinib and High-dose Ara-C Plus | NCT03989713 | R/R AML | Phase 2 | Recruiting |
| Mitoxantrone in Relapsed/Refractory AML | | | | |
| With FLT3-ITD (Q-HAM) | | | | |
| Quizartinib With Standard of Care | NCT02668653 | ND AML | Phase 3 | Active, not |
| Chemotherapy and as Continuation | | | | Recruiting |
| Therapy in Patients with Newly | | | | |
| Diagnosed FLT3-ITD (+) Acute Myeloid | | | | |
| Leukemia (AML) (QUANTUM-First) | | | | |
| Expanded Treatment Protocol for Adults | NCT03746912 | R/R AML | Expanded | No longer |
| with FLT3-ITD Mutated Relapsed or | | | access | available |
| Refractory Acute Myeloid Leukemia | | | | |
| (AML) to ReceiveQuizartinib | | | | |

### Chemotherapeutic Agents

In certain embodiments of the methods, compositions and fusion proteins as disclosed herein, one or more active agents comprises a chemotherapeutic agent. In certain embodiments, the chemotherapeutic agent is selected from adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof. In certain embodiments, the chemotherapeutic agent is gemcitabine. In certain embodiments, the chemotherapeutic agent is plerixafor.

Suitable quantities of chemotherapeutic agent can be about 1 mg to about 1500 mg, about 100 mg to about 350 mg, or about 15 mg to about 200 mg, but lower levels such as 1-15 mg can be employed. For example, about 1 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 37.7 mg, about 40 mg, about 45 mg, about 50 mg, about 54 mg, about 55 mg, about 60 mg, about 70 mg, about 80 mg, about 90 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 170 mg, about 175 mg, about 180 mg, about 185 mg, about 190 mg, about 195 mg, about 200 mg, about 250 mg, about 300 mg, about 400 mg, about 450 mg, about 500 mg, about 600 mg, about 700 mg, about 800 mg, about 900 mg, about 1000 mg, or more. In certain embodiments, about 100 mg to about 350 mg can be administered. In certain embodiments, a chemotherapeutic agent can be administered in the form of a pharmaceutical composition individually or together in any conventional oral, parenteral, rectal or transdermal dosage form. For oral administration a pharmaceutical composition may take the form of solutions, suspensions, tablets, pills, capsules, powders, and the like. In certain embodiments, multiple doses of a chemotherapeutic agent can be administered, for example, daily, every other day, bi-weekly, weekly, bi-monthly, monthly, or more or less often, as needed, for a time period sufficient to achieve the desired response. In certain embodiments, a chemotherapeutic agent can be administered twice daily, daily, every other day, once every three days, weekly, bi-weekly, monthly, bi-monthly, or longer. In certain embodiments, a chemotherapeutic agent can be administered daily. In certain embodiments, a chemotherapeutic agent can be administered for about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months or longer. In certain embodiments, a chemotherapeutic agent can be administered indefinitely, or as needed to effectively treat a patient.

In some embodiments, a chemotherapeutic agent is gemcitabine. Gemcitabine (Gemzar) is a synthetic pyrimidine nucleoside prodrug—a nucleoside analog, that was originally developed by Eli Lily. Suitable quantities of gemcitabine can be about 750 to 1500 mg/m2 administered by IV. In certain embodiments, gemcitabine can be administered for every 1 week, 2 weeks, 3 weeks, 4 weeks, for about 21 to 28 days.

### Treatments

As used herein, the terms “treat”, “treating” and “treatment” in the context of the administration of a therapy to a subject refer to the beneficial effects that a subject derives from a therapy, such as, but not limited to, the reduction or inhibition of the progression, spread and/or duration of a disease or disorder, the reduction or amelioration of the severity of a disease or disorder, amelioration of one or more symptoms of a disease or disorder, and/or the reduction in the duration of one or more symptom of a disease or disorder resulting from the administration of one or more therapies. In certain embodiments, such terms in the context of cancer include, but are not limited to, one, two, or three or more results following the administration of a therapy to a subject: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%; preferably the size of the tumor does not increase by more than 2%.

In certain embodiments, an IL-15 or an IL-15/IL-15Rα complex and one or more active agents that will be effective in the prevention, treatment and/or management of a disease that is affected by IL-15 function can be determined by standard clinical techniques. In vitro or in vivo assays may optionally be employed to help identify optimal dosage ranges. The precise dose to be employed will also depend, e.g., on the route of administration, the type of symptoms, and the seriousness of the symptoms, and should be decided according to the judgment of the practitioner and each patient's or subject's circumstances.

Provided herein is a method for preventing, treating and/or managing cancer in a subject, wherein prevention, treatment and/or management of cancer comprises administering compositions comprising an IL-15 or an IL-15/IL-15Rα complex and one or more active agents to a human subject. In certain embodiments, an IL-15 or an IL-15/IL-15Rα complex dose regimen is between 0.1 μg/kg and 20 μg/kg as determined based on the mass of single chain IL-15. In some embodiments, the methods described herein maintain plasma levels of IL-15 above basal levels for approximately 18 to 24 hours or approximately 24 to 36 hours, or approximately 36 to 38 hours following administration of an IL-15 or an IL-15/IL-15Rα complex. Basal plasma levels of IL-15 are approximately 1-2 μg/ml in humans, approximately 8-10 μg/ml in monkeys (such as macaques), and approximately 12 μg/m in rodents (such as mice). Thus, in certain embodiments, the methods described herein maintain plasma levels above approximately 1 μg/ml in humans, above approximately 8-10 μg/ml in monkeys (such macaques) and above 12 μg/ml in rodents (such as mice). Without being bound by any theory, prolonged increase of the IL-15 plasma levels in the absence of high concentration spikes maximizes lymphocyte growth and activation while minimizing any side effects associated with IL-15 administration. In some embodiments, the methods described herein achieve stable plasma levels of IL-15 above basal plasma levels by administering subcutaneously doses of approximately 0.1 μg/kg to approximately 10 μg/kg of an IL-15/IL-15Rα complex to a subject. In some embodiments, the methods described herein achieve plasma levels of IL-15 by administering subcutaneously doses of approximately 0.1 μg/kg to approximately 20 μg/kg, approximately 10 μg/kg to approximately 20 μg/kg, approximately 20 μg/kg to approximately 40 μg/kg, or approximately 25 μg/kg to 50 μg/kg of an IL-15 or an IL-15/IL-15Rα complex to a subject. In certain embodiments, an IL-15 or an IL-15/IL-15Rα complex dose can be 0.1 μg/kg, 0.25 μg/kg, 0.75 μg/kg, 1 μg/kg, 1.5 μg/kg, 2 μg/kg, 2.5 μg/kg, 3 μg/kg, 3.5 μg/kg, 4 μg/kg, 4.5 μg/kg, or 5 μg/kg, 10 μg/kg, 15 μg/kg, 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

In certain embodiments, the composition comprising an IL-15 or an IL-15/IL-15Rα complex and one or more active agents is administered to a subject, wherein an IL-15 or an IL-15/IL-15Rα complex is administered in a cyclical regimen, wherein each cycle of the cyclical regimen comprises: (a) administering a dose of an IL-15 or an IL-15/IL-15Rα complex to the subject at a certain frequency for a first period of time; and (b) no administration of an IL-15 or an IL-15/IL-15Rα complex for a second period of time. In certain embodiments, the cyclical regimen is repeated 2, 3, 4, 5, 6, 7, 8, 9, 10 or more times. In some embodiments, an IL-15 or an IL-15/IL-15Rα complex is administered at a frequency of every day, every other day, every 3, 4, 5, 6 or 7 days. In certain embodiments, an IL-15 or an IL-15/IL-15Rα complex is administered 1, 2, 3, 4, 5, 6 or 7 days per week. In some embodiments, the first and second periods of time are the same. In other embodiments, the first and second periods of time are different. In specific embodiments, the first period for administration of the an IL-15 or an IL-15/IL-15Rα complex is 1 week to 4 weeks long, 2 to 4 weeks, 2 to 3 weeks, or 1 to 2 weeks. In other embodiments, the first period for administration of the IL-15/IL-15Rα complex is 1 week, 2 weeks, 3 weeks or 4 weeks long. In some embodiments, the second period of time is 1 week to 2 months, 1 to 8 weeks, 2 to 8 weeks, 1 to 6 weeks, 2 to 6 weeks, 1 to 5 weeks, 2 to 5 weeks, 1 to 4 weeks, 2 to 4 weeks, 2 to 3 weeks, 1 to 2 weeks, 3 weeks, 2 weeks or 1 week long. In a specific embodiment, the dose of the first cycle and each subsequent cycle is 0.1 μg/kg to 1 μg/kg, 1 μg/kg to 5 μg/kg, or 5 μg/kg to 10 μg/kg. In another embodiment, the first dose of the first cycle and each subsequent cycle is 0.1 μg/kg to 0.5 μg/kg, 1 μg/kg to 2 μg/kg, 1 μg/kg to 3 μg/kg, 2 μg/kg to 5 μg/kg, or 2 μg/kg to 4 μg/kg. In another embodiment, the dose of the first cycle and each subsequent cycle is 0.1 μg/kg, 0.25 μg/kg, 0.5 μg/kg, 1 μg/kg, 1.25 μg/kg, 1.5 μg/kg, 1.75 μg/kg, 2 μg/kg, 2.25 μg/kg, 2.5 μg/kg, 2.75 μg/kg, 3 μg/kg, 3.25 μg/kg, 3.5 μg/kg, 4 μg/kg, 4.25 μg/kg, 4.5 μg/kg, 4.75 μg/kg, or 5 μg/kg. In certain embodiments, the dose of the first cycle differs from the dose used in one or more subsequent cycles of the cyclical regimen.

In another embodiment, provided herein is a method for preventing, treating and/or managing disorders in a subject, wherein enhancement of IL-15-mediated immune function is beneficial for the prevention, treatment and/or management of such disorders, the method comprising administering compositions comprising an IL-15 or an IL-15/IL-15Rα complex and one or more active agents to the human subject in an escalating IL-15 dose regimen beginning with an initial low dose of between 0.1 μg/kg and 20 μg/kg as determined based on the mass of single chain IL-15, and sequentially escalating the dose two to three times over the previous dose, wherein each dose comprising an IL-15 or an IL-15/IL-15Rα complex is administered at least once, twice, or thrice before elevating the dose to the next level, and wherein the concentration of free IL-15 in a sample (e.g., a plasma sample) obtained from the subject a certain period of time after the administration of a dose of the IL-15/IL-15Rα complex (e.g., approximately 24 hours to approximately 48 hours after the administration of a dose of the IL-15/IL-15Rα complex and before the administration of another dose of the IL-15/IL-15Rα complex) is monitored before elevating the dose to the next level.

In another embodiment, provided herein is a method for preventing, treating and/or managing cancer in a subject, method comprising administering a composition comprising an IL-15 or an IL-15/IL-15Rα complex and one or more active agents, wherein the IL-15/IL-15Rα complex is administered to the human subject in an escalating dose regimen beginning with an initial low dose of between 0.1 μg/kg and 20 μg/kg as determined based on the mass of single chain IL-15, and sequentially escalating the dose two to three times over the previous dose, wherein each dose is administered at least once, twice, or thrice before elevating the dose to the next level, and wherein the concentration of free IL-15 in a sample (e.g., a plasma sample) obtained from the subject a certain period of time after the administration of a dose of the IL-15/IL-15Rα complex (e.g., approximately 24 hours to approximately 48 hours after the administration of a dose of the IL-15/IL-15Rα complex and before the administration of another dose of the IL-15/IL-15Rα complex) is monitored before elevating the dose to the next level.

In certain embodiments, the initial low dose is 0.5 μg/kg as determined based on the mass of single chain IL-15. In some embodiments, the initial low dose is administered 1, 2, 3, 4, 5, or 6 times, or 1 to 3, 1 to 4, 2 to 4, 2 to 5, 2 to 6, 3 to 6, or 4 to 6 times over a 5 to 7 day, 5 to 10 day, 7 to 12 day, 7 to 14 day, 7 to 21 day or 14 to 21 day period of time. In certain embodiments, each dose is administered at least 1, 2, 3, 4, 5, or 6 or more times over a 5 to 7 day, 5 to 10 day, 7 to 12 day, 7 to 14 day, 7 to 21 day or 14 to 21 day period of time. In certain embodiments, the subject is monitored for signs of an enlarged lymph node(s) and/or an enlarged spleen. In specific embodiments, the dose is not increased if the trough concentration of free IL-15 in a sample (e.g., plasma sample) from the subject is above 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, or 100 μg/ml. In specific embodiments, the dose is not increased if the trough concentration of free IL-15 in a sample (e.g., plasma sample) from the subject is 50 μg/ml to 75 μg/ml, 60 μg/ml to 75 μg/ml, 75 μg/ml to 85 μg/ml, 75 μg/ml to 100 μg/ml, 85 μg/ml to 100 μg/ml or 50 μg/ml to 100 μg/ml. In some embodiments, the method further comprises administering a maintenance dose of the IL-15/IL-15Rα complex to the subject, wherein the maintenance dose reaches trough levels of free IL-15 concentration of approximately 5 to 50 μg/ml in a sample (e.g., a plasma sample) from the subject. In some embodiments, the method further comprises administering a maintenance dose of the IL-15/IL-15Rα complex to the subject, wherein the maintenance dose reaches trough levels of free IL-15 of approximately 1 to 50 μg/ml in a sample (e.g., a plasma sample) from the subject.

In another embodiment, provided herein is a method for preventing, treating and/or managing disorders in a subject, wherein enhancement of IL-15-mediated immune function is beneficial for the prevention, treatment and/or management of such disorders, the method comprising administering compositions comprising an IL-15 or an IL-15/IL-15Rα complex and one or more active agents to the human subject in an escalating dose regimen beginning with an initial low dose of an IL-15 or an IL-15/IL-15Rα complex between 0.1 μg/kg and 1 μg/kg as determined based on the mass of single chain IL-15, and sequentially escalating the dose 1.2, 1.3, 1.4, 1.5, 1.6, 1.7, 1.8, or 1.9 fold over the previous dose, wherein each dose is administered at least once, twice, or thrice before elevating the dose to the next level, and wherein the concentration of free IL-15 in a sample (e.g., a plasma sample) obtained from the subject a certain period of time after the administration of a dose of the composition comprising an IL-15 or an IL-15/IL-15Rα complex (e.g., approximately 24 hours to approximately 48 hours after the administration of a dose of an IL-15 or an IL-15/IL-15Rα complex and before the administration of another dose of an IL-15 or an IL-15/IL-15Rα complex) is monitored before elevating the dose to the next level.

In another embodiment, provided herein is a method for preventing, treating and/or managing cancer in a subject, wherein enhancement of IL-15-mediated immune function is beneficial for the prevention, treatment and/or management of such disorders, the method comprising administering compositions comprising an IL-15 or an IL-15/IL-15Rα complex and one or more active agents to the human subject in an escalating dose regimen beginning with an initial low dose of an IL-15 or an IL-15/IL-15Rα complex between 0.1 μg/kg and 1 μg/kg as determined based on the mass of single chain IL-15, and sequentially escalating the dose 1.2, 1.3, 1.4, 1.5, 1.6, 1.7, 1.8, or 1.9 fold over the previous dose, wherein each dose is administered at least once, twice, or thrice before elevating the dose to the next level, and wherein the concentration of free IL-15 in a sample (e.g., a plasma sample) obtained from the subject a certain period of time after the administration of a dose of the composition comprising an IL-15 or an IL-15/IL-15Rα complex (e.g., approximately 24 hours to approximately 48 hours after the administration of a dose of an IL-15 or an IL-15/IL-15Rα complex and before the administration of another dose of an IL-15 or an IL-15/IL-15Rα complex) is monitored before elevating the dose to the next level.

In a specific embodiment, the initial low dose of an IL-15 or an IL-15/IL-15Rα complex is 0.5 μg/kg as determined based on the mass of single chain IL-15. In some embodiments, the initial low dose is administered 1, 2, 3, 4, 5, or 6 times, or 1 to 3, 1 to 4, 2 to 4, 2 to 5, 2 to 6, 3 to 6, or 4 to 6 times over a 5 to 7 day, 5 to 10 day, 7 to 12 day, 7 to 14 day, 7 to 21 day or 14 to 21 day period of time. In specific embodiments, each dose is administered at least 1, 2, 3, 4, 5, or 6 or more times over a 5 to 7 day, 5 to 10 day, 7 to 12 day, 7 to 14 day, 7 to 21 day or 14 to 21 day period of time. In certain embodiments, the subject is monitored for signs of an enlarged lymph node(s) and/or an enlarged spleen. In specific embodiments, the dose is not increased if the trough concentration of free IL-15 in a sample (e.g., plasma sample) from the subject is above 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, or 100 μg/ml. In specific embodiments, the dose is not increased if the trough concentration of free IL-15 in a sample (e.g., plasma sample) from the subject is 50 μg/ml to 75 μg/ml, 60 μg/ml to 75 μg/ml, 75 μg/ml to 85 μg/ml, 75 μg/ml to 100 μg/ml, 85 μg/ml to 100 μg/ml or 50 μg/ml to 100 μg/ml. In some embodiments, the method further comprises administering a maintenance dose of an IL-15 or an IL-15/IL-15Rα complex to the subject, wherein the maintenance dose reaches trough levels of free IL-15 concentration of approximately 5 to 50 μg/ml in a sample (e.g., a plasma sample) from the subject. In some embodiments, the method further comprises administering a maintenance dose of an IL-15 or an IL-15/IL-15Rα complex to the subject, wherein the maintenance dose reaches trough levels of free IL-15 of approximately 1 to 50 μg/ml in a sample (e.g., a plasma sample) from the subject.

In specific embodiments, the methods described herein are not cyclical in nature. In other words, in specific embodiments, the methods described herein do not include a cyclical administration regimen, wherein the cycle comprises administering a dose of an IL-15 or an IL-15/IL-15Rα complex and one or more active agents fora certain period of time (e.g., 1 to 4 weeks) followed by another period of time when the subject is not administered a dose of an IL-15 or an IL-15/IL-15Rα complex and one or more active agents (e.g., 1 week to 2 months) and this cycle is repeated any number of times (e.g., the cycle is repeated 2, 3, 4, 5, 6, 7, 8, 9, 10 or more times).

This disclosure provides methods for preventing, treating, and/or managing cancer, comprising administering an effective amount of an IL-15 or an IL-15/IL-15Rα complex and one or more active agents to a subject in need thereof. In certain embodiments, types of cancer to be treated can include, but is not limited to breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer. In certain embodiments, this disclosure provides methods for preventing, treating, and/or managing metastatic cancer and/or metastatic disease derived from the any of the aforementioned cancers.

In some embodiments, administration of an IL-15 or an IL-15/IL-15Rα complex in combination with one more active agents to a subject with cancer reduces the size of a tumor by at least 2 fold, preferably at least 2.5 fold, at least 3 fold, at least 4 fold, at least 5 fold, at least 7 fold, or at least 10 fold relative to the growth of a tumor in a subject with cancer administered a negative control as measured using assays well known in the art. In another embodiment, administration an IL-15 or an IL-15/IL-15Rα complex in combination with one more active agents to a subject with reduces the size of a tumor by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to the growth of a tumor in a subject with cancer administered a negative control as measured using assays well known in the art.

In some embodiments, the compositions comprising an IL-15 or an IL-15/IL-15Rα complex in combination with one more active agents are generally administered in the form of a pharmaceutical composition comprising at least one of the compounds of as disclosed herein together with a pharmaceutically acceptable vehicle, diluent or carrier as described below. In some embodiments, the compositions comprising an IL-15 or an IL-15/IL-15Rα complex linked to IL-12 or a derivative thereof in combination with one more active agents are generally administered in the form of a pharmaceutical composition comprising at least one of the compounds of as disclosed herein together with a pharmaceutically acceptable vehicle, diluent or carrier as described below. Thus, the compositions provided herein may be administered individually or together in any conventional oral, parenteral, rectal or transdermal dosage form. As used herein, the term “therapeutic combination” or “combination” refers to a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

A therapeutic combination may be provided in a single pharmaceutical composition so that both the IL-15 therapeutic (e.g., an IL-15 or a derivative thereof, or an IL-15 receptor alpha complex or a derivative thereof, or a fusion protein of the foregoing with IL-12 or a derivative thereof) and one or more active agents (e.g., an activator of PPAR, an inhibitor of FLT3, and/or a chemotherapeutic agent) can be administered together. Administration of combinations can be by any method. In some embodiments, administration can be locoregional or intratumoral. In some embodiments, appropriate excipients such as hydrogels can be used for delivery.

Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Alternatively, a therapeutic combination may be provided using more than one pharmaceutical composition. In some embodiments, the IL-15 therapeutic may be provided in one pharmaceutical composition and the active agent may be provided in a second pharmaceutical composition so that the two compounds can be administered separately such as, for example, at different times, by different routes of administration. Thus, it also may be possible to provide the IL-15 therapeutic and the active agent(s) in different dosing regimens.

For oral administration a pharmaceutical composition may take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tableting purposes. Solid compositions of a similar type are also employed as filters in soft and hard-filled gelatin capsules; preferred materials in this connection also include lactose or milk sugar as well as high molecular weight polyethylene glycols. A preferred formulation is a solution or suspension in an oil, for example, a vegetable oil, such as olive oil; triglycerides such as those marketed under the name, Miglyol™, or mono- or diglycerides such as those marketed under the name, Capmul™, for example, in a soft gelatin capsule. Antioxidants may be added to prevent long-term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The compositions comprising an IL-15 or an IL-15/IL-15Rα complex in combination with one more active agents can be administered via any route known in the art. In certain embodiments, an IL-15 or an IL-15/IL-15Rα complex in combination with one more active agents are formulated with polymers are especially suited for local and/or locoregional delivery, but such formulations can also be for systemic administration.

In some embodiments, an IL-15 or an IL-15/IL-15Rα complex in combination with one more active agents can be administered orally, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to deliver an IL-15 or an IL-15/IL-15Rα complex in combination with one more active agents and pharmaceutically acceptable salts thereof.

Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, intratumoral, or topically, particularly to the ears, nose, eyes, or skin. The mode of administration is left to the discretion of the practitioner.

In certain embodiments, it may be desirable to administer an IL-15 or an IL-15/IL-15Rα complex in combination with one more active agents locally and/or locoregionally. This may be achieved, for example, and not by way of limitation, by local infusion, topical application, e.g., in conjunction with a wound dressing, by injection, by means of a catheter, by means of a suppository, or by means of an implant, said implant being of a porous, non-porous, or gelatinous material, including membranes, such as sialastic membranes, or fibers.

In some embodiments, pulmonary administration can also be employed, e.g., by use of an inhaler or nebulizer, and formulation with an aerosolizing agent, or via perfusion in a fluorocarbon or synthetic pulmonary surfactant.

In certain embodiments, an IL-15 or an IL-15/IL-15Rα complex in combination with one more active agents can be delivered in a vesicle, in particular a liposome.

In some embodiments, an IL-15 or an IL-15/IL-15Rα complex in combination with one more active agents can be delivered in a controlled release system. In certain embodiments, a pump may be used. In another embodiment, polymeric materials can be used. In an embodiment, a controlled-release system comprising an IL-15 or an IL-15/IL-15Rα complex in combination with one more active agents is placed in close proximity to the tissue affected by the cancer to be prevented, treated and/or managed.

In certain embodiments, an IL-15 or an IL-15/IL-15Rα complex dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In some embodiments, an IL-15 or an IL-15/IL-15Rα complex dosage is a concentration of about 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM. In certain embodiments, the dosage is about 0.25 μg/kg or more, about 0.5 μg/kg or more, about 1 μg/kg or more, about 2 μg/kg or more, about 3 μg/kg or more, about 4 μg/kg or more, about 5 μg/kg or more, about 6 μg/kg or more, about 7 μg/kg or more, about 8 μg/kg or more, about 9 μg/kg or more, about 10 μg/kg or more, about 25 μg/kg or more, about 50 μg/kg or more, about 100 μg/kg or more, about 250 μg/kg or more, about 500 μg/kg or more, about 1 mg/kg or more, about 5 mg/kg or more, about 6 mg/kg or more, about 7 mg/kg or more, about 8 mg/kg or more, about 9 mg/kg or more, or about 10 mg/kg or more of a patient's body weight.

In certain embodiments, an IL-15 or an IL-15/IL-15Rα complex dosage is a unit dose of about 5 mg, about 10 mg, about 50 mg, about 100 mg, about 150 mg, about 200 mg, about 250 mg, about 300 mg, about 350 mg, about 400 mg, about 500 mg, about 550 mg, about 600 mg, about 650 mg, about 700 mg, about 750 mg, about 800 mg or more. In another embodiment, the dosage is a unit dose that ranges from about 5 mg to about 100 mg, about 100 mg to about 200 mg, about 150 mg to about 300 mg, about 150 mg to about 400 mg, 250 mg to about 500 mg, about 500 mg to about 800 mg, about 500 mg to about 1000 mg, or about 5 mg to about 1000 mg.

In certain embodiments, a dose of an IL-15 or an IL-15/IL-15Rα complex or composition thereof is administered to a subject every day, every other day, every couple of days, every third day, once a week, twice a week, three times a week, or once every two weeks. In other embodiments, two, three or four doses of the an IL-15 or an IL-15/IL-15Rα complex or composition thereof is administered to a subject every day, every couple of days, every third day, once a week or once every two weeks. In some embodiments, a dose(s) of an IL-15 or an IL-15/IL-15Rα complex or composition thereof is administered for 2 days, 3 days, 5 days, 7 days, 14 days, or 21 days. In certain embodiments, a dose of an IL-15 or an IL-15/IL-15Rα complex or composition thereof is administered for 1 month, 1.5 months, 2 months, 2.5 months, 3 months, 4 months, 5 months, 6 months or more.

The above-described administration schedules are provided for illustrative purposes only and should not be considered limiting. A person of ordinary skill in the art will readily understand that all doses are within the scope of the invention.

Without limiting the disclosure, a number of embodiments of the disclosure are described below for purpose of illustration.

The subject matter will be further described in the following examples, which do not limit the scope of the subject matter described in the claims.

### EXAMPLES

The Examples that follow are illustrative of specific embodiments of the disclosure, and various uses thereof. They are set forth for explanatory purposes only and should not be construed as limiting the scope of the claimed subject matter in any way.

### Materials and Methods

Mouse models. C57BL/6 female mice were purchased from Envigo International Holdings, Inc. Mice at 6-8 weeks of age were randomly assigned to treatment or control groups. For the orthotopic mouse EO771 breast model, cells were purchased from CH3 BioSystems or ATCC, respectively. Cell lines were cultured in complete RPMI 1640 medium supplemented with 10% fetal calf serum, 50 mM 2-mercaptoethanol, 100 U/ml Penicillin and 100 μg/ml Streptomycin. Murine EO771 cells (3×10⁵) were orthotopically inoculated at the 4th mammary fat pad of 6-8 weeks old mice. The cells were resuspended in PBS. Matrigel (Corning Inc.) was added at 1:3 dilution to facilitate the inoculation process. Matrigel, an extract of basement membrane proteins, was used as cell carrier medium for the cell transplantation studies forming a 3D gel at 37° C. facilitating the inoculation. Tumor size was measured using a digital caliper and tumor volume (mm³) was calculated by the following equation: L*W*H*π/6.

Immunotherapy of EO771 tumor-bearing mice. Treatment was initiated when tumors reached ˜20 mm³. Animals were treated with hetIL-15, which is a heterodimer comprising the IL-15 chain and soluble extracellular portion of IL-15 Receptor alpha chain. In some experiments, the hetIL-15Fc molecule was used, which is a fusion of hetIL-15 to the Fc fragment of human immunoglobulin G1 (IgG1), with similar results. hetIL-15 was administered in Matrigel (Corning Inc.), used in 1:4 dilution, every 4 days peritumorally at 5 μg/mouse in PBS. In the survival studies, mice were sacrificed when the primary tumor reached a 2 cm diameter or any other humane endpoints listed in the ACUC-approved animal protocol, such as 20% weight loss or acute morbidity.

### Example 1: HetIL-15 Treated Tumors Show Enhanced Activation and Proliferation CD8⁺T and NK Cells

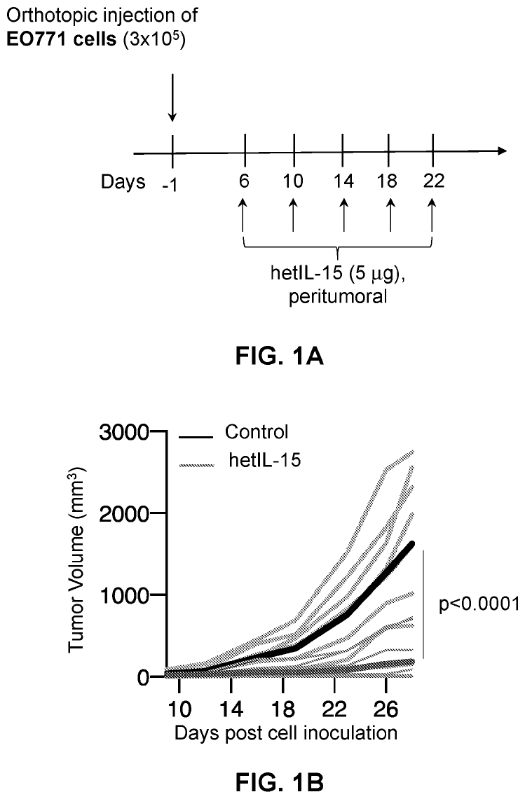

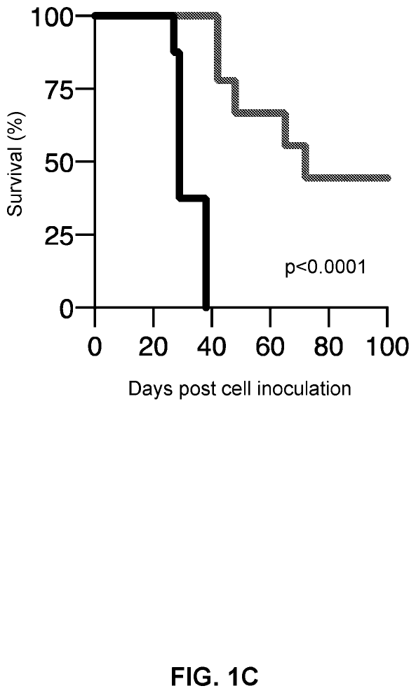

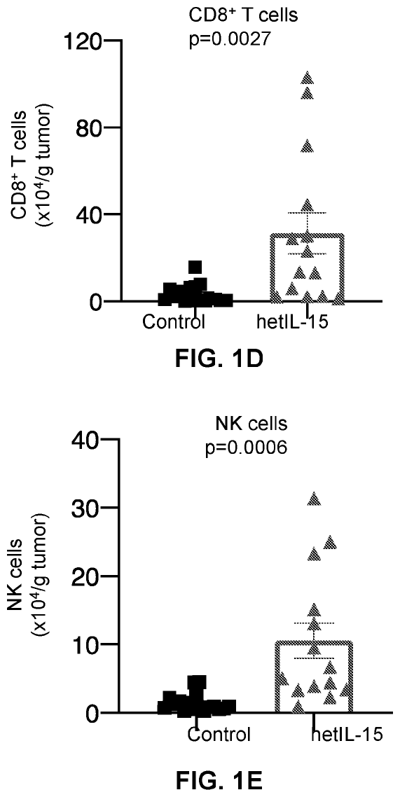

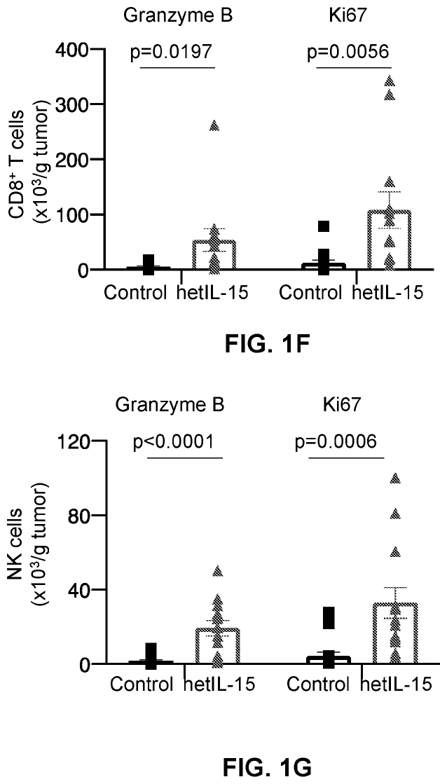

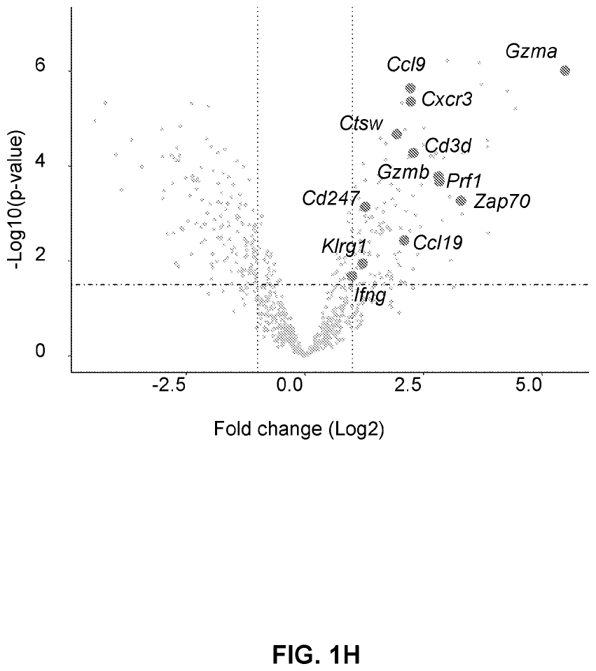

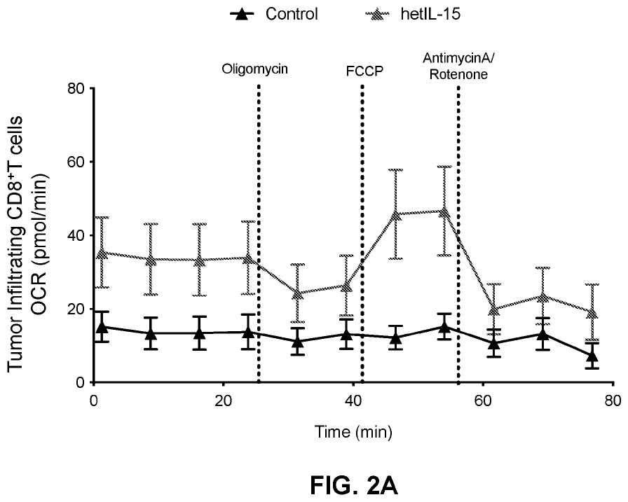

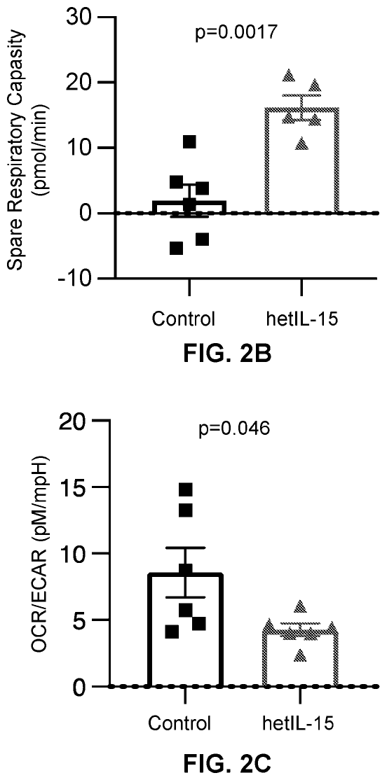

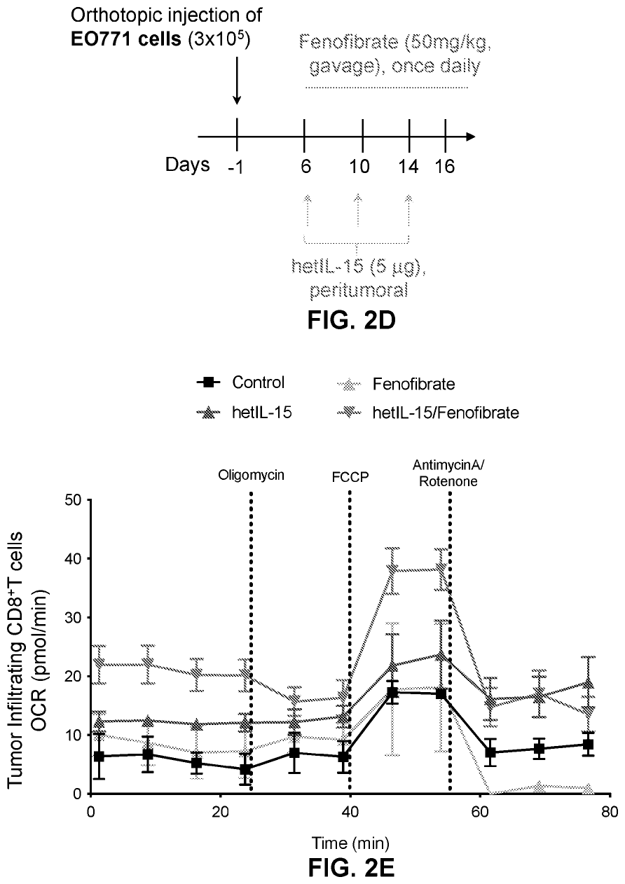

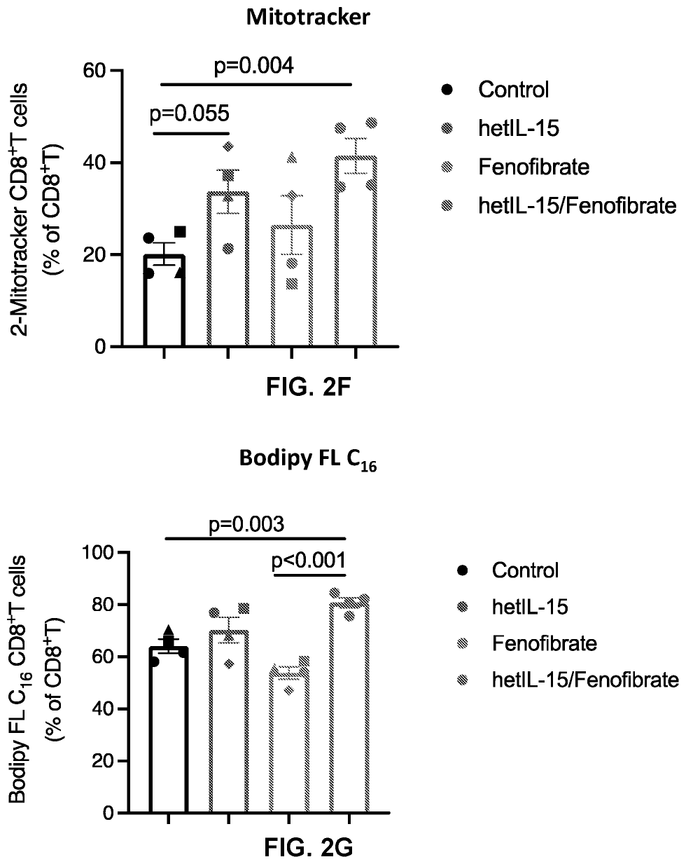

Administration of drug combinations in mouse orthotopic breast cancer models can be adjusted to optimize efficiency. hetIL-15 peritumoral administration resulted complete regression in approximately 40% of the treated animals and increased survival (FIGS. 1A-C). The tumor microenvironment was analyzed using transcriptomics and metabolic measurements. For the transcriptomic analysis tumors were mechanically disrupted in RLT buffer (QIAGEN) and RNA extraction was performed with RNeasy (QIAGEN) including on-column DNase I digestion, according to the manufacturer's instructions. nCounter PanCancer Immune Profiling Panel (NanoString Technologies) was used to monitor the expression of a panel of 770 genes related to immuno-oncology. The mRNA molecules were counted with the NanoString nCounter at the Laboratory of Molecular Technology Advanced Technology Program, Frederick National Laboratory). Analysis was performed with a workflow written in R and through a user interface developed on the Foundry Platform (Palantir Technologies). Seahorse XFe96 analyser was used to measure metabolic profiles, according to the manufacturer's instructions. Briefly, isolated tumor-infiltrating CD8⁺T cells (3×10⁵/well) were transferred to Seahorse assay plates and adhered using a Cell-Tak solution (Corning) in complete XF assay medium. Oligomycin (1 μM), FCCP (1.5 μM), and rotenone/antimycin A (0.5 μM) were injected. Data were collected in Wave software and analyzed using GraphPad Prism. CD8⁺T and NK cells were found increased in hetIL-15 treated tumors and showed enhanced activation and proliferation (FIGS. 1D-G). Transcriptomic analysis confirmed the activated state of the T and NK cells (FIG. 1H), whereas metabolic flux analysis of the tumor infiltrated CD8⁺T cells from hetIL-15 treated mice confirmed a rise in oxygen consumption rate (OCR) (FIG. 2A) with substantial increase of spare respiratory capacity (FIG. 2B), which supports an activated/non exhausted phenotype of these hetIL-15 treated effector cells. Furthermore, tumor infiltrated CD8⁺T cells from hetIL-15 treated mice presented pronounced shift in the OCR to ECAR ratio in comparison to control (FIG. 2C), confirming their increased proliferating status.

### Example 2: Combination of IL-15 and PPAR Activator Resulted in Tumor Growth Delay and Eradication of Tumors

Since, further promoting fatty acid (FA) catabolism improves the tumor-infiltrated CD8⁺T cells' ability to slow tumor progression, hetIL-15 immunotherapy was combined with a PPAR activator (fenofibrate; “FF”) (FIG. 2D). Combination IL-15 and FF therapy resulted in increased OCR (FIG. 2E), mitochondrial function (FIG. 2F), and FA uptake (FIG. 2G), revealing a more metabolically active phenotype compared to the tumor-infiltrating CD8⁺T cells in the hetIL-15 group.

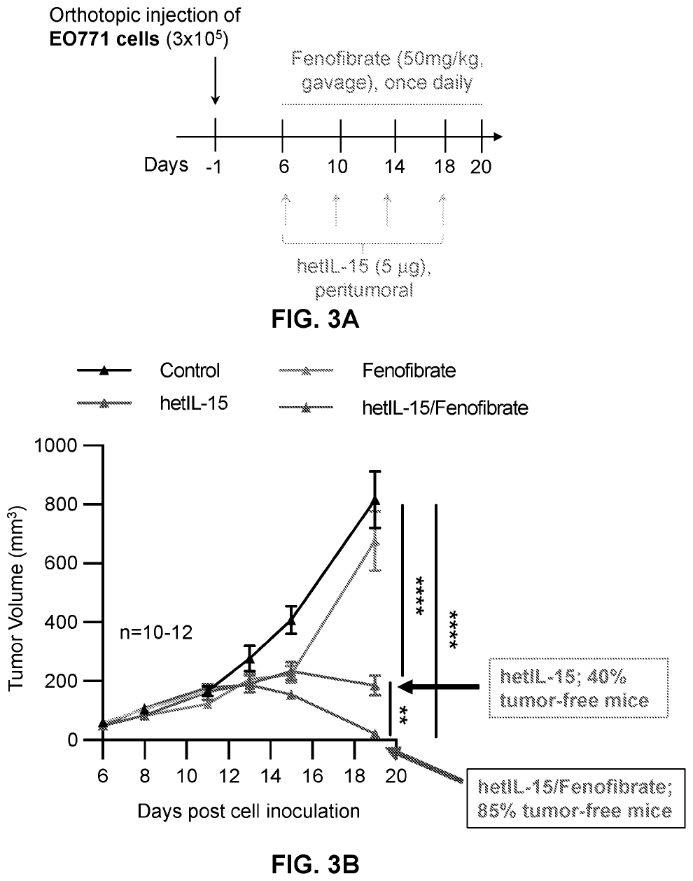

In addition, combined treatment of IL-15 immunotherapy and FF resulted in statistically significant EO771 tumor growth delay and complete eradication of the tumors in 85% of mice (FIGS. 3A, B). Together, these results indicate that FF maintains the number of functional tumor-infiltrated CD8⁺T cells by activating mitochondrial and cellular metabolism, leading in turn to enhanced antitumor immunity during hetIL-15 treatment. Furthermore, hetIL-15 synergizes with metabolic reprogramming of T cells to achieve superior antitumor efficacy and even complete cures.

### Example 3: Locoregional hetIL-15 Treatment Result in Increased Tumor Infiltration of CD103⁺cDC1s

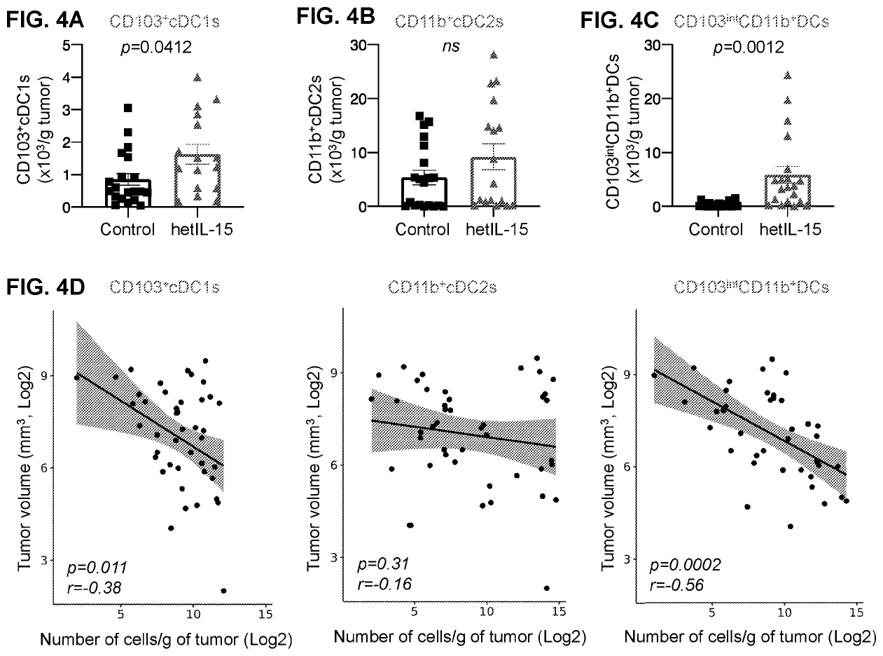

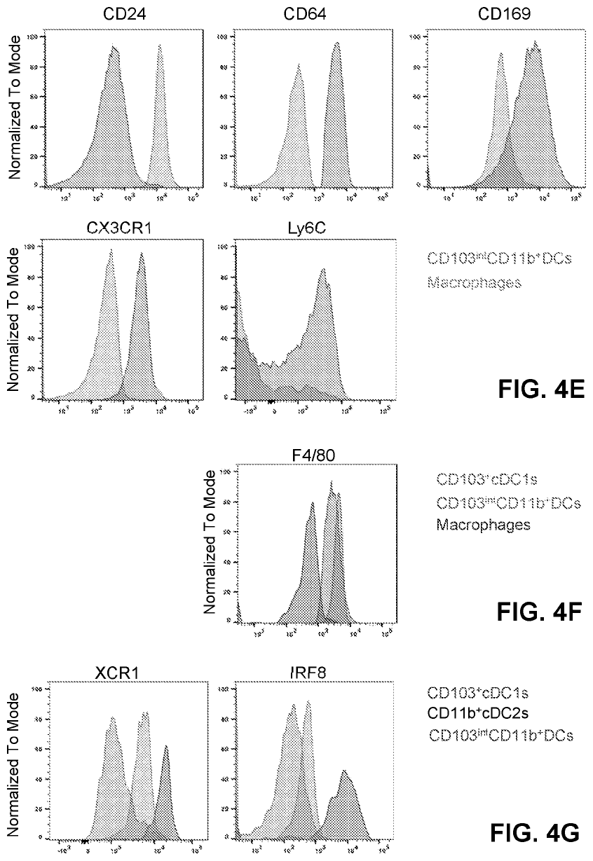

Locoregional hetIL-15 treatment resulted in increased tumor infiltration of CD103⁺cDC1s (FIG. 4A), whereas no significant difference was observed in the number of CD11b⁺cDC2s (FIG. 4B). An additional DC population was also observed, defined as CD103^{int}CD11b⁺DC (FIG. 4C), with phenotypical features distinct from the DC subsets previously reported in tumor mouse models. This population represented a minority of MHCII⁺CD11c⁺ cells under basal conditions, but became much more abundant in hetIL-15-treated tumors (FIG. 4C). Importantly, tumor infiltration by both CD103⁺cDC1s and CD103^{int}CD11b⁺DCs inversely correlated with the EO771 tumor size in hetIL-15 treated animals 48 hrs after the 3^{rd} hetIL-15 injection (FIG. 4D). In contrast, no correlation between intratumoral CD11b⁺cDC2s and tumor size was observed (FIG. 4D). Phenotypic profiling of the CD103^{int}CD11b⁺DCs in hetIL-15-treated tumors revealed that the cells express the dendritic cell marker CD24 and lack expression of the macrophage markers (CD64 (Fcgr1), CD169, CX3CR1 and Ly6C (Ly6c1)) (FIG. 4e), although they express F4/80 (FIG. 4F), suggesting they are not of macrophage lineage. Tumor-infiltrating CD103^{int}CD11b⁺DCs were also characterized by intermediate expression of XCR1 and IRF8 (FIG. 4G).

### Example 4: Combination of IL-15 and FLT3 Inhibitor Resulted in Tumor Growth Delay and Eradication of Tumors

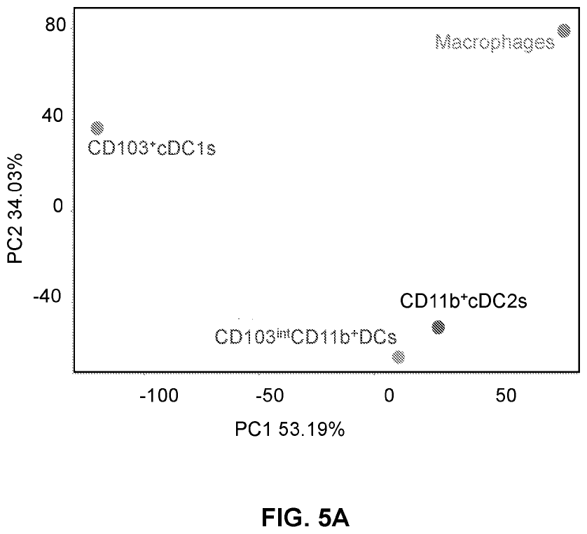

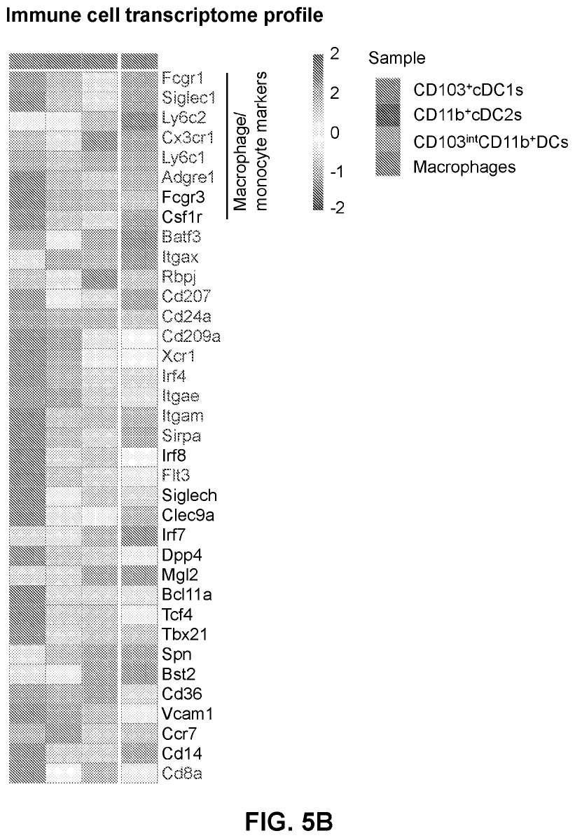

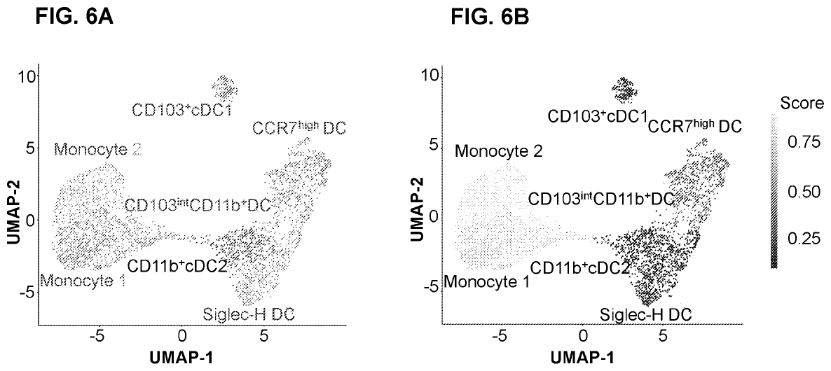

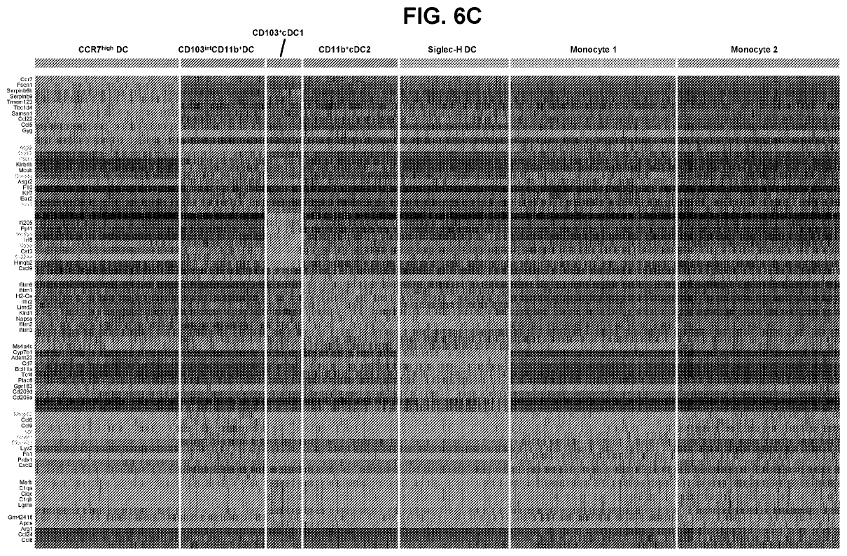

Further characterization of the tumor infiltrating CD103^{int}CD11b⁺DCs, was achieved by performing bulk and single-cell RNA sequencing (scRNA-seq). Principal component analysis (PCA) of the different sorted populations (CD103⁺cDC1, CD11b⁺cDC2, CD103^{int}CD11b⁺DC and macrophages) based on their transcriptome, revealed segregation of CD103^{int}CD11b⁺DCs; they showed a transcriptomic profile close to CD11b⁺cDC2s and mapped away from the macrophages in PCA space (FIG. 5A). Comparison with immune cell transcriptome profiles reported by Brown et al. [85], confirmed that tumor infiltrating CD103^{int}CD11 b⁺DCs showed low expression of the key macrophage genes Fcgr1, Cx3cr1, Siglec1, Ly6c1, Ly6c2, whereas DC markers (CD24a, Xcr1, Itgae, Itgam, Itgax, Sirpa, Irf4, Cd207 and CD209a) were highly or intermediately expressed in tumor-infiltrating CD103^{int}CD11b⁺DCs. This cell population has also increased Rbpj and Batf3 gene expression but low expression of F/t3 and CD8a (FIG. 5B). To further characterize the tumor infiltrating CD103^{int}CD11b⁺DCs, scRNA-seq was performed on sorted CD11c⁺ cells obtained from tumors of hetIL-15 treated or control EO771-tumor bearing mice. A total of 10,195 single-cell transcriptomes were generated after pre-processing. Unsupervised clustering was performed using Seurat v3.1.5 and Louvain method [86]. Clusters were serially annotated with SingleR using reference data generated form Brown et al. [85] and RNA-seq matrices from the sorted DC populations. After removal of cell-cycle signals, scRNA-seq of the CD11c⁺CD64^{reg} cells identified 7 distinct clusters visualized using UMAP (FIG. 6A). The cell identity of each cluster was established through the analysis of canonical DC gene expression similarity with reference genes from Brown et al. [85]. The CD103^{int}CD11b⁺DC population in hetIL-15 treated tumors was enriched in the sample density UMAP plot (FIG. 6B, yellow). In addition, CD103^{int}CD11b⁺DC population expressed a unique gene signature. Shared gene expression among individual clusters (FIG. 6C) revealed that CD103^{int}CD11 b⁺DCs possess a gene profile similar to monocytes and with several highly expressed (mo)DC/DC markers (mgl2, Ccl17, Plet1, Clec4n, CD24a, mmp12, clec4b1, and Anxa1) [87-92], suggesting a possible monocytic origin for this DC subset. CD103^{int}CD11b⁺DCs expressed the highest levels of Mgl2 and Ccl17 among the different DC subtypes. Plet1, a specific marker of cDC2 in the gastrointestinal tract, and Mmp12, which is expressed in both resting and activated human moDCs [91], were also highly expressed in CD103^{int}CD11b⁺DC cluster. In addition, CD103^{int}CD11b⁺DCs were characterized by high levels of Lpl, like the human moDCs [93], and Clec4b1, a protein that is selectively expressed in mouse CD11b⁺CD11c^{int}MHCII⁺ monocyte-derived cells [88]. The increased expression of genes related to antigen-processing machinery of DCs such as Wdfy4, Naaa and Annexin1 [87, 94, 95] was also verified in the CD103^{int}CD11b⁺DCs. Overall, the data demonstrated that these cells express canonical DC markers (CD24⁺CD11c⁺MHCII⁺CD103^{int}CD11b⁺), form a unique cell cluster which is different from other cDCs (cDC1s and cDC2s) and macrophages, are likely of monocytic origin and express genes associated with antigen presentation features.

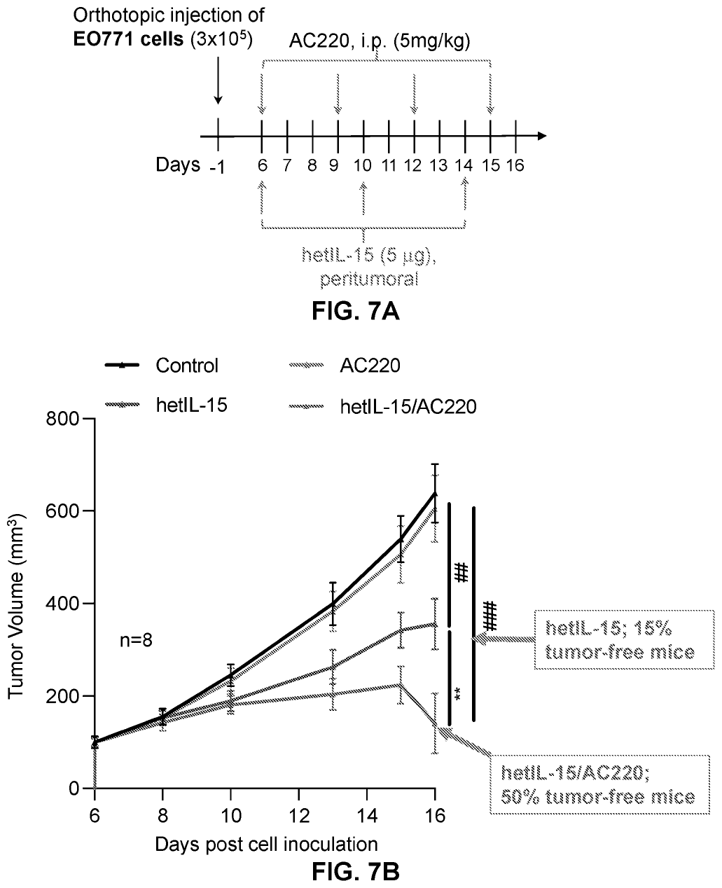

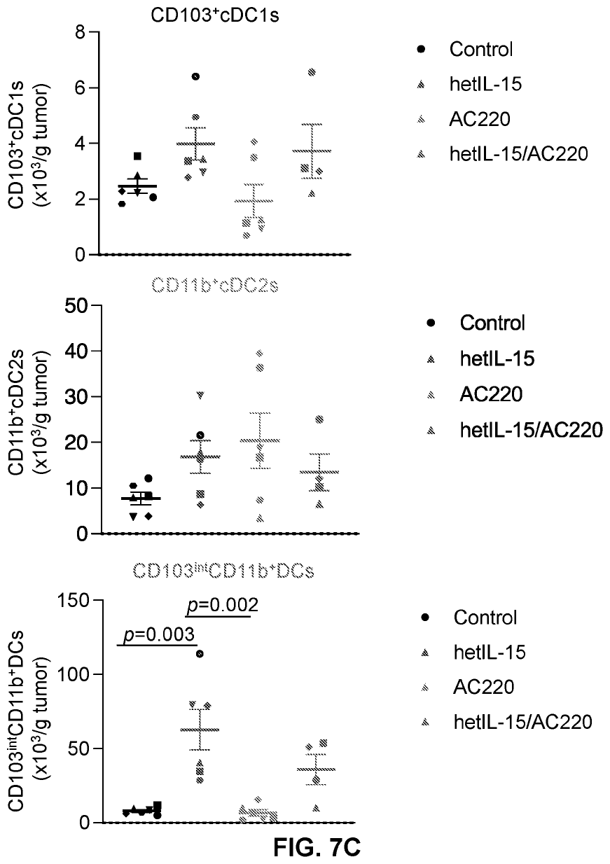

To explore the possibility of Flt3 dependence of tumor infiltrating CD103^{int}CD11b⁺DCs, EO771 tumor-bearing mice were treated with Quizartinib (AC220), a Flt3 specific inhibitor, in combination with hetIL-15 treatment (FIG. 7A). Combination therapy resulted in statistically significant EO771 tumor growth delay and complete eradication of the tumors in 50% of mice (FIG. 7B), after 10 days of treatment. In addition, flow cytometric analysis of the tumor-infiltrating DCs revealed that AC220 administration resulted in a minor effect on CD103+cDC1s, decreasing this DC population, and increased infiltration of CD11b⁺cDC2s, whereas no significant difference was observed in the number of CD103^{int}CD11b⁺DCs (FIG. 7C). Furthermore, combined treatment of hetIL-15 and AC220 resulted in increased tumor infiltration of CD103⁺cDC1s and CD103^{int}CD11b⁺DCs (FIG. 7C). The data demonstrate that locoregional administration of hetIL-15 activates the immune system and coordinates an effective immune response against EO771 tumors, promoting cancer cell killing by CD8+T and NK cells and increasing tumor infiltration of cDC1 and of a unique moDC population, the CD103^{int}CD11b⁺DCs, which is not responding to the Flt3 inhibition. Overall, hetIL-15 synergizes with Flt3 signaling to achieve superior antitumor efficacy and even complete cures.

### Example 5: Complete Tumor Regression by hetIL-15 Locoregional Administration is Associated with Intratumoral Accumulation of a Novel CD103^{int}CD11b⁺ Dendritic Cell Population

As shown in Appendix I (see ¶ [0344), locoregional administration of heterodimeric IL-15 (hetIL-15) in a triple-negative breast cancer (TNBC) orthotopic mouse model resulted in tumor eradication in 40% of treated mice, reduction of metastasis and induction of immunological memory against breast cancer cells. etIL-15 re-shaped the tumor microenvironment by promoting the intratumoral accumulation of cytotoxic lymphocytes, conventional type 1 dendritic cells (cDC1s) and a novel DC population expressing both CD11b and CD103 markers. These CD103^{int}CD11b⁺DCs share phenotypic and gene expression characteristics with both cDC1s and cDC2s, have transcriptomic profiles similar to monocyte-derived DCs (moDCs) and correlate with tumor regression. Therefore, hetIL-15, a cytokine directly affecting lymphocytes and inducing cytotoxic cells, has also an indirect rapid and significant effect on the recruitment of myeloid cells, initiating a cascade for tumor elimination though innate and adoptive immune mechanisms. The novel intratumoral DC population induced by hetIL-15 could be targeted for the development of effective immunotherapy approaches for the treatment of cancer.

Locoregional therapy with hetIL-15 is an effective therapy that holds promise as a future therapeutic option for cancer (in particular triple negative breast cancer). hetIL-15 coordinates an effective local and systemic immune response against the EO771 and 4T1 tumors, promoting tumor growth control by CD8⁺T and NK cells and increasing tumor infiltration of cDC1 and of a unique CD103^{int}CD11b⁺DC subpopulation most closely related to moDC. These cells may have a complementary role with the cDC1s in the anti-tumoral immune response. This treatment demonstrates that hetIL-15 administration enhanced the intratumoral interaction between DC and lymphocytes, which leads to the generation of a long-lasting, specific and protective anti-tumoral immune response. These properties can lead to additional therapeutic options for breast cancer patients.

### Example 6: Therapeutic Efficacy of hetIL-15 in Combination with Chemotherapy in Different Mouse Models of Pancreatic Ductal Adenocarcinoma (PDA) and Triple Negative Breast Cancer (TNBC) Models

Locoregional administration of hetIL-15 in the area of the tumor significantly reduced the primary tumors on both 4T1 breast cancer and KPC pancreatic mouse models. In contrast, systemic administration of hetIL-15 showed low or marginal benefit on the primary tumors, in both models.

Cell lines were cultured in complete RPMI 1640 medium supplemented with 10% fetal bovine serum, 100 IU/mL Penicillin and 100 mg/mL Streptomycin.

Genetically engineered mouse mode (GEMM) KPC: GEMM KPC: Pdx1-Cre;KrasG12D;Trp53R172H is one of the most clinically relevant and available animal models of pancreatic cancer (see Hingorani S R et al (2005) Trp53R172H and KrasG12D cooperate to promote chromosomal instability and widely metastatic pancreatic ductal adenocarcinoma in mice. Cancer Cell 7(5):469-483). GEMM KPC mice develop pancreatic tumors at the age of around 15-weeks-old. Tumor growth was measured via ultrasound imaging.

Orthotopic and heterotopic PDAC models: Pancreatic tumor cell line was derived from a KPC (Pdx-1-Cretg/+,Kras<tm4Tyj>/Jki/+,p53 LSL R172Hki/+) mouse on a C57B/6 mice.

Orthotopic tumor implantation: Female C57BL/6 mice aged 6-8 weeks were used for establishing orthotopic PDAC models. 4×10⁵ viable KPC cells suspended in a 3:1 PBS to Matrigel solution were directly injected (10 uL/injection) into the pancreas. Tumor growth was measured via ultrasound imaging.

Subcutaneous or mammary fat pad tumor implantation: Implanted KPC tumors were generated by injecting 4×10⁵ viable KPC cells (50 uL/injection) suspended in a 3:1 PBS to Matrigel solution into the right flank or 4th mammary fat pad of female C57BL/6 mice aged 6-8 weeks. Mice were randomized into different treatment groups when the tumor were palpable. The tumor size was measured using a digital caliper. Tumor volume (mm³) was calculated by the following equation: L*W*H*p/6.

Intravenous (I.V.)-KPC metastatic model: The IV model is used to evaluate the effects of the treatment directly on the metastatic disease. KPC cells, which are inoculated through the tail vein, preferably colonize the lungs. Viable KPC cells (3×10⁵) cells suspended in PBS were injected into the lateral tail vein of female C57BL/6 mice aged 6-8 weeks. Each mice received 50 uL/injection and 21 days post inoculation, the mice were euthanized and the metastatic disease into the lungs, was studied.

Orthotopic breast cancer model: 3×10⁵ viable EO771 cells suspended in a 3:1 PBS to Matrigel solution were directly injected (50 uL/injection) into the 4th mammary pad of the female C57BL/6 mice aged 6-8 weeks. The tumor size was measured using a digital caliper. Tumor volume (mm³) was calculated by the following equation: L*W*H*p/6.

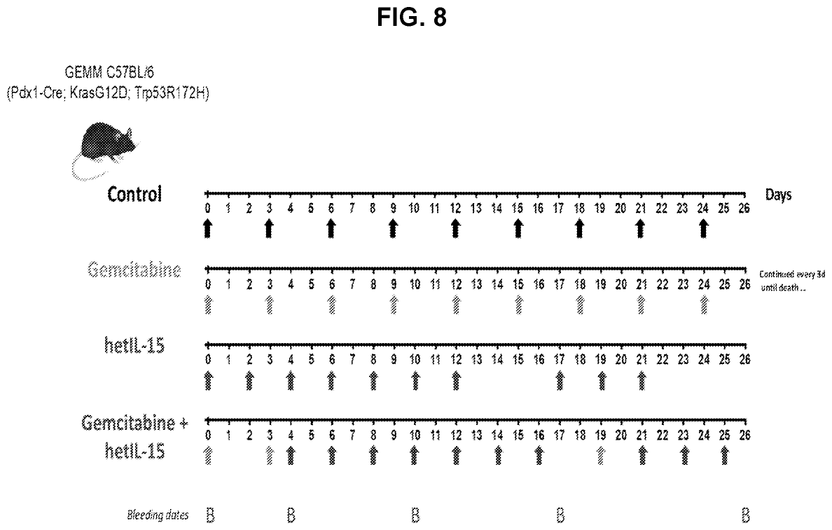

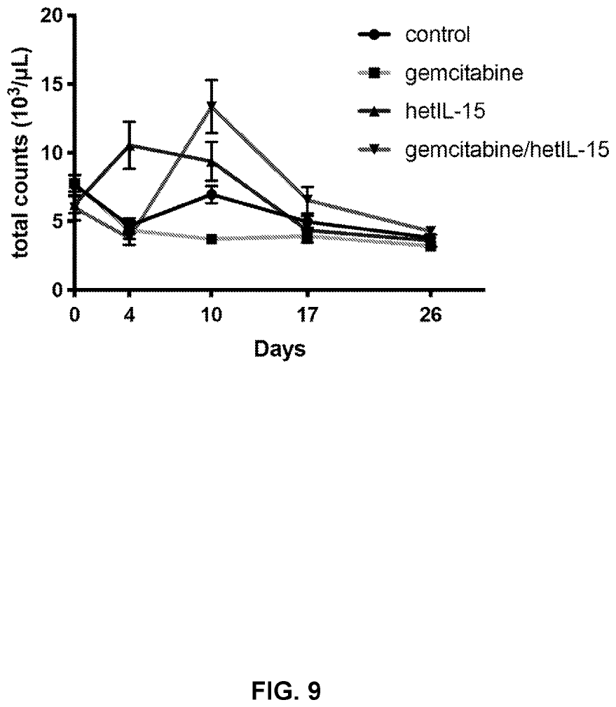

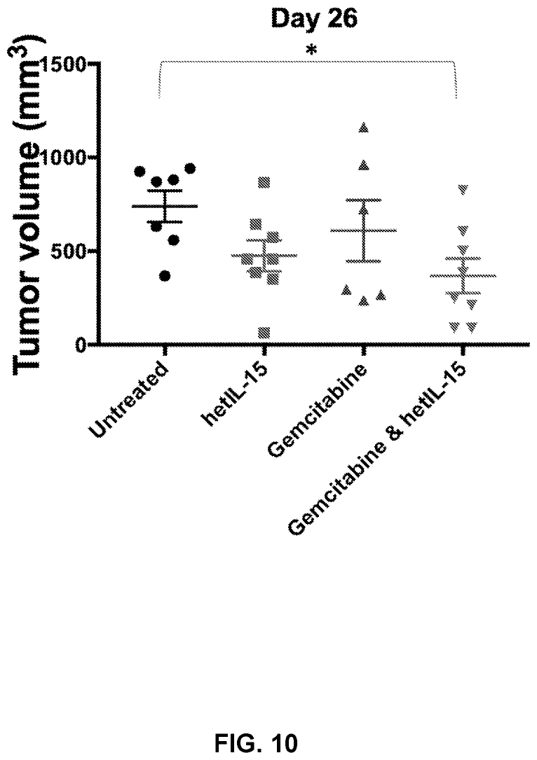

hetIL-15 Immunotherapy of PDA and TNBC models: Treatment Schedule of the GEMM Model: GEMM KPC mouse model of pancreatic cancer was used to test hetIL-15 anti-tumor activity as single agent and in combination with the chemotherapeutic agent gemcitabine. Tumor growth was measured via ultrasound imaging and when the tumor reached the size of around 40 mm³, the mice were randomized in four groups: (i) control (PBS), (ii) gemcitabine (100 mg/kg), (iii) hetIL-15 (3 ug) and (iv) gemcitabine plus hetIL-15. Gemcitabine monotherapy was given sequentially as this treatment scheme is followed in the clinic (FIG. 8)

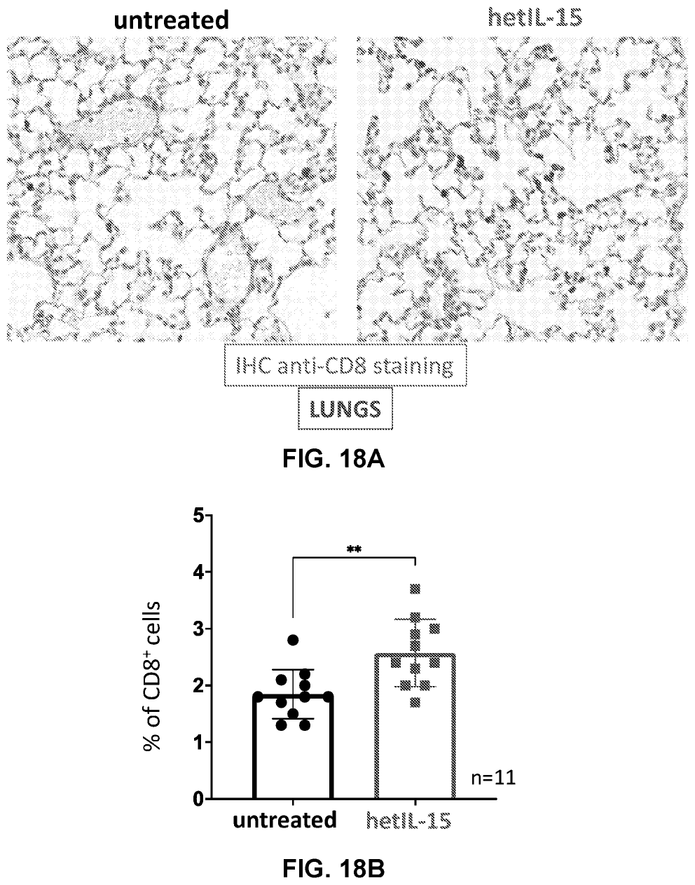

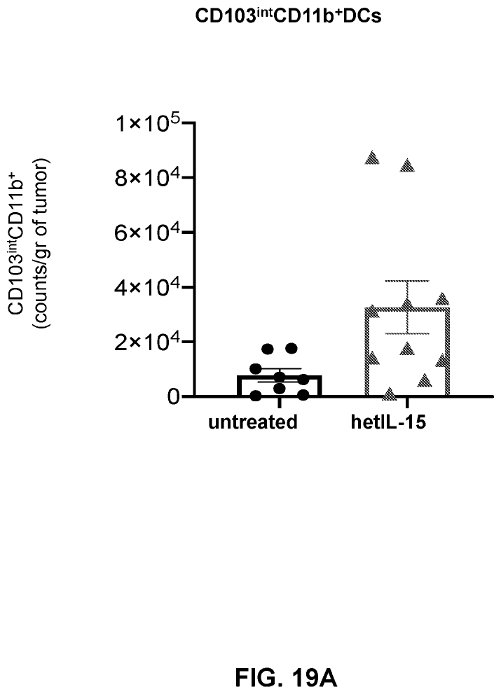

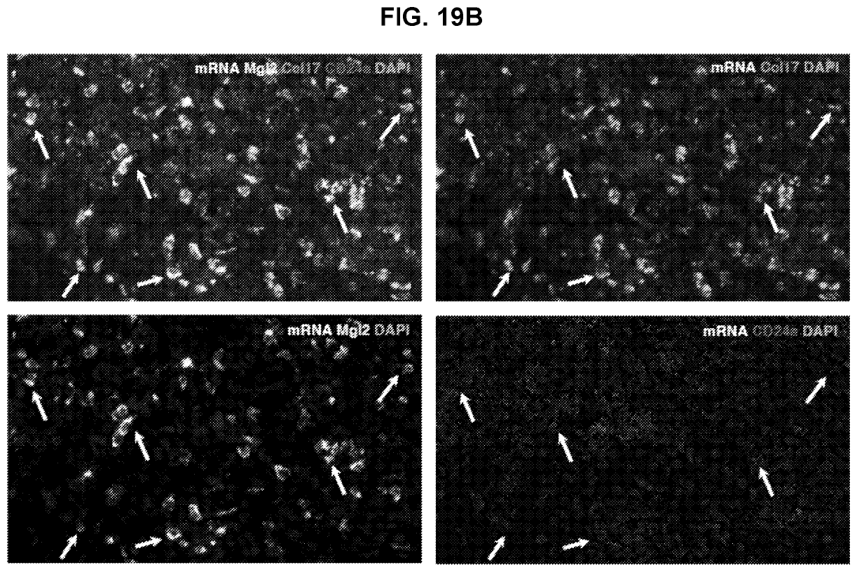

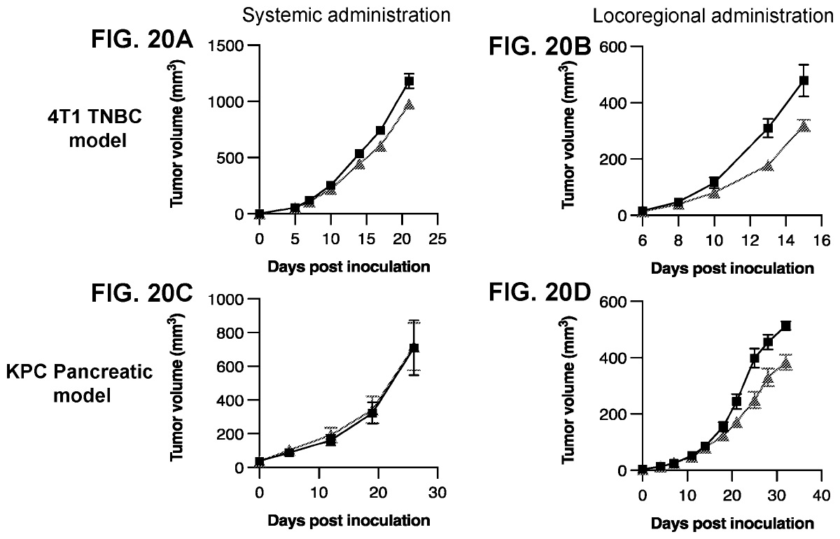

Intravenous (I.V.)-KPC metastatic model. The treatment was initiated 4 days post inoculation. hetIL-15 was administered IP (9 IP injections—3 ug/mouse every other day) (FIGS. 18 and 19)

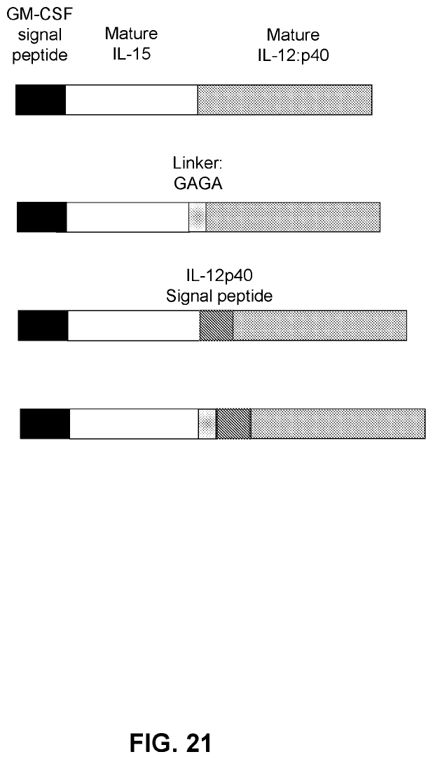

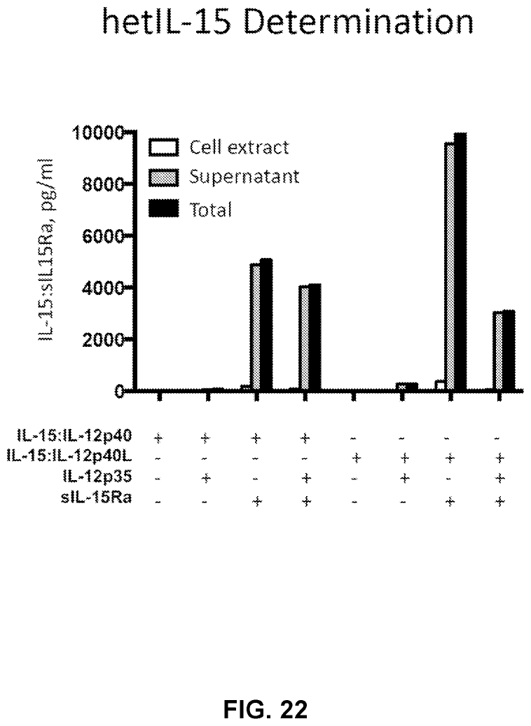

Combined treatment of IL-15 immunotherapy and Fenofibrate. Mice with EO771 TNBC tumors around 20 mm³ were distributed in different groups and treated with three locoregional hetIL-15 injections (3 μg/mouse/dose) every 4 days or/and Fenofibrate (50 mg/kg) daily by gavage (FIG. 21)

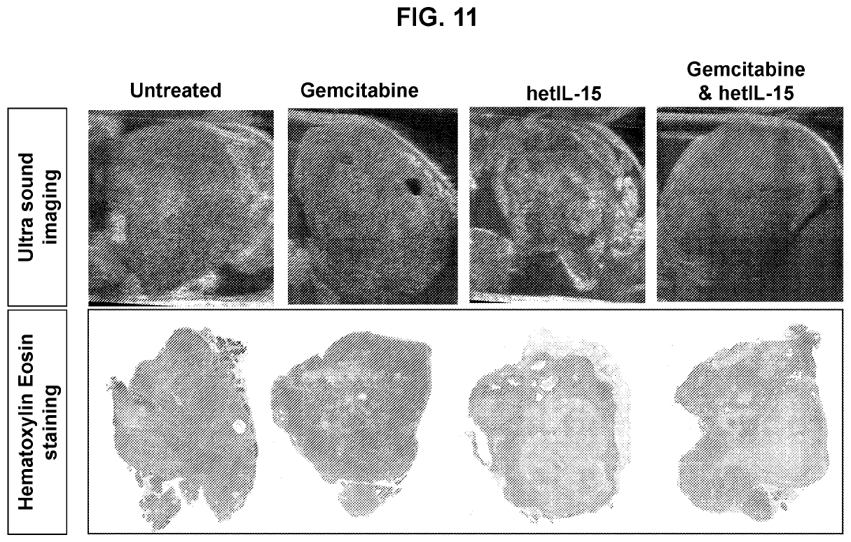

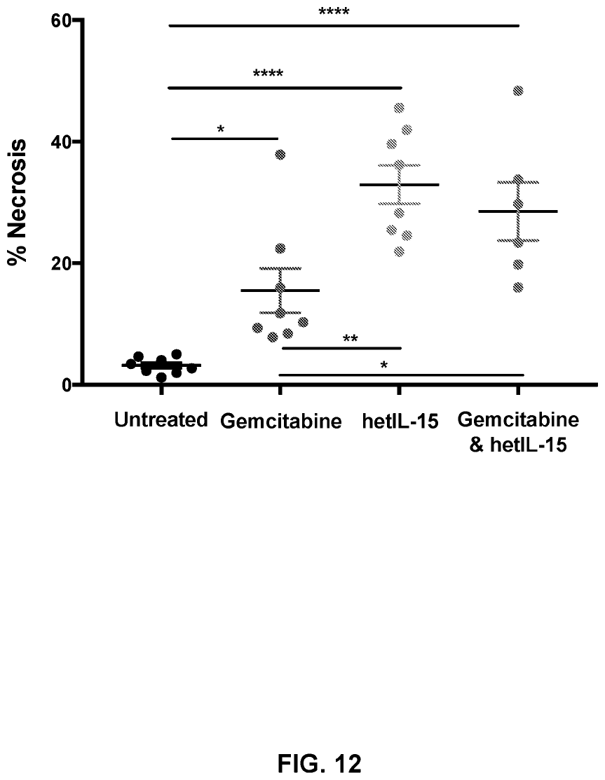

Evaluation of the necrosis. Tumors from GEMM KPC models were H&E-stained and the areas of necrosis was calculated for each group. The evaluation of necrosis with H&E staining is possible as the necrotic areas are depicted with a paler pink derived from the eosin-stained proteins that are released by the necrotic cells. Similar size tumor comparison of mice with endpoint from day 26-50 showed extensive intratumoral necrosis upon hetIL-15 monotherapy. (FIGS. 11 and 12)

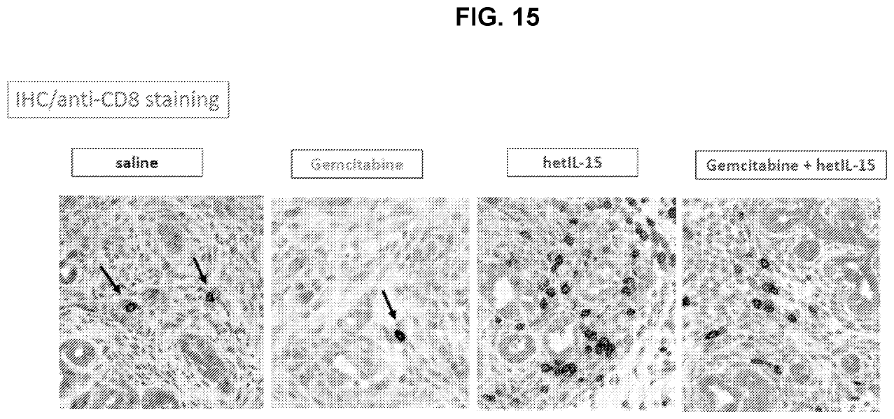

Histological analysis. For both the primary tumors and the lungs, the analysis was performed as follows. The primary pancreatic tumors and lungs were fixed in 10% neutral buffered formalin (NBF, Sigma, #HT501128) and paraffin embedded. Sections were stained with hematoxylin/eosin (H&E) or processed for immunohistochemistry (IHC). IHC automated staining was performed on Leica Biosystems' Bond RX with the following conditions: Epitope Retrieval 1 (Citrate) 20′ for CD8a (eBioscience, #14-0808-82, 1:50) The Bond Polymer Refine Detection Kit (Leica Biosystems, #DS9800) with the omission of the Post Primary Reagent was used, and an anti-rat secondary antibody (Vector Labs, #BA-4001) was included. Isotype rat IgG2a antibody (BD Bioscience, #559073) was used in place of the primary antibodies for the negative controls. H&E and IHC slides were scanned using an Aperio AT2 scanner (Leica Biosystems, Buffalo Grove, IL) into whole slide digital images (one section was used for the analysis). Image analysis of positive-stained cells in lung tissue was performed using HALO image analysis software (v3.3.2541.300; Indica Labs, Corrales, NM). Positive-stained cells located in vessels or areas of artifact such as folds and tears were excluded from the analysis (FIGS. 15 and 18)

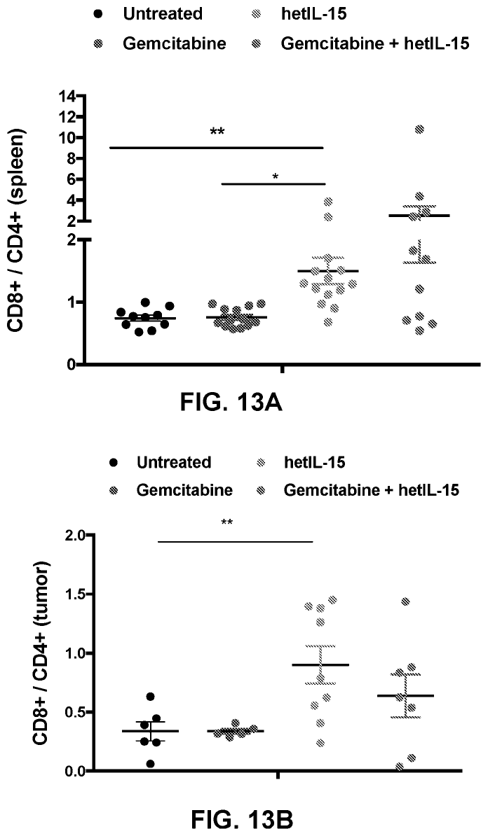

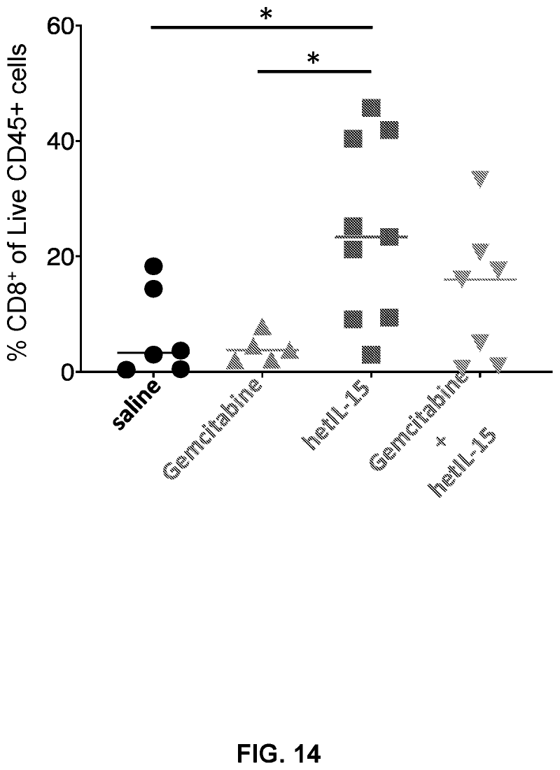

Flow cytometric analysis. At necropsy, tumors were processed for flow cytometric analysis. All tumors were weighed before the start of the process. To generate single cell suspensions, tumors were enzymatically digested using the tumor dissociation kit (Miltenyi Biotec Inc.) and mechanically dissociated using the GentleMACS™ Dissociator (Miltenyi Biotec Inc.). Tissues were passed through a 100 μm cell strainer (Falcon) and washed with PBS before proceeding with antibody mediated staining. dLNs were dissociated using a 100 μm cell strainer and washed with PBS. Surface staining was performed using the following anti-mouse antibodies: CD45 (clone 30-F11), CD3 (clone 145-2C11), CD8a (clone 53-6.6), CD19 (clone 1D3), NK1.1 (clone PK136), B220 (clone RA3-6B2), XCR1 (clone ZET), MHCII (clone M5/114.15.2), CD11c (clone N418), CD24a (clone M1/69), CD64 (clone X55-5/7.1), F4/80 (clone BM8), CD103 (clone M290), CD11b (clone M1/70) and CD172a (clone P84). For intracellular staining, cells were fixed and permeabilized using the Foxp3 staining buffer. Samples were stained with IRF8 (clone V3GYWCH). The samples were acquired on a Fortessa (BD Biosciences) flow cytometer, and the data were analyzed using the FlowJo software (Tree Star, Ashland, OR, USA) (FIGS. 13, 14 and 19 left panel).

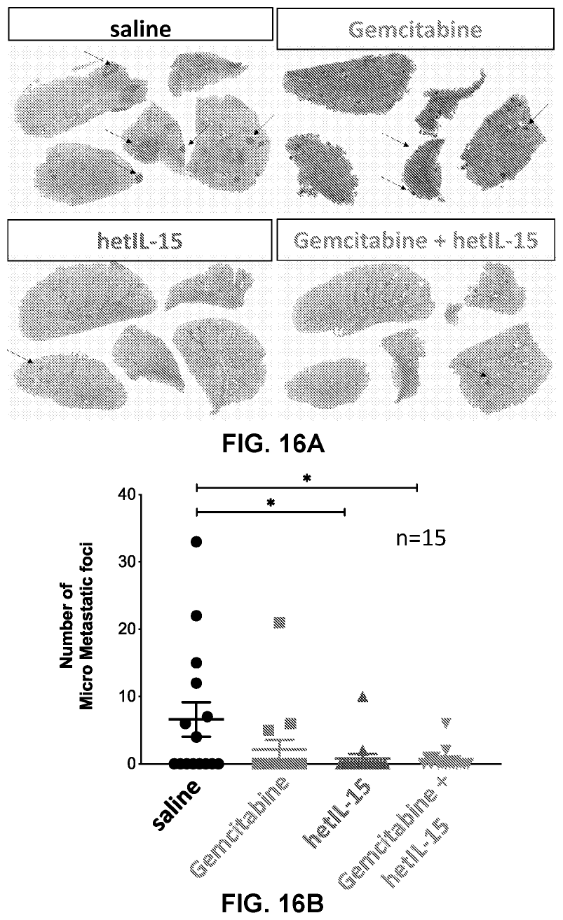

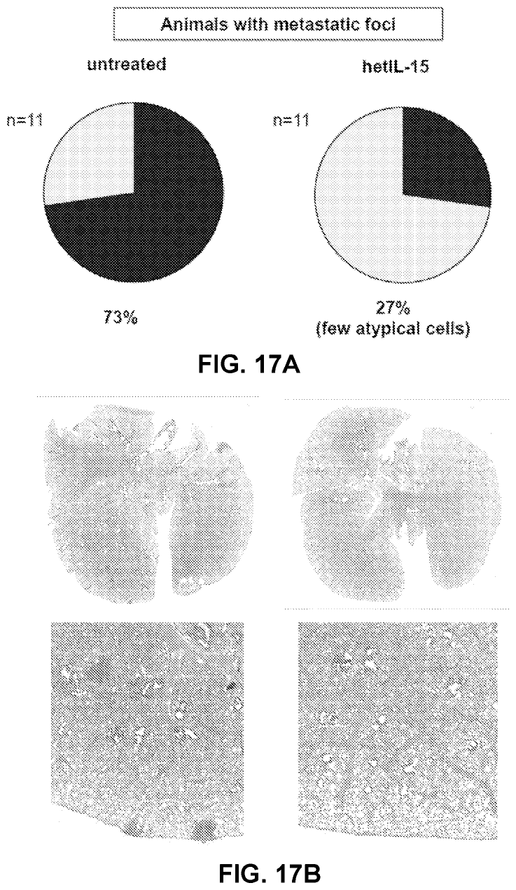

The evaluation of metastasis. The anti-metastatic effect of hetIL-15 treatment was evaluated, with H&E-staining of the lungs sections of mice of KPC pancreatic cancer model. The H&E-staining revealed that hetIL-15 monotherapy decreased the total number of the metastatic foci in GEMM KPC and also in the IV KPC model (FIGS. 16 and 17).

Multiplex RNA in situ hybridization staining. CD24a, Mgl2, and Ccl17 expression was detected by staining 5 mm FFPE tissue sections with RNAscope 2.5 LS Probe-Mm-CD24a-C1 (ACD, Cat #432698), RNAscope 2.5 LS Probe-Mm-Mgl2-01 (ACD, Cat #822908-C2), RNAscope 2.5 LS Probe-Mm-Cc117-C3 (ACD, Cat #428498-C3), and the RNAscope LS Multiplex Fluorescent Assay (ACD, Cat #322800) using the Bond RX auto-stainer (Leica Biosystems) with a tissue pretreatment of 15 min at 95° C. with Bond Epitope Retrieval Solution 2 (Leica Biosystems), 15 min of Protease III (ACD, Cat #322340) at 40° C., and 1:750 dilution of TSA-Cyanine 5 Plus, TSA-Fluorescein Plus and TSA-Cyanine 3 Plus (AKOYA), respectively. The RNAscope 3-plex LS Multiplex Negative Control Probe (Bacillus subtilis dihydrodipicolinate reductase (dapB) gene in channels C1, C2, and C3, Cat #320878) was used as a negative control. The RNAscope LS 2.5 3-plex Positive Control Probe-Hs was used as a technical control to ensure the RNA quality of tissue sections was suitable for staining. Slides were digitally imaged using an Aperio ScanScope FL Scanner (Leica Biosystems) (FIG. 19, right panel).

### Example 7: Anti-Tumor Effects of IL-15:IL-12p40 Chimera Fusion Protein

Additional hybrid molecules having IL-12 and IL-15 activity are examples of molecules that can be used as alternatives to hetIL-15. These molecules can be administered as proteins or as nucleic acids for production in a subject. Nucleic acids can have the property for restricted expression only at the site of production and display at the surface of the producing cells (for example tumors). This is achieved by using forms of IL-15Rα which are plasma membrane associated, anchoring the produced protein to the surface of the cell. Other embodiments can be secreted in the area of production and have local and systemic activity. Some embodiments use formulations of IL-15 not including the IL-15Rα component. For example, FIG. 21 shows a schematic of an IL-15:IL-12 chimera that can be generated with either mouse or human sequences comprising IL-15 fused with IL-12p40. This chimera has properties of both IL-15 and IL-12 and locoregional administration of the combination at the area of the tumor enhances therapeutic activity and decreases systemic exposure and toxicity.

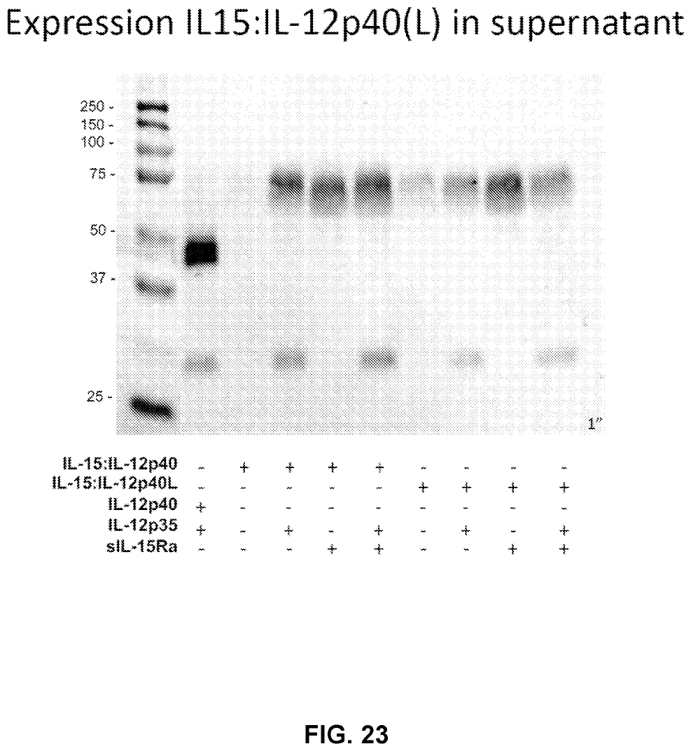

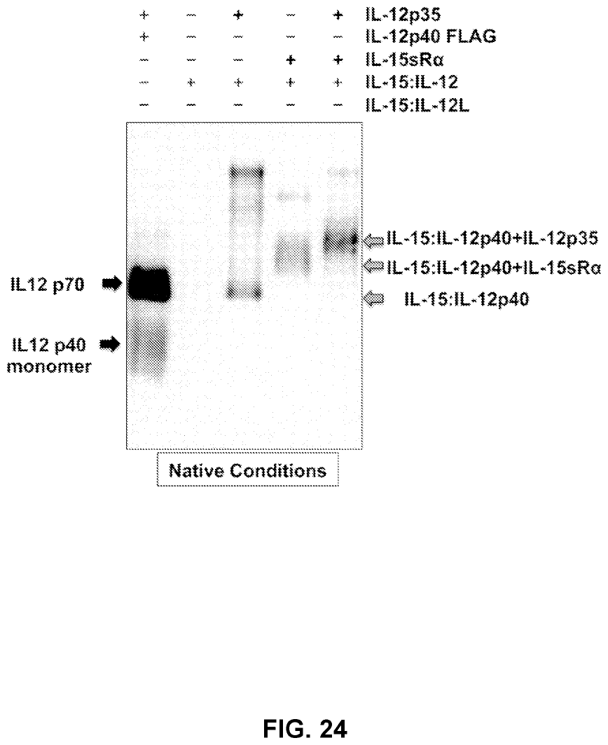

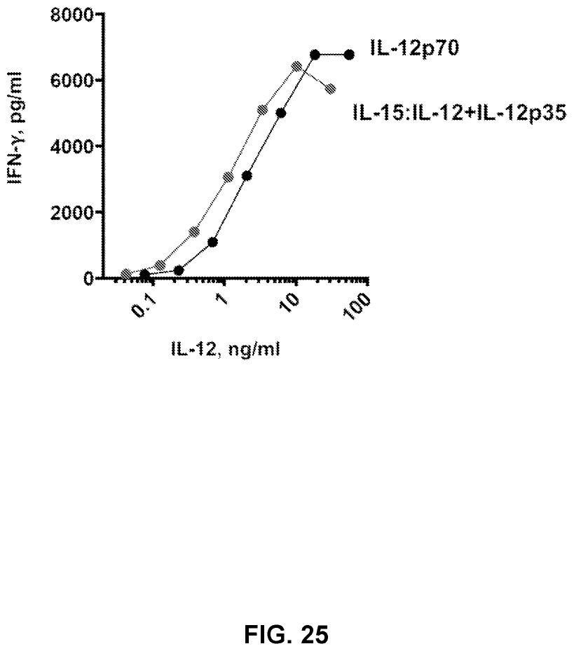

An IL-15:IL-12p40(L) fusion protein chimera demonstrated expression of the IL-15:IL-12p40(L) chimera in supernatant of embryonic fiberblasts (see FIG. 23), and the IL-15:IL-12p40 fusion protein maintained the ability to interact with IL-12p35 and IL-15sRα (see FIG. 24). Stimulation of NK-92 cells with the IL-15:IL-12p40 fusion protein or by IL-12p70 results in IFN-γ production by the NK-92 cells (see FIG. 25).

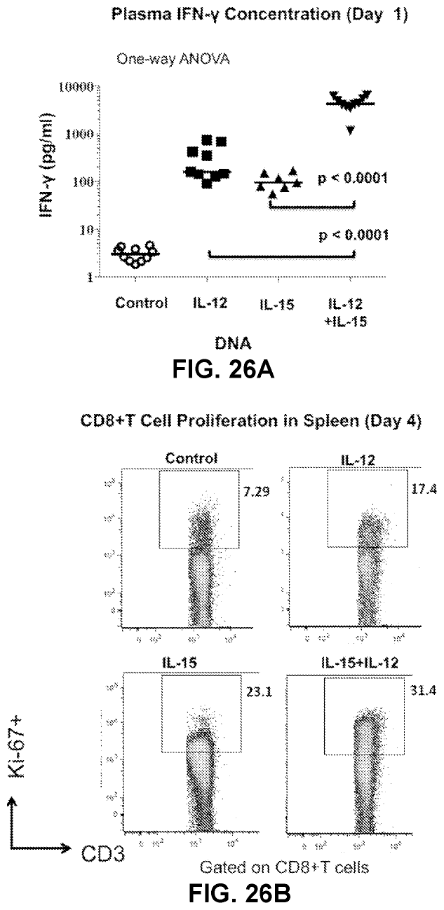

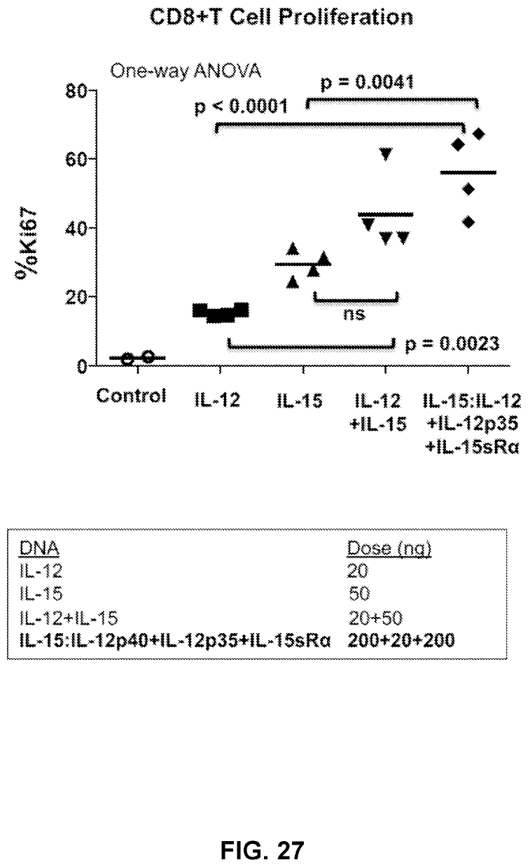

Co-delivery of IL-12 and IL-15 into BALB/c mice resulted in synergistic effects on IFN-γ production and CD8+T cell proliferation (see FIG. 26A and FIG. 26B). Co-delivery of an IL-15:IL-12 fusion protein and IL-12p35 promoted CD8+T cell proliferation similar to the combination of heterodimeric IL-15 and IL-12 (FIG. 27).

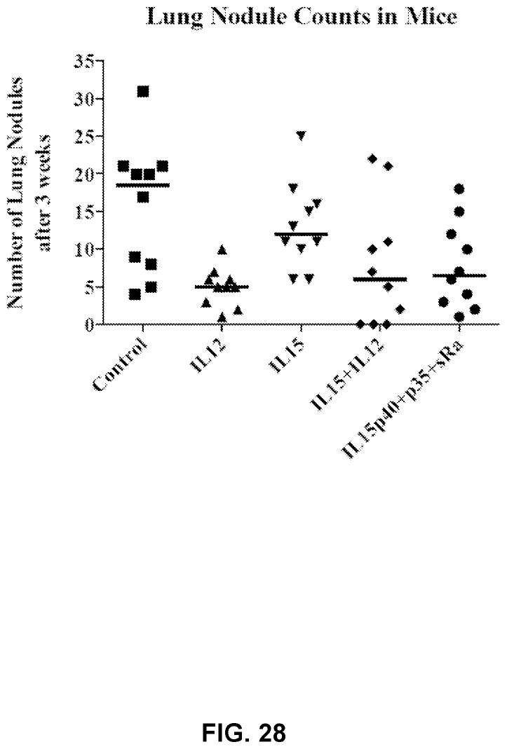

Finally, the IL-15:IL-12p40 fusion protein+IL12p35+IL15sRa chimera (CS70) demonstrated anti-tumor effects in mice (see FIG. 28). The results demonstrated a greater than 2-fold decrease in metastases to the lung.

Having described the subject matter of the disclosure in detail and by reference to specific embodiments thereof, it will be apparent that modifications and variations are possible without departing from the scope of the claimed subject matter. More specifically, although some aspects of the present disclosure are identified herein as particularly advantageous, it is contemplated that the present subject matter is not necessarily limited to these particular aspects of the claimed subject matter.

### APPENDIX I—TUMOR ERADICATION BY HETIL-15 LOCOREGIONAL ADMINISTRATION IS RELATED TO INTRATUMORAL ACCUMULATION OF A NOVEL CD103INTCD11B+DENDRITIC CELL POPULATION

### Submitted Manuscript: Confidential

Tumor Eradication by hetIL-15 Locoregional Administration is Related to Intratumoral Accumulation of a Novel CD103^{int}CD11b⁺Dendritic Cell Population

Abstract: Locoregional administration of heterodimeric IL-15 (hetIL-15) in a triple-negative breast cancer (TNBC) orthotopic mouse model resulted in tumor eradication in 40% of treated mice, reduction of metastasis and induction of immunological memory against breast cancer cells. hetIL-15 re-shaped the tumor microenvironment by promoting the intratumoral accumulation of cytotoxic lymphocytes, conventional type 1 dendritic cells (cDC1s) and a novel DC population expressing both CD103 and CD11b markers. These CD103^{int}CD11b⁺DCs share phenotypic and gene expression characteristics with both cDC1s and cDC2s, have transcriptomic profiles similar to monocyte-derived DCs (moDCs) and correlate with tumor regression. Therefore, hetIL-15, a cytokine directly affecting lymphocytes and inducing cytotoxic cells, has also an indirect rapid and significant effect on the recruitment of myeloid cells, initiating a cascade for tumor elimination though innate and adoptive immune mechanisms. The novel intratumoral DC population induced by hetIL-15 could be targeted for the development of effective immunotherapy approaches for the treatment of cancer.

### INTRODUCTION

Triple-negative breast cancer (TNBC) accounts for 10% to 20% of all breast cancer cases, is highly metastatic and associated with poor prognosis and survival (Dent et al., 2007; Siegel et al., 2018). Chemotherapy remains the standard of care for the TNBC. Immunotherapy has emerged as a promising treatment option for many cancer types and is rapidly being adopted in the clinic. FDA has approved chemo-immunotherapy combinations (atezolizumab or pembrolizumab) for the treatment of TNBC, showing that immunotherapy can be effective in breast cancer (Torres and Emens, 2022). The presence of tumor-infiltrating lymphocytes (TILs), especially CD8⁺ cytotoxic T cells, is widely recognized as a predictor of good prognosis in TNBC (Criscitiello et al., 2016; Loi et al., 2019). Additionally, peripheral granulocytic and monocytic expansion as well as impaired differentiation and reduction of conventional type 1 dendritic cells (cDC1s) are hallmarks of tumor progression (Casbon et al., 2015; Zhang et al., 2016). In surgical specimens from patients with TNBC tumors, the presence of CD11c⁺DCs significantly correlated with CD4⁺ and CD8⁺ T cell counts and TIL levels (Lee et al., 2018).

cDC1s, cDC2s and plasmacytoid DCs (pDCs) are defined by expression of cell surface markers and develop from well-known common DC and pre-cDC progenitors through the action of lineage-defining transcription factors (Bosteels and Scott, 2020; Guilliams et al., 2016; Murphy et al., 2016). Interferon regulatory factor 8 (IRF8) and Batf3 drive the development of chemokine receptor XCR1-expressing cDC1s, which have the capacity to present and cross-present antigens to CD8⁺T cells. On the other hand, IRF4 drives the development and terminal differentiation of the CD11b⁺CD172a⁺-expressing cDC2 lineage, which is more specialized in polarizing CD4⁺T helper (Th) cell responses (Durai and Murphy, 2016; Kumamoto et al., 2013; Williams et al., 2013). Moreover, upon development of tissue inflammation, Ly6C^{hi}CD11b⁺CD172a⁺ monocytes enter antigen-exposed barrier sites and lymph nodes (LNs). Monocytes can then rapidly upregulate the expression of Major Histocompatibility Complex class II (MHCII) and CD11c while downregulating expression of Ly6C. These cells, known as monocyte-derived DCs (moDCs) (Plantinga et al., 2013; Tamoutounour et al., 2013), perform well in ex vivo antigen presentation assays, leading to their classification as professional antigen-presenting cells (APCs) (Cheong et al., 2010; Kool et al., 2008; Wu et al., 2016).

Interleukin-15 (IL-15), a homeostatic cytokine belonging to the gamma-chain family of cytokines (Waldmann, 2006; Waldmann et al., 2020), has been shown to regulate a wide range of immune functions, including development of natural killer (NK) cells and the maintenance of memory T cells. IL-15 is also capable of enhancing the in vivo antitumor activity of adoptively transferred, tumor-reactive CD8⁺T cells and promotes infiltration and proliferation of adoptively transferred cells specifically in the tumor, in an antigen-specific way (Berard et al., 2003; Carson et al., 1994; Klebanoff et al., 2004; Ng et al., 2017). IL-15 has shown anticancer activity in many preclinical model systems (Bergamaschi et al., 2020; Mathios et al., 2016; Xu et al., 2013; Yu et al., 2012; Yu et al., 2010) and is presently being tested in multiple clinical trials for cancer immunotherapy (Conlon et al., 2021b; Conlon et al., 2015; Conlon et al., 2019; Cooley et al., 2019; Margolin et al., 2018; Miller et al., 2018; Romee et al., 2018).

We have previously shown that bioactive IL-15 in vivo comprises a complex of the IL-15 polypeptide chain with the IL-15 receptor alpha chain that are together named heterodimeric IL-15 (hetIL-15) (Bergamaschi et al., 2009; Bergamaschi et al., 2008). This heterodimer is either cell associated or in a soluble form, freely circulating in blood (Bergamaschi et al., 2012; Chertova et al., 2013). In this study, we investigated the effect of hetIL-15 monotherapy after locoregional administration in orthotopically implanted murine TNBC tumors. We identified hetIL-15-triggered interactions between tumor infiltrating lymphoid and myeloid cells and characterized a previously unrecognized, novel, population of tumor-infiltrating DCs, which is increased upon hetIL-15 administration and correlated with the anti-tumoral immune responses, the generation of anti-tumoral memory and the disease outcome, eliminating both the primary and the metastatic tumors.

### Results

hetIL-15 Locoregional Administration Eradicates EO771 Tumors

To evaluate the anti-cancer effect of hetIL-15-based immunotherapy, we used the EO771 model of TNBC. We performed orthotopic inoculation of EO771 cancer cells in the fourth mammary fat pad of C57BL/6 mice. Treatment was initiated when tumors reached ˜20 mm³. hetIL-15 was provided every 4 days locoregionally (in proximity to the tumor) at a dose of 5 μg/injection. Only 3 injections of the cytokine (FIG. 1A) completely eradicated the tumors in 33% (18 out of 54) of the hetIL-15-treated mice (FIG. 1B). Longer-term evaluation of the treatment (total of 5 hetIL-15 injections) showed 40% tumor eradication (FIG. S1A) and increased survival (FIG. 1C) as the animals did not develop tumor regrowth or signs of morbidity that could implicate metastatic disease. Further support of this anti-metastatic indication was provided by examining the lungs of the hetIL-15-treated mice. The lungs revealed significant reduction in the number of metastatic foci, as was shown by H&E histological analysis (FIG. S1B), supporting a beneficial role of hetIL-15 also in the control of metastatic burden.

EO771 tumors were analyzed by flow cytometry and immunohistochemistry (IHC) to explore the changes in the tumor immune phenotype upon hetIL-15 treatment. The shorter treatment schedule consisting of 3 hetIL-15 injections was used for these analyses and the tumors were assessed 48 h after the last injection (FIG. 1A). Flow cytometric analysis revealed significant accumulation of both CD8⁺T and NK cells (FIGS. 1D and 1E) in the hetIL-15 treated tumors. The tumor-infiltrating CD8⁺T and NK cells were characterized by higher content of the cytotoxic marker Granzyme B and increased proliferation, as evaluated by the expression of Ki67 (FIGS. 1F and 1G). Furthermore, IHC analysis verified these results, showing increased accumulation of CD8⁺T and NK cells (FIG. 1H) in the hetIL-15-treated tumors. Overall, hetIL-15 administration altered the tumor microenvironment by promoting the intratumoral infiltration of activated cytotoxic T and NK cells, as we previously reported (Bergamaschi et al., 2020; Ng et al., 2017).

To better understand the contribution of the innate and adaptive immunity in hetIL-15-anti-tumor effect, we evaluated the treatment using Rag-1 knock-out (ko) (FIGS. S1C and S1D) and NK cell-depleted C57BL/6 mice (FIGS. S1E and S1F). Six hetIL-15 injections resulted in significant tumor growth delay compared to the control group in Rag-1 ko mice, but none of them achieved complete tumor regression (FIG. S1C). In contrast, treatment with the same number of hetIL-15 injections in NK cell-depleted mice resulted in 20% complete tumor regression and significant tumor growth control in the rest of the animals (FIG. S1E). Beneficial effects of hetIL-15 treatment on metastatic burden were observed in both Rag-1 ko (FIG. S1D) and NK cell-depleted mice (FIG. S1F). Thus, both T and NK cells contributed to the anti-tumor effect of hetIL-15 on tumor growth delay and metastatic disease in the EO771 model, however, tumor eradication required the presence of T cells.

hetIL-15 Treatment Enhanced the Intratumoral Expression of Genes Associated with Lymphocyte Migration, Activation, and Cytotoxicity

To gain more detailed understanding of the function of tumor-infiltrating lymphocytes (TILs), we performed gene expression analysis of EO771 tumors excised 48 hrs after either the 1^{st}, 2^{nd} or 3^{rd} hetIL-15 administration (treatment schedule, FIG. 1A), using a panel of 780 immune-oncology related gene probes (Nanostring Technology). We identified ˜300 differentially expressed genes (log 2 fold-change>1, adjusted p<0.05) in tumors from hetIL-15-treated mice in comparison to control animals, at all three analyzed time points (FIGS. 2A-2C). Genes associated with a cytotoxic phenotype, such as Gzmb, Gzma, Prf1, Ctsw and Klrg1 (red dots), were among the most significantly overexpressed genes in hetIL-15-treated mice (FIGS. 2A-C). In addition, expression of Zap70, Cd247, Cd3d and Ifng (green dots), as well as Cxcr3, Ccl9, Ccl19 (blue dots) was also increased, highlighting the stimulation of pathways related to T cell activation/TCR signaling and leukocyte migration. GO pathway enrichment analysis of the Nanostring data showed that the T cell co-stimulation (GO:0031295), the antigen receptor mediated signaling (GO:0050851) and the positive regulation of T cell activation (GO:0050870) pathways ranked in the top 10 canonical pathways upregulated upon hetIL-15 treatment (FIG. 2D). The upregulated genes that are associated with these pathways is depicted in FIGS. 2E-G.

To analyze the systemic effects of locoregional hetIL-15 treatment, we also evaluated the gene expression pattern in draining lymph nodes (dLN), 48 hrs after the 1^{st}, 2^{nd} or 3^{rd} hetIL-15 injection. Transcriptomic analysis from the dLN (FIGS. S2A-S2C) further supported the findings that hetIL-15 enhanced T cell cytotoxicity (Gzmb, Gzma, Prf1, Ctsw and Klrg1), TCR activation (Zap70, Ifng) and chemotaxis of immune cell chemotaxis (Cxcr3, Ccr5, Cxcl9, Ccl9). GO pathway enrichment analysis revealed that leukocyte migration (GO:0050900, p=0.03; 2^{nd} injection, p=0.01; 3^{rd} injection) and T cell activation (GO:0002286, p=0.0015; 2^{nd} injection) ranked among the top upregulated canonical pathways (FIGS. S2D and S2E). Flow cytometric analysis of dLN also showed an increased frequency of CD8⁺T and NK cells (FIGS. S2F and S2G). Overall, these data demonstrate that hetIL-15 induced a cascade of transcriptional events triggering the cytotoxic capacity and activation of T and NK cells, as well as their accumulation within the tumors and dLNs.

hetIL-15 Locoregional Administration Induced the Accumulation of a Novel CD103^{int}CD11b⁺ Population of DCs in Different Breast Cancer Models

Our initial transcriptomic data analysis showed that hetIL-15 treatment also affects the myeloid cell composition of the tumors. hetIL-15 monotherapy was associated with a significant upregulation of the gene expression profile of cytotoxic cells, NK, CD8⁺T, Th1 cells, macrophages and DCs (FIG. 3A). Guided by our transcriptomic data and our recent report (Bergamaschi et al., 2020), we established a flow cytometry staining protocol (FIG. 3B) that allows distinction of different myeloid cell populations (Bottcher et al., 2018; Guilliams et al., 2016). CD103⁺cDC1s were defined as Lin(NK1.1,CD19,B220,CD3)^{neg}CD64⁻MHCII⁺CD11c⁺CD103⁺CD11b⁻; CD11b⁺cDC2s were defined as Lin(NK1.1,CD19,B220,CD3)^{neg}CD64⁻MHCII⁺CD11c⁺CD103⁻CD11b⁺ and macrophages were defined as Lin(NK1.1, CD19,B220,CD3)^{neg}CD64⁺F4/80⁺. Locoregional hetIL-15 treatment resulted in increased tumor infiltration of CD103⁺cDC1s (FIG. 3C), whereas no significant difference was found in the number of CD11b⁺cDC2s (FIG. 3D). Surprisingly, flow cytometry analysis revealed a novel DC population that was distinct from the DC subsets previously reported in tumor mouse models. This DC population, referred to as CD103^{int}CD11b⁺DC, shows a unique phenotypic expression of the CD103 and CD11b markers (FIG. 3B). This population represented a minority of MHCII⁺CD11c⁺ cells in the untreated tumors but became much more prominent upon hetIL-15 treatment (FIG. 3E). Importantly, tumor infiltration by both CD103⁻cDC1s and CD103^{int}CD11b⁺DCs inversely correlated with the EO771 tumor size in hetIL-15 treated animals 48 hrs after the 3^{rd} hetIL-15 injection (FIG. 3F). In contrast, no correlation between intratumoral CD11b⁺cDC2s and tumor size was observed (FIG. 3F).

t-Distributed Stochastic Neighbor Embedding (t-SNE) analysis from 6 control and 5 hetIL-15 treated-concatenated tumors revealed that the different DC subtypes form unique distinct clusters, i.e., CD103⁺cDC1, CD11b⁺cDC2, CD103^{int}CD11b⁺DC (FIG. 4A). Phenotypic profiling of the CD103^{int}CD11b⁺DCs in hetIL-15-treated tumors revealed that the cells express strongly and uniformly the dendritic cell marker CD24 (Guilliams et al., 2016), while they lack the expression of the macrophage markers [CD64 (Fcgr1), CD169, CX3CR1 and Ly6C] (FIG. 4B), with the exception of the F4/80 marker (FIG. 4C), suggesting they are not of macrophage lineage. CD24a is absent on macrophages but significantly expressed on DC (Guilliams et al., 2016; Schlitzer et al., 2013) and on monocytic-derived DC (moDC) (Qu et al., 2014) populations and its role has been associated with promoting the differentiation of naive CD8⁻T cells into effector- or memory-CD8⁺T cells (Kim et al., 2014). Tumor-infiltrating CD103^{int}CD11b⁺DCs were also characterized by intermediate expression of XCR1 and IRF8 (FIG. 4D). Moreover, CD103^{int}CD11b⁺DCs found positive for the TREM1 and CD101 markers, in comparison to the cDC1s and cDC2s (FIG. 4E), showing similarities with a population of CD103⁺CD11b⁺DCs found only in the intestinal lamina propria (Bain et al., 2017; Persson et al., 2013).

We confirmed our results in an additional TNBC mouse model, 4T1, which is syngeneic to Balb/c mice. After orthotopic implantation of 4T1 cells in Balb/c mice and the establishment of the tumors, the mice were treated locoregionally with 3 hetIL-15 injections (FIG. S3A). hetIL-15 treatment resulted in a significant decrease of the primary tumor volume (FIG. S3B). Flow cytometric analysis of TILs showed increased infiltration of both CD8⁺T and NK cells (FIGS. S3C and S3D). Upon hetIL-15 treatment, CD103⁺cDC1s were not affected by hetIL-15 in this model, in contrast with CD11b⁻cDC2s that were significantly increased (FIGS. S3E and S3F). Importantly, the novel CD103^{int}CD11b⁺DCs were found to be accumulated intratumorally upon hetIL-15 treatment (FIG. S3G). Verifying the previous results of the EO771 tumor model, the 4T1 tumor infiltrating CD103^{int}CD11 b⁺DCs were also characterized by the intermediate expression of CD103, IRF8 and XCR1 (FIG. S3H). These data show that hetIL-15 administration increased the number of the tumor-infiltrating CD103^{int}CD11b⁺DCs in two different mouse models, indicating that this is a general hetIL-15 induced effect, independent of the mouse strains.

CD103^{int}CD11b⁺DCs Displayed a Transcriptional Signature Similar to Monocyte-Derived DCs (moDCs)

To better characterize the properties of the different DC subsets localized in tumors, we performed RNA-sequencing (RNA-seq) on sorted tumor-infiltrating myeloid cell subsets. Principal component analysis (PCA) of the different sorted populations (CD103⁺cDC1, CD11b⁻cDC2, CD103^{int}CD11b⁺DC and macrophages) based on their transcriptome profile, revealed segregation of CD103^{int}CD11b⁺DCs. They showed a transcriptomic profile close to CD11b⁻cDC2s and mapped away from the macrophages in PCA space (FIG. S4A). Comparison with immune cell transcriptome profiles reported by Brown et al. (Brown et al., 2019) confirmed that infiltrating CD103^{int}CD11b⁺DCs showed low expression of the key macrophage genes Fcgr1, Siglec1, Ly6c2, Cx3cr1, Ly6c1, whereas DC-expressed markers CD24a, Xcr1, Itgae, Itgam, Itgax, Sirpa, Irf4, Cd207 and CD209a (FIG. S4B) were highly or intermediately expressed in tumor-infiltrating CD103^{int}CD11b⁺DCs. This cell population has also increased Rbpj and Batf3 gene expression but showed low expression of Flt3 and CD8a (FIG. S4B). Furthermore, a heatmap of the antigen presentation pathway, using reference genes from Kaczanowska et al. (Kaczanowska et al., 2021), revealed that many genes implicated in antigen processing and presentation (Wdfy4, Ciita, Naaa, Batf3, H2-DMa, H2-Aa, Cd74, H2-Ab1, and H2-Eb1) (Kaczanowska et al., 2021; Santambrogio et al., 2019; Theisen et al., 2018) were upregulated in CD103^{int}CD11b⁺DCs compared to other DC subsets or macrophages (FIG. S4C). The high expression of genes involved in the antigen presenting process as well as the correlation between the abundance of CD103^{int}CD11b⁺DCs in hetIL-15-treated tumors and tumor growth control (FIG. 3F) led to investigation of whether these cells contributed to the activation of CD8⁺T cells within the tumor. Ex vivo co-culture of isolated splenic CD8⁺T cells from naïve mice with sorted CD103^{int}CD11b⁺DCs from hetIL-15-treated tumors led to induction of IFN-γ production in CD8⁺ T cells from naïve mice (FIG. S4D). Overall, these results showed that tumor-infiltrating CD103^{int}CD11b⁺DCs have a unique transcriptome profile, which differs from macrophages. Their signature includes genes encoding DC markers and contributing to DC functions, including genes involved in antigen presentation.

To further characterize the tumor infiltrating CD103^{int}CD11b⁺DCs, we performed single-cell RNA-sequencing (scRNA-seq) on sorted CD11c⁺ cells obtained from tumors of hetIL-15 treated or control EO771-tumor bearing mice. A total of 10,195 single-cell transcriptomes were generated after pre-processing. Unsupervised clustering was performed using Seurat v3.1.5 and Louvain method (Blondel D. Vincent, 2008). Clusters were serially annotated with SingleR using reference data generated from Brown et al. (Brown et al., 2019) and RNA-seq matrices from our own sorted DC populations. After removal of cell-cycle signals, scRNA-seq of the CD11c⁺CD64^{neg} cells identified 7 distinct clusters visualized using UMAP (FIG. 5A). We established the cell identity of each cluster through the analysis of canonical DC gene expression similarity with reference genes from Brown et al. (Brown et al., 2019). The CD103^{int}CD11b⁺DC population in hetIL-15 treated tumors was enriched in the sample density UMAP plot (FIG. 5B, yellow). Cellular indexing of transcriptome and epitopes sequencing (CITE-seq) confirmed the high gene and protein expression of CD24 in CD103⁺cDC1s and CD103^{int}CD11b⁺DCs (FIG. S5A, yellow). In addition, the CD103^{int}CD11b⁻DC population expressed a unique gene signature. Shared gene expression among individual clusters revealed that CD103^{int}CD11b⁺DCs possess a gene profile with similarities to monocytes (monocyte 1 cluster, FIG. 5C), with several highly expressed (mo)DC/DC markers [Mgl2, Ccl17, Plet1, Clec4n (Dectin2), CD24a, mmp12, clec4b1 (DCAR), and Anxa1 (Annexin1)] (Bonnardel et al., 2015; Kis-Toth et al., 2013; Napoletano et al., 2007; Qu et al., 2014; Toyonaga et al., 2016; Tzelepis et al., 2015), suggesting a possible monocytic origin for this DC subset. CD103^{int}CD11b⁺DCs expressed the highest levels of Mgl2 and Ccl17 among the different DC subtypes. Plet1, a specific marker of cDC2 in the gastrointestinal tract, and Mmp12, which is expressed in both resting and activated human moDCs (Kis-Toth et al., 2013), were also highly expressed in the CD103^{int}CD11b⁺DC cluster. In addition, CD103^{int}CD11b⁺DCs were characterized by high levels of Clec4b1, a protein that is selectively expressed in mouse CD11b⁺CD11^{int}MHCII⁺ monocyte-derived cells (Toyonaga et al., 2016), and Lpl, like in the human moDCs (Le Naour et al., 2001). The increased expression of genes related to antigen-processing machinery of DCs such as Naaa, Wdfy4 and Annexin1 (Santambrogio et al., 2019; Theisen et al., 2018; Tzelepis et al., 2015) was also verified in the CD103^{int}CD11b⁺DCs. Bubble plot of canonical DC and selected macrophage/monocyte markers (Brown et al., 2019) verified the absence of macrophage markers (Cx3cr1, Ly6c1, Siglec1), the decreased expression of Flt3, which is absent from the moDCs (Karsunky et al., 2003), and the increased expression of (mo)DC/DC markers (CD24a, Itgam, Itgax, Sirpa and Lamp2) in CD103^{int}CD11b⁺DCs (FIG. S5B). Interestingly, single-sample gene set enrichment analysis (ssGSEA) revealed enrichment of pathways involved in DC migration and maturation, as well as myeloid DC differentiation and activation (Ashburner et al., 2000; Gene Ontology, 2021; Le Naour et al., 2001; Lindstedt et al., 2002; Shaffer et al., 2008) in the tumor-infiltrating CD103^{int}CD11b⁺DC cluster after hetIL-15 treatment (FIG. 5D).

Our scRNAseq results demonstrated that CD103^{int}CD11b⁺DCs have the highest expression levels of Mgl2 and Ccl17 among the different DC subtypes and monocytes and also express CD24a. We reason that co-expression of these highly express markers will identify the group of CD103^{int}CD11b⁺DCs. We therefore performed in situ RNA hybridization (RNAscope), using probes that target these 3 markers, in paraffin-embedded tumor tissues to further identify the presence and localization of this unique DC population. RNAscope analysis confirmed the presence of CD103^{int}CD11b⁺DCs in the tumors of hetIL-15 treated mice, whereas these cells could not be detected in the tumors from control mice (FIG. 6), confirming our flow cytometric results (FIG. 3). Furthermore, transcriptomic cytokine profiling of cDCs and CD103^{int}CD11b⁺DCs revealed that CD103^{int}CD11b⁺DCs expressed higher levels of inflammatory chemokines Ccl6, Ccl9, Cxcl2, Ccl17, Ccl2, Ccl4, Ccl22 and Ccl24 in comparison to other DC subtypes (FIG. S5C). Overall, our RNA-seq data demonstrated that CD103^{int}CD11b⁺DCs formed a distinct cluster with a transcriptional profile with similarities to moDCs and may have a functional, intratumoral role due to the expression of genes associated with antigen presentation.

hetIL-15 Locoregional Administration Resulted in a Long-Lasting Specific Anti-Tumor Immunity

We also examined the development of anti-tumor memory T cells upon locoregional hetIL-15 administration. Mice that had previously eradicated EO771 tumors were re-challenged with the same tumor cell line 68 and 158 days after the last hetIL-15 dose (FIG. 7A). Age-matched control mice developed EO771 tumors as expected, whereas tumors failed to be established in mice with a previous history of tumor eradication after hetIL-15 therapy, suggesting development of protective anti-tumor immunity (FIG. 7B). To verify the specificity of the anti-tumor immune response, mice were also challenged using the syngeneic pancreatic KPC tumor cells (challenge #2, FIG. 7A). KPC tumors developed at the same rate in both groups (FIG. 7C), supporting the conclusion that hetIL-15 treated mice were able to develop and maintain specific immunity against EO771 tumor.

Next, we performed adoptive cell transfer of purified CD8⁺T cells from hetIL-15 treated mice that had previously eradicated EO771 tumors and successfully rejected EO771 tumors upon subsequent re-challenge (FIG. 7D). Recipients were treated with hetIL-15 every 2 days to support the adoptively transferred CD8⁺T cells. Transfer of the CD8⁺T cells into lymphodepleted EO771 tumor-bearing mice reduced tumor growth (FIG. 7E, left panel) and increased the survival of the recipient mice (FIG. 7E, right panel) compared to mice receiving CD8⁺T cells from mice never exposed to EO771 tumor cells. We also monitored the development of lung metastasis in mice that underwent adoptive cell transfer of CD8⁺T cells. The number of lung tumor foci in mice that received CD8⁺T cells from donors previously cured from EO771 was significantly reduced (FIG. S6), suggesting that hetIL-15-induced memory CD8⁺T cells could reduce or control metastatic disease in the lungs. Overall, our findings indicated that monotherapy using locoregional hetIL-15 administration caused the development of specific long-lasting anti-tumor immunity, which resulted in complete tumor eradication and protection from subsequent exposure.

### DISCUSSION

The present study provides evidence that hetIL-15 administration, in proximity to the tumor, is a therapeutic approach with strong activity against TNBC that exerts both local and systemic effects. These effects include cures of the tumor-bearing mice (˜40%), prolonged survival and induction of immunological memory against breast cancer cells. Reduction or complete elimination of metastatic disease was also observed. Another important conclusion of this work is that hetIL-15 re-shaped the tumor microenvironment by promoting the intratumoral accumulation of cytotoxic lymphocytes, cDC1s and a novel DC population, defined as CD103^{int}CD11b⁺DC. This distinct DC population has phenotypic and transcriptional similarities with cDCs and monocyte-derived DCs (moDCs) and correlates with tumor regression.

There is an increased interest on exploring local delivery of immune modulators for the treatment of solid tumors. Several studies reviewed by Marabelle et al. (Marabelle et al., 2014) have shown that intratumoral administration of immune-stimulating drugs allows for higher concentrations in the tumor microenvironment than systemic deliveries, resulting in improved therapeutic effects and lower toxicities. As a result, the number of trials investigating local administration of cancer therapies has experienced rapid growth (Champiat et al., 2021). Different forms of IL-15 have been investigated as cancer immunotherapeutics in several mouse cancer models (Bergamaschi et al., 2020; Emma Kurz, 2022; Mathios et al., 2016; Xu et al., 2013; Yu et al., 2012; Yu et al., 2010) and they are currently tested in several clinical trials (Conlon et al., 2021b; Conlon et al., 2015; Conlon et al., 2019; Cooley et al., 2019; Margolin et al., 2018; Miller et al., 2018; Romee et al., 2018). The first-in-human trial with hetIL-15 delivered systemically by subcutaneous injection in patients with metastatic or unresectable cancer, showed disease stabilization in 3 of the 14 participants, as the best observed clinical response (Conlon et al., 2021a), but have not yet formally been evaluated in human breast cancer. In the present study, we model locoregional administration of hetIL-15 in an orthotopic mouse breast cancer model. We showed that locoregional administration increase therapeutic effects by resulting in complete tumor regression and elimination or decrease in metastasis. hetIL-15 was given using Matrigel as vehicle, which might contribute to the efficacy by retaining the cytokine in the tumor area. Additionally, hetIL-15 monotherapy stimulated CD8⁺T and NK cells trafficking into the tumors and promoted their proliferation and cytotoxicity. These data agree with preclinical and clinical studies where the antitumor responses induced by IL-15 were linked to expansion and activation of NK and CD8⁺T cells (Bergamaschi et al., 2020; Berger et al., 2009; Conlon et al., 2015; Waldmann et al., 2011). Importantly, our depletion experiments suggested that the effects of hetIL-15 treatment in controlling tumor growth and metastasis in a TNBC model were mediated through both T and NK cells. Significant tumor growth delay was observed after locoregional injection of hetIL-15 in Rag-1 ko mice and NK cell-depleted C57BL/6 mice. However, complete tumor regression was not achieved in Rag-1 ko mice, suggesting that adaptive immunity is required for the curative effect of hetIL-15. We also demonstrated that EO771-tumor elimination in mice after hetIL-15 monotherapy provided T cell-dependent protection from subsequent rechallenge with EO771 tumor. These data support the conclusion that hetIL-15 elicits long-term T cell memory against tumor cells. The preserved T cell responses were specific for the EO711 cells because those animals failed to control challenge with an unrelated syngeneic pancreatic tumor line.

Effects of IL-15 on DC phenotypic characteristics and functions have been previously reported (Mattei et al., 2001; Tourkova et al., 2005; Tourkova et al., 2002). Here, we show that locoregional administration of hetIL-15 increases tumor-infiltrating CD103⁺cDC1s in EO771 orthotopic breast cancer model and this accumulation is inversely correlated with tumor size. These results agree with our previous report where systemic hetIL-15 delivery increased the intratumoral CD103⁺cDC1s of MC-38 and TC-1 tumors (Bergamaschi et al., 2020). Importantly, we identified for the first time a discrete CD103^{int}CD11b⁺DC subset, which greatly increased upon hetIL-15 treatment while it is present in very low numbers in the control group. This population, like CD103⁺cDC1s, also inversely correlated with EO771 tumor size. Moreover, the CD103^{int}CD11b⁺DCs were also identified in the 4T1 mouse model of TNBC and found elevated upon hetIL-15 treatment, suggesting a general effect of hetIL-15 on expanding this novel DC population in TNBC models.

Combination of scRNA-seq, bulk RNA-seq and flow cytometric analysis showed that CD103^{int}CD11b⁺DC population form a unique cell cluster, which expresses canonical DC markers and is different from macrophages. They lack many key macrophage markers [Fcgr1 (CD64), CD169, Cx3cr1 (CX3CR1), Ly6c1 (L6c1) and Siglec1] but they express F4/80. F4/80, a common macrophage marker, has however found to be expressed by another DC subset, the monocyte-derived migratory antigen-presenting cells, F4/80^{high}APCs (Sheng et al., 2017). Our CD103^{int}CD11b⁺DCs have similarities to the F4/80^{high}APCs, but they do not express CD64 and CD169. Further characterization of CD103^{int}CD11b⁺DCs showed that they have intermediate expression of XCR1 and IRF8. These markers distinguish CD103^{int}CD11b⁺DCs from both the cDC1 and cDC2 populations. XCR1 expression is strongly associated with the ability of cDC1s to interact with CD8⁺T cells (Calabro et al., 2016), whereas IRF8 strongly correlated with their cross-presenting phenotype (Sichien et al., 2016). In addition, CD103^{int}CD11b⁺DCs have increased Rbpj and Batf3 gene expression. It has been shown that the transcription factor RBP-J-mediated signaling is essential for DCs to evoke efficient anti-tumor immune responses in mice (Feng et al., 2010) whereas Batf3-lineage CD103⁺DCs are necessary for recruitment of effector CD8⁺T cells within the tumor (Spranger et al., 2017). The tumor-infiltrating CD103^{int}CD11b⁺DCs have a distinct expression pattern of CD64, CD24, F4/80, CD103 and XCR1 genes that distinguishes them from the most DC populations that have been previously reported in the literature, including the inflammatory cDC2s (Bosteels et al., 2020), the tumor moDC3s (Diao et al., 2018), the PDAC-associated CD11c⁻DCs (Kenkel et al., 2017) or the mouse dermal moDCs (Tamoutounour et al., 2013). Nevertheless, this distinct expression pattern of the tumor-infiltrating CD103^{int}CD11b⁺DCs shows similarities with the intestinal CD103⁻CD11b⁺DCs and some types of moDCs. CD103⁺CD11b⁺DCs in the intestinal lamina propria express high levels of Gp2, Cd101 and Trem1 (Bain et al., 2017; Persson et al., 2013). We examined whether these markers were expressed in the hetIL-15-induced CD103^{int}CD11b⁺DCs. Although we could not detect any surface expression of GP2, the expression of TREM1 and CD101 was higher in comparison to cDC1 and cDC2 subtypes. Furthermore, the CD103^{int}CD11b⁺DCs express low levels of the chemokine receptor CX3CR1, that was also observed in intestinal CD103⁺CD11b⁻DCs (Persson et al., 2013), supporting the notion that these cells are not tissue resident macrophages.

Single cell transcriptomic analysis also showed that CD103^{int}CD11b⁺DCs induced by hetIL-15 share similarities in transcribed genes with the moDCs and more specifically with the CD64⁻MHC⁺CD11c⁺Ly6C^{lo}CX3CR1^{int}moDCs, that display migratory and antigen-presenting features (Bain et al., 2013; Tamoutounour et al., 2012; Zigmond et al., 2012), and with the Ly6C^{lo}CD209a⁺moDCs, which are powerful migratory antigen-capturing and -presenting cells (Cheong et al., 2010). Several reports have provided evidence that the immune system uses monocytes as DC precursors for efficient antigenic presentation in the periphery during inflammation (Le Borgne et al., 2006; Nakano et al., 2009; Wakim et al., 2008) and suggesting that moDCs are important players in the development of an adaptive immune response (Qu et al., 2014). Many genes associated with antigen processing and presentation were highly expressed in hetIL-15-induced tumor-infiltrating CD103^{int}CD11b⁺DC, suggesting the antigen-presenting properties of those cells. Their possible involvement in intratumoral antigen presentation relevant for tumor control needs to be further investigated. Importantly, transcriptomic cytokine profiling revealed high expression of Cxcl2, Ccl17 and Ccl22 suggesting that CD103^{int}CD11b⁺DCs may be activated or mature (mo)DCs/cDCs (Alferink et al., 2003; Kobayashi et al., 2013; Lee et al., 2009) and are involved in the recruitment of activated and memory T cells, as well as B lymphocytes (Alferink et al., 2003; Eberlein et al., 2010; Henry et al., 2008; McColl, 2002; Plantinga et al., 2013; Semmling et al., 2010). The GSEA of our CD103^{int}CD11b⁺DCs scRNA-seq data strengthen this hypothesis since it revealed the upregulation of many cellular processes involved in the maturation and activation of DCs. Furthermore, the high expression of Mgl2 in CD103^{int}CD11b⁺DCs raises the question whether Mgl2⁺dermal DC, previously reported in the skin and the draining popliteal LN (Kumamoto et al., 2013), are akin to CD103^{int}CD11b⁺DCs. Mgl2⁻DC in the lamina propria have been identified as the APCs responsible for driving tissue-resident memory CD8⁺T cells-mediated protection after HSV-2 infection (Shin et al., 2016). Moreover, CLEC10A, the human homolog to the Mg12, is a key marker for the CD1c⁺DCs (Heger et al., 2018). When properly activated, human CD1c (BDCA-1)⁺myeloid derived DCs (myDCs) secrete high levels of interleukin-12 (IL-12) and potently prime CTL responses (Nizzoli et al., 2013). Therapeutic potential of cellular vaccines that contain antigen-loaded CD1c (BDCA-1)⁺myDCs has already been under investigation in early clinical trials in patients with metastatic melanoma or prostate cancer indicating objective tumor responses and immunogenicity (Schreibelt et al., 2016; Tel et al., 2013; Westdorp et al., 2019). These properties of the human CD1c⁺DCs further suggest possible involvement of CD103^{int}CD11b⁺DCs in antigen presentation and immune response and underscore a role of hetIL-15 in these processes.

In conclusion, locoregional therapy with hetIL-15 is an effective therapy that holds promise as a future therapeutic option for TNBC. hetIL-15 coordinates an effective local and systemic immune response against the EO771 and 4T1 tumors, promoting tumor growth control by CD8⁺T and NK cells and increasing tumor infiltration of cDC1 and of a unique CD103^{int}CD11b⁺DC subpopulation most closely related to moDC. These cells may have a complementary role with the cDC1s in the anti-tumoral immune response. This report demonstrate that hetIL-15 administration enhanced the intratumoral interaction between DC and lymphocytes, which leads to the generation of a long-lasting specific and protective anti-tumoral immune response. These properties, if reproduced in humans, might lead to additional therapeutic options for breast cancer patients.

### Experimental Procedure

### Mouse Models

All studies were approved by the National Cancer Institute-Frederick Animal Care and Use Committee. NCI-Frederick is accredited by AAALAC International and follows the Public Health Service Policy for the Care and Use of Laboratory Animals. Animal care was provided in accordance with the procedures outlined in the “Guide for Care and Use of Laboratory Animals (National Research Council; 1996; National Academy Press; Washington, D.C.). C57BL/6 and BALB/c or Rag-1 ko (B6.129S7-Rag1tm1Mom/J) female mice were purchased from Envigo International Holdings, Inc., or Jackson Laboratory, respectively. Mice at 6-8 weeks of age were randomly assigned to treatment or control groups. For the orthotopic mouse EO771 or 4T1 breast model, cells were purchased from CH3 BioSystems or ATCC, respectively. Cell lines were cultured in complete RPMI 1640 medium supplemented with 10% fetal calf serum, 50 mM 2-mercaptoethanol, 100 U/ml Penicillin and 100 mg/ml Streptomycin. Murine EO771 or 4T1 cells (3×10⁵) were orthotopically inoculated at the fourth mammary fat pad of 6-8 weeks old mice. The cells were resuspended in PBS. Matrigel (Corning Inc.) was added at 1:3 dilution to facilitate the inoculation process. Matrigel, an extract of basement membrane proteins, was used as cell carrier medium for the cell transplantation studies forming a 3D gel at 37° C. facilitating the inoculation (Kleinman and Martin, 2005). Tumor size was measured using a digital caliper and tumor volume (mm³) was calculated by the following equation: L*W*H*π/6.

### Immunotherapy of EO771 or 4T1 Tumor-Bearing Mice

Treatment was initiated when tumors reached ˜20 mm³. Animals were treated with hetIL-15 (Bergamaschi et al., 2008; Chertova et al., 2013), which is a heterodimer comprising the IL-15 chain and soluble extracellular portion of IL-15 Receptor alpha chain. In some experiments, the hetIL-15Fc molecule was used, which is a fusion of hetIL-15 to the Fc fragment of human immunoglobulin G1 (IgG1), with similar results. hetIL-15 was administered in Matrigel (Corning Inc.), used in 1:4 dilution, every 4 days peritumorally at 5 μg/mouse in PBS. In the survival studies, mice were sacrificed when the primary tumor reached a 2 cm diameter or any other humane endpoints listed in the ACUC-approved animal protocol, such as 20% weight loss or acute morbidity.

### NK Cell Depletion In Vivo

EO771 tumor-bearing C57BL/6 mice were treated locoregionally with vehicle (control) or hetIL-15. For NK cell depletion, mice received 100 g of anti-NK1.1 mAb (clone PK13) or control IgG2a (BioXCell) delivered by intraperitoneal injection. Anti-NK1.1 or isotype was administered through intraperitoneal (i.p.) injection for four consecutive days before the inoculation of murine EO771 cells. Thereafter, anti-NK1.1 mAb or IgG2a were injected every 4 days for the remainder of the experiment. Depletion of NK cells were confirmed through flow cytometry analysis of spleen and was consistently >95%.

### Adoptive Cell Transfer

Recipient naïve mice were challenged with 3×10⁵ EO771 cells on day 0 and seven days later the mice were irradiated with 600 cGy (whole body irradiation; X-ray source, 1.29 Gy/minute, 137-cesium chloride irradiator). Eight days after tumor challenge, CD8⁺T cells from spleen of naïve or hetIL-15 treated mice rechallenged with EO771 tumor cells were injected into the EO771 tumor-bearing mice. Recipient mice were boosted with hetIL-15 i.p. injections (5 μg/dose/mouse) every 3 days until the end point.

### Rechallenge Experiments

C57BL/6 mice were inoculated with 3×10⁵ EO771 cells. When palpable tumor had formed 7 days later, mice were treated with hetIL-15 injections, as described in the figure legends. On day 68, long-term surviving tumor-free mice were rechallenged with 5×10⁴ EO771 cells. The mice remained tumor-free after the first rechallenge and on day 158, the mice were rechallenged again for a second time with 5×10⁴ EO771 cells (4^{th} right mammary pad) and by injection of 5×10⁴ KPC cells (3^{rd} left mammary pad). Growth of individual EO771 and KPC tumors were monitored from the day of the 2^{nd} rechallenge until the end point.

### Histology and Immunohistochemistry Staining

Tissue samples, including tumors, were fixed in 10% neutral buffered formalin (NBF, Sigma) then routinely processed and paraffin embedded. Tumor and lung sections were dewaxed and rehydrated and then were stained with hematoxylin and eosin (H&E). For immunohistochemistry, sections were antigen-retrieved with heat-induced or enzymatic method. Peroxidase activity was blocked using 1.5% hydrogen peroxide. Sections were blocked with different blocking protocols, depending on the antibody. Staining was performed using the following anti-mouse antibodies: anti-CD8a (clone 53-6.7; eBioscience) and NK1.1 (clone 30-F11, BD Biosciences). Polymer-based detection kit, which consists of horseradish peroxidase-conjugated polymers was used for the detection.

### Splenic CD8a⁺T Cells Isolation

Single-cell suspension of murine splenocytes were collected through a 100-mm cell strainer. The CD8a⁺T cells isolation Kit (Miltenyi Biotec Inc.) was used for the isolation, according to the manufacturer protocol. Cells were isolated through negative selection using AutoMACS® Pro Separator (Miltenyi Biotec Inc.).

### Tumor-Infiltrating CD11c⁺ Cell Isolation

EO771 tumors from control and hetIL-15 treated animals were enzymatically digested using the tumor dissociation kit (Miltenyi Biotec Inc.) and mechanically dissociated using the GentleMACS™ Dissociator (Miltenyi Biotec Inc.). Tissues were passed through 100-mm cell strainers (Falcon) and washed with PBS before proceeding to the isolation step. The CD11c⁺ cells isolation Kit (Miltenyi Biotec Inc.) was used, according to the manufacturer protocol. Cells were isolated through positive selection using AutoMACS® Pro Separator (Miltenyi Biotec Inc.).

### Flow Cytometry

At necropsy tumors and dLNs were processed for flow cytometric analysis. All tumors were weighed before the start of the process. To generate single cell suspensions, tumors were enzymatically digested using the tumor dissociation kit (Miltenyi Biotec Inc.) and mechanically dissociated using the gentleMACS™ Dissociator (Miltenyi Biotec Inc.). Tissues were passed through a 100 mm cell strainer (Falcon) and washed with PBS before proceeding with antibody mediated staining. LN were dissociated using a 100 mm cell strainer and washed with PBS. Surface staining was performed using the following anti-mouse antibodies: CD45 (clone 30-F11), CD3 (clone 145-2C11), CD8a (clone 53-6.6), CD19 (clone 1D3), NK1.1 (clone PK136), B220 (clone RA3-6B2), XCR1 (clone ZET), MHCII (clone M5/114.15.2), CD11c (clone N418), CD64 (clone X55-5/7.1), F4/80 (clone BM8), CD103 (clone M290), CD11b (clone M1/70), CD172a (clone P84), Ly6C (clone HK1.4), TREM-1 (clone TR3MBL1), CD101 (clone Moushi101), CX3CR1 (clone SA011F11) and GP2 (clone 2F11-C3). For intracellular staining, cells were fixed and permeabilized using the Foxp3 staining buffer. Samples were stained with Ki67 (clone B56), Granzyme B (clone GB12), IRF8 (clone V3GYWCH) and INF-γ (clone XMG1.2). The samples were acquired on a Fortessa (BD Biosciences) flow cytometer, and the data were analyzed using the FlowJo software (Tree Star).

Gene Expression Analysis by nCounter PanCancer Immune Profiling Panel

Tumors were mechanically disrupted in RLT buffer (QIAGEN) and RNA extraction was performed with RNeasy (QIAGEN) including on-column DNase I digestion, according to the manufacturer's instructions. nCounter PanCancer Immune Profiling Panel (NanoString Technologies) was used to monitor the expression of a panel of 770 genes related to immuno-oncology. The mRNA molecules were counted with the NanoString nCounter at the Laboratory of Molecular Technology Advanced Technology Program, Frederick National Laboratory). Analysis was performed with a workflow written in R and through a user interface developed on the Foundry Platform (Palantir Technologies). Filtering was performed on raw reads to genes with low counts leaving 769 from the array. Log-transformed counts were quantile normalized and tested for differential expression with limma-voom (Ritchie et al., 2015). Pathway enrichment analysis was performed with the Fisher's Exact Test using the GO database and the top 150 positively and negatively differentially expressed genes as defined by t-statistic (https://github.com/CCBR/l2p). Pathways were correlated with survival in breast cancer patients by performing survival analyses on TCGA BRCA datasets. Transcriptome and clinical data were accessed using RTCGA and RTCGA clinical (https://rtcga.github.io/RTCGA). Immune cell populations were scored by taking the geometric mean expression of reference marker genes within each sample, with makers for cytotoxic and dendritic cells taken from Dahaher et al (Danaher et al., 2017).

### Bulk RNA Sequencing

Tumor-infiltrating DC subpopulations (CD103⁺cDC1s, CD11b⁺cDC2s and CD103^{int}CD11b⁺DCs) and macrophages were sorted on a BD FACSAria II. For each cell subset, (4,000-20,000) viable cells were sorted directly into RTL buffer, flash frozen and stored at −80° C. until RNA extraction. RNA was isolated using RNeasy Mini Kit (Qiagen) and removal of genomic DNA (gDNA) was performed with the DNase I enzyme (Qiagen), according to manufacturer's recommendations. Library preparation was performed using NEBNext® Ultra™ II Directional RNA Library Prep Kit. At least 100 million reads per sample were used following the standard operating procedure at the Sequencing Facility—Illumina (CCR). Preprocessing, alignment, and gene-wise quantification steps were performed using the CCBR Pipeliner (https://github.com/CCBR/Pipeliner) as implemented by NIH HPC Biowulf cluster (http://hpc.nih.gov). Downstream analysis and visualization were performed in R on the NIH Integrated Data Analysis Platform.

### Single-Cell RNA Sequencing

Isolated tumor-infiltrating CD11c⁺ populations from control and hetIL15-treated EO771-tumor bearing mice were processed into single-cell suspension. Approximately 10,000 cells from every sample were then loaded on one channel of the 10× chip and GEMs (Gel Beads-in-emulsion) were generated using the 10× Genomics Chromium Controller. 3′ mRNA-seq gene expression libraries were then prepared using the Chromium Next GEM Single Cell 3′ Reagent Kits v3.1 These libraries were pooled and first run on NextSeq500 as asymmetric paired-end run with a read length of 28 bp for Read 1.55 bp for Read 2, and 8 bp for the sample index read. The data from this run was used to calculate the re-pooling ratios for better balancing of the libraries, and the new pool of the six gene expression libraries was sequenced on a NovaSeq SP (100 cycle) run as asymmetric paired-end run with a read length of 28 bp for Read 1, 75 bp for Read 2, and 8 bp for the sample index read. The data from the two sequencing runs for gene expression libraries was pooled for the final analysis. Cellranger v4.0.0 count matrices were analyzed on the NIH Integrated Data Analysis Platform. Quality control, merging, and clustering was performed using Seurat v3.1.5. Cells were serially annotated with scRNA-seq reference datasets from Brown et al and our own bulk RNA-seq dataset from FACS-purified populations with SingleR v1.0.0. Single-sample GSEA analysis was performed on cluster average gene expression using the GSVA v1.30.0 R package against dendritic cell pathways extracted from all collections in MSigDB (v6.2). Normalized enrichment scores were row scaled and plotted with heatmap v1.0.12. MSigDB dendritic cell pathways and Seurat clusters were clustered within the heatmap using Euclidean distances.

### Multiplex RNA In Situ Hybridization Staining

CD24a, Mgl2, and Ccl17 expression was detected by staining 5 μm FFPE tissue sections with RNAscope® 2.5 LS Probe-Mm-CD24a-C1 (ACD, Cat #432698), RNAscope® 2.5 LS Probe-Mm-Mgl2-O1 (ACD, Cat #822908-C2), RNAscope® 2.5 LS Probe-Mm-Ccl17-C3 (ACD, Cat #428498-C3), and the RNAscope LS Multiplex Fluorescent Assay (ACD, Cat #322800) using the Bond RX auto-stainer (Leica Biosystems) with a tissue pretreatment of 15 minutes at 95° C. with Bond Epitope Retrieval Solution 2 (Leica Biosystems), 15 minutes of Protease III (ACD) at 40° C., and 1:750 dilution of TSA-Cyanine 5 Plus, TSA-Fluorescein Plus and TSA-Cyanine 3 Plus (AKOYA), respectively. The RNAscope® 3-plex LS Multiplex Negative Control Probe (Bacillus subtilis dihydrodipicolinate reductase (dapB) gene in channels C1, C2, and C3, Cat #320878) was used as a negative control. The RNAscope® LS 2.5 3-plex Positive Control Probe-Hs was used as a technical control to ensure the RNA quality of tissue sections was suitable for staining. Slides were digitally imaged using an Aperio ScanScope FL Scanner (Leica Biosystems).

Direct Co-Culture of DC with CD8⁺T Cells

Sorted tumor-infiltrating CD103^{int}CD11b⁺DCs were co-cultured with isolated splenic CD8⁺T cells from naïve mice (ratio DC:CD8⁺T cells, 1:10) in RPMI1640 supplemented with 10% FBS, 1% penicillin—streptomycin, GM-CSF (100 U/mL) and IL-2 (30 IU/mL). After 24 hrs incubation, the cells were harvested, washed, and analyzed by flow cytometry to determine IFN-γ expression.

### Statistical Analysis

Statistical analyses and graph generation were performed with GraphPad Prism 9.2.0 (San Diego, CA, USA). Tumor areas were plotted as mean±standard error of the mean (SEM) for each data point, and tumor growth curves were compared using mixed effects ANOVA. Log-rank tests were applied for Kaplan-Meier survival curves. Differences were evaluated by 1-way ANOVA or unpaired parametric Student's t test. The p-values were calculated for multiple comparisons using Tukey's multiple comparisons test or using the MAST algorithm (Finak et al., 2015). Pearson correlation was used to test the relationship between cell count and tumor volume.

### List of Supplemental Information

FIGS. S1-S6

### Acknowlegements

We thank A. Valentin for support and discussions; K. Klarmann, CCR-Frederick Flow Cytometry Core Laboratory; T. Bao, J. Sheety, and M. Mehta, Sequencing Facility; Andrew Warner, Molecular Histopathology Laboratory; X. Wu and N. Bubunenko, Genomics Laboratory, Frederick National Laboratory for Cancer Research for technical assistance; members of Pavlakis and Felber labs for discussions and support; and T. Jones for editorial assistance.

### Funding

This work was funded by the Intramural Research Program of the National Cancer Institute, National Institutes of Health (NCI/NIH) (BKF, GNP).

This project has been funded in part with Federal funds from the National Cancer Institute, National Institutes of Health, under Contract No. HHSN261201500003I. The content of this publication does not necessarily reflect the views or policies of the Department of Health and Human Services, nor does mention of trade names, commercial products, or organizations imply endorsement by the U.S. Government. This contract number represents work performed within the scope of work of the non-severable IDIQ contract (SK, BAN).

### Author Contributions

Conceptualization: D.S., S.K., G.N.P.; Performed experiments: D.S., S.K., V.S., B.A.N.; Analyzed data: D.S., S.K., V.S.; Analyzed transcriptomic data: M.A.; Investigation: D.S., S.K., V.S.; Visualization: D.S., S.K., G.N.P.; Funding acquisition: B.K.F., G.N.P.; Supervision: G.N.P.; Writing—original draft: D.S., S.K.; Writing—review & editing: D.S., S.K., V.S., C.B., B.K.F, G.N.P.; Review final manuscript: D.S., S.K., V.S., M.A., B.A.N., C.B., B.K.F., G.N.P.

### Declaration of Interests

Each author contests that they have no competing interests, except as disclosed C.B., B.K.F., and G.N.P. are inventors on US Government-owned patents related to hetIL-15.

### Data and Material Availability

All data associated with this study are present in the paper or the Supplementary Materials. Sequence data has been deposited in the Gene Expression Omnibus under the accession GSE180695. Analysis code is available at https://github.com/NCI-VB/TNBC_hetIL-15.

### REFERENCES

Alferink, J., Lieberam, I., Reindl, W., Behrens, A., Weiss, S., Huser, N., Gerauer, K., Ross, R., Reske-Kunz, A. B., Ahmad-Nejad, P., et al. (2003). Compartmentalized production of CCL17 in vivo: strong inducibility in peripheral dendritic cells contrasts selective absence from the spleen. J Exp Med 197, 585-599. 10.1084/jem.20021859.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat Genet 25, 25-29. 10.1038/75556.
Bain, C. C., Montgomery, J., Scott, C. L., Kel, J. M., Girard-Madoux, M. J. H., Martens, L., Zangerle-Murray, T. F. P., Ober-Blobaum, J., Lindenbergh-Kortleve, D., Samsom, J. N., et al. (2017). TGFbetaR signalling controls CD103(+)CD11b(+) dendritic cell development in the intestine. Nat Commun 8, 620. 10.1038/s41467-017-00658-6.
Bain, C. C., Scott, C. L., Uronen-Hansson, H., Gudjonsson, S., Jansson, O., Grip, O., Guilliams, M., Malissen, B., Agace, W. W., and Mowat, A. M. (2013). Resident and pro-inflammatory macrophages in the colon represent alternative context-dependent fates of the same Ly6Chi monocyte precursors. Mucosal Immunol 6, 498-510. 10.1038/mi.2012.89.
Berard, M., Brandt, K., Bulfone-Paus, S., and Tough, D. F. (2003). IL-15 promotes the survival of naive and memory phenotype CD8+ T cells. J Immunol 170, 5018-5026. 10.4049/jimmunol.170.10.5018.
Bergamaschi, C., Bear, J., Rosati, M., Beach, R. K., Alicea, C., Sowder, R., Chertova, E., Rosenberg, S. A., Felber, B. K., and Pavlakis, G. N. (2012). Circulating IL-15 exists as heterodimeric complex with soluble IL-15Ralpha in human and mouse serum. Blood 120, e1-8. 10.1182/blood-2011-10-384362.
Bergamaschi, C., Jalah, R., Kulkarni, V., Rosati, M., Zhang, G. M., Alicea, C., Zolotukhin, A. S., Felber, B. K., and Pavlakis, G. N. (2009). Secretion and biological activity of short signal peptide IL-15 is chaperoned by IL-15 receptor alpha in vivo. J Immunol 183, 3064-3072. 10.4049/jimmunol.0900693.
Bergamaschi, C., Pandit, H., Nagy, B. A., Stellas, D., Jensen, S. M., Bear, J., Cam, M., Valentin, A., Fox, B. A., Felber, B. K., and Pavlakis, G. N. (2020). Heterodimeric IL-15 delays tumor growth and promotes intratumoral CTL and dendritic cell accumulation by a cytokine network involving XCL1, IFN-gamma, CXCL9 and CXCL10. J Immunother Cancer 8. 10.1136/jitc-2020-000599.
Bergamaschi, C., Rosati, M., Jalah, R., Valentin, A., Kulkarni, V., Alicea, C., Zhang, G. M., Patel, V., Felber, B. K., and Pavlakis, G. N. (2008). Intracellular Interaction of Interleukin-15 with Its Receptor {alpha}during Production Leads to Mutual Stabilization and Increased Bioactivity. J Biol Chem 283, 4189-4199.
Berger, C., Berger, M., Hackman, R. C., Gough, M., Elliott, C., Jensen, M. C., and Riddell, S. R. (2009). Safety and immunologic effects ofIL-15 administration in nonhuman primates. Blood 114, 2417-2426. 10.1182/blood-2008-12-189266.
Blondel D. Vincent, G. J.-L., Lambiotte Renaud, Lefebvre Etienne (2008). Fast unfolding of communities in large networks. Journal of Statistical Mechanics. 10.1088/1742-5468/2008/10/P10008.
Bonnardel, J., Da Silva, C., Henri, S., Tamoutounour, S., Chasson, L., Montanana-Sanchis, F., Gorvel, J. P., and Lelouard, H. (2015). Innate and adaptive immune functions of peyer's patch monocyte-derived cells. Cell Rep 11, 770-784. 10.1016/j.celrep.2015.03.067.
Bosteels, C., Neyt, K., Vanheerswynghels, M., van Helden, M. J., Sichien, D., Debeuf, N., De Prijck, S., Bosteels, V., Vandamme, N., Martens, L., et al. (2020). Inflammatory Type 2 cDCs Acquire Features of cDCis and Macrophages to Orchestrate Immunity to Respiratory Virus Infection. Immunity 52, 1039-1056 e1039. 10.1016/j.immuni.2020.04.005.
Bosteels, C., and Scott, C. L. (2020). Transcriptional regulation of DC fate specification. Mol Immunol 121, 38-46. 10.1016/j.molimm.2020.02.021.
Bottcher, J. P., Bonavita, E., Chakravarty, P., Blees, H., Cabeza-Cabrerizo, M., Sammicheli, S., Rogers, N.C., Sahai, E., Zelenay, S., and Reis e Sousa, C. (2018). NK Cells Stimulate Recruitment of cDC1 into the Tumor Microenvironment Promoting Cancer Immune Control. Cell 172, 1022-1037 e1014. 10.1016/j.cell.2018.01.004.
Brown, C. C., Gudjonson, H., Pritykin, Y., Deep, D., Lavallee, V. P., Mendoza, A., Fromme, R., Mazutis, L., Ariyan, C., Leslie, C., et al. (2019). Transcriptional Basis of Mouse and Human Dendritic Cell Heterogeneity. Cell 179, 846-863 e824. 10.1016/j.cell.2019.09.035.
Calabro, S., Liu, D., Gallman, A., Nascimento, M. S., Yu, Z., Zhang, T. T., Chen, P., Zhang, B., Xu, L., Gowthaman, U., et al. (2016). Differential Intrasplenic Migration of Dendritic Cell Subsets Tailors Adaptive Immunity. Cell Rep 16, 2472-2485. 10.1016/j.celrep.2016.07.076.
Carson, W. E., Giri, J. G., Lindemann, M. J., Linett, M. L., Ahdieh, M., Paxton, R., Anderson, D., Eisenmann, J., Grabstein, K., and Caligiuri, M. A. (1994). Interleukin (IL) 15 is a novel cytokine that activates human natural killer cells via components of the IL-2 receptor. J Exp Med 180, 1395-1403.
Casbon, A. J., Reynaud, D., Park, C., Khuc, E., Gan, D. D., Schepers, K., Passegue, E., and Werb, Z. (2015). Invasive breast cancer reprograms early myeloid differentiation in the bone marrow to generate immunosuppressive neutrophils. Proc Natl Acad Sci USA 112, E566-575. 10.1073/pnas.1424927112.
Champiat, S., Tselikas, L., Farhane, S., Raoult, T., Texier, M., Lanoy, E., Massard, C., Robert, C., Ammari, S., De Baere, T., and Marabelle, A. (2021). Intratumoral Immunotherapy: From Trial Design to Clinical Practice. Clin Cancer Res 27, 665-679. 10.1158/1078-0432.CCR-20-0473.
Cheong, C., Matos, I., Choi, J. H., Dandamudi, D. B., Shrestha, E., Longhi, M. P., Jeffrey, K. L., Anthony, R. M., Kluger, C., Nchinda, G., et al. (2010). Microbial stimulation fully differentiates monocytes to DC-SIGN/CD209(+) dendritic cells for immune T cell areas. Cell 143, 416-429. 10.1016/j.cell.2010.09.039.
Chertova, E., Bergamaschi, C., Chertov, O., Sowder, R., Bear, J., Roser, J. D., Beach, R. K., Lifson, J. D., Felber, B. K., and Pavlakis, G. N. (2013). Characterization and favorable in vivo properties of heterodimeric soluble IL-15. IL-15Ralpha cytokine compared to IL-15 monomer. J Biol Chem 288, 18093-18103. 10.1074/jbc.M113.461756.
Conlon, K., Watson, D. C., Waldmann, T. A., Valentin, A., Bergamaschi, C., Felber, B. K., Peer, C. J., Figg, W. D., Potter, E. L., Roederer, M., et al. (2021a). Phase I study of single agent NIZ985, a recombinant heterodimeric IL-15 agonist, in adult patients with metastatic or unresectable solid tumors. Journal for Immunotherapy of Cancer 9, e003388. 10.1136/jitc-2021-003388.
Conlon, K., Watson, D. C., Waldmann, T. A., Valentin, A., Bergamaschi, C., Felber, B. K., Peer, C. J., Figg, W. D., Potter, E. L., Roederer, M., et al. (2021b). Phase I study of single agent NIZ985, a recombinant heterodimeric IL-15 agonist, in adult patients with metastatic or unresectable solid tumors. J Immunother Cancer 9. 10.1136/jitc-2021-003388.
Conlon, K. C., Lugli, E., Welles, H. C., Rosenberg, S. A., Fojo, A. T., Morris, J. C., Fleisher, T. A., Dubois, S. P., Perera, L. P., Stewart, D. M., et al. (2015). Redistribution, hyperproliferation, activation of natural killer cells and CD8 T cells, and cytokine production during first-in-human clinical trial of recombinant human interleukin-15 in patients with cancer. J Clin Oncol 33, 74-82. 10.1200/JCO.2014.57.3329.
Conlon, K. C., Potter, E. L., Pittaluga, S., Lee, C. R., Miljkovic, M. D., Fleisher, T. A., Dubois, S., Bryant, B. R., Petrus, M., Perera, L. P., et al. (2019). IL15 by Continuous Intravenous Infusion to Adult Patients with Solid Tumors in a Phase I Trial Induced Dramatic NK-Cell Subset Expansion. Clin Cancer Res 25, 4945-4954. 10.1158/1078-0432.CCR-18-3468.
Cooley, S., He, F., Bachanova, V., Vercellotti, G. M., DeFor, T. E., Curtsinger, J. M., Robertson, P., Grzywacz, B., Conlon, K. C., Waldmann, T. A., et al. (2019). First-in-human trial of rhIL-15 and haploidentical natural killer cell therapy for advanced acute myeloid leukemia. Blood Adv 3, 1970-1980. 10.1182/bloodadvances.2018028332.
Criscitiello, C., Esposito, A., Trapani, D., and Curigliano, G. (2016). Prognostic and predictive value of tumor infiltrating lymphocytes in early breast cancer. Cancer Treat Rev 50, 205-207. 10.1016/j.ctrv.2016.09.019.
Danaher, P., Warren, S., Dennis, L., D'Amico, L., White, A., Disis, M. L., Geller, M. A., Odunsi, K., Beechem, J., and Fling, S. P. (2017). Gene expression markers of Tumor Infiltrating Leukocytes. J Immunother Cancer 5, 18. 10.1186/s40425-017-0215-8.
Dent, R., Trudeau, M., Pritchard, K. I., Hanna, W. M., Kahn, H. K., Sawka, C. A., Lickley, L. A., Rawlinson, E., Sun, P., and Narod, S. A. (2007). Triple-negative breast cancer: clinical features and patterns of recurrence. Clin Cancer Res 13, 4429-4434. 10.1158/1078-0432.CCR-06-3045.
Diao, J., Gu, H., Tang, M., Zhao, J., and Cattral, M. S. (2018). Tumor Dendritic Cells (DCs) Derived from Precursors of Conventional DCs Are Dispensable for Intratumor CTL Responses. J Immunol 201, 1306-1314. 10.4049/jimmunol.1701514.
Durai, V., and Murphy, K. M. (2016). Functions of Murine Dendritic Cells. Immunity 45, 719-736. 10.1016/j.immuni.2016.10.010.
Eberlein, J., Nguyen, T. T., Victorino, F., Golden-Mason, L., Rosen, H. R., and Homann, D. (2010). Comprehensive assessment of chemokine expression profiles by flow cytometry. J Clin Invest 120, 907-923. 10.1172/JC140645.
Emma Kurz, C. A. H., Tanner Dalton, Sorin Alberto Shadaloey, Alireza Khodadadi-Jamayran, George Miller, Sumedha Pareek, Hajar Rajaei, Chirayu Mohindroo, Seyda Baydogan, An Ngo-Huang, Nathan Parker, Matthew H. G. Katz, Maria Petzel, Emily Vucic, Florencia McAllister, Keri Schadler, Rafael Winograd, Dafna Bar-Sagi (2022). Exercise-induced engagement of the IL-15/IL-15Rα axis promotes anti-tumor immunity in pancreatic cancer. Cancer Cell. https://doi.org/10.1016/j.ccell.2022.05.006
Feng, F., Wang, Y. C., Hu, X. B., Liu, X. W., Ji, G., Chen, Y. R., Wang, L., He, F., Dou, G. R., Liang, L., et al. (2010). The transcription factor RBP-J-mediated signaling is essential for dendritic cells to evoke efficient anti-tumor immune responses in mice. Mol Cancer 9, 90. 10.1186/1476-4598-9-90.
Finak, G., McDavid, A., Yajima, M., Deng, J., Gersuk, V., Shalek, A. K., Slichter, C. K., Miller, H. W., McElrath, M. J., Prlic, M., et al. (2015). MAST: a flexible statistical framework for assessing transcriptional changes and characterizing heterogeneity in single-cell RNA sequencing data. Genome Biol 16, 278. 10.1186/s13059-015-0844-5.
Gene Ontology, C. (2021). The Gene Ontology resource: enriching a GOld mine. Nucleic Acids Res 49, D325-D334. 10.1093/nar/gkaa1113.
Guilliams, M., Dutertre, C. A., Scott, C. L., McGovern, N., Sichien, D., Chakarov, S., Van Gassen, S., Chen, J., Poidinger, M., De Prijck, S., et al. (2016). Unsupervised High-Dimensional Analysis Aligns Dendritic Cells across Tissues and Species. Immunity 45, 669-684. 10.1016/j.immuni.2016.08.015.
Heger, L., Balk, S., Luhr, J. J., Heidkamp, G. F., Lehmann, C. H. K., Hatscher, L., Purbojo, A., Hartmann, A., Garcia-Martin, F., Nishimura, S. I., et al. (2018). CLEC10A Is a Specific Marker for Human CD1c(+) Dendritic Cells and Enhances Their Toll-Like Receptor 7/8-Induced Cytokine Secretion. Front Immunol 9, 744. 10.3389/fimmu.2018.00744.
Henry, C. J., Ornelles, D. A., Mitchell, L. M., Brzoza-Lewis, K. L., and Hiltbold, E. M. (2008). IL-12 produced by dendritic cells augments CD8+ T cell activation through the production of the chemokines CCL1 and CCL17. J Immunol 181, 8576-8584. 10.4049/jimmunol.181.12.8576.
Kaczanowska, S., Beury, D. W., Gopalan, V., Tycko, A. K., Qin, H., Clements, M. E., Drake, J., Nwanze, C., Murgai, M., Rae, Z., et al. (2021). Genetically engineered myeloid cells rebalance the core immune suppression program in metastasis. Cell 184, 2033-2052 e2021. 10.1016/j.cell.2021.02.048.
Karsunky, H., Merad, M., Cozzio, A., Weissman, I. L., and Manz, M. G. (2003). Flt3 ligand regulates dendritic cell development from Flt3+ lymphoid and myeloid-committed progenitors to Flt3+ dendritic cells in vivo. J Exp Med 198, 305-313. 10.1084/jem.20030323.
Kenkel, JA., Tseng, W. W., Davidson, M. G., Tolentino, L. L., Choi, O., Bhattacharya, N., Seeley, E. S., Winer, D. A., Reticker-Flynn, N. E., and Engleman, E. G. (2017). An Immunosuppressive Dendritic Cell Subset Accumulates at Secondary Sites and Promotes Metastasis in Pancreatic Cancer. Cancer Res 77, 4158-4170. 10.1158/0008-5472.CAN-16-2212.
Kim, T. S., Gorski, S. A., Hahn, S., Murphy, K. M., and Braciale, T. J. (2014). Distinct dendritic cell subsets dictate the fate decision between effector and memory CD8(+) T cell differentiation by a CD24-dependent mechanism. Immunity 40, 400-413. 10.1016/j.immuni.2014.02.004.
Kis-Toth, K., Bacskai, I., Gogolak, P., Mazlo, A., Szatmari, I., and Rajnavolgyi, E. (2013). Monocyte-derived dendritic cell subpopulations use different types of matrix metalloproteinases inhibited by GM6001. Immunobiology 218, 1361-1369. 10.1016/j.imbio.2013.06.012.
Klebanoff, C. A., Finkelstein, SE., Surman, DR., Lichtman, M. K., Gattinoni, L., Theoret, MR., Grewal, N., Spiess, P. J., Antony, P. A., Palmer, D. C., et al. (2004). IL-15 enhances the in vivo antitumor activity of tumor-reactive CD8+ T cells. Proc Natl Acad Sci USA 101, 1969-1974. 10.1073/pnas.0307298101.
Kleinman, H. K., and Martin, G. R. (2005). Matrigel: basement membrane matrix with biological activity. Semin Cancer Biol 15, 378-386. 10.1016/j.semcancer.2005.05.004.
Kobayashi, H., Watanabe, R., and Choyke, P. L. (2013). Improving conventional enhanced permeability and retention (EPR) effects; what is the appropriate target? Theranostics 4, 81-89. 10.7150/thno.7193.
Kool, M., Soullie, T., van Nimwegen, M., Willart, M. A., Muskens, F., Jung, S., Hoogsteden, H. C., Hammad, H., and Lambrecht, B. N. (2008). Alum adjuvant boosts adaptive immunity by inducing uric acid and activating inflammatory dendritic cells. J Exp Med 205, 869-882. 10.1084/jem.20071087.
Kumamoto, Y., Linehan, M., Weinstein, J. S., Laidlaw, B. J., Craft, J. E., and Iwasaki, A. (2013). CD301b(+) dermal dendritic cells drive T helper 2 cell-mediated immunity. Immunity 39, 733-743. 10.1016/j.immuni.2013.08.029.
Le Borgne, M., Etchart, N., Goubier, A., Lira, S. A., Sirard, J. C., van Rooijen, N., Caux, C., Ait-Yahia, S., Vicari, A., Kaiserlian, D., and Dubois, B. (2006). Dendritic cells rapidly recruited into epithelial tissues via CCR6/CCL20 are responsible for CD8+ T cell crosspriming in vivo. Immunity 24, 191-201. 10.1016/j.immuni.2006.01.005.
Le Naour, F., Hohenkirk, L., Grolleau, A., Misek, D. E., Lescure, P., Geiger, J. D., Hanash, S., and Beretta, L. (2001). Profiling changes in gene expression during differentiation and maturation of monocyte-derived dendritic cells using both oligonucleotide microarrays and proteomics. J Biol Chem 276, 17920-17931. 10.1074/jbc.M100156200.
Lee, H., Lee, H. J., Song, I. H., Bang, W. S., Heo, S. H., Gong, G., and Park, I. A. (2018). CD11c-Positive Dendritic Cells in Triple-negative Breast Cancer. In Vivo 32, 1561-1569. 10.21873/invivo.11415.
Lee, J. Y., Kim, H., Cha, M. Y., Park, H. G., Kim, Y. J., Kim, I. Y., and Kim, J. M. (2009). Clostridium difficile toxin A promotes dendritic cell maturation and chemokine CXCL2 expression through p38, IKK, and the NF-kappaB signaling pathway. J Mol Med (Berl) 87, 169-180. 10.1007/s00109-008-0415-2.
Lindstedt, M., Johansson-Lindbom, B., and Borrebaeck, C. A. (2002). Global reprogramming of dendritic cells in response to a concerted action of inflammatory mediators. Int Immunol 14, 1203-1213. 10.1093/intimm/dxf082.
Loi, S., Drubay, D., Adams, S., Pruneri, G., Francis, P. A., Lacroix-Triki, M., Joensuu, H., Dieci, M. V., Badve, S., Demaria, S., et al. (2019). Tumor-Infiltrating Lymphocytes and Prognosis: A Pooled Individual Patient Analysis of Early-Stage Triple-Negative Breast Cancers. J Clin Oncol 37, 559-569. 10.1200/JCO.18.01010.
Marabelle, A., Kohrt, H., Caux, C., and Levy, R. (2014). Intratumoral immunization: a new paradigm for cancer therapy. Clin Cancer Res 20, 1747-1756. 10.1158/1078-0432.CCR-13-2116.
Margolin, K., Morishima, C., Velcheti, V., Miller, J. S., Lee, S. M., Silk, A. W., Holtan, S. G., Lacroix, A. M., Fling, S. P., Kaiser, J. C., et al. (2018). Phase I Trial of ALT-803, A Novel Recombinant IL15 Complex, in Patients with Advanced Solid Tumors. Clin Cancer Res 24, 5552-5561. 10.1158/1078-0432.CCR-18-0945.
Mathios, D., Park, C. K., Marcus, W. D., Alter, S., Rhode, P. R., Jeng, E. K., Wong, H. C., Pardoll, D. M., and Lim, M. (2016). Therapeutic administration of IL-15 superagonist complex ALT-803 leads to long-term survival and durable antitumor immune response in a murine glioblastoma model. Int J Cancer 138, 187-194. 10.1002/ijc.29686.
Mattei, F., Schiavoni, G., Belardelli, F., and Tough, D. F. (2001). IL-15 is expressed by dendritic cells in response to type I IFN, double-stranded RNA, or lipopolysaccharide and promotes dendritic cell activation. J Immunol 167, 1179-1187. 10.4049/jimmunol.167.3.1179.
McColl, S. R. (2002). Chemokines and dendritic cells: a crucial alliance. Immunol Cell Biol 80, 489-496. 10.1046/j.1440-1711.2002.01113.x.
Miller, J. S., Morishima, C., McNeel, D. G., Patel, M. R., Kohrt, H. E. K., Thompson, J. A., Sondel, P. M., Wakelee, H. A., Disis, M. L., Kaiser, J. C., et al. (2018). A First-in-Human Phase I Study of Subcutaneous Outpatient Recombinant Human IL15 (rhIL15) in Adults with Advanced Solid Tumors. Clin Cancer Res 24, 1525-1535. 10.1158/1078-0432.CCR-17-2451.
Murphy, T. L., Grajales-Reyes, G. E., Wu, X., Tussiwand, R., Briseno, C. G., Iwata, A., Kretzer, N. M., Durai, V., and Murphy, K. M. (2016). Transcriptional Control of Dendritic Cell Development. Annu Rev Immunol 34, 93-119. 10.1146/annurev-immunol-032713-120204.
Nakano, H., Lin, K. L., Yanagita, M., Charbonneau, C., Cook, D. N., Kakiuchi, T., and Gunn, M. D. (2009). Blood-derived inflammatory dendritic cells in lymph nodes stimulate acute T helper type 1 immune responses. Nat Immunol 10, 394-402. 10.1038/ni.1707.
Napoletano, C., Rughetti, A., Agervig Tarp, M. P., Coleman, J., Bennett, E. P., Picco, G., Sale, P., Denda-Nagai, K., Irimura, T., Mandel, U., et al. (2007). Tumor-associated Tn-MUC1 glycoform is internalized through the macrophage galactose-type C-type lectin and delivered to the HLA class I and II compartments in dendritic cells. Cancer Res 67, 8358-8367. 10.1158/0008-5472.CAN-07-1035.
Ng, S. S. M., Nagy, B. A., Jensen, S. M., Hu, X., Alicea, C., Fox, B. A., Felber, B. K., Bergamaschi, C., and Pavlakis, G. N. (2017). Heterodimeric IL15 Treatment Enhances Tumor Infiltration, Persistence, and Effector Functions of Adoptively Transferred Tumor-specific T Cells in the Absence of Lymphodepletion. Clin Cancer Res 23, 2817-2830. 10.1158/1078-0432.Ccr-16-1808.
Nizzoli, G., Krietsch, J., Weick, A., Steinfelder, S., Facciotti, F., Gruarin, P., Bianco, A., Steckel, B., Moro, M., Crosti, M., et al. (2013). Human CD1c+ dendritic cells secrete high levels of IL-12 and potently prime cytotoxic T-cell responses. Blood 122, 932-942. 10.1182/blood-2013-04-495424.
Persson, E. K., Scott, C. L., Mowat, A. M., and Agace, W. W. (2013). Dendritic cell subsets in the intestinal lamina propria: ontogeny and function. Eur J Immunol 43, 3098-3107. 10.1002/eji.201343740.
Plantinga, M., Guilliams, M., Vanheerswynghels, M., Deswarte, K., Branco-Madeira, F., Toussaint, W., Vanhoutte, L., Neyt, K., Killeen, N., Malissen, B., et al. (2013). Conventional and monocyte-derived CD11b(+) dendritic cells initiate and maintain T helper 2 cell-mediated immunity to house dust mite allergen. Immunity 38, 322-335. 10.1016/j.immuni.2012.10.016.
Qu, C., Brinck-Jensen, N. S., Zang, M., and Chen, K. (2014). Monocyte-derived dendritic cells: targets as potent antigen-presenting cells for the design of vaccines against infectious diseases. Int J Infect Dis 19, 1-5. 10.1016/j.ijid.2013.09.023.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res. 43, e47. 10.1093/nar/gkv007.
Romee, R., Cooley, S., Berrien-Elliott, M. M., Westervelt, P., Verneris, M. R., Wagner, J. E., Weisdorf, D. J., Blazar, B. R., Ustun, C., DeFor, T. E., et al. (2018). First-in-human phase 1 clinical study of the IL-15 superagonist complex ALT-803 to treat relapse after transplantation. Blood 131, 2515-2527. 10.1182/blood-2017-12-823757.
Santambrogio, L., Berendam, S. J., and Engelhard, V. H. (2019). The Antigen Processing and Presentation Machinery in Lymphatic Endothelial Cells. Front Immunol 10, 1033. 10.3389/fimmu.2019.01033.
Schlitzer, A., McGovern, N., Teo, P., Zelante, T., Atarashi, K., Low, D., Ho, A. W., See, P., Shin, A., Wasan, P. S., et al. (2013). IRF4 transcription factor-dependent CD11b+ dendritic cells in human and mouse control mucosal IL-17 cytokine responses. Immunity 38, 970-983. 10.1016/j.immuni.2013.04.011.
Schreibelt, G., Bol, K. F., Westdorp, H., Wimmers, F., Aarntzen, E. H., Duiveman-de Boer, T., van de Rakt, M. W., Scharenborg, N. M., de Boer, A. J., Pots, J. M., et al. (2016). Effective Clinical Responses in Metastatic Melanoma Patients after Vaccination with Primary Myeloid Dendritic Cells. Clin Cancer Res 22, 2155-2166. 10.1158/1078-0432.CCR-15-2205.
Semmling, V., Lukacs-Kornek, V., Thaiss, C. A., Quast, T., Hochheiser, K., Panzer, U., Rossjohn, J., Perlmutter, P., Cao, J., Godfrey, D. I., et al. (2010). Alternative cross-priming through CCL17-CCR4-mediated attraction of CTLs toward NKT cell-licensed DCs. Nat Immunol 11, 313-320. 10.1038/ni.1848.
Shaffer, A. L., Emre, N.C., Lamy, L., Ngo, V. N., Wright, G., Xiao, W., Powell, J., Dave, S., Yu, X., Zhao, H., et al. (2008). IRF4 addiction in multiple myeloma. Nature 454, 226-231. 10.1038/nature07064.
Sheng, J., Chen, Q., Soncin, I., Ng, S. L., Karjalainen, K., and Ruedl, C. (2017). A Discrete Subset of Monocyte-Derived Cells among Typical Conventional Type 2 Dendritic Cells Can Efficiently Cross-Present. Cell Rep 21, 1203-1214. 10.1016/j.celrep.2017.10.024.
Shin, H., Kumamoto, Y., Gopinath, S., and Iwasaki, A. (2016). CD301b+ dendritic cells stimulate tissue-resident memory CD8+ T cells to protect against genital HSV-2. Nat Commun 7, 13346. 10.1038/ncomms13346.
Sichien, D., Scott, C. L., Martens, L., Vanderkerken, M., Van Gassen, S., Plantinga, M., Joeris, T., De Prijck, S., Vanhoutte, L., Vanheerswynghels, M., et al. (2016). IRF8 Transcription Factor Controls Survival and Function of Terminally Differentiated Conventional and Plasmacytoid Dendritic Cells, Respectively. Immunity 45, 626-640. 10.1016/j.immuni.2016.08.013.
Siegel, R. L., Miller, K. D., and Jemal, A. (2018). Cancer statistics, 2018. CA Cancer J Clin 68, 7-30. 10.3322/caac.21442.
Spranger, S., Dai, D., Horton, B., and Gajewski, T. F. (2017). Tumor-Residing Batf3 Dendritic Cells Are Required for Effector T Cell Trafficking and Adoptive T Cell Therapy. Cancer Cell 31, 711-723 e714. 10.1016/j.ccell.2017.04.003.
Tamoutounour, S., Guilliams, M., Montanana Sanchis, F., Liu, H., Terhorst, D., Malosse, C., Pollet, E., Ardouin, L., Luche, H., Sanchez, C., et al. (2013). Origins and functional specialization of macrophages and of conventional and monocyte-derived dendritic cells in mouse skin. Immunity 39, 925-938. 10.1016/j.immuni.2013.10.004.
Tamoutounour, S., Henri, S., Lelouard, H., de Bovis, B., de Haar, C., van der Woude, C. J., Woltman, A. M., Reyal, Y., Bonnet, D., Sichien, D., et al. (2012). CD64 distinguishes macrophages from dendritic cells in the gut and reveals the Th1-inducing role of mesenteric lymph node macrophages during colitis. Eur J Immunol 42, 3150-3166. 10.1002/eji.201242847.
Tel, J., Aarntzen, E. H., Baba, T., Schreibelt, G., Schulte, B. M., Benitez-Ribas, D., Boerman, O. C., Croockewit, S., Oyen, W. J., van Rossum, M., et al. (2013). Natural human plasmacytoid dendritic cells induce antigen-specific T-cell responses in melanoma patients. Cancer Res 73, 1063-1075. 10.1158/0008-5472.CAN-12-2583.
Theisen, D. J., Davidson, J. T. t., Briseno, C. G., Gargaro, M., Lauron, E. J., Wang, Q., Desai, P., Durai, V., Bagadia, P., Brickner, J. R., et al. (2018). WDFY4 is required for cross-presentation in response to viral and tumor antigens. Science 362, 694-699. 10.1126/science.aat5030.
Torres, E. T. R., and Emens, L. A. (2022). Emerging combination immunotherapy strategies for breast cancer: dual immune checkpoint modulation, antibody-drug conjugates and bispecific antibodies. Breast Cancer Res Treat 191, 291-302. 10.1007/s10549-021-06423-0.
Tourkova, I. L., Shurin, G. V., Chatta, G. S., Perez, L., Finke, J., Whiteside, T. L., Ferrone, S., and Shurin, M. R. (2005). Restoration by IL-15 of MHC class I antigen-processing machinery in human dendritic cells inhibited by tumor-derived gangliosides. J Immunol 175, 3045-3052. 10.4049/jimmunol.175.5.3045.
Tourkova, I. L., Yurkovetsky, Z. R., Gambotto, A., Makarenkova, V. P., Perez, L., Balkir, L., Robbins, P. D., Shurin, M R., and Shurin, G. V. (2002). Increased function and survival of IL-15-transduced human dendritic cells are mediated by up-regulation of IL-15Ralpha and Bcl-2. J Leukoc Biol 72, 1037-1045.
Toyonaga, K., Torigoe, S., Motomura, Y., Kamichi, T., Hayashi, J. M., Morita, Y. S., Noguchi, N., Chuma, Y., Kiyohara, H., Matsuo, K., et al. (2016). C-Type Lectin Receptor DCAR Recognizes Mycobacterial Phosphatidyl-Inositol Mannosides to Promote a Th1 Response during Infection. Immunity 45, 1245-1257. 10.1016/j.immuni.2016.10.012.
Tzelepis, F., Verway, M., Daoud, J., Gillard, J., Hassani-Ardakani, K., Dunn, J., Downey, J., Gentile, M. E., Jaworska, J., Sanchez, A. M., et al. (2015). Annexin1 regulates DC efferocytosis and cross-presentation during Mycobacterium tuberculosis infection. J Clin Invest 125, 752-768. 10.1172/JCI77014.
Wakim, L. M., Waithman, J., van Rooijen, N., Heath, W. R., and Carbone, F. R. (2008). Dendritic cell-induced memory T cell activation in nonlymphoid tissues. Science 319, 198-202. 10.1126/science.1151869.
Waldmann, T. A. (2006). The biology of interleukin-2 and interleukin-15: implications for cancer therapy and vaccine design. Nat Rev Immunol 6, 595-601. 10.1038/nri1901.
Waldmann, T. A., Lugli, E., Roederer, M., Perera, L. P., Smedley, J. V., Macallister, R. P., Goldman, C. K., Bryant, B. R., Decker, J. M., Fleisher, T. A., et al. (2011). Safety (toxicity), pharmacokinetics, immunogenicity, and impact on elements of the normal immune system of recombinant human IL-15 in rhesus macaques. Blood 117, 4787-4795. 10.1182/blood-2010-10-311456.
Waldmann, T. A., Miljkovic, M. D., and Conlon, K. C. (2020). Interleukin-15 (dys)regulation of lymphoid homeostasis: Implications for therapy of autoimmunity and cancer. J Exp Med 217. 10.1084/jem.20191062.
Westdorp, H., Creemers, J. H. A., van Oort, I. M., Schreibelt, G., Gorris, M. A. J., Mehra, N., Simons, M., de Goede, A. L., van Rossum, M. M., Croockewit, A. J., et al. (2019). Blood-derived dendritic cell vaccinations induce immune responses that correlate with clinical outcome in patients with chemo-naive castration-resistant prostate cancer. J Immunother Cancer 7, 302. 10.1186/s40425-019-0787-6.
Williams, J. W., Tjota, M. Y., Clay, B. S., Vander Lugt, B., Bandukwala, H. S., Hrusch, C. L., Decker, D. C., Blaine, K. M., Fixsen, B. R., Singh, H., et al. (2013). Transcription factor IRF4 drives dendritic cells to promote Th2 differentiation. Nat Commun 4, 2990. 10.1038/ncomms3990.
Wu, X., Briseno, C. G., Durai, V., Albring, J. C., Haldar, M., Bagadia, P., Kim, K. W., Randolph, G. J., Murphy, T. L., and Murphy, K. M. (2016). Mafb lineage tracing to distinguish macrophages from other immune lineages reveals dual identity of Langerhans cells. J Exp Med 213, 2553-2565. 10.1084/jem.20160600.
Xu, W., Jones, M., Liu, B., Zhu, X., Johnson, C. B., Edwards, A. C., Kong, L., Jeng, EK., Han, K., Marcus, W. D., et al. (2013). Efficacy and mechanism-of-action of a novel superagonist interleukin-15: interleukin-15 receptor alphaSu/Fc fusion complex in syngeneic murine models of multiple myeloma. Cancer Res 73, 3075-3086. 10.1158/0008-5472.CAN-12-2357.
Yu, P., Steel, J. C., Zhang, M., Morris, J. C., Waitz, R., Fasso, M., Allison, J. P., and Waldmann, T. A. (2012). Simultaneous inhibition of two regulatory T-cell subsets enhanced Interleukin-15 efficacy in a prostate tumor model. Proc Natl Acad Sci USA 109, 6187-6192. 10.1073/pnas.1203479109.
Yu, P., Steel, J. C., Zhang, M., Morris, J. C., and Waldmann, T. A. (2010). Simultaneous blockade of multiple immune system inhibitory checkpoints enhances antitumor activity mediated by interleukin-15 in a murine metastatic colon carcinoma model. Clin Cancer Res 16, 6019-6028. 10.1158/1078-0432.CCR-10-1966.
Zhang, S., Ma, X., Zhu, C., Liu, L., Wang, G., and Yuan, X. (2016). The Role of Myeloid-Derived Suppressor Cells in Patients with Solid Tumors: A Meta-Analysis. PLoS One 11, e0164514. 10.1371/journal.pone.0164514.
Zigmond, E., Varol, C., Farache, J., Elmaliah, E., Satpathy, A. T., Friedlander, G., Mack, M., Shpigel, N., Boneca, I. G., Murphy, K. M., et al. (2012). Ly6C hi monocytes in the inflamed colon give rise to proinflammatory effector cells and migratory antigen-presenting cells. Immunity 37, 1076-1090. 10.1016/j.immuni.2012.08.026.

### FIG. 1. HetIL-15 Administration Resulted in Significant EO771 Tumor Growth Delay and Increased Survival

(A) Treatment timeline. On day −1, C57BL/6 mice were inoculated with 3×10⁵ EO771 cells orthotopically in the 4^{th} mammary pad. Mice with palpable tumors were distributed in different groups 7 days later and treated locoregionally with hetIL-15 injections (5 g/mouse/dose) in the mammary fat pad in the vicinity of the tumor every 4 days. Orange arrows indicate days of tumor and draining LNs collection.

(B) Tumor growth over time from three independent experiments combined. Control mice (n=21) and hetIL-15-treated mice (n=54) received 3 injections (5 g/dose/mouse every 4 days), starting at day 0.

(C) Kaplan-Meier survival curve of EO771 tumor-bearing C57BL/6 mice treated with 6 hetIL-15 doses (5 g/mouse/dose) or vehicle (control) every 4 days. Data shown are from one experiment with 8-10 mice per group.

(D-G) Tumor immune cell infiltrates were analyzed by flow cytometry to determine absolute numbers of cells per gram of tissue: CD8⁺T (D), NK (E), Granzyme Wand Ki67⁺CD8⁺T (F) or Granzyme B⁺ and Ki67⁺NK (G) cells. Data of three independent experiments with 4-6 mice per group were combined; bars represent mean±SEM. Each symbol refers to one mouse.

(H) EO771 tumor sections from mice treated with 3 injections of hetIL-15 or vehicle (control), as indicated. TTLs were identified by immunohistochemical staining using antibodies specific for CD8⁺ or NK.1.1 cells. A representative image from one mouse/group is shown.

FIG. 2. Transcriptomic Profile of hetIL-15 Treated Tumors Revealed an Activated Tumor-Infiltrating Immune Cell Profile

Gene expression analysis of EO771 tumors recovered from mice treated with either PBS (n=3) or hetIL-15 (5 g/dose/mouse every 4 days) (n=2-3) was performed by the Nanostring technology using a panel of 780 immune-oncology related gene probes (PanCancer Immune Profiling Panel). The analysis was conducted 48 hrs post the 1^{st}, 2^{nd} and 3^{rd} hetIL-15 injection.

(A-C) Volcano plot depicts differentially expressed genes between the two treatment groups. The genes marked in red, green and blue are associated with T and NK cell cytotoxicity, enhanced T cell activation/TCR signaling and lymphocyte migration, respectively. Dashed line represents adjusted p-value=0.05 and dotted lines represent log 2(FC)=1 and log 2(FC)=−1.

(D) GO tumor enriched pathways from differentially expressed genes are presented.

(E-G) Heatmaps of T cell co-stimulation (E), antigen receptor-mediated signaling (F), positive regulation of T cell activation (G) pathways indicate the upregulated genes in hetIL-15 treated tumors, in comparison to control tumors.

FIG. 3. A Novel Dendritic Cell Population is Detected in the hetIL-15 Treated Tumors

(A) Heatmap representing the estimated immune cell composition of tumors upon hetIL-15 treatment. Cell scores were calculated for different immune cell subsets as described in Material and Methods.

(B) Gating and staining strategy used to identify distinct DC populations in the EO771 tumors. The CD103⁺cDC1 (red), CD11b⁺cDC2 (blue) and the novel population CD103^{int}CD11b⁺DC (green) are indicated in the contour plots.

(C-E) Flow cytometric analysis of intratumoral CD103⁺cDC1 (C), CD11b⁺cDC2 (D) and CD103^{int}CD11b⁺DC (E) populations in controls and hetIL-15 treated mice. Data in graphs are given as absolute numbers of cells per gram of tissue and represented as mean±SEM.

(F) Pearson correlation analysis between tumor volume (mm³) and number of tumor-infiltrating DCs per gram of tissue. Data shown in (C-F) are pooled from three different experiments with n=4-6 mice per group.

### FIG. 4. Phenotypic Analysis of the Novel Tumor Infiltrated DC Population

(A) t-SNE analysis of CD103⁺cDC1, CD11b⁺cDC2, CD103^{int}CD11b⁺DC populations and macrophages based on multi-color flow cytometry generated from 11 concatenated samples; 5 samples from control group and 6 samples from hetIL-15 treated group.

(B-E) Histogram plots show the expression levels of CD24, CD64, CD169, CXC3R1, Ly6C (B), F4/80 (C), XCR1, IRF8 (D) and TREM, CD101 (E) on CD103⁺cDC1 (red), CD11b⁺cDC2 (blue), CD103^{int}CD11b⁺DC (green) populations and macrophages (gray). Data are from three (B to D) or two (E) independent replicate experiments.

FIG. 5. Single Cell-RNA Sequencing Analysis Revealed that hetIL-15 Induced CD103^{int}CD11b⁺DCs Share Transcriptional Similarities with moDCs and cDCs

Isolated tumor-infiltrating CD11c⁺ populations from control and hetIL15-treated EO771-tumor bearing mice were processed into single-cell suspension.

(A) UMAP plot of scRNA-seq analysis of CD11c⁺ tumor-infiltrating cells serially annotated with SingleR (Brown et al., 2019).

(B) Scaled density UMAP plot showing sample origin of clustered cells in each cluster. 0 to 1 indicating 100% of cells originating from control sample or hetIL15-treated sample, respectively.

(C) Heatmap reporting scaled, imputed expression of the top 10 differentially expressed genes for each cluster across all cells, identified in FIG. 5a. Genes of interest are shown in red.

(D) Heatmap showing DC different pathways (Ashburner et al., 2000; Gene Ontology, 2021; Le Naour et al., 2001; Lindstedt et al., 2002; Shaffer et al., 2008) enriched in the integrated tumor-infiltrating CD11c⁺ clusters by GSEA analysis, colored by z-score transformed mean GSEA scores.

FIG. 6. Triple RNA In Situ Hybridization (RNAscope) of EO771 Cancer Samples Verified the Presence of the CD103^{int}CD11b⁺DCs in the Tumors of the hetIL-15 Treated Mice

Triple RNA in situ hybridization of EO771 cancer samples verified the presence of the CD103^{int}CD11b⁺DCs in the tumors of the hetIL-15 treated mice. Low-magnification images (2 mm, upper panel) and 20× images (200 μm; control group and 100 um; hetIL-15 group, middle panel) showing the expression of Mgl2 (yellow), Ccl17 (green) and CD24a (pink) mRNA in paraffin embedded tissue. High-magnification (40×, 50 μm, bottom) of areas (1), (2) and (3) individual images showing CD103^{int}CD11b⁺DCs expressing Mgl2, Ccl17 and CD24a. Nuclear staining using DAPI (blue). LN; lymph node. White arrows indicate the CD103^{int}CD11b⁺DCs. Representative images from one experiment with n=5.

### FIG. 7. HetIL-15 Locoregional Administration Provided Long-Lasting Specific Anti-Tumor Immunity

(A) Timeline of repetitive tumor challenge. On day −5, C57BL/6 mice were inoculated with EO771 cells (3×10⁵, SC in the 4^{th} mammary pad). Starting six days later, mice were treated with 5 hetIL-15 injections (5 g/mouse/dose) every 4 days. On day 90, long-term surviving tumor-free mice were rechallenged (challenge #1) by injection of EO771 cells (5×10⁴, SC in the 4^{th} mammary pad). No tumor growth was detected and on day 180, the mice were rechallenged (challenge #2) by injection of EO771 cells (5×10⁴, SC in the 4^{th} mammary pad) and by injection of KPC cells (5×10⁴, SC in the 3^{rd} mammary pad). The endpoint time of this experiment was day 196.

(B and C) Growth of individual EO771 (B) and KPC tumors (C) were monitored from day 180 (challenge #2) until the endpoint. Data are pooled from 2 experiments with 5 mice per group.

(D) Schematic representation of adoptive transfer of tumor immunity. Recipient mice were challenged with EO771 cells (5×10⁴, SC in the 4^{th} mammary pad) on day 0; 6 days later, the mice were irradiated with 600 cGy. Eight days after tumor challenge, CD8⁺T cells from spleen of naïve or rechallenged mice from FIG. 7A were isolated and injected into the EO771 tumor-bearing mice. Recipient mice were then boosted with hetIL-15 i.p. injections (5 μg/dose/mouse) every 2 days until the end point.

(E) EO771 tumor size (left panel) and survival curve (right panel) following adoptive transfer. Data shown in (E, left) are pooled from 2 experiments with 5-9 mice per group and represented as mean±SEM; **p<0.01. Data in (E, right) are from one representative experiment performed twice.

### Supplemental Information

### Supplemental Information Includes 6 Supplemental Figures

FIG. S1 (related to FIG. 1)

FIG. S2 (related to FIG. 2)

FIG. S3 (related to FIG. 4)

FIG. S4 (related to FIG. 5)

FIG. S5 (related to FIG. 5)

FIG. S6 (related to FIG. 7)

FIG. S1. Comparison of EO771 tumor growth and metastasis in orthotopic mouse models of different immunological backgrounds (related to FIG. 1). (A, C and E) Tumor growth curves (bold lines represent average values) of C57BL/6 (A), C57BL/6 Rag-1 ko (C) or C57BL/6 NK depleted (E) mice. hetIL-15 (5 μg/dose/mouse) was injected every 4 days for a total of 5 (A) or 6 (C and E) doses. Injections of anti-NK1.1 mAb or IgG2a (100 g/dose/mouse, i.p.) were performed as described in Star Methods. (B, D and F) H&E representative staining images of EO771 lung metastases in control or hetIL-15 treated C57BL/6 (B), C57BL/6 Rag-1 ko (D) or C57BL/6 NK depleted (F) mice (right panel). Data represented as mean±SEM are from one representative of two independent replicates (n=5) (B), one experiment with n=10 (D), one experiment with n=5-6 mice (F). Arrows in images indicate the metastatic foci. Scale bar=4 mm.

FIG. S2. hetIL-15 treatment altered gene expression of the draining lymph nodes (related to FIG. 2). (A-C) Volcano plot depicts differentially expressed genes in the two groups, after the 1^{st}, 2^{nd} and 3^{rd} locoregional hetIL-15 injection. Dashed line represents adjusted p-value=0.05 and dotted lines represent log 2(FC)=1 and log 2(FC)=−1. The genes marked in red, green and blue are associated with T and NK cell cytotoxicity, enhanced T cell activation/TCR signaling and lymphocyte migration, respectively. (D and E) Heatmaps of differentially expressed genes in the lymphocyte migration and T cell activation pathways. (F and G) dLN was also analyzed by flow cytometry to determine the percentage of: CD8⁺T (F) and NK (G) cells. Data are from one experiment with n=5-6 mice and shown as mean±SEM.

FIG. S3. The novel, hetIL-15 associated CD103^{int}CD11b⁺DC population is present in 4T1 orthotopic tumors, upon hetIL-15 treatment (related to FIG. 4). Balb/c female mice were implanted with 3×10⁵ 4T1 cells orthotopically into the 4^{th} inguinal mammary fat pad and when palpable tumor had formed, mice were treated with hetIL-15 or vehicle (control). (A) Treatment schedule. Injections of het-IL15 (5 μg/dose/mouse) were performed every 4 days for a total of 3 doses. (B) Tumor growth was monitored overtime. Data are from one experiment with n=8-12, shown as mean±SEM. 4T1 tumor-bearing mice were sacrificed at day 16 after treatment with either saline or hetIL-15 (48 hrs after the 3^{rd} administration). (C-G) Tumor immune infiltrates were analyzed by flow cytometry to determine the absolute number of the cells per gram of tissue of: CD8⁺T cells (C), NK cells (D) and CD103⁺cDC1 (E), CD11b⁺cDC2 (F) and CD103^{int}CD11b⁺DC (G) populations. Data are from one experiment with n=5-9, plotted as mean±SEM. (H) Flow cytometric analysis of CD103, IRF8 and XCR1 expression. Histogram overlays show the expression of CD103, IRF8 and XCR1 by intratumoral CD103⁺cDC1 (red), CD11b⁺cDC2 (blue) and CD103^{int}CD11b⁺DC (green) populations from a representative hetIL-15 treated mouse.

FIG. S4. Transcriptional analysis highlights distinct profile of tumor CD103^{int}CD11b⁺DC (related to FIG. 5). Tumor-infiltrating DC subpopulations (CD103⁺cDC1, CD11b⁺cDC2 and CD103^{int}CD11b⁺DC) and macrophages were sorted, based on the gating strategy from FIG. 3b. RNA isolation and bulk RNA-seq analysis was performed to the sorted populations. (A) PCA of CD103⁺cDC1, CD11b⁺cDC2, CD103^{int}CD11b⁺DC populations and macrophages based on RNA-seq global transcriptional profiles. (B) Heat map of log 2-transformed expression from RNA-seq across populations for DC canonical markers (Brown et al., 2019) as well as from macrophage/monocyte markers. Red and green gene names indicate genes that are upregulated and downregulated, respectively. (C) Heatmap of genes in the antigen presentation pathway (Kaczanowska et al., 2021) among DC cell subsets and macrophages. Red gene names indicate upregulated genes. (D) IFN-γ production in isolated splenic CD8⁺T cells from naïve mice upon ex vivo co-culture with sorted tumor-infiltrating CD103^{int}CD11b⁺DC. Data shown are from one experiment, presented as mean±SEM.

FIG. S5. Sc-RNA sequencing analysis reveals unique profile of the hetIL-15 associated CD103^{int}CD11b⁺DC population (related to FIG. 5). Isolated tumor-infiltrating CD11c⁺ populations from control and hetIL15-treated EO771-tumor bearing mice were processed into single-cell suspension. (A) CD24 expression in scRNA-seq SCT (red) and CITE-seq protein (green) assays were quantile filtered and scaled from 0 to 1. (B) Average expression bubble plot of genes in the canonical DC marker (Brown et al., 2019) gene set as well as from macrophage/monocyte markers, among clusters in the scRNA-seq dataset colored by average gene expression of SCTransformed scaled counts. Due to high gene expression in CD103⁺cDC1, this cluster was removed to explore finer differences between the remaining clusters. (C) Gene expression levels of chemokines are shown for CD103⁺cDC1, CD11b⁺cDC2 and CD103^{int}CD11b⁺DC clusters. P-values for differences between CD103^{int}CD11b⁺DC and CD103⁺cDC1 or CD11b⁺cDC2 were <0.001 and were determined using the MAST algorithm.

FIG. S6. IL-15-induced memory CD8⁺T cells control metastatic disease in the lungs (related to FIG. 7). H&E representative staining of lung metastases and the corresponding actual counts per slide of lung metastatic foci in C57BL/6 recipient mice that underwent adoptive cell transfer of CD8⁺T cells from FIG. 7D. Data shown are from one experiment with n=5 mice per group and represented as mean±SEM.

Graphical Abstract

TABLE 4
IL-15 sequences

Immature/precursor form of native human IL-15
MRISKPHLRSISIQCYLCLLLNSHFLTEAGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEEKNIKEFLQSFV
HIVQMFINTS (SEQ ID NO: 01)
Immature form of the native full length human IL-15 receptor alpha
MAPRRARGCRTLGLPALLLLLLLRPPATRGITCPPPMSVEHADIWVKSYSLYSRERYICNSGFKRKAGTSSLTECV
LNKATNVAHWTTPSLKCIRDPALVHQRPAPPSTVTTAGVTPQPESLSPSGKEPAASSPSSNNTAATTAAIVPGSQL
MPSKSPSTGTTEISSHESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL (SEQ ID NO: 03)
Immature form of the native soluble human IL-15 receptor alpha
MAPRRARGCRTLGLPALLLLLLLRPPATRGITCPPPMSVEHADIWVKSYSLYSRERYICNSGFKRKAGTSSLTECV
LNKATNVAHWTTPSLKCIRDPALVHQRPAPPSTVTTAGVTPQPESLSPSGKEPAASSPSSNNTAATTAAIVPGSQL
MPSKSPSTGTTEISSHESSHGTPSQTTAKNWELTASASHQPPGVYPQGHSDTT (SEQ ID NO: 04)
AG32 huIL15opt-amino acid sequence of optimized human IL-15
MRISKPHLRSISIQCYLCLLLNSHFLTEAGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEEKNIKEFLQSFV
HIVQMFINTS (SEQ ID NO: 01)
AG59 CMV huIL15tPA6-amino acid sequence of optimized human IL-15
MDAMKRGLCCVLLLCGAVFVSPSQEIHARFRRGARNWVNVISDLKKIEDLIQSMHIDATLYTESDVHPSCKVTAMK
CFLLELQVISLESGDASIHDTVENLIILANNSLSSNGNVTESGCKECEELEEKNIKEFLQSFVHIVQMFINTS
(SEQ ID NO: 06)
AG79 huIL15Rα-amino acid sequence of optimized human IL-15Rα
MAPRRARGCRTLGLPALLLLLLLRPPATRGITCPPPMSVEHADIWVKSYSLYSRERYICNSGFKRKAGTSSLTECV
LNKATNVAHWTTPSLKCIRDPALVHQRPAPPSTVTTAGVTPQPESLSPSGKEPAASSPSSNNTAATTAAIVPGSQL
MPSKSPSTGTTEISSHESSHGTPSQTTAKNWELTASASHQPPGVYPQGHSDTTVAISTSTVLLCGLSAVSLLACYL
KSRQTPPLASVEMEAMEALPVTWGTSSRDEDLENCSHHL (SEQ ID NO: 07)
AG98 CMV hu sIL15Rα-amino acid sequence of optimized human IL-15Rα
MAPRRARGCRTLGLPALLLLLLLRPPATRGITCPPPMSVEHADIWVKSYSLYSRERYICNSGFKRKAGTSSLTECV
LNKATNVAHWTTPSLKCIRDPALVHQRPAPPSTVTTAGVTPQPESLSPSGKEPAASSPSSNNTAATTAAIVPGSQL
MPSKSPSTGTTEISSHESSHGTPSQTTAKNWELTASASHQPPGVYPQGHSDTT (SEQ ID NO: 08)
AG151 huIL-15 huGM-CSF-amino acid sequence of optimized human IL-15 with a
signal peptide of human GM-CSF
MWLQSLLLLGTVACSISNWVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINTS (SEQ ID NO: 09)
IL-15-114 aa sequence of mature human IL-15 polypeptide plus the 48 aa
signal peptide underlined
MRISKPHLRSISIQCYLCLLLNSHFLTEAGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEEKNIKEFLQSFV
HIVQMFINTS (SEQ ID NO: 01)
>Mature secreted IL-15 protein
NWVNVISDLKKIEDLIQSMHIDATLYTESDVHPSCKVTAMKCFLLELQVISLESGDASIHDTVENLIILANNSLSS
NGNVTESGCKECEELEEKNIKEFLQSFVHIVQMFINTS (SEQ ID NO: 02)
>AG98 human IL-15sRa
signal peptide 30 AA underlined
175 AA IL-15sRa mature, cleaved, secreted
MAPRRARGCRTLGLPALLLLLLLRPPATRGITCPPPMSVEHADIWVKSYSLYSRERYICNSGFKRKAGTSSLTECV
LNKATNVAHWTTPSLKCIRDPALVHQRPAPPSTVTTAGVTPQPESLSPSGKEPAASSPSSNNTAATTAAIVPGSQL
MPSKSPSTGTTEISSHESSHGTPSQTTAKNWELTASASHQPPGVYPQGHSDTT (SEQ ID NO: 08)
ITCPPPMSVEHADIWVKSYSLYSRERYICNSGFKRKAGTSSLTECVLNKATNVAHWTTPSLKCIRDPALVHQRPAP
PSTVTTAGVTPQPESLSPSGKEPAASSPSSNNTAATTAAIVPGSQLMPSKSPSTGTTEISSHESSHGTPSQTTAKN
WELTASASHQPPGVYPQGHSDTT (SEQ ID NO: 10)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQG (SEQ ID NO: 11)
MAPRRARGCR TLGLPALLLL LLLRPPATRG ITCPPPMSVE HADIWVKSYS LYSRERYICN SGFKRKAGTS
SLTECVLNKA TNVAHWTTPS LKCIRDPALV HQRPAPPSTV TTAGVTPQPE SLSPSGKEPA ASSPSSNNTA
ATTAAIVPGS QLMPSKSPST GTTEISSHES SHGTPSQTTA KNWELTASAS HQPPGVYPQGH (SEQ ID
NO: 12)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQGH (SEQ ID NO: 13)
MAPRRARGCR TLGLPALLLL LLLRPPATRG ITCPPPMSVE HADIWVKSYS LYSRERYICN SGFKRKAGTS
SLTECVLNKA TNVAHWTTPS LKCIRDPALV HQRPAPPSTV TTAGVTPQPE SLSPSGKEPA ASSPSSNNTA
ATTAAIVPGS QLMPSKSPST GTTEISSHES SHGTPSQTTA KNWELTASAS HQPPGVYPQGHS (SEQ ID
NO: 14)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQGHS (SEQ ID NO: 15)
MAPRRARGCR TLGLPALLLL LLLRPPATRG ITCPPPMSVE HADIWVKSYS LYSRERYICN SGFKRKAGTS
SLTECVLNKA TNVAHWTTPS LKCIRDPALV HQRPAPPSTV TTAGVTPQPE SLSPSGKEPA ASSPSSNNTA
ATTAAIVPGS QLMPSKSPST GTTEISSHES SHGTPSQTTA KNWELTASAS HQPPGVYPQGHSD (SEQ ID
NO: 16)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQGHSD (SEQ ID NO: 17)
MAPRRARGCR TLGLPALLLL LLLRPPATRG ITCPPPMSVE HADIWVKSYS LYSRERYICN SGFKRKAGTS
SLTECVLNKA TNVAHWTTPS LKCIRDPALV HQRPAPPSTV TTAGVTPQPE SLSPSGKEPA ASSPSSNNTA
ATTAAIVPGS QLMPSKSPST GTTEISSHES SHGTPSQTTA KNWELTASAS HQPPGVYPQGHSDT (SEQ
ID NO: 18)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQGHSDT (SEQ ID NO: 19)
MAPRRARGCR TLGLPALLLL LLLRPPATRG ITCPPPMSVE HADIWVKSYS LYSRERYICN SGFKRKAGTS
SLTECVLNKA TNVAHWTTPS LKCIRDPALV HQRPAPPSTV TTAGVTPQPE SLSPSGKEPA ASSPSSNNTA
ATTAAIVPGS QLMPSKSPST GTTEISSHES SHGTPSQTTA KNWELTASAS HQPPGVYPQGHSDTT (SEQ
ID NO: 20)
ITCPPPMSVE HADIWVKSYS LYSRERYICN SGFKRKAGTS SLTECVLNKA TNVAHWTTPS LKCIRDPALV
HQRPAPPSTV TTAGVTPQPE SLSPSGKEPA ASSPSSNNTA ATTAAIVPGS QLMPSKSPST GTTEISSHES
SHGTPSQTTA KNWELTASAS HQPPGVYPQGHSDTT (SEQ ID NO: 21)
nucleic acid sequence that encodes an IL-15Rα polypeptide:

| | |
|---|---|
| atggccccgc ggcgggcgcg cggctgccgg accctcggtc tcccggcgct gctactgctg | 60 |
| ctgctgctcc ggccgccggc gacgcggggc atcacgtgcc ctccccccat gtccgtggaa | 120 |
| cacgcagaca tctgggtcaa gagctacagc ttgtactcca gggagcggta catttgtaac | 180 |
| tctggtttca agcgtaaagc cggcacgtcc agcctgacgg agtgcgtgtt gaacaaggcc | 240 |
| acgaatgtcg cccactggac aacccccagt ctcaaatgca ttagagaccc tgccctggtt | 300 |
| caccaaaggc cagcgccacc ctccacagta acgacggcag gggtgacccc acagccagag | 360 |
| agcctctccc cttctggaaa agagcccgca gcttcatctc ccagctcaaa caacacagcg | 420 |
| gccacaacag cagctattgt cccgggctcc cagctgatgc cttcaaaatc accttccaca | 480 |
| ggaaccacag agataagcag tcatgagtcc tcccacggca ccccctctca gacaacagcc | 540 |
| aagaactggg aactcacagc atccgcctcc caccagccgc caggtgtgta tccacagggc | 600 |
| cacagcgaca ccactgtggc tatctccacg tccactgtcc tgctgtgtgg gctgagcgct | 660 |
| gtgtctctcc tggcatgcta cctcaagtca aggcaaactc ccccgctggc cagcgttgaa | 720 |
| atggaagcca tggaggctct gccggtgact tgggggacca gcagcagaga tgaagacttg | 780 |
| gaaaactgct ctcaccacct atga | 804 |
| (SEQ ID NO: 22) | |

nucleic acid sequence that encodes an IL-15Rα polypeptide:

| | |
|---|---|
| atggccccgc ggcgggcgcg cggctgccgg accctcggtc tcccggcgct gctactgctg | 60 |
| ctgctgctcc ggccgccggc gacgcggggc atcacgtgcc ctccccccat gtccgtggaa | 120 |
| cacgcagaca tctgggtcaa gagctacagc ttgtactcca gggagcggta catttgtaac | 180 |
| tctggtttca agcgtaaagc cggcacgtcc agcctgacgg agtgcgtgtt gaacaaggcc | 240 |
| acgaatgtcg cccactggac aacccccagt ctcaaatgca ttagagaccc tgccctggtt | 300 |
| caccaaaggc cagcgccacc ctccacagta acgacggcag gggtgacccc acagccagag | 360 |
| agcctctccc cttctggaaa agagcccgca gcttcatctc ccagctcaaa caacacagcg | 420 |
| gccacaacag cagctattgt cccgggctcc cagctgatgc cttcaaaatc accttccaca | 480 |
| ggaaccacag agataagcag tcatgagtcc tcccacggca ccccctctca gacaacagcc | 540 |
| aagaactggg aactcacagc atccgcctcc caccagccgc caggtgtgta tccacagggc | 615 |
| cacagcgaca ccact | 600 |
| (SEQ ID NO: 23) | |

nucleic acid sequence that encodes an IL-15 polypeptide:

| | |
|---|---|
| atgagaattt cgaaaccaca tttgagaagt atttccatcc agtgctactt gtgtttactt | 60 |
| ctaaacagtc attttctaac tgaagctggc attcatgtct tcattttggg ctgtttcagt | 120 |
| gcagggcttc ctaaaacaga agccaactgg gtgaatgtaa taagtgattt gaaaaaaatt | 180 |
| gaagatctta ttcaatctat gcatattgat gctactttat atacggaaag tgatgttcac | 240 |
| cccagttgca aagtaacagc aatgaagtgc tttctcttgg agttacaagt tatttcactt | 300 |
| gagtccggag atgcaagtat tcatgataca gtagaaaatc tgatcatcct agcaaacaac | 360 |
| agtttgtctt ctaatgggaa tgtaacagaa tctggatgca aagaatgtga ggaactggag | 420 |
| gaaaaaaata ttaaagaatt tttgcagagt tttgtacata ttgtccaaat gttcatcaac | 480 |
| acttcttga (SEQ ID NO: 24) | |

### REFERENCES

1. Tagaya, Y., et al., IL-15: a pleiotropic cytokine with diverse receptor/signaling pathways whose expression is controlled at multiple levels. Immunity, 1996. 4(4): p. 329-36.
2. Ma, A., D. L. Boone, and J. P. Lodolce, The pleiotropic functions of interleukin 15: not so interleukin 2-like after all. J Exp Med, 2000. 191(5): p. 753-6.
3. Bergamaschi, C., et al., Circulating IL-15 exists as heterodimeric complex with soluble IL-15Ralpha in human and mouse serum. Blood, 2012. 120(1): p. e1-8.
4. Bergamaschi, C., et al., Intracellular Interaction of Interleukin-15 with Its Receptor {alpha}during Production Leads to Mutual Stabilization and Increased Bioactivity. J Biol Chem, 2008. 283(7): p. 4189-99.
5. Bergamaschi, C., et al., Secretion and biological activity of short signal peptide IL-15 is chaperoned by IL-15 receptor alpha in vivo. J Immunol, 2009. 183(5): p. 3064-72.
6. Schluns, K. S., K. D. Klonowski, and L. Lefrancois, Transregulation of memory CD8 T-cell proliferation by IL-15Rαlpha+ bone marrow-derived cells. Blood, 2004. 103(3): p. 988-94.
7. Sandau, M. M., et al., Cutting edge: transpresentation of IL-15 by bone marrow-derived cells necessitates expression of IL-15 and IL-15R alpha by the same cells. J Immunol, 2004. 173(11): p. 6537-41.
8. Koka, R., et al., Interleukin (IL)-15R[alpha]-deficient natural killer cells survive in normal but not IL-15R[alpha]-deficient mice. J Exp Med, 2003. 197(8): p. 977-84.
9. Dubois, S., et al., IL-15Ralpha recycles and presents IL-15 In trans to neighboring cells. Immunity, 2002. 17(5): p. 537-47.
10. Burkett, P. R., et al., Coordinate expression and trans presentation of interleukin (IL)-15Rαlpha and IL-15 supports natural killer cell and memory CD8⁺T cell homeostasis. J Exp Med, 2004. 200(7): p. 825-34.
11. Duitman, E. H., et al., How a cytokine is chaperoned through the secretory pathway by complexing with its own receptor: lessons from interleukin-15 (IL-15)/IL-15 receptor alpha. Mol Cell Biol, 2008. 28(15): p. 4851-61.
12. Mathios, D., et al., Therapeutic administration of IL-15 superagonist complex ALT-803 leads to long-term survival and durable antitumor immune response in a murine glioblastoma model. Int J Cancer, 2016. 138(1): p. 187-94.
13. Xu, W., et al., Efficacy and mechanism-of-action of a novel superagonist interleukin-15: interleukin-15 receptor alphaSu/Fc fusion complex in syngeneic murine models of multiple myeloma. Cancer Res, 2013. 73(10): p. 3075-86.
14. Kobayashi, H., et al., Role of trans-cellular IL-15 presentation in the activation of NK cell-mediated killing, which leads to enhanced tumor immunosurveillance. Blood, 2005. 105(2): p. 721-7.
15. Stoklasek, T. A., K. S. Schluns, and L. Lefrancois, Combined IL-15/IL-15Rαlpha immunotherapy maximizes IL-15 activity in vivo. J Immunol, 2006. 177(9): p. 6072-80.
16. Romee, R., et al., First-in-human phase 1 clinical study of the IL-15 superagonist complex ALT-803 to treat relapse after transplantation. Blood, 2018. 131(23): p. 2515-2527.
17. Ng, S. S. M., et al., Heterodimeric IL15 Treatment Enhances Tumor Infiltration, Persistence, and Effector Functions of Adoptively Transferred Tumor-specific T Cells in the Absence of Lymphodepletion. Clin Cancer Res, 2017. 23(11): p. 2817-2830.
18. Kim, P. S., et al., IL-15 superagonist/lL-15RαlphaSushi-Fc fusion complex (IL-15SA/IL-15RαlphaSu-Fc; ALT-803) markedly enhances specific subpopulations of NK and memory CD8⁺T cells, and mediates potent anti-tumor activity against murine breast and colon carcinomas. Oncotarget, 2016. 7(13): p. 16130-45.
19. Epardaud, M., et al., Interleukin-15/interleukin-15R alpha complexes promote destruction of established tumors by reviving tumor-resident CD8⁺T cells. Cancer Res, 2008. 68(8): p. 2972-83.
20. Conlon, K. C., et al., Redistribution, hyperproliferation, activation of natural killer cells and CD8 T cells, and cytokine production during first-in-human clinical trial of recombinant human interleukin-15 in patients with cancer. J Clin Oncol, 2015. 33(1): p. 74-82.
21. Aliberti, J., et al., Essential role for ICSBP in the in vivo development of murine CD8alpha+dendritic cells. Blood, 2003. 101(1): p. 305-10.
22. Bergamaschi, C., et al., Heterodimeric IL-15 delays tumor growth and promotes intratumoral CTL and dendritic cell accumulation by a cytokine network involving XCL1, IFN-gamma, CXCL9 and CXCL10. J Immunother Cancer, 2020. 8(1).
23. Tenenbaum, A. and E. Z. Fisman, Fibrates are an essential part of modern anti-dyslipidemic arsenal: spotlight on atherogenic dyslipidemia and residual risk reduction. Cardiovasc Diabetol, 2012. 11: p. 125.
24. Yang, L. P. and G. M. Keating, Fenofibric acid: in combination therapy in the treatment of mixed dyslipidemia. Am J Cardiovasc Drugs, 2009. 9(6): p. 401-9.
25. Pettersen, J. C., et al., The PPARalpha agonists fenofibrate and CP-778875 cause increased beta-oxidation, leading to oxidative injury in skeletal and cardiac muscle in the rat. Toxicol Pathol, 2012. 40(3): p. 435-47.
26. Rakhshandehroo, M., et al., Comparative analysis of gene regulation by the transcription factor PPARalpha between mouse and human. PLoS One, 2009. 4(8): p. e6796.
27. Staels, B., et al., Fibrates downregulate apolipoprotein C-Ill expression independent of induction of peroxisomal acyl coenzyme A oxidase. A potential mechanism for the hypolipidemic action of fibrates. J Clin Invest, 1995. 95(2): p. 705-12.
28. Chen, Y., et al., Therapeutic effects of PPARalpha agonists on diabetic retinopathy in type 1 diabetes models. Diabetes, 2013. 62(1): p. 261-72.
29. Cheng, Y., et al., Up-regulation of Nrf2 is involved in FGF21-mediated fenofibrate protection against type 1 diabetic nephropathy. Free Radic Biol Med, 2016. 93: p. 94-109.
30. Noonan, J. E., et al., An update on the molecular actions of fenofibrate and its clinical effects on diabetic retinopathy and other microvascular end points in patients with diabetes. Diabetes, 2013. 62(12): p. 3968-75.
31. Dai, M., et al., Inhibition of JNK signalling mediates PPARalpha-dependent protection against intrahepatic cholestasis by fenofibrate. Br J Pharmacol, 2017. 174(18): p. 3000-3017.
32. Balakumar, P., A. Rohilla, and N. Mahadevan, Pleiotropic actions of fenofibrate on the heart. Pharmacol Res, 2011. 63(1): p. 8-12.
33. Tabernero, A., et al., Activation of the peroxisome proliferator-activated receptor alpha protects against myocardial ischaemic injury and improves endothelial vasodilatation. BMC Pharmacol, 2002. 2: p. 10.
34. Salehi, E., M. Khazaei, and B. Rashidi, Role of fenofibrate in restoring angiogenesis in diabetic and control hind limb ischemic rats. Gen Physiol Biophys, 2012. 31(3): p. 255-60.
35. Laurora, S., et al., Peroxisome proliferator-activated receptor ligands affect growth-related gene expression in human leukemic cells. J Pharmacol Exp Ther, 2003. 305(3): p. 932-42.
36. Scatena, R., et al., Bezafibrate as differentiating factor of human myeloid leukemia cells. Cell Death Differ, 1999. 6(8): p. 781-7.
37. Liu, H., et al., Growth inhibition and apoptosis in human Philadelphia chromosome-positive lymphoblastic leukemia cell lines by treatment with the dual PPARalpha/gamma ligand TZD18. Blood, 2006. 107(9): p. 3683-92.
38. Panigrahy, D., et al., PPARalpha agonist fenofibrate suppresses tumor growth through direct and indirect angiogenesis inhibition. Proc Natl Acad Sci USA, 2008. 105(3): p. 985-90.
39. Muzio, G., et al., PPARalpha and PP2A are involved in the proapoptotic effect of conjugated linoleic acid on human hepatoma cell line SK-HEP-1. Int J Cancer, 2007. 121(11): p. 2395-401.
40. Shigeto, T., et al., Peroxisome proliferator-activated receptor alpha and gamma ligands inhibit the growth of human ovarian cancer. Oncol Rep, 2007. 18(4): p. 833-40.
41. Huang, J., et al., The PPARalpha agonist fenofibrate suppresses B-cell lymphoma in mice by modulating lipid metabolism. Biochim Biophys Acta, 2013. 1831(10): p. 1555-65.
42. Wilk, A., et al., Molecular mechanisms of fenofibrate-induced metabolic catastrophe and glioblastoma cell death. Mol Cell Biol, 2015. 35(1): p. 182-98.
43. Lian, X., et al., Fenofibrate inhibits mTOR-p70S6K signaling and simultaneously induces cell death in human prostate cancer cells. Biochem Biophys Res Commun, 2018. 496(1): p. 70-75.
44. Zhao, H., et al., Fenofibrate down-regulates the expressions of androgen receptor (AR) and AR target genes and induces oxidative stress in the prostate cancer cell line LNCaP. Biochem Biophys Res Commun, 2013. 432(2): p. 320-5.
45. Li, T., et al., Fenofibrate induces apoptosis of triple-negative breast cancer cells via activation of NF-kappaB pathway. BMC Cancer, 2014. 14: p. 96.
46. Nguyen, C. H., et al., Fenofibrate inhibits tumour intravasation by several independent mechanisms in a 3-dimensional co-culture model. Int J Oncol, 2017. 50(5): p. 1879-1888.
47. Sun, J., et al., Fenofibrate potentiates chemosensitivity to human breast cancer cells by modulating apoptosis via AKT/NF-kappaB pathway. Onco Targets Ther, 2019. 12: p. 773-783.
48. Piwowarczyk, K., et al., Fenofibrate enhances barrier function of endothelial continuum within the metastatic niche of prostate cancer cells. Expert Opin Ther Targets, 2015. 19(2): p. 163-76.
49. Su, C., et al., Fenofibrate suppressed proliferation and migration of human neuroblastoma cells via oxidative stress dependent of TXNIP upregulation. Biochem Biophys Res Commun, 2015. 460(4): p. 983-8.
50. Wybieralska, E., et al., Fenofibrate attenuates contact-stimulated cell motility and gap junctional coupling in DU-145 human prostate cancer cell populations. Oncol Rep, 2011. 26(2): p. 447-53.
51. Yamasaki, D., et al., Fenofibrate suppresses growth of the human hepatocellular carcinoma cell via PPARalpha-independent mechanisms. Eur J Cell Biol, 2011. 90(8): p. 657-64.
52. Grabacka, M., et al., Peroxisome proliferator-activated receptor alpha activation decreases metastatic potential of melanoma cells in vitro via down-regulation of Akt. Clin Cancer Res, 2006. 12(10): p. 3028-36.
53. Grabacka, M., et al., Peroxisome proliferator-activated receptor alpha (PPARalpha) contributes to control of melanogenesis in B16 F10 melanoma cells. Arch Dermatol Res, 2017. 309(3): p. 141-157.
54. Tsai, S. C., et al., AMPK-dependent signaling modulates the suppression of invasion and migration by fenofibrate in CAL 27 oral cancer cells through NF-kappaB pathway. Environ Toxicol, 2016. 31(7): p. 866-76.
55. Liang, H., et al., Differential effects on lung cancer cell proliferation by agonists of glucocorticoid and PPARalpha receptors. Mol Carcinog, 2014. 53(9): p. 753-63.
56. Majeed, Y., et al., Potent and PPARalpha-independent anti-proliferative action of the hypolipidemic drug fenofibrate in VEGF-dependent angiosarcomas in vitro. Sci Rep, 2019. 9(1): p. 6316.
57. Hu, D., et al., Fenofibrate inhibited pancreatic cancer cells proliferation via activation of p53 mediated by upregulation of LncRNA MEG3. Biochem Biophys Res Commun, 2016. 471(2): p. 290-5.
58. Chowdhury, P. S., et al., PPAR-Induced Fatty Acid Oxidation in T Cells Increases the Number of Tumor-Reactive CD8(+) T Cells and Facilitates Anti-PD-1 Therapy. Cancer Immunol Res, 2018. 6(11): p. 1375-1387.
59. Zhang, Y., et al., Enhancing CD8(+) T Cell Fatty Acid Catabolism within a Metabolically Challenging Tumor Microenvironment Increases the Efficacy of Melanoma Immunotherapy. Cancer Cell, 2017. 32(3): p. 377-391 e9.
60. Manzo, T., et al., Accumulation of long-chain fatty acids in the tumor microenvironment drives dysfunction in intrapancreatic CD8+ T cells. J Exp Med, 2020. 217(8).
61. Kazi, J. U. and L. Ronnstrand, FMS-like Tyrosine Kinase 3/FLT3: From Basic Science to Clinical Implications. Physiol Rev, 2019. 99(3): p. 1433-1466.
62. Sitnicka, E., et al., Key role of flt3 ligand in regulation of the common lymphoid progenitor but not in maintenance of the hematopoietic stem cell pool. Immunity, 2002. 17(4): p. 463-72.
63. Daver, N., et al., Targeting FLT3 mutations in AML: review of current knowledge and evidence. Leukemia, 2019. 33(2): p. 299-312.
64. Chao, Q., et al., Identification of N-(5-tert-butyl-isoxazol-3-yl)-N′-{4-[7-(2-morpholin-4-yl-ethoxy)imidazo[2,1-b][1,3]benzothiazol-2-yl]phenyl}urea dihydrochloride (AC220), a uniquely potent, selective, and efficacious FMS-like tyrosine kinase-3 (FLT3) inhibitor. J Med Chem, 2009. 52(23): p. 7808-16.
65. Zarrinkar, P. P., et al., AC220 is a uniquely potent and selective inhibitor of FLT3 for the treatment of acute myeloid leukemia (AML). Blood, 2009. 114(14): p. 2984-92.
66. Usuki, K., et al., Safety and pharmacokinetics of quizartinib in Japanese patients with relapsed or refractory acute myeloid leukemia in a phase 1 study. Int J Hematol, 2019. 110(6): p. 654-664.
67. Cortes, J. E., et al., Phase I study of quizartinib administered daily to patients with relapsed or refractory acute myeloid leukemia irrespective of FMS-like tyrosine kinase 3-internal tandem duplication status. J Clin Oncol, 2013. 31(29): p. 3681-7.
68. Sandmaier, B. M., et al., Results of a phase 1 study of quizartinib as maintenance therapy in subjects with acute myeloid leukemia in remission following allogeneic hematopoietic stem cell transplant. Am J Hematol, 2018. 93(2): p. 222-231.
69. Altman, J. K., et al., Phase 1 study of quizartinib in combination with induction and consolidation chemotherapy in patients with newly diagnosed acute myeloid leukemia. Am J Hematol, 2018. 93(2): p. 213-221.
70. Cortes, J. E., et al., Phase 2b study of 2 dosing regimens of quizartinib monotherapy in FLT3-ITD-mutated, relapsed or refractory AML. Blood, 2018. 132(6): p. 598-607.
71. Cortes, J., et al., Quizartinib, an FLT3 inhibitor, as monotherapy in patients with relapsed or refractory acute myeloid leukaemia: an open-label, multicentre, single-arm, phase 2 trial. Lancet Oncol, 2018. 19(7): p. 889-903.
72. Cortes, J. E., et al., Quizartinib versus salvage chemotherapy in relapsed or refractory FLT3-ITD acute myeloid leukaemia (QuANTUM-R): a multicentre, randomised, controlled, open-label, phase 3 trial. Lancet Oncol, 2019. 20(7): p. 984-997.
73. Ginhoux, F., et al., The origin and development of nonlymphoid tissue CD103+ DCs. J Exp Med, 2009. 206(13): p. 3115-30.
74. Karsunky, H., et al., Flt3 ligand regulates dendritic cell development from Flt3+ lymphoid and myeloid-committed progenitors to Flt3+ dendritic cells in vivo. J Exp Med, 2003. 198(2): p. 305-13.
75. Sichien, D., et al., Development of conventional dendritic cells: from common bone marrow progenitors to multiple subsets in peripheral tissues. Mucosal Immunol, 2017. 10(4): p. 831-844.
76. Audiger, C. and S. Lesage, FLT3 Ligand Is Dispensable for the Final Stage of Type 1 Conventional Dendritic Cell Differentiation. J Immunol, 2020. 205(8): p. 2117-2127.
77. Broz, M. L., et al., Dissecting the tumor myeloid compartment reveals rare activating antigen-presenting cells critical for T cell immunity. Cancer Cell, 2014. 26(5): p. 638-52.
78. Spranger, S., et al., Tumor-Residing Batf3 Dendritic Cells Are Required for Effector T Cell Trafficking and Adoptive T Cell Therapy. Cancer Cell, 2017. 31(5): p. 711-723 e4.
79. Barry, K. C., et al., A natural killer-dendritic cell axis defines checkpoint therapy-responsive tumor microenvironments. Nat Med, 2018. 24(8): p. 1178-1191.
80. Salmon, H., et al., Expansion and Activation of CD103(+) Dendritic Cell Progenitors at the Tumor Site Enhances Tumor Responses to Therapeutic PD-L1 and BRAF Inhibition. Immunity, 2016. 44(4): p. 924-38.
81. Esche, C., et al., FLT3 ligand administration inhibits tumor growth in murine melanoma and lymphoma. Cancer Res, 1998. 58(3): p. 380-3.
82. Lai, J., et al., Adoptive cellular therapy with T cells expressing the dendritic cell growth factor FIt3L drives epitope spreading and antitumor immunity. Nat Immunol, 2020. 21(8): p. 914-926.
83. Hammerich, L., et al., Systemic clinical tumor regressions and potentiation of PD1 blockade with in situ vaccination. Nat Med, 2019. 25(5): p. 814-824.
84. Sanchez-Paulete, A. R., et al., Cancer Immunotherapy with Immunomodulatory Anti-CD137 and Anti-PD-1 Monoclonal Antibodies Requires BATF3-Dependent Dendritic Cells. Cancer Discov, 2016. 6(1): p. 71-9.
85. Brown, C. C., et al., Transcriptional Basis of Mouse and Human Dendritic Cell Heterogeneity. Cell, 2019. 179(4): p. 846-863 e24.
86. Blondel D. Vincent, G. J.-L., Lambiotte Renaud, Lefebvre Etienne, Fast unfolding of communities in large networks. Journal of Statistical Mechanics, 2008.
87. Tzelepis, F., et al., Annexin1 regulates DC efferocytosis and cross-presentation during Mycobacterium tuberculosis infection. J Clin Invest, 2015. 125(2): p. 752-68.
88. Toyonaga, K., et al., C-Type Lectin Receptor DCAR Recognizes Mycobacterial Phosphatidyl-Inositol Mannosides to Promote a Th1 Response during Infection. Immunity, 2016. 45(6): p. 1245-1257.
89. Qu, C., et al., Monocyte-derived dendritic cells: targets as potent antigen-presenting cells for the design of vaccines against infectious diseases. Int J Infect Dis, 2014. 19: p. 1-5.
90. Napoletano, C., et al., Tumor-associated Tn-MUC1 glycoform is internalized through the macrophage galactose-type C-type lectin and delivered to the HLA class I and II compartments in dendritic cells. Cancer Res, 2007. 67(17): p. 8358-67.
91. Kis-Toth, K., et al., Monocyte-derived dendritic cell subpopulations use different types of matrix metalloproteinases inhibited by GM6001. Immunobiology, 2013. 218(11): p. 1361-9.
92. Bonnardel, J., et al., Innate and adaptive immune functions of peyer's patch monocyte-derived cells. Cell Rep, 2015. 11(5): p. 770-84.
93. Le Naour, F., et al., Profiling changes in gene expression during differentiation and maturation of monocyte-derived dendritic cells using both oligonucleotide microarrays and proteomics. J Biol Chem, 2001. 276(21): p. 17920-31.
94. Santambrogio, L., S. J. Berendam, and V. H. Engelhard, The Antigen Processing and Presentation Machinery in Lymphatic Endothelial Cells. Front Immunol, 2019. 10: p. 1033.
95. Theisen, D. J., et al., WDFY4 is required for cross-presentation in response to viral and tumor antigens. Science, 2018. 362(6415): p. 694-699.

1-82. (canceled)

83. A composition, comprising a fusion protein, comprising:
(a) an IL-15 or a derivative thereof or an IL-15/IL-15 receptor alpha (IL-15Rα) complex or a derivative thereof; and
(b) an IL-12 or a derivative thereof.

84. The composition of claim 83, further comprising one or more active agents.

85. The composition of claim 84, wherein the one or more active agents comprises a chemotherapeutic agent.

86-89. (canceled)

90. The composition of claim 85, wherein the chemotherapeutic agent is adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

91. The composition of claim 83, wherein the IL-15 or the derivative thereof or the IL-15/IL-15 receptor alpha (IL-15Rα) complex or the derivative thereof comprises any of the sequences as disclosed in Table 1 or Table 4.

92. The composition of claim 83, wherein the IL-15 or the derivative thereof or the IL-15/IL15Rα complex or the derivative thereof dose in the composition is about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

93-94. (canceled)

95. A method for treating cancer in a subject, comprising administering to the subject the composition of claim 83.

96. The method of claim 95, wherein the method further comprises administering to the subject one or more active agents.

97. The method of claim 96, wherein the one or more active agents comprises a chemotherapeutic agent.

98-101. (canceled)

102. The method of claim 95, wherein the the chemotherapeutic agent is adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, plerixafor, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combinations thereof.

103. The method of claim 95, wherein the IL-15 or the derivative thereof or the IL-15/IL-15 receptor alpha (IL-15Rα) complex or the derivative thereof comprises any of the sequences as disclosed in Table 1 or Table 4.

104. The method of claim 95, wherein the IL-15 or the derivative thereof or the IL-15/IL15Rα complex or the derivative thereof is administered at a dose of about 0.1 μg/kg, about 0.25 μg/kg, about 0.5 μg/kg, about 0.75 μg/kg, about 1 μg/kg, about 1.5 μg/kg, about 2 μg/kg, about 2.5 μg/kg, about 3 μg/kg, about 3.5 μg/kg, about 4 μg/kg, about 4.5 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, or higher.

105-106. (canceled)

107. The method of claim 95, wherein the IL-15 or the derivative thereof or the IL-15/IL15Rα complex or the derivative thereof is administered via locoregional administration to the cancer.

108. The method of claim 95, wherein the IL-15 or the derivative thereof or the IL15-/IL15Rα complex or the derivative thereof is administered subcutaneously, intramuscularly, intravenously, by peritumoral injection, or by intratumoral injection.

109. The method of claim 95, wherein the cancer is selected from breast cancer, triple negative breast cancer, pancreatic cancer, glioblastoma, colon cancer, prostate cancer, angiosarcoma, melanoma, renal clear cell carcinoma, astrocytoma, atypical carcinoid lung cancer, basal cell carcinoma, B-cell acute lymphocytic leukemia, B-cell acute lymphoblastic leukemia/lymphoma, bladder cancer, brain cancer, bronchial cancer, Burkitt's lymphoma, cancer of the bile duct, cancer of unknown primary origin, cervical cancer, chronic myeloproliferative disorder, diffuse large cell lymphoma, endometrial cancer, ependymoma, esophageal cancer, gastric cancer, glioma, head and neck cancer, hemangiopericytoma, hepatocellular carcinoma, Hodgkin's lymphoma, Kaposi's sarcoma, kidney cancer, large cell neuroendocrine carcinoma, large granular lymphocytic leukemia, leukemia, liver cancer, lung cancer, lymphoma, medulloblastoma, myelodysplastic syndrome, nasopharyngeal cancer, neuroblastoma, non-Hodgkin's lymphoma, esophageal squamous cell carcinoma, osteosarcoma, ovarian cancer, retinoblastoma, rhabdomyosarcoma, sarcoma, small cell lung carcinoma, testicular cancer, urachal cancer, uterine cancer, and vaginal cancer.

110. A fusion protein for the treatment of cancer in a subject, wherein the fusion protein comprises:
(a) an IL-15 or a derivative thereof or an IL-15/IL-15 receptor alpha (IL-15Rα) complex or a derivative thereof; and
(b) an IL-12 or a derivative thereof.

111-152. (canceled)


SEQUENCE LISTING (54 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 205 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 5 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 6 - protein, 149 aa, Homo sapiens
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 267 aa, Homo sapiens; identical to SEQ ID NO: 3

SEQ ID NO: 8 - protein, 205 aa, Homo sapiens; identical to SEQ ID NO: 4

SEQ ID NO: 9 - protein, 131 aa, Homo sapiens
MWLQSLLLLGTVACSISNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 175 aa, Homo sapiens; identical to SEQ ID NO: 5

SEQ ID NO: 11 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 12 - protein, 201 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGH

SEQ ID NO: 13 - protein, 171 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGH

SEQ ID NO: 14 - protein, 202 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHS

SEQ ID NO: 15 - protein, 172 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHS

SEQ ID NO: 16 - protein, 203 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSD

SEQ ID NO: 17 - protein, 173 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSD

SEQ ID NO: 18 - protein, 204 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDT

SEQ ID NO: 19 - protein, 174 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDT

SEQ ID NO: 20 - protein, 205 aa, Homo sapiens; identical to SEQ ID NO: 4

SEQ ID NO: 21 - protein, 175 aa, Homo sapiens; identical to SEQ ID NO: 5

SEQ ID NO: 22 - DNA, 804 bp, Homo sapiens
  source 1..804: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 23 - DNA, 615 bp, Homo sapiens
  source 1..615: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccact

SEQ ID NO: 24 - DNA, 489 bp, Homo sapiens
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 25 - protein, 4 aa, synthetic construct
PQGH

SEQ ID NO: 26 - protein, 5 aa, synthetic construct
PQGHS

SEQ ID NO: 27 - protein, 6 aa, synthetic construct
PQGHSD

SEQ ID NO: 28 - protein, 7 aa, synthetic construct
PQGHSDT

SEQ ID NO: 29 - protein, 8 aa, synthetic construct
PQGHSDTT

SEQ ID NO: 30 - protein, 19 aa, synthetic construct
NWELTASASHQPPGVYPQG

SEQ ID NO: 31 - protein, 17 aa, synthetic construct
ITCPPPMSVEHADIWVK

SEQ ID NO: 32 - protein, 31 aa, synthetic construct
ITCPPPMSVEHADIWVKSYSLYSRERYICNS

SEQ ID NO: 33 - protein, 4 aa, synthetic construct
GGGS

SEQ ID NO: 34 - protein, 5 aa, synthetic construct
GGGGS

SEQ ID NO: 35 - protein, 8 aa, synthetic construct
GGGSGGGS

SEQ ID NO: 36 - protein, 12 aa, synthetic construct
GGGSGGGSGGGS

SEQ ID NO: 37 - protein, 16 aa, synthetic construct
GGGSGGGSGGGSGGGS

SEQ ID NO: 38 - protein, 10 aa, synthetic construct
GGGGSGGGGS

SEQ ID NO: 39 - protein, 15 aa, synthetic construct
GGGGSGGGGSGGGGS

SEQ ID NO: 40 - protein, 20 aa, synthetic construct
GGGGSGGGGSGGGGSGGGGS

SEQ ID NO: 41 - protein, 4 aa, synthetic construct
RVRR

SEQ ID NO: 42 - protein, 4 aa, synthetic construct
GAGA

SEQ ID NO: 43 - protein, 4 aa, synthetic construct
AGAG

SEQ ID NO: 44 - protein, 4 aa, synthetic construct
KGKG

SEQ ID NO: 45-46 - skipped (2 entries)

SEQ ID NO: 47 - protein, 444 aa, synthetic construct
MWLQNLLFLGIVVYSLSNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 48 - DNA, 1338 bp, synthetic construct
  source 1..1338: mol_type = other DNA
atgtggctccagaacctgctattcctggggatcgtggtctacagcctctcgaactggatcgatgtgcgctacgacctggagaagatcgagtccctcatccagtcgatccacatcgacaccacgctgtacacggactcggacttccacccgtcgtgcaaggtcacggcgatgaactgcttcctcctggagctgcaagtcatcctgcacgagtactcgaacatgaccctcaacgagacggtgcgcaacgtgctgtacctggcgaactcgaccctgtcgtcgaacaagaacgtcgccgagtcgggctgcaaggagtgcgaggagctggaggagaagaccttcacggagttcctgcagtcgttcatccgcatcgtccagatgttcatcaacacgtcgatgtgggagctggagaaggacgtctacgtcgtggaggtggactggacgcccgacgcccccggggagacggtgaacctcacctgcgacacgccggaggaggacgacatcacctggaccagcgaccagcggcacggcgtcatcggcagcggcaagaccctgacgatcaccgtcaaggagttcctcgacgccggccagtacacctgccacaagggcggcgagacgctgagccacagccacctgctgctccacaagaaggagaacggcatctggtccaccgagatcctcaagaacttcaagaacaagaccttcctgaagtgcgaggcgcccaactactccggccggttcacgtgctcctggctggtgcagcgcaacatggacctcaagttcaacatcaagtcgtccagcagttcccccgactcccgggcggtgacctgcggcatggcgtccctgtcggcggagaaggtcacgctggaccagagggactacgagaagtactcggtgtcctgccaggaggacgtcacctgccccacggccgaggagaccctgcccatcgagctggcgctggaggcccggcagcagaacaagtacgagaactacagcaccagcttcttcatccgggacatcatcaagcccgacccgcccaagaacctgcagatgaagcccctgaagaacagccaggtggaggtcagctgggagtacccggactcctggagcacgccccactcctacttcagcctgaagttcttcgtcagaatccagcgcaagaaagagaagatgaaggagacggaggaggggtgcaaccagaagggcgcgttcctcgtcgagaagacctccaccgaggtccagtgcaagggcgggaacgtctgcgtgcaggctcaggaccgctactacaactccagctgcagcaagtgggcctgcgtcccctgcagggtccggtcctagtaa

SEQ ID NO: 49 - protein, 422 aa, synthetic construct
MWLQNLLFLGIVVYSLSNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSTVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 50 - protein, 459 aa, synthetic construct
MWLQSLLLLGTVACSISNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 51 - DNA, 1383 bp, synthetic construct
  source 1..1383: mol_type = other DNA
atgtggctccagagcctgctactcctggggacggtggcctgcagcatctcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgatgtgccaccagcagctcgtgatcagctggttcagcctcgtcttcctcgcgtcgccgctggtcgcgatctgggagctgaagaaggacgtctacgtggtggagctggactggtatcccgacgcgcccggggagatggtcgtgctgacgtgcgacacgccggaggaggacgggatcacgtggacgctcgaccagtcctcggaggtgctcgggtccgggaagacgctcacgatccaggtcaaggagttcggcgacgccgggcagtacacgtgccacaagggcggcgaggtcctctcgcactccctgctcctcctgcacaagaaggaggacggcatctggagcacggacatcctgaaggaccagaaggagccgaagaacaagacgttcctgcgctgcgaggcgaagaactactccgggcgcttcacgtgctggtggctcaccacgatctcgacggacctgacgttctcggtgaagtcgtcgcggggctcctcggacccgcaaggcgtcacctgcggcgcagcgacgctgtcggcggagcgcgtgcggggcgacaacaaggagtacgagtactcggtcgagtgccaggaggactcggcgtgcccggcagcggaggagtcgctgccgatcgaggtgatggtcgacgcggtccacaagctgaagtacgagaactacacgtcgtccttcttcatccgcgacatcatcaagcccgacccgccgaagaacctccagctcaagccgctcaagaactcgcgccaggtcgaggtgtcgtgggagtaccccgacacgtggtcgacgccccactcctacttctcgctcaccttctgcgtccaagtgcagggcaagtcgaagcgggagaagaaggaccgcgtgttcaccgacaagaccagcgcgaccgtgatctgccggaagaacgcgtcgatctcggtgcgggcgcaggaccggtactactcgtcgtcgtggtcggagtgggcctcggtgccgtgcagctagtaa

SEQ ID NO: 52 - protein, 437 aa, synthetic construct
MWLQSLLLLGTVACSISNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 53 - DNA, 5363 bp, synthetic construct
  source 1..5363: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaggaattctctagaaagaaatgtggctccagaacctgctattcctggggatcgtggtctacagcctctcgaactggatcgatgtgcgctacgacctggagaagatcgagtccctcatccagtcgatccacatcgacaccacgctgtacacggactcggacttccacccgtcgtgcaaggtcacggcgatgaactgcttcctcctggagctgcaagtcatcctgcacgagtactcgaacatgaccctcaacgagacggtgcgcaacgtgctgtacctggcgaactcgaccctgtcgtcgaacaagaacgtcgccgagtcgggctgcaaggagtgcgaggagctggaggagaagaccttcacggagttcctgcagtcgttcatccgcatcgtccagatgttcatcaacacgtcgatgtgggagctggagaaggacgtctacgtcgtggaggtggactggacgcccgacgcccccggggagacggtgaacctcacctgcgacacgccggaggaggacgacatcacctggaccagcgaccagcggcacggcgtcatcggcagcggcaagaccctgacgatcaccgtcaaggagttcctcgacgccggccagtacacctgccacaagggcggcgagacgctgagccacagccacctgctgctccacaagaaggagaacggcatctggtccaccgagatcctcaagaacttcaagaacaagaccttcctgaagtgcgaggcgcccaactactccggccggttcacgtgctcctggctggtgcagcgcaacatggacctcaagttcaacatcaagtcgtccagcagttcccccgactcccgggcggtgacctgcggcatggcgtccctgtcggcggagaaggtcacgctggaccagagggactacgagaagtactcggtgtcctgccaggaggacgtcacctgccccacggccgaggagaccctgcccatcgagctggcgctggaggcccggcagcagaacaagtacgagaactacagcaccagcttcttcatccgggacatcatcaagcccgacccgcccaagaacctgcagatgaagcccctgaagaacagccaggtggaggtcagctgggagtacccggactcctggagcacgccccactcctacttcagcctgaagttcttcgtcagaatccagcgcaagaaagagaagatgaaggagacggaggaggggtgcaaccagaagggcgcgttcctcgtcgagaagacctccaccgaggtccagtgcaagggcgggaacgtctgcgtgcaggctcaggaccgctactacaactccagctgcagcaagtgggcctgcgtcccctgcagggtccggtcctagtaaagatctgatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 54 - DNA, 5375 bp, synthetic construct
  source 1..5375: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaggaattctctagaaagaaatgtggctccagaacctgctattcctggggatcgtggtctacagcctctcgaactggatcgatgtgcgctacgacctggagaagatcgagtccctcatccagtcgatccacatcgacaccacgctgtacacggactcggacttccacccgtcgtgcaaggtcacggcgatgaactgcttcctcctggagctgcaagtcatcctgcacgagtactcgaacatgaccctcaacgagacggtgcgcaacgtgctgtacctggcgaactcgaccctgtcgtcgaacaagaacgtcgccgagtcgggctgcaaggagtgcgaggagctggaggagaagaccttcacggagttcctgcagtcgttcatccgcatcgtccagatgttcatcaacacgtcgggagcaggtgctatgtgggagctggagaaggacgtctacgtcgtggaggtggactggacgcccgacgcccccggggagacggtgaacctcacctgcgacacgccggaggaggacgacatcacctggaccagcgaccagcggcacggcgtcatcggcagcggcaagaccctgacgatcaccgtcaaggagttcctcgacgccggccagtacacctgccacaagggcggcgagacgctgagccacagccacctgctgctccacaagaaggagaacggcatctggtccaccgagatcctcaagaacttcaagaacaagaccttcctgaagtgcgaggcgcccaactactccggccggttcacgtgctcctggctggtgcagcgcaacatggacctcaagttcaacatcaagtcgtccagcagttcccccgactcccgggcggtgacctgcggcatggcgtccctgtcggcggagaaggtcacgctggaccagagggactacgagaagtactcggtgtcctgccaggaggacgtcacctgccccacggccgaggagaccctgcccatcgagctggcgctggaggcccggcagcagaacaagtacgagaactacagcaccagcttcttcatccgggacatcatcaagcccgacccgcccaagaacctgcagatgaagcccctgaagaacagccaggtggaggtcagctgggagtacccggactcctggagcacgccccactcctacttcagcctgaagttcttcgtcagaatccagcgcaagaaagagaagatgaaggagacggaggaggggtgcaaccagaagggcgcgttcctcgtcgagaagacctccaccgaggtccagtgcaagggcgggaacgtctgcgtgcaggctcaggaccgctactacaactccagctgcagcaagtgggcctgcgtcccctgcagggtccggtcctagtaaagatctgatatcggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg